(19) United States
(12) Patent Application Publication
(10) Pub. No.: US 2026/0209730 A1
(43) Pub. Date: Jul. 23, 2026

(54) COMPOSITIONS AND METHODS OF GENOME EDITING

(71) Applicant: EPIGENIC THERAPEUTICS PTE. LTD., Robinson Square (SG)

(72) Inventors: Changyang ZHOU, Shanghai (CN); Yidi SUN, Shanghai (CN); Shaoshuai MAO, Shanghai (CN); Wenbo PENG, Shanghai (CN)

(73) Assignee: EPIGENIC THERAPEUTICS PTE. LTD., Robinson Square (SG)

(21) Appl. No.: 19/434,484

(22) Filed: Dec. 29, 2025

Related U.S. Application Data
(63) Continuation of application No. 18/823,429, filed on Sep. 3, 2024, now abandoned.
Application No. 18/823,429 is a continuation of application No. PCT/CN2023/079543, filed on Mar. 3, 2023.

(30) Foreign Application Priority Data
Mar. 4, 2022 (WO) PCT/CN2022/079232

(51) Int. Cl. C12N 9/22; C12N 9/10; C12N 15/11; C12N 15/90

(52) U.S. Cl. CPC C12N 9/22; C12N 9/1007; C12N 15/11; C12N 15/907; C12Y 201/01037; C07K 2319/00; C12N 2310/20

(57) ABSTRACT

This disclosure provides CRISPR/Cas9 based fusion molecules and guide RNAs for use in in vivo targeted reduction or elimination of VEGFA gene products. This disclosure also relates to formulations, methods of production and methods of use thereof.

### CROSS-REFERENCE TO RELATED APPLICATIONS

This application is a Continuation of U.S. application Ser. No. 18/823,429 filed on Sep. 3, 2024, which is a Continuation and claims priority under 35 U.S.C. § 111 to Patent Cooperation Treaty application PCT/CN2023/079543, filed Mar. 3, 2023, which claims the benefit of Patent Cooperation Treaty application PCT/CN2022/079232, filed Mar. 4, 2022. Priority is claimed to both of these applications and the disclosures of these prior applications are considered part of the disclosure of this application and to the extent allowed the entire contents of the aforementioned applications are incorporated herein.

### REFERENCE TO SEQUENCE LISTING

The contents of the electronic Sequence Listing (19434484seglist.xml); 718,280 bytes in size, was created on Aug. 26, 2024 is herein incorporated by reference in its entirety.

### FIELD OF THE DISCLOSURE

The present disclosure relates generally to the fields of molecular biology, immunology, and medicine. More particularly, it relates to CRISPR/Cas9 based fusion molecules for use in targeted reduction or elimination of gene products in vivo and methods of use thereof.

### BACKGROUND

Engineered DNA-binding proteins that can be customized to target any gene in mammalian cells have enabled rapid advances in biomedical research and are a promising platform for gene therapies. The RNA-guided CRISPR-Cas9 system has emerged as a promising platform for programmable targeted gene regulation. Fusion of catalytically inactive, “dead” Cas9 (dCas9) to the Kruppel-associated box (KRAB) domain generates a synthetic repressor capable of highly specific and potent modulation or silencing of target genes in cell culture experiments.

However, persistent modulation and silencing of endogenous genes using synthetic dCas9-KRAB fusion proteins have presented challenges for use in in vivo therapies. Synthetic repressors exceed size packaging limits of viral vector delivery methods. Safety, toxicity, immunogenicity, and off target effects are other challenges that limit the use of synthetic repressors in vivo. There is a need in the art for alternative approaches for generating genetically engineered synthetic gene repressors and in vivo delivery of the synthetic gene repressors for use as therapeutics. The present disclosure addresses this unmet need in the art.

### SUMMARY

The disclosure provides a construct of Formula I: 5′-(Aₘ₁-Bₘ₂)ₙ₁-CasN-(Aₘ₃-Bₘ₄)ₙ₂-CasC-Tₚ-E-3′ (I), wherein: one of A and B is a polynucleotide encoding DNMT3A or a portion thereof, and the other of A and B is a polynucleotide encoding DNMT3L or a portion thereof, CasN is a polynucleotide encoding a N-terminal portion of dCas9; CasC is a polynucleotide encoding a C-terminal portion of dCas9; E is 5′-(Aₘ₅-Bₘ₆)ₙ₃-Kᵣ-D_{q}-3′ or 5′-Kᵣ-D_{q}-(Aₘ₅-Bₘ₆)ₙ₃-3′; K is a polynucleotide encoding KRAB or a portion thereof, D is a polynucleotide encoding a modulator of gene expression; T comprises a polynucleotide encoding (i) an epitope capable of binding to an antibody or an antigen binding fragment thereof or (ii) a polypeptide sequence capable of binding to a nucleic acid structural element; m1, m2, m3, m4, m5, and m6 are each independently an integer selected from 0 to 3; n1, n2, and n3 are each independently an integer selected from 0 to 2; p is an integer selected from 0 to 20; q is an integer selected from 0 to 5; r is an integer selected from 0 to 5; and wherein when p is 0, at least one of m1, m2, m3, and m4 is not 0, at least one of n1 and n2 is not 0, and at least one of q and r is not 0.

In some embodiments, the construct comprises Formula II: 5′-CasN-(Aₘ₃-Bₘ₄)ₙ₂-CasC-Kᵣ-D_{q}-3′ (II), wherein: n2 is an integer selected from 1 and 2; r is an integer selected from 1 to 5; q is an integer selected from 0 to 5; and at least one of m3 and m4 is not 0. In some embodiments, the construct comprises Formula IIa: 5′-CasN-(A-B)-CasC-Kᵣ-D_{q}-3′ (IIa).

In some embodiments, the construct comprises Formula III: 5′-(Aₘ₁-Bₘ₂)ₙ₁-CasN-(Aₘ₃-Bₘ₄)ₙ₂-CasC-Tₚ-E-3′ (III), wherein p is an integer selected from 1 to 20. In some embodiments, the construct comprises of Formula IIIa: 5′-CasN-CasC-Tₚ-3′ (IIIa).

In some embodiments, the construct comprises Formula IIIb: 5′-(Aₘ₁-Bₘ₂)-CasN-CasC-Tₚ-E-3′ (IIIb), wherein at least one of m1 and m2 is not 0. In some embodiments, the construct comprises Formula IIIb-1: 5′-(A-B)-CasN-CasC-Tₚ-Kᵣ-D_{q}-3′ (IIIb-1), wherein: r is an integer selected from 1 to 5; and q is an integer selected from 0 to 5.

In some embodiments, the construct comprises Formula IIIc: 5′-CasN-CasC-Tₚ-E-3′(IIIc), wherein: n3 is an integer selected from 1 and 2; r is an integer selected from 1 to 5; and q is an integer selected from 0 to 5; and at least one of m5 and m6 is not 0. In some embodiments, the construct comprises Formula IIIc-1: 5′-CasN-CasC-Tₚ-(Aₘ₅-Bₘ₆)ₙ₃-Kᵣ-D_{q}-3′ (IIIc-1). In some embodiments, the construct comprises Formula IIIc-2: 5′-CasN-CasC-Tₚ-Kᵣ-D_{q}-(Aₘ₅-Bₘ₆)ₙ₃-3′ (IIIc-2).

In some embodiments, the T comprises (a) a polynucleotide encoding an epitope capable of binding to an antibody or antigen binding fragment thereof, and further comprises (b) a polynucleotide encoding a self cleaving peptide at the 3′ end of the polynucleotide in (a). In some embodiments, the 3′ end of the nucleotide encoding the self cleaving peptide further comprises polynucleotide encoding (c) an antibody or antigen binding fragment thereof that is capable of binding to the epitope.

In some embodiments, the T comprises (a) a polynucleotide encoding the polypeptide sequence capable of binding to a nucleotide sequence element, and further comprises a polynucleotide encoding a self cleaving peptide at the 3′ end of the polynucleotide in (a).

In some embodiments, the self cleaving peptide is selected from the group consisting of a T2A, a P2A, a E2A and a F2A self cleaving peptide. In some embodiments, the self cleaving peptide is T2A. In some embodiments, the T2A comprises the amino acid sequence of SEQ ID NO: 99.

In some embodiments, the DNMT3A comprises the amino acid sequence of SEQ ID NO: 69. In some embodiments, the polynucleotide encoding the DNMT3A comprises the nucleic acid sequence of SEQ ID NO: 83.

In some embodiments, the DNMT3L comprises a Homo sapiens DNMT3L, a Mus musculus DNMT3L, a Mus caroli DNMT3L, a Mus pahari DNMT3L, a Rattus norvegicus DNMT3L, a Rattus rattus DNMT3L, a Arvicanthis niloticus DNMT3L, a Grammomys surdaster DNMT3L or a Mastomys coucha DNTM3L. In some embodiments, the DNMT3L comprises the amino acid sequence of any one of SEQ ID NOs: 74-82. In some embodiments, the polynucleotide encoding the DNMT3L comprises the nucleic acid sequence of any one of SEQ ID NO: 84-92.

In some embodiments, the KRAB comprises the amino acid sequence of SEQ ID NO: 51, 53 or 230-241. In some embodiments, the polynucleotide encoding the KRAB comprises the nucleic acid sequence of SEQ ID NO: 52, 54, 206, 208, 210, 212, 214, 216, 218, 220, 222, 224, 226 or 228.

In some embodiments, the modulator of gene expression comprises a Kruppel-associated suppression box (KRAB), a Enhancer of zete homolog 2 (EZH2), a G9A, a Lysine-Specific histone Demethylase 1A (LSD1), a Heterochromatin Protein 1 (HP1), a Friend of GATA protein 1 (FOG1), a Histone Deacetylase (HDAC3) and/or a DOT1L. In some embodiments, the modulator of gene expression comprises the amino acid sequence of SEQ ID NOs: 51, 53, 55, 57, 59, 61, 63, 65, or 67. In some embodiments, the polynucleotide encoding the modulator of gene expression comprises the nucleic acid sequence of SEQ ID NOs: 52, 54, 56, 58, 60, 62, 64, 66, or 68.

In some embodiments, the dCas9 comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum (e.g., strain B510) dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis (e.g., strain DSM 16511) dCas9, a Campylobacter lari (e.g., strain CF89-12) dCas9, a Streptococcus thermophilus (e.g., strain LMD-9) dCas9. In some embodiments, the dCas9 comprises the amino acid sequence of any one of SEQ ID NOs: 106-122. In some embodiments, the polynucleotide encoding the dCas9 comprises the nucleic acid sequence of SEQ ID NO: 26.

In some embodiments, the dCas9 comprises the amino acid sequence of SEQ ID NO: 1. In some embodiments, the N-terminal portion of dCas9 comprises the amino acid sequence of SEQ ID NOs: 2, 4, 6, 8, 10, 12, 14, 16, 18, 20, 22, or 24. In some embodiments, the C-terminal portion of dCas9 comprises the amino acid sequence of SEQ ID NOs: 3, 5, 7, 9, 11, 13, 15, 17, 19, 21, 23, or 25.

In some embodiments, the polynucleotide encoding the dCas9 comprises the nucleic acid sequence of SEQ ID NO: 26. In some embodiments, the polynucleotide encoding the N-terminal portion of dCas9 comprises the amino acid sequence of SEQ ID NOs: 27, 29, 31, 33, 35, 37, 39, 41, 43, 45, 47, or 49. In some embodiments, the polynucleotide encoding the C-terminal portion of dCas9 comprises the amino acid sequence of SEQ ID NOs: 28, 30, 32, 34, 36, 38, 40, 42, 44, 46, 48, or 50.

In some embodiments, the polypeptide capable of binding to an antibody or an antigen binding fragment thereof is selected from a group consisting of GCN4, T2A, 10×GCN4, and 10×GFP-11. In some embodiments, the GCN4 comprises an amino acid sequence of SEQ ID NO: 97. In some embodiments, the polynucleotide encoding the GCN4 comprises the nucleic acid sequence of SEQ ID NO: 101.

In some embodiments, the antibody or antigen binding fragment is a single domain antibody, a scFv, a Fab, a VH, a VHH or an antibody mimetic. In some embodiments, the antigen binding fragment is a scFv.

In some embodiments, the polypeptide sequence capable of binding to a nucleic acid structural element is selected from a group consisting of MS2 bacteriophage coat protein (MCP), PP7, and PCP. In some embodiments, the polypeptide sequence capable of binding to a nucleic acid structural element is MCP. In some embodiments, the MCP comprises an amino acid sequence of SEQ ID NO: 100. In some embodiments, a polynucleotide encoding the MCP comprises the nucleic acid sequence of SEQ ID NO: 105.

In some embodiments, the nucleic acid structural element is a RNA hairpin motif. In some embodiments, the RNA hairpin motif is selected from a group consisting of MS2 and PP7. In some embodiments, the RNA hairpin motif is a MS2 RNA hairpin motif. In some embodiments, the MS2 RNA hairpin motif comprises the nucleic acid sequence of SEQ ID NO: 104.

In some embodiments, the construct comprises the nucleic acid sequence of SEQ ID NOs: 134-162.

The disclosure provides a polypeptide expressed by any one of the constructs described herein. The disclosure provides a vector comprising any one of the constructs described herein.

In some embodiments, the vector further comprises a polynucleotide encoding a single guide RNA (sgRNA). In some embodiments, the polynucleotide encoding a sgRNA further comprises 2-20 copies of the nucleic acid structural element.

The disclosure provides a cell comprising any one of the constructs described herein. The disclosure provides a cell comprising any one of the polypeptides described herein. The disclosure provides a cell comprising any one of the vectors described herein.

In some embodiments, the cell further comprises at least one sgRNA. In some embodiments, the at least one sgRNA further comprises 2-20 copies of the nucleic acid structural element.

The disclosure provides a composition comprising any one of the constructs described herein. The disclosure provides a composition comprising any one of the polypeptides described herein. The disclosure provides a composition comprising any one of the vectors described herein.

In some embodiments, the composition further comprises at least one sgRNA. In some embodiments, the sgRNA further comprises 2-20 copies of the nucleic acid structural element. In some embodiments, the composition further comprises a pharmaceutically acceptable carrier.

The disclosure provides a method of modifying the expression of a gene product and minimizing off-target modifications in a population of cells comprising the step of introducing into the population of cells: i) any one of the constructs described herein or a polypeptide(s) expressed by the construct; and ii) at least one sgRNA, wherein the KRAB and/or modulator of gene expression provides a modification of at least one nucleotide near the gene and/or within a regulatory element of the gene, thereby modifying the expression of the gene product.

In some embodiments, (a) the polypeptide of i) comprises the 5′-(Aₘ₁-Bₘ₂)ₙ₁-CasN-(Aₘ₃-Bₘ₄)ₙ₂-CasC-Tₚ-3′ portion of Formula I, the 5′-(Aₘ₁-Bₘ₂)ₙ₁-CasN-(Aₘ₃-Bₘ₄)ₙ₂-CasC-Tₚ-3′ portion of Formula III, the 5′-(Aₘ₁-Bₘ₂)-CasN-CasC-Tₚ-3′ portion of Formula IIIb, the 5′-(A-B)-CasN-CasC-Tₚ-3′ portion of Formula IIIb-1, the 5′-CasN-CasC-Tₚ-3′ portion of Formula IIIc, the 5′-CasN-CasC-Tₚ-3′ portion of Formula IIIc-1, or the 5′-CasN-CasC-Tₚ-3′ portion of Formula IIIc-2, and the sgRNA of ii) are recruited to a genomic loci, and (b) multiple copies of the polypeptide of i) comprises the 5′-E-3′ portion of Formula I, the 5′-E-3′-portion of Formula III, the 5′-E-3′ portion of Formula IIIb, the 5′-Kᵣ-D_{q}-3′-portion of Formula IIIb-1, the 5′-E-3′-portion of Formula IIIc, the 5′-(Aₘ₅-Bₘ₆)ₙ₃-Kᵣ-D_{q}-3′ portion of Formula IIIc-1, or the 5′-Kᵣ-D_{q}-(Aₘ₅-Bₘ₆)ₙ₃-3′ portion of Formula IIIc-2 are recruited to a genomic loci via binding of the antibody or antigen binding fragment thereof to the epitope, thereby recruiting the polypeptides expressed by the construct to the genomic loci and modifying the expression of a gene product in a population of cells.

In some embodiments, the method further comprises introducing to the cells: iii) a second construct comprising the 5′-(Aₘ₅-Bₘ₆)ₙ₃-Kᵣ-D_{q}-3′ or 5′-Kᵣ-D_{q}-(Aₘ₅-Bₘ₆)ₙ₃-3′ of E or a polypeptide expressed by the second construct, wherein the polypeptide of i) comprising the 5′-CasN-CasC-Tₚ-3′ portion of Formula IIIa and the sgRNA are recruited to a genomic loci, wherein multiple copies of the polypeptide of iii) are recruited to a genomic loci via binding of the antibody or antigen binding fragment thereof to the epitope, thereby recruiting the polypeptides expressed by the construct of i) and the construct of iii) to the genomic loci, and modifying the expression of a gene product in a population of cells.

In some embodiments, (a) the polypeptide of i) comprises the 5′-(Aₘ₁-Bₘ₂)ₙ₁-CasN-(Aₘ₃-Bₘ₄)ₙ₂-CasC-Tₚ-3′ portion of Formula I, the 5′-(Aₘ₁-Bₘ₂)ₙ₁-CasN-(Aₘ₃-Bₘ₄)ₙ₂-CasC-Tₚ-3′ portion of Formula III, the 5′-(Aₘ₁-Bₘ₂)-CasN-CasC-Tₚ-3′ portion of Formula IIIb, the 5′-(A-B)-CasN-CasC-Tₚ-3′ portion of Formula IIIb-1, the 5′-CasN-CasC-Tₚ-3′ portion of Formula IIIc, the 5′-CasN-CasC-Tₚ-3′ portion of Formula IIIc-1, or the 5′-CasN-CasC-Tₚ-3′ portion of Formula IIIc-2, and the sgRNA of ii) are recruited to a genomic loci, and (b) multiple copies of the polypeptide of i) comprises the 5′-E-3′ portion of Formula I, the 5′-E-3′-portion of Formula III, the 5′-E-3′ portion of Formula IIIb, the 5′-Kᵣ-D_{q}-3′-portion of Formula IIIb-1, the 5′-E-3′-portion of Formula IIIc, the 5′-(Aₘ₅-Bₘ₆)ₙ₃-Kᵣ-D_{q}-3′ portion of Formula IIIc-1, or the 5′-Kᵣ-D_{q}-(Aₘ₅-Bₘ₆)ₙ₃-3′ portion of Formula IIIc-2 are recruited to a genomic loci via binding of the polypeptide sequence capable of binding to a nucleic acid structural element to the nucleic acid structural element, thereby recruiting the polypeptides expressed by the construct to the genomic loci and modifying the expression of a gene product in a population of cells.

In some embodiments, the method further comprising introducing to the cells: iii) a second construct comprising the 5′-(Aₘ₅-Bₘ₆)ₙ₃-Kᵣ-D_{q}-3′ or 5′-Kᵣ-D_{q}-(Aₘ₅-Bₘ₆)ₙ₃-3′ of E or a polypeptide expressed by the second construct, wherein the polypeptide of i) comprising the 5′-CasN-CasC-Tₚ-3′ portion of Formula IIIa and the sgRNA are recruited to a genomic loci, wherein multiple copies of the polypeptide of iii) are recruited to a genomic loci via binding of the polypeptide sequence capable of binding to a nucleic acid structural element to the nucleic acid structural element, thereby recruiting the polypeptides expressed by the construct to the genomic loci and modifying the expression of a gene product in a population of cells.

The disclosure provides an in vivo method of reducing or eliminating the expression of a gene product in a subject, comprising the step of introducing to a cell of the subject: i) any one of the constructs described herein or a polypeptide(s) expressed by the construct; and ii) at least one sgRNA, wherein the KRAB and/or modulator of gene expression provides a modification of at least one nucleotide near the gene and/or within a regulatory element of the gene, thereby modifying the expression of the gene product in the subject.

The disclosure provides a method for treating or alleviating a symptom of a gene product related disorder in a subject, comprising the step of introducing to a cell of the subject: i) any one of the constructs described herein or a polypeptide(s) expressed by the construct; and ii) at least one sgRNA, wherein the KRAB and/or modulator of gene expression provides a modification of at least one nucleotide near the gene and/or within a regulatory element of the gene, thereby modifying the expression of the gene product and treating or alleviating a symptom of the gene product related disorder in the subject.

In some embodiments, the expression of the gene product is reduced by 50-100% in the plurality of modified cells in comparison to a wildtype population of cells. In some embodiments, a ratio of on-site modification of the gene product to off-site modification of the gene product is about 10:1.

In some embodiments, the modification of at least one nucleotide is a DNA methylation or a histone modification. In some embodiments, the modification of at least one nucleotide is a DNA methylation.

In some embodiments, the gene regulatory element is a core promoter, a proximal promoter, a distal enhancer, a silencer, an insulator element, a boundary element or a locus control region.

In some embodiments, the modification of at least one nucleotide near the gene and/or within the regulatory element of the gene is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the transcription start site of the gene.

In some embodiments, the modification of at least one nucleotide near the gene and/or within the regulatory element of the gene is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the transcription start site of the gene.

In some embodiments, the construct is a deoxyribonucleic acid (DNA). In some embodiments, the construct is a messenger ribonucleic acid (mRNA). In some embodiments, the construct is formulated in a liposome or a lipid nanoparticle. In some embodiments, the construct and the sgRNA are formulated in a liposome or a lipid nanoparticle. In some embodiments, the construct and the sgRNA are formulated in the same liposome or lipid nanoparticle. In some embodiments, the construct and the sgRNA are formulated in different liposome or lipid nanoparticle.

In some embodiments, the liposome or lipid nanoparticle comprises of ionizable lipids (20%-70%, molar ratio), PEGylated lipids (0%-30%, molar ratio), supporting lipids (30%-50%, molar ratio), and cholesterol (10%-50%, molar ratio). In some embodiments, the ionizable lipid is selected from a group consisting of pH-responsive ionizable lipids, thermal-responsive ionizable lipids and light-responsive ionizable lipids.

In some embodiments, the construct is formulated in an AAV vector. In some embodiments, the construct and the sgRNA are formulated in an AAV vector. In some embodiments, the construct and the sgRNA are formulated in the same AAV vector. In some embodiments, the construct and the sgRNA are formulated in different AAV vectors.

In some embodiments, the construct or the polypeptide(s) expressed by the construct is delivered to the cell by local injection, systemic infusion, or a combination thereof.

In some embodiments, the gene product is selected from the group consisting of a VEGFA gene product, PCSK9 gene product, ANGPTL3 gene product, PTBP1 gene product, TTR gene product, Ube3a-ATS gene product, Ptp1b gene product, APOC3 gene product, hsd17b13 gene product, bcl11a gene product, and a TGF-beta gene product.

In some embodiments, the subject is a human. In some embodiments, the disease or disorder is selected from the group consisting of Familial hypercholesterolemia (FH), non-alcoholic steatohepatitis (NASH), Parkinson disease, hepatic fibrosis (HF), age-related macular disease (AMD), Angelman Syndrome (AS), Type II diabetes, 0-thalassemia, and hepatocellular carcinoma.

### BRIEF DESCRIPTION OF THE DRAWINGS

FIG. 1A is a schematic diagram showing the interaction of constructs comprising DNMT3A, DNMT3L, KRAB, and either intact or split dCas9 with DNA. FIG. 1B shows schematics of the constructs. FIG. 1C shows the editing efficiency on repressing two targeted gene expressions with gRNAs targeting CD81 or CD151 respectively for the constructs shown in FIG. 1A-1B, wherein: Dnmt-dCas9-Krab denotes Dnmt3A-Dnmt3L-dCas9-Krab; 768 INS denotes dCas9N-2-Dnmt3A-Dnmt3L-dCas9C-2-Krab; 776 INS denotes dCas9N-3-Dnmt3A-Dnmt3L-dCas9C-3-Krab; 1009 INS denotes dCas9N-4-Dnmt3A-Dnmt3L-dCas9C-4-Krab; 1048-1063 INS denotes dCas9N-5-Dnmt3A-Dnmt3L-dCas9C-5-Krab; 1072 INS denotes dCas9N-6-Dnmt3A-Dnmt3L-dCas9C-6-Krab; 1246 INS denotes dCas9N-7-Dnmt3A-Dnmt3L-dCas9C-7-Krab; 1248 INS denotes dCas9N-8-Dnmt3A-Dnmt3L-dCas9C-8-Krab; 1260 INS denotes dCas9N-10-Dnmt3A-Dnmt3L-dCas9C-10-Krab; Control denotes Dnmt3A-Dnmt3L-dCas9-Krab with Nt gRNA.

FIG. 2A is a schematic diagram showing the recruitment of DNMT3A, DNMT3L, and KRAB to dCas9 via an scFv bound to 10×GCN4. FIG. 2B shows schematics of the constructs. FIG. 2C-2E shows the editing efficiency on repressing two targeted gene expressions with gRNAs targeting CD81 or CD151 respectively for the constructs shown in FIG. 2A-2B, wherein: Suntag denotes Dnmt3A-Dnmt3L-dCas9-10×GCN4-T2A-scFv-KRAB; Dnmt-dCas9+scFv-Krab also denotes Dnmt3A-Dnmt3L-dCas9-10×GCN4-T2A-scFv-KRAB; dCas9+scFv-Dnmt-Krab denotes dCas9-10×GCN4-T2A-scFv-Dnmt3A-Dnmt3L-KRAB; dCas9+scFv-Dnmt+scFv-Krab denotes dCas9-10×GCN4-T2A-scFv-Dnmt3A-Dnmt3L-scFv-KRAB; Control denotes Dnmt3A-Dnmt3L-dCas9-Krab in FIG. 1 with Nt gRNA.

FIG. 3A is a schematic showing a construct comprising dCas9 and a guideRNA comprising a MS2, which binds to DNMT3A, DNMT3L, and KRAB via MCP. FIG. 3B shows schematics of constructs comprising DNMT3A, DNMT3L, MCP, dCas9, and KRAB.

FIG. 4A is a schematic showing the interaction between complexes comprising dCas9, DNMT3A, and DNMT3L as well as a modulator of gene expression selected from Kruppel-associated suppression box (KRAB), a Enhancer of zete homolog 2 (EZH2), a G9A, a Lysine-Specific histone Demethylase 1A (LSD1), a Heterochromatin Protein 1 (HP1), a Friend of GATA protein 1 (FOG1), a Histone Deacetylase (HDAC3) and/or a DOT1L with their DNA target site. FIG. 4B shows schematics of the constructs comprising dCas9, DNMT3A, and DNMT3L as well as a modulator of gene expression selected from Kruppel-associated suppression box (KRAB), a Enhancer of zete homolog 2 (EZH2), a G9A, a Lysine-Specific histone Demethylase 1A (LSD1), a Heterochromatin Protein 1 (HP1), a Friend of GATA protein 1 (FOG1), a Histone Deacetylase (HDAC3) and/or a DOT1L. FIG. 4C-4D shows the editing efficiency on repressing target gene expression for the constructs shown in FIG. 4A-4B, wherein the modulator of gene expression is selected from the listed types on the right of the figure. FIG. 4E-4F shows the editing efficiency on repressing target gene expression for the constructs shown in FIG. 4A-4B, wherein the KRAB modulator is selected from the listed origins on the right of the figure. Control denotes Dnmt3A-Dnmt3L-dCas9-Krab in FIG. 1 with Nt gRNA.

FIG. 5A is a schematic showing the interaction between a complex comprising dCas9, DNMT3A, and DNMT3L from various species. FIG. 5B shows schematics of the constructs. FIG. 5C-5D shows the editing efficiency on repressing target gene expression for the constructs shown in FIG. 5A-5B, wherein the DNMT3A and DNMT 3L are selected from the listed origins on the right of the figure. Control denotes Dnmt3A-Dnmt3L-dCas9-Krab in FIG. 1 with Nt gRNA.

FIG. 6A shows three examples of the constructs of the application. FIG. 6B shows the editing efficiency on repressing targeted gene expression for the three constructs.

### DETAILED DESCRIPTION

The present disclosure overcomes problems associated with current technologies by providing constructs comprising DNMT3A, DNMT3L, dCas9, and KRAB for targeted modification of the expression of a gene product. For example, the targeted modification of gene product in a cell for use in in vivo gene therapy.

### I. Definitions

As used herein, the term “coding sequence” or “encoding nucleic acid” means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

The term “complement” or “complementary” as used herein with reference to a nucleic acid can mean Watson-Crick (e.g., A-T/U and C-G) or Hoogsteen base pairing between nucleotides or nucleotide analogs of nucleic acid molecules. “Complementarity” refers toa property shared between two nucleic acid sequences, such that when they are aligned antiparallel to each other, the nucleotide bases at each position will be complementary.

The term “correcting”, “genome editing” and “restoring” refers to changing a mutant gene that encodes a mutant protein, a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

As used herein, the term “donor DNA”, “donor template” and “repair template” refers to a double-stranded DNA fragment or molecule that includes at least a portion of the gene of interest. The donor DNA may encode a full-functional protein or a partially-functional protein.

As used herein, the terms “frameshift” or “frameshift mutation” are used interchangeably and refer to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

As used herein, the term “functional” and “full-functional” describes a protein that has biological activity. A “functional gene” refers to a gene transcribed to mRNA, which is translated to a functional protein.

As used herein, the term “fusion protein” refers to a chimeric protein created through the covalent or non-covalent joining of two or more genes, directly or indirectly, that originally coded for separate proteins. In some embodiments, the translation of the fusion gene results in a single polypeptide with functional properties derived from each of the original proteins.

As used herein, the term “genetic construct” refers to the DNA or RNA molecules that comprise a nucleotide sequence that encodes a protein. The coding sequence includes initiation and termination signals operably linked to regulatory elements including a promoter and polyadenylation signal capable of directing expression in cells.

The term “Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery will repair the break by homologous recombination, which is enhanced several orders of magnitude in the presence of DNA cleavage. When the homologous DNA piece is absent, nonhomologous end joining may take place instead.

The term “genome editing” as used herein refers to changing a gene. Genome editing may include correcting or restoring a mutant gene. Genome editing may include knocking out a gene, such as a mutant gene or a normal gene. Genome editing may be used to treat disease by changing the gene of interest.

The term “identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al, SIAM J. Applied Math. 48, 1073 (1988), herein incorporated by reference in their entirety.

As used herein, the terms “mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

As used herein, the term “modulator of epigenetic modification” refers to an agent that targets gene expression via epigenetic modification (e.g., via histone acetylation or methylation, or DNA methylation at a regulatory element of target gene, e.g., a promoter, enhancer or transcription start site). Chromatin remodeling and DNA methylation are two main mechanisms for regulating gene transcription. Specific epigenetic marks (e.g., DNA methylation) structurally or biochemically direct gene transcription or gene silencing/repression. For example, DNA methylation of regions that regulate transcriptional activities alter gene expression without changing the underlying DNA sequence. Transcriptional regulation using epigenetic modification (e.g., DNA methylation) allows for targeted modulation of gene expression, without affecting the expression of other gene products.

The term “non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

The term “normal gene” as used herein refers to a gene that has not undergone a change, such as a loss, gain, or exchange of genetic material. The normal gene undergoes normal gene transmission and gene expression.

The term “nuclease mediated NHEJ” as used herein refers to NHEJ that is initiated after a nuclease, such as a cas9, cuts double stranded DNA.

As used herein, the term “nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

As used herein, the term “operably linked” means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term “partially-functional” as used herein describes a protein that is encoded by a mutant gene and has less biological activity than a functional protein but more than a non-functional protein. In one embodiment, a partially-functional protein shows a biological activity that is less than 95%, 90%, 85%, 80%, 75%, 70%, 65%, 60%, 55%, 50%, 45%, 40%, 35%, or 30% of that of a corresponding functional protein.

The term “premature stop codon” or “out-of-frame stop codon” as used interchangeably herein refers to nonsense mutation in a sequence of DNA, which results in a stop codon at a location not normally found in the wild-type gene. A premature stop codon may cause a protein to be truncated or shorter compared to the full-length version of the protein.

The term “promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of a nucleic acid. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, and CMV IE promoter.

The term “target gene” as used herein refers to any nucleotide sequence encoding a known or putative gene product. The target gene may be a mutated gene involved in a genetic disease or disorder.

The term “target region” as used herein refers to the region of the target gene to which the site-specific nuclease is designed to bind.

As used herein, the term “transgene” refers to a gene or genetic material containing a gene sequence that has been isolated from one organism and is introduced into a different organism. Alternatively, the term “transgene” also refers to a gene or genetic material that is chemically synthesized and introduced into an organism. This non-native segment of DNA may retain the ability to produce RNA or protein in the transgenic organism, or it may alter the normal function of the transgenic organism's genetic code. The introduction of a transgene has the potential to change the phenotype of an organism.

As used herein, the term “variant” when used with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. “Variant” with respect to a peptide or polypeptide that differs in amino acid sequence by the insertion, deletion, or conservative substitution of amino acids, but retain at least one biological activity.

Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157: 105-132(1982), incorporated herein by reference in its entirety. The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, the term “vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, such as a DNA plasmid.

As used herein, the terms “gene transfer,” “gene delivery,” and “gene transduction” refer to methods or systems for reliably inserting a particular nucleotide sequence (e.g., DNA or RNA), fusion protein, polypeptide and the like into targeted cells.

As used herein, the terms “adenoviral associated virus (AAV) vector,” “AAV gene therapy vector,” and “gene therapy vector” refer to a vector having functional or partly functional ITR sequences and transgenes. As used herein, the term “ITR” refers to inverted terminal repeats (ITR). The ITR sequences may be derived from an adeno-associated virus serotype, including without limitation, AAV-1, AAV-2, AAV-3, AAV-4, AAV-5, and AAV-6. The ITRs, however, need not be the wild-type nucleotide sequences, and may be altered (e.g., by the insertion, deletion or substitution of nucleotides), so long as the sequences retain function to provide for functional rescue, replication and packaging. AAV vectors may have one or more of the AAV wild-type genes deleted in whole or part, preferably the rep and/or cap genes but retain functional flanking ITR sequences. Functional ITR sequences function to, for example, rescue, replicate and package the AAV virion or particle. Thus, an “AAV vector” is defined herein to include at least those sequences required for insertion of the transgene into a subject's cells. Optionally included are those sequences necessary in cis for replication and packaging (e.g., functional ITRs) of the virus.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

The “transgene” may contain a transgenic sequence or a native or wild-type DNA sequence. The transgene may become part of the genome of the primate subject. A transgenic sequence can be partly or entirely species-heterologous, i.e., the transgenic sequence, or a portion thereof, can be from a species which is different from the cell into which it is introduced.

As used herein, the term “stably maintained” refers to characteristics of transgenic subject (e.g., a human or non-human primate) that maintain at least one of their transgenic elements (i.e., the element that is desired) through multiple generations of cells. For example, it is intended that the term encompass many cell division cycles of the originally transfected cell. The term “stable transfection” or “stably transfected” refers to the introduction and integration of foreign DNA into the genome of the cell. The term “stable transfectant” refers to a cell that has stably integrated foreign DNA into the genomic DNA.

As used herein, the terms “transgene encoding,” “nucleic acid molecule encoding,” “DNA sequence encoding,” and “DNA encoding” refer to the order or sequence of deoxyribonucleotides along a strand of deoxyribonucleic acid. The order of these deoxyribonucleotides may, for example, determine the order of amino acids along the polypeptide (protein) chain. The DNA sequence thus may code for the amino acid sequence.

As used herein, the term “wild type” (wt) refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants may be isolated, which are identified by the acquisition of altered characteristics when compared to the wild-type gene or gene product.

As used herein, the term “transfection” refers to the uptake of a foreign nucleic acid (e.g., DNA or RNA) by a cell. A cell has been “transfected” when an exogenous nucleic acid (DNA or RNA) has been introduced inside the cell membrane. A number of transfection techniques are generally known in the art (See, e.g., Graham et al., Virol., 52:456 (1973); Sambrook et al., Molecular Cloning, a Laboratory Manual, Cold Spring Harbor Laboratories, New York (1989); Davis et al., Basic Methods in Molecular Biology, Elsevier, (1986); and Chu et al., Gene 13:197 (1981), incorporated herein by reference in their entirety). Such techniques may be used to introduce one or more exogenous DNA moieties, such as a gene transfer vector and other nucleic acid molecules, into suitable recipient cells.

As used herein, the terms “stable transfection” and “stably transfected” refers to the introduction and integration of foreign DNA into the genome of the transfected cell. The term “stable transfectant” refers to a cell, which has stably integrated foreign DNA into the genomic DNA.

As used herein, the term “transient transfection” or “transiently transfected” refers to the introduction of foreign DNA into a cell wherein the foreign DNA fails to integrate into the genome of the transfected cell and is maintained as an episome. During this time the foreign DNA is subject to the regulatory controls that govern the expression of endogenous genes in the chromosomes. The term “transient transfectant” refers to cells which have taken up foreign DNA but have failed to integrate this DNA. As used herein, the term “transduction” denotes the delivery of a DNA molecule to a recipient cell either in vivo or in vitro, via a replication-defective viral vector, such as via a recombinant AAV virion.

As used herein, the term “recipient cell” refers to a cell which has been transfected or transduced, or is capable of being transfected or transduced, by a nucleic acid construct or vector bearing a selected nucleotide sequence of interest. The term includes the progeny of the parent cell, whether or not the progeny are identical in morphology or in genetic make-up to the original parent, so long as the selected nucleotide sequence is present. The recipient cell may be the cells of a subject to which the gene therapy particles and/or gene therapy vector has been administered.

As used herein, the term “recombinant DNA molecule” refers to a DNA molecule which is comprised of segments of DNA joined together by means of molecular biological techniques.

As used herein, the term “regulatory element” refers to a genetic element which can control the expression of nucleic acid sequences. For example, a promoter is a regulatory element that facilitates the initiation of transcription of an operably linked coding region. Other regulatory elements are splicing signals, polyadenylation signals, termination signals, etc.

The term DNA “control sequences” refers collectively to regulatory elements such as promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need be present.

Transcriptional control signals in eukaryotes generally comprise “promoter” and “enhancer” elements. Promoters and enhancers consist of short arrays of DNA sequences that interact specifically with cellular proteins involved in transcription (Maniatis et al., Science 236:1237 (1987), incorporated herein by reference in its entirety). Promoter and enhancer elements have been isolated from a variety of eukaryotic sources including genes in yeast, insect and mammalian cells and viruses (analogous control sequences, i.e., promoters, are also found in prokaryotes). The selection of a particular promoter and enhancer depends on the recipient cell type. Some eukaryotic promoters and enhancers have a broad host range while others are functional in a limited subset of cell types (See e.g., Voss et al., Trends Biochem. Sci., 11:287 (1986); and Maniatis et al., supra, for reviews, incorporated herein by reference in their entirety). For example, the SV40 early gene enhancer is very active in a variety of cell types from many mammalian species and has been used to express proteins in a broad range of mammalian cells (Dijkema et al, EMBO J. 4:761 (1985), incorporated herein by reference in its entirety). Promoter and enhancer elements derived from the human elongation factor 1-alpha gene (Uetsuki et al., J. Biol. Chem., 264:5791 (1989); Kim et al., Gene 91:217 (1990); and Mizushima and Nagata, Nucl. Acids. Res., 18:5322 (1990)), the long terminal repeats of the Rous sarcoma virus (Gorman et al., Proc. Natl. Acad. Sci. U.S.A. 79:6777 (1982)), and the human cytomegalovirus (Boshart et al., Cell 41:521 (1985)) are also of utility for expression of proteins in diverse mammalian cell types, incorporated herein by reference in their entirety. Promoters and enhancers can be found naturally, alone or together. For example, retroviral long terminal repeats comprise both promoter and enhancer elements. Generally promoters and enhancers act independently of the gene being transcribed or translated. Thus, the enhancer and promoter used can be “endogenous,” “exogenous,” or “heterologous” with respect to the gene to which they are operably linked. An “endogenous” enhancer/promoter is one which is naturally linked with a given gene in the genome. An “exogenous” or “heterologous” enhancer or promoter is one which is placed in juxtaposition to a gene by means of genetic manipulation (i.e., molecular biological techniques) such that transcription of that gene is directed by the linked enhancer/promoter.

As used herein, the term “tissue specific” refers to regulatory elements or control sequences, such as a promoter, an enhancer, etc., wherein the expression of the nucleic acid sequence is substantially greater in a specific cell type(s) or tissue(s).

The presence of “splicing signals” on an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York (1989), pp. 16.7-16.8, incorporated herein by reference in its entirety). A commonly used splice donor and acceptor site is the splice junction from the 16S RNA of SV40.

Transcription termination signals are generally found downstream of a polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which has been isolated from one gene and operably linked to the 3′ end of another gene. A commonly used heterologous poly A signal is the SV40 poly A signal. The SV40 poly A signal is contained on a 237 bp BamHI/BclI restriction fragment and directs both termination and polyadenylation (Sambrook et al., supra, at 16.6-16.7, incorporated herein by reference in its entirety).

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

As defined herein, a “therapeutically effective amount” or “therapeutic effective dose” is an amount or dose of a fusion protein, polypeptide, nucleic acid, lipid nanoparticle, liposome, AAV particle(s), or virion(s) capable of producing sufficient amounts of a desired protein to modulate the activity of the protein in a desired manner, thus providing a palliative tool for clinical intervention. In some embodiments, a therapeutically effective amount or dose of a transfected fusion protein, polypeptide, nucleic acid, AAV particle(s), or virion(s) as described herein is enough to confer suppression of a gene targeted by the fusion protein/gene therapy construct.

As used herein, the term “treat”, e.g., a disorder, means that a subject (e.g., a human) who has a disorder, is at risk of having a disorder, and/or experiences a symptom of a disorder, will, in an embodiment, suffer a less severe symptom and/or will recover faster, when a fusion molecule or a nucleic acid that encodes the fusion molecule, and/or a gRNA or a nucleic acid that encodes the gRNA, e.g., as described herein, is administered than if the fusion molecule or a nucleic acid that encodes the fusion molecule, and/or the gRNA or a nucleic acid that encodes the gRNA, were never administered.

### II. Constructs

In one aspect, provided herein are construct comprising a polynucleotide encoding DNMT3A or a portion thereof, a polynucleotide encoding DNMT3L or a portion thereof, a polynucleotide encoding dCas9, a polynucleotide encoding KRAB or a portion thereof, a polynucleotide encoding a modulator of gene expression, and a polynucleotide encoding (i) an epitope capable of binding to an antibody or antigen-binding fragment thereof or (ii) a polypeptide sequence capable of binding to a nucleic acid structural element. In some embodiments, a construct provided herein has the structure of Formula I: 5′-(Aₘ₁-Bₘ₂)ₙ₁-CasN-(Aₘ₃-Bₘ₄)ₙ₂-CasC-Tₚ-E-3′.

In Formula I, one of A and B is a polynucleotide encoding DNMT3A or a portion thereof, and the other of A and B is a polynucleotide encoding DNMT3L or a portion thereof. CasN and CasC are polynucleotides encoding a N-terminal portion of dCas9 and a C-terminal portion of dCas9, respectively. E is 5′-(Aₘ₅-Bₘ₆)ₙ₃-Kᵣ-D_{q}-3′ or 5′-Kᵣ-D_{q}-(Aₘ₅-Bₘ₆)ₙ₃-3′. K is a polynucleotide encoding KRAB or a portion thereof. D is a polynucleotide encoding a modulator of gene expression. A modulator of gene expression may be, for example, a Kruppel-associated suppression box (KRAB), a Enhancer of zete homolog 2 (EZH2), a G9A, a Lysine-Specific histone Demethylase 1A (LSD1), a Heterochromatin Protein 1 (HP1), a Friend of GATA protein 1 (FOG1), a Histone Deacetylase (HDAC3) and/or a DOT1L. T comprises a polynucleotide encoding (i) an epitope capable of binding to an antibody or an antigen binding fragment thereof (e.g., a single domain antibody, a scFv, a Fab, a VH, a VHH or an antibody mimetic) or (ii) a polypeptide sequence capable of binding to a nucleic acid structural element. A polypeptide capable of binding to an antibody or an antigen binding fragment thereof may be, for example, GCN4. A polypeptide sequence capable of binding to a nucleic acid structural element may be, for example, MS2 bacteriophage coat protein (MCP). Polypeptides encoding DNMT3A, DNMT3L, CasN, CasC, KRAB, and modulators of gene expression are described in more detail below.

In Formula I, m1, m2, m3, m4, m5, and m6 are each independently an integer selected from 0 to 3; n1, n2, and n3 are each independently an integer selected from 0 to 2; p is an integer selected from 0 to 20; q is an integer selected from 0 to 5; r is an integer selected from 0 to 5; and when p is 0, at least one of m1, m2, m3, and m4 is not 0, at least one of n1 and n2 is not 0, and at least one of q and r is not 0.

In some embodiments, a construct provided herein has the structure of Formula II: 5′-CasN-(Aₘ₃-Bₘ₄)ₙ₂-CasC-Kᵣ-D_{q}-3′, wherein: n2 is an integer selected from 1 and 2; r is an integer selected from 1 to 5; q is an integer selected from 0 to 5; and at least one of m3 and m4 is not 0.

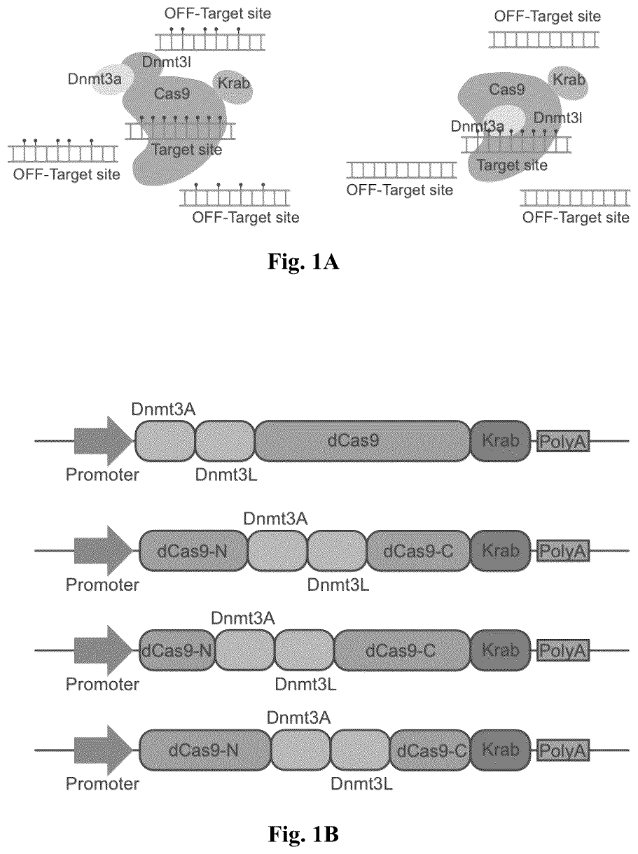

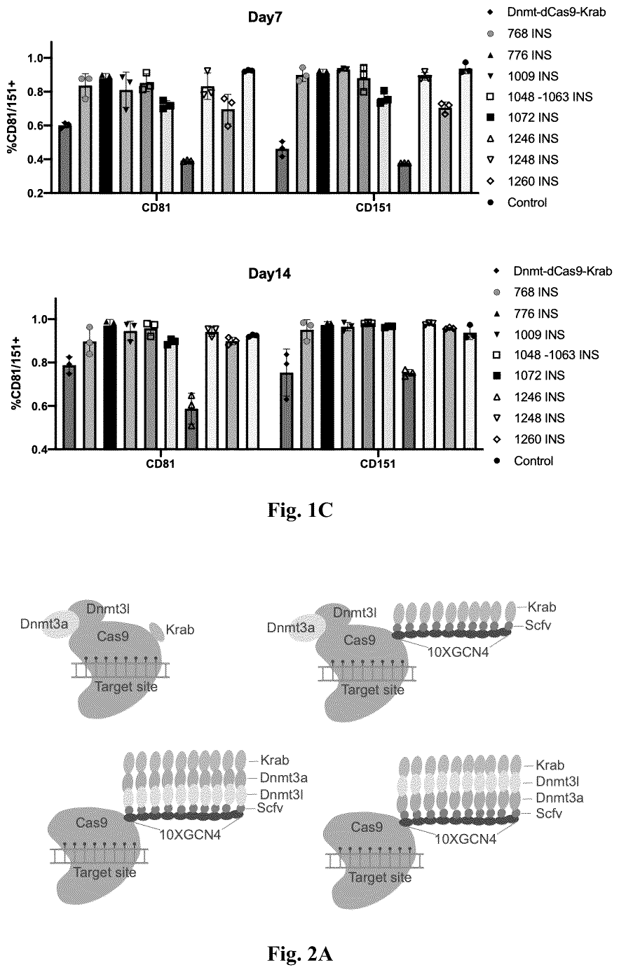

In some embodiments, the construct comprises the structure of Formula IIa: 5′-CasN-(A-B)-CasC-Kᵣ-D_{q}-3′. Exemplary constructs having the structure of Formula IIa are shown in second, third and fourth schematic of FIG. 1B.

In some embodiments, a construct provided herein has the structure of Formula III: 5′-(Aₘ₁-Bₘ₂)ₙ₁-CasN-(Aₘ₃-Bₘ₄)ₙ₂-CasC-Tₚ-E-3′, wherein p is an integer selected from 1 to 20.

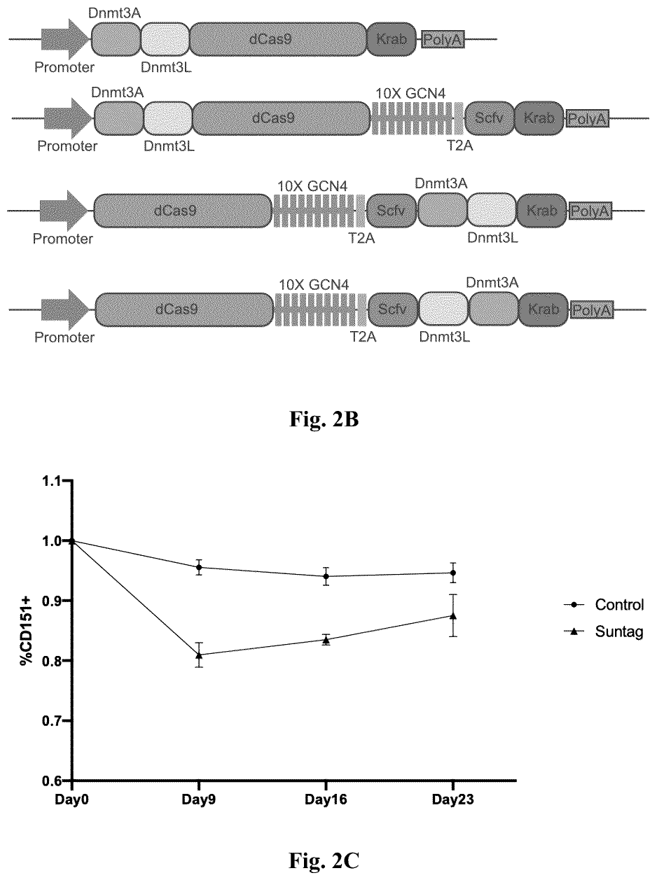

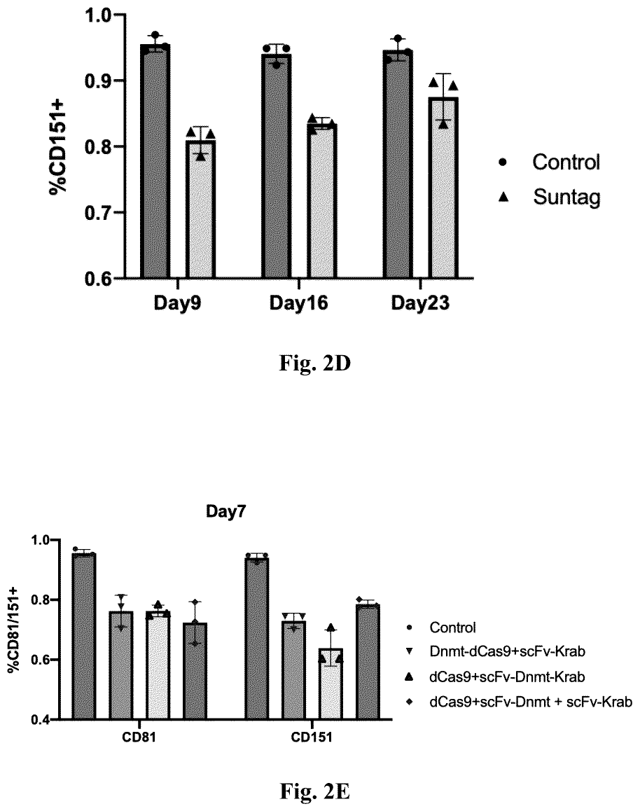

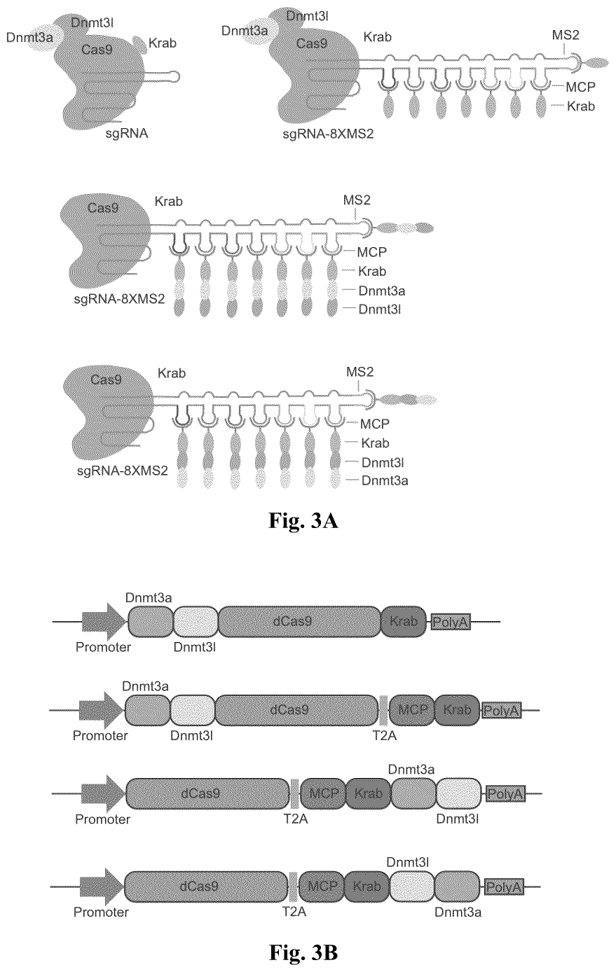

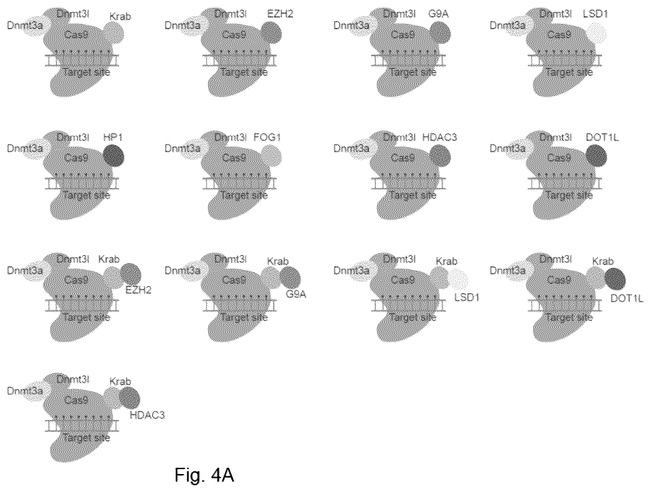

Exemplary constructs having the structure of Formula III are shown in FIGS. 2B and 3B. In some embodiments, the construct has the structure of Formula IIIa: 5′-CasN-CasC-Tₚ-3′. In the case of constructs of Formula IIIa, DNMT3A, DNMT3L, and KRAB are expressed on a separate construct.

In some embodiments, a construct provided herein has the structure of Formula IIIb: 5′-(Aₘ₁-Bₘ₂)-CasN-CasC-Tₚ-E-3′, wherein at least one of m1 and m2 is not 0. Exemplary constructs having the structure of Formula IIIb are shown in the second schematic in FIG. 2B and the second schematic in FIG. 3B. In some embodiments, the construct has the structure of Formula IIIb-1: 5′-(A-B)-CasN-CasC-Tₚ-Kᵣ-D_{q}-3′, wherein: r is an integer selected from 1 to 5; and q is an integer selected from 0 to 5.

In some embodiments, a construct provided herein has the structure of Formula IIc: 5′-CasN-CasC-Tₚ-E-3′, wherein: n3 is an integer selected from 1 and 2; r is an integer selected from 1 to 5; and q is an integer selected from 0 to 5; and at least one of m5 and m6 is not 0. Exemplary constructs having the structure of Formula IIc are shown in the third and fourth schematic in FIG. 2B and in the third and fourth schematic in FIG. 3B. In some embodiments, the construct has the structure of Formula IIIc-1: 5′-CasN-CasC-Tₚ-(Aₘ₅-Bₘ₆)ₙ₃-Kᵣ-D_{q}-3′. In some embodiments, the construct has the structure of Formula IIIc-2: 5′-CasN-CasC-Tₚ-Kᵣ-D_{q}-(Aₘ₅-Bₘ₆)ₙ₃-3′.

The sequences of the constructs described herein are set forth in

Table 1 with dCas9 shown in bold underlined, DNMT3A shown in italics, DNMT3L shown underlined, KRAB shown in bold italics, and EZH2, G9A, LSD1, HP1, FOG1, DHAC3, DOTML, 10×GCN4, MCP and ScFV shown in bold.

TABLE 1
Exemplary Sequences of Constructs

| Construct | Amino Acid | Nucleic Acid |
|---|---|---|
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| KRAB | QVDRYIASEVCEDSIT | CTGAAGGACCTGGGCATCCAAGTGGACCGCTACATTGCC |
| (FIG. | VGMVRHQGKIMYVG | TCCGAGGTGTGTGAGGACTCCATCACGGTGGGCATGGTG |
| 1B; | DVRSVTQKHIQEWG | CGGCACCAGGGAAAGATCATGTACGTCGGGGACGTCCG |
| FIG. | PFDLVIGGSPCNDLS | CAGCGTCACACAGAAGCATATCCAGGAGTGGGGCCCAT |
| 1C | IVNPARKGLYEGTGR | TCGACCTGGTGATTGGAGGCAGTCCCTGCAATGACCTCT |
| (Dnmt- | LFFEFYRLLHDARPK | CCATTGTCAACCCTGCCCGCAAGGGACTTTATGAGGGTA |
| dCas9- | EGDDRPFFWLFENV | CTGGCCGCCTCTTCTTTGAGTTCTACCGCCTCCTGCATGA |
| Krab)) | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESRTLVTFKD | CTCAGCTGGGAGGCGACGCCTATCCCTATGACGTGCCCG |
| | VFVDFTREEWKLL | ATTATGCCAGCCTGGGCAGCGGCTCCCCCAAGAAAAAA |
| | DTAQQIVYRNVMLE | CGCAAGGTGGAAGATCCTAAGAAAAAGCGGAAAGTGGA |
| | NYKNLVSLGYQLTK | CGGCagcggcagcgagacccccggtaccagcgagagcgccacccccgagagcCG |
| | PDVILRLEKGEEP | GACACTGGTGACCTTCAAGGATGTATTTGTGGACTTCAC |
| | (SEQ ID NO: 134) | CAGGGAGGAGTGGAAGCTGCTGGACACTGCTCAGCAGA |
| | | TCGTGTACAGAAATGTGATGCTGGAGAACTATAAGAACC |
| | | TGGTTTCCTTGGGTTATCAGCTTACTAAGCCAGATGTGAT |
| | | CCTCCGGTTGGAGAAGGGAGAAGAGCCC (SEQ ID NO: |
| | | 162) |
| dCas9N- | PKKKRKVMDKKYS | CCAAAAAAGAAGAGAAAGGTAATGGACAAGAAGTACA |
| 1- | IGLAIGTNSVGWA | GCATCGGCCTGGCCATCGGCACCAACTCTGTGGGCTGGG |
| DNMT3A- | VITDEYKVPSKKF | CCGTGATCACCGACGAGTACAAGGTGCCCAGCAAGAAA |
| DNMT3L- | KVLGNTDRHSIKK | TTCAAGGTGCTGGGCAACACCGACCGGCACAGCATCAA |
| dCas9C- | NLIGALLFDSGETA | GAAGAACCTGATCGGCGCCCTGCTGTTCGACAGCGGAG |
| 1- | EATRLKRTARRRY | AAACAGCCGAGGCCACCCGGCTGAAGAGAACCGCCAGA |
| KRAB | TRRKNRICYLQEIF | AGAAGATACACCAGACGGAAGAACCGGATCTGCTATCT |
| (FIG. | SNEMAKVDDSFFH | GCAAGAGATCTTCAGCAACGAGATGGCCAAGGTGGACG |
| 1B) | RLEESFLVEEDKK | ACAGCTTCTTCCACAGACTGGAAGAGTCCTTCCTGGTGG |
| | HERHPIFGNIVDEV | AAGAGGATAAGAAGCACGAGCGGCACCCCATCTTCGGC |
| | AYHEKYPTIYHLR | AACATCGTGGACGAGGTGGCCTACCACGAGAAGTACCC |
| | KKLVDSTDKADLR | CACCATCTACCACCTGAGAAAGAAACTGGTGGACAGCA |
| | LIYLALAHMIKFR | CCGACAAGGCCGACCTGCGGCTGATCTATCTGGCCCTGG |
| | GHFLIEGDLNPDNS | CCCACATGATCAAGTTCCGGGGCCACTTCCTGATCGAGG |
| | DVDKLFIQLVQTY | GCGACCTGAACCCCGACAACAGCGACGTGGACAAGCTG |
| | NQLFEENPINASGV | TTCATCCAGCTGGTGCAGACCTACAACCAGCTGTTCGAG |
| | DAKAILSARLSKSR | GAAAACCCCATCAACGCCAGCGGCGTGGACGCCAAGGC |
| | RLENLIAQLPGEK | CATCCTGTCTGCCAGACTGAGCAAGAGCAGACGGCTGG |
| | KNGLFGNLIALSL | AAAATCTGATCGCCCAGCTGCCCGGCGAGAAGAAGAAT |
| | GLTPNFKSNFDLA | GGCCTGTTCGGCAACCTGATTGCCCTGAGCCTGGGCCTG |
| | EDAKLQLSKDTYD | ACCCCCAACTTCAAGAGCAACTTCGACCTGGCCGAGGAT |
| | DDLDNLLAQIGDQ | GCCAAACTGCAGCTGAGCAAGGACACCTACGACGACGA |
| | YADLFLAAKNLSD | CCTGGACAACCTGCTGGCCCAGATCGGCGACCAGTACGC |
| | AILLSDILRVNTEIT | CGACCTGTTTCTGGCCGCCAAGAACCTGTCCGACGCCAT |
| | KAPLSASMIKRYD | CCTGCTGAGCGACATCCTGAGAGTGAACACCGAGATCAC |
| | EHHQDLTLLKALV | CAAGGCCCCCCTGAGCGCCTCTATGATCAAGAGATACGA |
| | RQQLPEKYKEIFF | CGAGCACCACCAGGACCTGACCCTGCTGAAAGCTCTCGT |
| | DQSKNGYAGYIDG | GCGGCAGCAGCTGCCTGAGAAGTACAAAGAGATTTTCTT |
| | GASQEEFYKFIKPI | CGACCAGAGCAAGAACGGCTACGCCGGCTACATCGATG |
| | LEKMDGTEELLVK | GCGGAGCCAGCCAGGAAGAGTTCTACAAGTTCATCAAG |
| | LNREDLLRKQRTF | CCCATCCTGGAAAAGATGGACGGCACCGAGGAACTGCT |
| | DNGSIPHQIHLGEL | CGTGAAGCTGAACAGAGAGGACCTGCTGCGGAAGCAGC |
| | HAILRRQEDFYPFL | GGACCTTCGACAACGGCAGCATCCCCCACCAGATCCACC |
| | KDNREKIEKILTFR | TGGGAGAGCTGCACGCCATTCTGCGGCGGCAGGAAGAT |
| | IPYYVGPLARGNS | TTTTACCCATTCCTGAAGGACAACCGGGAAAAGATCGAG |
| | RFAWMTRKSEETI | AAGATCCTGACCTTCCGCATCCCCTACTACGTGGGCCCT |
| | TPWNFEEVVDKGA | CTGGCCAGGGGAAACAGCAGATTCGCCTGGATGACCAG |
| | SAQSFIERMTNFD | AAAGAGCGAGGAAACCATCACCCCCTGGAACTTCGAGG |
| | KNLPNEKVLPKHS | AAGTGGTGGACAAGGGCGCCAGCGCCCAGAGCTTCATC |
| | LLYEYFTVYNELT | GAGCGGATGACCAACTTCGATAAGAACCTGCCCAACGA |
| | KVKYVTGGPSSGA | GAAGGTGCTGCCCAAGCACAGCCTGCTGTACGAGTACTT |
| | PPPSGGSPAGSPTST | CACCGTGTACAACGAGCTGACCAAAGTGAAATACGTGA |
| | EEGTSESATPESGPG | CCggagggccgagctctggcgcacccccaccaagtggagggtctcctgccgggtcccca |
| | TSTEPSEGSAPGSPA | acatctactgaagaaggcaccagcgaatccgcaacgcccgagtcaggccctggtacctcca |
| | GSPTSTEEGTSTEPS | cagaaccatctgaaggtagtgcgcctggttccccagctggaagccctacttccaccgaagaa |
| | EGSAPGTSTEPSEM | ggcacgtcaaccgaaccaagtgaaggatctgcccctgggaccagcactgaaccatctgag |
| | NHDQEFDPPKVYPP | ATGAACCATGACCAGGAATTTGACCCCCCAAAGGTTTAC |
| | VPAEKRKPIRVLSLF | CCACCTGTGCCAGCTGAGAAGAGGAAGCCCATCCGCGT |
| | DGIATGLLVLKDLGI | GCTGTCTCTCTTTGATGGGATTGCTACAGGGCTCCTGGTG |
| | QVDRYIASEVCEDSIT | CTGAAGGACCTGGGCATCCAAGTGGACCGCTACATTGCC |
| | VGMVRHQGKIMYVG | TCCGAGGTGTGTGAGGACTCCATCACGGTGGGCATGGTG |
| | DVRSVTQKHIQEWG | CGGCACCAGGGAAAGATCATGTACGTCGGGGACGTCCG |
| | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMERVFGFPVHYTD | AGGACCATTACCACCAGGTCAAACTCTATAAAGCAGGG |
| | VSNMSRLARQRLL | CAAAGACCAGCATTTCCCCGTCTTCATGAACGAGAAGGA |
| | GRSWSVPVIRHLFA | GGACATCCTGTGGTGCACTGAAATGGAAAGGGTGTTTGG |
| | PLKEYFACVSSGNS | CTTCCCCGTCCACTACACAGACGTCTCCAACATGAGCCG |
| | NANSRGPSFSSGLV | CTTGGCGAGGCAGAGACTGCTGGGCCGATCGTGGAGCG |
| | PLSLRGSHMGPMEI | TGCCGGTCATCCGCCACCTCTTCGCTCCGCTGAAGGAAT |
| | YKTVSAWKRQPVR | ATTTTGCTTGTGTGtctagcggcaatagtaacgctaacagccgcgggccgagct |
| | VLSLFRNIDKVLKS | tcagcagcggcctggtgccgttaagcttgcgcggcagccatATGggccctatggagatat |
| | LGFLESGSGSGGGT | acaagacagtgtctgcatggaagagacagccagtgcgggtactgagcctcttcagaaacatc |
| | LKYVEDVTNVVRR | gacaaggtactaaagagtttgggcttcttGGAAAGCGGTTCTGGTTCTGG |
| | DVEKWGPFDLVYG | GGGAGGAACGCTGAAGTACGTGGAAGATGTCACAAATG |
| | STQPLGSSCDRCPG | TCGTGAGGAGAGACGTGGAGAAATGGGGCCCCTTTGAC |
| | WYMFQFHRILQYA | CTGGTGTACGGCTCGACGCAGCCCCTAGGCAGCTCTTGT |
| | LPRQESQRPFFWIF | GATCGCTGTCCCGGCTGGTACATGTTCCAGTTCCACCGG |
| | MDNLLLTEDDQETT | ATCCTGCAGTATGCGCTGCCTCGCCAGGAGAGTCAGCGG |
| | TRFLQTEAVTLQDV | CCCTTCTTCTGGATATTCATGGACAATCTGCTGCTGACTG |
| | RGRDYQNAMRVW | AGGATGACCAAGAGACAACTACCCGCTTCCTTCAGACAG |
| | SNIPGLKSKHAPLTP | AGGCTGTGACCCTCCAGGATGTCCGTGGCAGAGACTACC |
| | KEEEYLQAQVRSRS | AGAATGCTATGCGGGTGTGGAGCAACATTCCAGGGCTG |
| | KLDAPKVDLLVKN | AAGAGCAAGCATGCGCCCCTGACCCCAAAGGAAGAAGA |
| | CLLPLREYFKYFSQ | GTATCTGCAAGCCCAAGTCAGAAGCAGGAGCAAGCTGG |
| | NSLPLGGPSSGAPPP | ACGCCCCGAAAGTTGACCTCCTGGTGAAGAACTGCCTTC |
| | SGGSPAGSPTSTEEG | TCCCGCTGAGAGAGTACTTCAAGTATTTTTCTCAAAACT |
| | TSESATPESGPGTST | CACTTCCTCTTggagggccgagctctggcgcacccccaccaagtggagggtctcc |
| | EPSEGSAPGSPAGSP | tgccgggtccccaacatctactgaagaaggcaccagcgaatccgcaacgcccgagtcaggc |
| | TSTEEGTSTEPSEGS | cctggtacctccacagaaccatctgaaggtagtgcgcctggttccccagctggaagccctact |
| | APGTSTEPSEEGMR | tccaccgaagaaggcacgtcaaccgaaccaagtgaaggatctgcccctgggaccagcactg |
| | KPAFLSGEQKKAI | aaccatctgagGAGGGAATGAGAAAGCCCGCCTTCCTGAGCG |
| | VDLLFKTNRKVTV | GCGAGCAGAAAAAAGCCATCGTGGACCTGCTGTTCAAG |
| | KQLKEDYFKKIEC | ACCAACCGGAAAGTGACCGTGAAGCAGCTGAAAGAGGA |
| | FDSVEISGVEDRFN | CTACTTCAAGAAAATCGAGTGCTTCGACTCCGTGGAAAT |
| | ASLGTYHDLLKIIK | CTCCGGCGTGGAAGATCGGTTCAACGCCTCCCTGGGCAC |
| | DKDFLDNEENEDI | ATACCACGATCTGCTGAAAATTATCAAGGACAAGGACTT |
| | LEDIVLTLTLFEDR | CCTGGACAATGAGGAAAACGAGGACATTCTGGAAGATA |
| | EMIEERLKTYAHL | TCGTGCTGACCCTGACACTGTTTGAGGACAGAGAGATGA |
| | FDDKVMKQLKRR | TCGAGGAACGGCTGAAAACCTATGCCCACCTGTTCGACG |
| | RYTGWGRLSRKLI | ACAAAGTGATGAAGCAGCTGAAGCGGCGGAGATACACC |
| | NGIRDKQSGKTIL | GGCTGGGGCAGGCTGAGCCGGAAGCTGATCAACGGCAT |
| | DFLKSDGFANRNF | CCGGGACAAGCAGTCCGGCAAGACAATCCTGGATTTCCT |
| | MQLIHDDSLTFKE | GAAGTCCGACGGCTTCGCCAACAGAAACTTCATGCAGCT |
| | DIQKAQVSGQGDS | GATCCACGACGACAGCCTGACCTTTAAAGAGGACATCCA |
| | LHEHIANLAGSPAI | GAAAGCCCAGGTGTCCGGCCAGGGCGATAGCCTGCACG |
| | KKGILQTVKVVDE | AGCACATTGCCAATCTGGCCGGCAGCCCCGCCATTAAGA |
| | LVKVMGRHKPENI | AGGGCATCCTGCAGACAGTGAAGGTGGTGGACGAGCTC |
| | VIEMARENQTTQK | GTGAAAGTGATGGGCCGGCACAAGCCCGAGAACATCGT |
| | GQKNSRERMKRIE | GATCGAAATGGCCAGAGAGAACCAGACCACCCAGAAGG |
| | EGIKELGSQILKEH | GACAGAAGAACAGCCGCGAGAGAATGAAGCGGATCGAA |
| | PVENTQLQNEKLY | GAGGGCATCAAAGAGCTGGGCAGCCAGATCCTGAAAGA |
| | LYYLQNGRDMYV | ACACCCCGTGGAAAACACCCAGCTGCAGAACGAGAAGC |
| | DQELDINRLSDYD | TGTACCTGTACTACCTGCAGAATGGGCGGGATATGTACG |
| | VDAIVPQSFLKDDS | TGGACCAGGAACTGGACATCAACCGGCTGTCCGACTACG |
| | IDNKVLTRSDKNR | ATGTGGACGCTATCGTGCCTCAGAGCTTTCTGAAGGACG |
| | GKSDNVPSEEVVK | ACTCCATCGATAACAAAGTGCTGACTCGGAGCGACAAG |
| | KMKNYWRQLLNA | AACCGGGGCAAGAGCGACAACGTGCCCTCCGAAGAGGT |
| | KLITQRKFDNLTK | CGTGAAGAAGATGAAGAACTACTGGCGCCAGCTGCTGA |
| | AERGGLSELDKAG | ATGCCAAGCTGATTACCCAGAGGAAGTTCGACAATCTGA |
| | FIKRQLVETRQITK | CCAAGGCCGAGAGAGGCGGCCTGAGCGAACTGGATAAG |
| | HVAQILDSRMNTK | GCCGGCTTCATCAAGAGACAGCTGGTGGAAACCCGGCA |
| | YDENDKLIREVKVI | GATCACAAAGCACGTGGCACAGATCCTGGACTCCCGGAT |
| | TLKSKLVSDFRKD | GAACACTAAGTACGACGAGAACGACAAACTGATCCGGG |
| | FQFYKVREINNYH | AAGTGAAAGTGATCACCCTGAAGTCCAAGCTGGTGTCCG |
| | HAHDAYLNAVVG | ATTTCCGGAAGGATTTCCAGTTTTACAAAGTGCGCGAGA |
| | TALIKKYPKLESEF | TCAACAACTACCACCACGCCCACGACGCCTACCTGAACG |
| | VYGDYKVYDVRK | CCGTCGTGGGAACCGCCCTGATCAAAAAGTACCCTAAGC |
| | MIAKSEQEIGKAT | TGGAAAGCGAGTTCGTGTACGGCGACTACAAGGTGTAC |
| | AKYFFYSNIMNFF | GACGTGCGGAAGATGATCGCCAAGAGCGAGCAGGAAAT |
| | KTEITLANGEIRKR | CGGCAAGGCTACCGCCAAGTACTTCTTCTACAGCAACAT |
| | PLIETNGETGEIVW | CATGAACTTTTTCAAGACCGAGATTACCCTGGCCAACGG |
| | DKGRDFATVRKVL | CGAGATCCGGAAGCGGCCTCTGATCGAGACAAACGGCG |
| | SMPQVNIVKKTEV | AAACAGGCGAGATCGTGTGGGATAAGGGCCGGGACTTT |
| | QTGGFSKESILPKR | GCCACCGTGCGGAAAGTGCTGTCTATGCCCCAAGTGAAT |
| | NSDKLIARKKDWD | ATCGTGAAAAAGACCGAGGTGCAGACAGGCGGCTTCAG |
| | PKKYGGFDSPTVA | CAAAGAGTCTATCCTGCCCAAGAGGAACAGCGACAAGC |
| | YSVLVVAKVEKGK | TGATCGCCAGAAAGAAGGACTGGGACCCTAAGAAGTAC |
| | SKKLKSVKELLGI | GGCGGCTTCGACAGCCCCACCGTGGCCTATTCTGTGCTG |
| | TIMERSSFEKNPID | GTGGTGGCCAAAGTGGAAAAGGGCAAGTCCAAGAAACT |
| | FLEAKGYKEVKKD | GAAGAGTGTGAAAGAGCTGCTGGGGATCACCATCATGG |
| | LIIKLPKYSLFELE | AAAGAAGCAGCTTCGAGAAGAATCCCATCGACTTTCTGG |
| | NGRKRMLASAGE | AAGCCAAGGGCTACAAAGAAGTGAAAAAGGACCTGATC |
| | LQKGNELALPSKY | ATCAAGCTGCCTAAGTACTCCCTGTTCGAGCTGGAAAAC |
| | VNFLYLASHYEKL | GGCCGGAAGAGAATGCTGGCCTCTGCCGGCGAACTGCA |
| | KGSPEDNEQKQLF | GAAGGGAAACGAACTGGCCCTGCCCTCCAAATATGTGA |
| | VEQHKHYLDEIIE | ACTTCCTGTACCTGGCCAGCCACTATGAGAAGCTGAAGG |
| | QISEFSKRVILADA | GCTCCCCCGAGGATAATGAGCAGAAACAGCTGTTTGTGG |
| | NLDKVLSAYNKHR | AACAGCACAAACACTACCTGGACGAGATCATCGAGCAG |
| | DKPIREQAENIIHL | ATCAGCGAGTTCTCCAAGAGAGTGATCCTGGCCGACGCT |
| | FTLTNLGAPAAFK | AATCTGGACAAGGTGCTGAGCGCCTACAACAAGCACAG |
| | YFDTTIDRKRYTST | AGACAAGCCTATCAGAGAGCAGGCCGAGAATATCATCC |
| | KEVLDATLIHQSIT | ACCTGTTTACCCTGACCAATCTGGGAGCCCCTGCCGCCT |
| | GLYETRIDLSQLG | TCAAGTACTTTGACACCACCATCGACCGGAAGAGGTACA |
| | GDYPYDVPDYASL | CCAGCACCAAAGAGGTGCTGGACGCCACCCTGATCCACC |
| | GSGSPKKKRKVEDP | AGAGCATCACCGGCCTGTACGAGACACGGATCGACCTGT |
| | KKKRKVDGSGSETP | CTCAGCTGGGAGGCGACTATCCCTATGACGTGCCCGATT |
| | GTSESATPESRTLVT | ATGCCAGCCTGGGCAGCGGCTCCCCCAAGAAAAAACGC |
| | FKDVFVDFTREEW | AAGGTGGAAGATCCTAAGAAAAAGCGGAAAGTGGACGG |
| | KLLDTAQQIVYRNV | CagcggcagcgagacccccggtaccagcgagagcgccacccccgagagcCGGAC |
| | MLENYKNLVSLGYQ | ACTGGTGACCTTCAAGGATGTATTTGTGGACTTCACCAG |
| | LTKPDVILRLEKGE | GGAGGAGTGGAAGCTGCTGGACACTGCTCAGCAGATCG |
| | EP* (SEQ ID NO: | TGTACAGAAATGTGATGCTGGAGAACTATAAGAACCTG |
| | 135) | GTTTCCTTGGGTTATCAGCTTACTAAGCCAGATGTGATCC |
| | | TCCGGTTGGAGAAGGGAGAAGAGCCCTAA (SEQ ID NO: |
| | | 163) |
| dCas9N- | PKKKRKVMDKKYS | CCAAAAAAGAAGAGAAAGGTAATGGACAAGAAGTACA |
| 2- | IGLAIGTNSVGWA | GCATCGGCCTGGCCATCGGCACCAACTCTGTGGGCTGGG |
| DNMT3A- | VITDEYKVPSKKF | CCGTGATCACCGACGAGTACAAGGTGCCCAGCAAGAAA |
| DNMT3L- | KVLGNTDRHSIKK | TTCAAGGTGCTGGGCAACACCGACCGGCACAGCATCAA |
| dCas9C- | NLIGALLFDSGETA | GAAGAACCTGATCGGCGCCCTGCTGTTCGACAGCGGAG |
| 2- | EATRLKRTARRRY | AAACAGCCGAGGCCACCCGGCTGAAGAGAACCGCCAGA |
| KRAB | TRRKNRICYLQEIF | AGAAGATACACCAGACGGAAGAACCGGATCTGCTATCT |
| (FIG. | SNEMAKVDDSFFH | GCAAGAGATCTTCAGCAACGAGATGGCCAAGGTGGACG |
| 1B) | RLEESFLVEEDKK | ACAGCTTCTTCCACAGACTGGAAGAGTCCTTCCTGGTGG |
| | HERHPIFGNIVDEV | AAGAGGATAAGAAGCACGAGCGGCACCCCATCTTCGGC |
| | AYHEKYPTIYHLR | AACATCGTGGACGAGGTGGCCTACCACGAGAAGTACCC |
| | KKLVDSTDKADLR | CACCATCTACCACCTGAGAAAGAAACTGGTGGACAGCA |
| | LIYLALAHMIKFR | CCGACAAGGCCGACCTGCGGCTGATCTATCTGGCCCTGG |
| | GHFLIEGDLNPDNS | CCCACATGATCAAGTTCCGGGGCCACTTCCTGATCGAGG |
| | DVDKLFIQLVQTY | GCGACCTGAACCCCGACAACAGCGACGTGGACAAGCTG |
| | NQLFEENPINASGV | TTCATCCAGCTGGTGCAGACCTACAACCAGCTGTTCGAG |
| | DAKAILSARLSKSR | GAAAACCCCATCAACGCCAGCGGCGTGGACGCCAAGGC |
| | RLENLIAQLPGEK | CATCCTGTCTGCCAGACTGAGCAAGAGCAGACGGCTGG |
| | KNGLFGNLIALSL | AAAATCTGATCGCCCAGCTGCCCGGCGAGAAGAAGAAT |
| | GLTPNFKSNFDLA | GGCCTGTTCGGCAACCTGATTGCCCTGAGCCTGGGCCTG |
| | EDAKLQLSKDTYD | ACCCCCAACTTCAAGAGCAACTTCGACCTGGCCGAGGAT |
| | DDLDNLLAQIGDQ | GCCAAACTGCAGCTGAGCAAGGACACCTACGACGACGA |
| | YADLFLAAKNLSD | CCTGGACAACCTGCTGGCCCAGATCGGCGACCAGTACGC |
| | AILLSDILRVNTEIT | CGACCTGTTTCTGGCCGCCAAGAACCTGTCCGACGCCAT |
| | KAPLSASMIKRYD | CCTGCTGAGCGACATCCTGAGAGTGAACACCGAGATCAC |
| | EHHQDLTLLKALV | CAAGGCCCCCCTGAGCGCCTCTATGATCAAGAGATACGA |
| | RQQLPEKYKEIFF | CGAGCACCACCAGGACCTGACCCTGCTGAAAGCTCTCGT |
| | DQSKNGYAGYIDG | GCGGCAGCAGCTGCCTGAGAAGTACAAAGAGATTTTCTT |
| | GASQEEFYKFIKPI | CGACCAGAGCAAGAACGGCTACGCCGGCTACATCGATG |
| | LEKMDGTEELLVK | GCGGAGCCAGCCAGGAAGAGTTCTACAAGTTCATCAAG |
| | LNREDLLRKQRTF | CCCATCCTGGAAAAGATGGACGGCACCGAGGAACTGCT |
| | DNGSIPHQIHLGEL | CGTGAAGCTGAACAGAGAGGACCTGCTGCGGAAGCAGC |
| | HAILRRQEDFYPFL | GGACCTTCGACAACGGCAGCATCCCCCACCAGATCCACC |
| | KDNREKIEKILTFR | TGGGAGAGCTGCACGCCATTCTGCGGCGGCAGGAAGAT |
| | IPYYVGPLARGNS | TTTTACCCATTCCTGAAGGACAACCGGGAAAAGATCGAG |
| | RFAWMTRKSEETI | AAGATCCTGACCTTCCGCATCCCCTACTACGTGGGCCCT |
| | TPWNFEEVVDKGA | CTGGCCAGGGGAAACAGCAGATTCGCCTGGATGACCAG |
| | SAQSFIERMTNFD | AAAGAGCGAGGAAACCATCACCCCCTGGAACTTCGAGG |
| | KNLPNEKVLPKHS | AAGTGGTGGACAAGGGCGCCAGCGCCCAGAGCTTCATC |
| | LLYEYFTVYNELT | GAGCGGATGACCAACTTCGATAAGAACCTGCCCAACGA |
| | KVKYVTEGMRKP | GAAGGTGCTGCCCAAGCACAGCCTGCTGTACGAGTACTT |
| | AFLSGEQKKAIVD | CACCGTGTACAACGAGCTGACCAAAGTGAAATACGTGA |
| | LLFKTNRKVTVKQ | CCGAGGGAATGAGAAAGCCCGCCTTCCTGAGCGGCGAG |
| | LKEDYFKKIECFDS | CAGAAAAAAGCCATCGTGGACCTGCTGTTCAAGACCAA |
| | VEISGVEDRFNASL | CCGGAAAGTGACCGTGAAGCAGCTGAAAGAGGACTACT |
| | GTYHDLLKIIKDK | TCAAGAAAATCGAGTGCTTCGACTCCGTGGAAATCTCCG |
| | DFLDNEENEDILED | GCGTGGAAGATCGGTTCAACGCCTCCCTGGGCACATACC |
| | IVLTLTLFEDREMI | ACGATCTGCTGAAAATTATCAAGGACAAGGACTTCCTGG |
| | EERLKTYAHLFDD | ACAATGAGGAAAACGAGGACATTCTGGAAGATATCGTG |
| | KVMKQLKRRRYT | CTGACCCTGACACTGTTTGAGGACAGAGAGATGATCGAG |
| | GWGRLSRKLINGI | GAACGGCTGAAAACCTATGCCCACCTGTTCGACGACAAA |
| | RDKQSGKTILDFL | GTGATGAAGCAGCTGAAGCGGCGGAGATACACCGGCTG |
| | KSDGFANRNFMQL | GGGCAGGCTGAGCCGGAAGCTGATCAACGGCATCCGGG |
| | IHDDSLTFKEDIQK | ACAAGCAGTCCGGCAAGACAATCCTGGATTTCCTGAAGT |
| | AQVSGQGDSLHEH | CCGACGGCTTCGCCAACAGAAACTTCATGCAGCTGATCC |
| | IANLAGSPAIKKGI | ACGACGACAGCCTGACCTTTAAAGAGGACATCCAGAAA |
| | LQTVKVVDELVKV | GCCCAGGTGTCCGGCCAGGGCGATAGCCTGCACGAGCA |
| | MGRHKPENIVIEM | CATTGCCAATCTGGCCGGCAGCCCCGCCATTAAGAAGGG |
| | ARENQGGPSSGAPP | CATCCTGCAGACAGTGAAGGTGGTGGACGAGCTCGTGA |
| | PSGGSPAGSPTSTEE | AAGTGATGGGCCGGCACAAGCCCGAGAACATCGTGATC |
| | GTSESATPESGPGTS | GAAATGGCCAGAGAGAACCAGggagggccgagctctggcgcaccccc |
| | TEPSEGSAPGSPAGS | accaagtggagggtctcctgccgggtccccaacatctactgaagaaggcaccagcgaatcc |
| | PTSTEEGTSTEPSEG | gcaacgcccgagtcaggccctggtacctccacagaaccatctgaaggtagtgcgcctggttc |
| | SAPGTSTEPSEMNH | cccagctggaagccctacttccaccgaagaaggcacgtcaaccgaaccaagtgaaggatct |
| | DQEFDPPKVYPPVP | gcccctgggaccagcactgaaccatctgagATGAACCATGACCAGGAAT |
| | AEKRKPIRVLSLFDGI | TTGACCCCCCAAAGGTTTACCCACCTGTGCCAGCTGAGA |
| | ATGLLVLKDLGIQVD | AGAGGAAGCCCATCCGCGTGCTGTCTCTCTTTGATGGGA |
| | RYIASEVCEDSITVGM | TTGCTACAGGGCTCCTGGTGCTGAAGGACCTGGGCATCC |
| | VRHQGKIMYVGDVR | AAGTGGACCGCTACATTGCCTCCGAGGTGTGTGAGGACT |
| | SVTQKHIQEWGPFD | CCATCACGGTGGGCATGGTGCGGCACCAGGGAAAGATC |
| | LVIGGSPCNDLSIVNP | ATGTACGTCGGGGACGTCCGCAGCGTCACACAGAAGCA |
| | ARKGLYEGTGRLFFE | TATCCAGGAGTGGGGCCCATTCGACCTGGTGATTGGAGG |
| | FYRLLHDARPKEGD | CAGTCCCTGCAATGACCTCTCCATTGTCAACCCTGCCCG |
| | DRPFFWLFENVVAM | CAAGGGACTTTATGAGGGTACTGGCCGCCTCTTCTTTGA |
| | GVSDKRDISRFLESN | GTTCTACCGCCTCCTGCATGATGCGCGGCCCAAGGAGGG |
| | PVMIDAKEVSAAHRA | AGATGATCGCCCCTTCTTCTGGCTCTTTGAGAATGTGGTG |
| | RYFWGNLPGMNRPL | GCCATGGGCGTTAGTGACAAGAGGGACATCTCGCGATTT |
| | ASTVNDKLELQECLE | CTTGAGTCTAACCCCGTGATGATTGACGCCAAAGAAGTG |
| | HGRIAKFSKVRTITTR | TCTGCTGCACACAGGGCCCGTTACTTCTGGGGTAACCTT |
| | SNSIKQGKDQHFPVF | CCTGGCATGAACAGGCCTTTGGCATCCACTGTGAATGAT |
| | MNEKEDILWCTEME | AAGCTGGAGCTGCAAGAGTGTCTGGAGCACGGCAGAAT |
| | RVFGFPVHYTDVSN | AGCCAAGTTCAGCAAAGTGAGGACCATTACCACCAGGT |
| | MSRLARQRLLGRS | CAAACTCTATAAAGCAGGGCAAAGACCAGCATTTCCCCG |
| | WSVPVIRHLFAPLK | TCTTCATGAACGAGAAGGAGGACATCCTGTGGTGCACTG |
| | EYFACVSSGNSNAN | AAATGGAAAGGGTGTTTGGCTTCCCCGTCCACTACACAG |
| | SRGPSFSSGLVPLSL | ACGTCTCCAACATGAGCCGCTTGGCGAGGCAGAGACTGC |
| | RGSHMGPMEIYKT | TGGGCCGATCGTGGAGCGTGCCGGTCATCCGCCACCTCT |
| | VSAWKRQPVRVLS | TCGCTCCGCTGAAGGAATATTTTGCTTGTGTGtctagcggcaat |
| | LFRNIDKVLKSLGF | agtaacgctaacagccgcgggccgagcttcagcagcggcctggtgccgttaagcttgcgcg |
| | LESGSGSGGGTLKY | gcagccatATGggccctatggagatatacaagacagtgtctgcatggaagagacagccag |
| | VEDVTNVVRRDVE | tgcgggtactgagcctcttcagaaacatcgacaaggtactaaagagtttgggcttcttGGAA |
| | KWGPFDLVYGSTQ | AGCGGTTCTGGTTCTGGGGGAGGAACGCTGAAGTACGTG |
| | PLGSSCDRCPGWY | GAAGATGTCACAAATGTCGTGAGGAGAGACGTGGAGAA |
| | MFQFHRILQYALPR | ATGGGGCCCCTTTGACCTGGTGTACGGCTCGACGCAGCC |
| | QESQRPFFWIFMDN | CCTAGGCAGCTCTTGTGATCGCTGTCCCGGCTGGTACAT |
| | LLLTEDDQETTTRF | GTTCCAGTTCCACCGGATCCTGCAGTATGCGCTGCCTCG |
| | LQTEAVTLQDVRG | CCAGGAGAGTCAGCGGCCCTTCTTCTGGATATTCATGGA |
| | RDYQNAMRVWSNI | CAATCTGCTGCTGACTGAGGATGACCAAGAGACAACTAC |
| | PGLKSKHAPLTPKE | CCGCTTCCTTCAGACAGAGGCTGTGACCCTCCAGGATGT |
| | EEYLQAQVRSRSKL | CCGTGGCAGAGACTACCAGAATGCTATGCGGGTGTGGA |
| | DAPKVDLLVKNCL | GCAACATTCCAGGGCTGAAGAGCAAGCATGCGCCCCTG |
| | LPLREYFKYFSQNS | ACCCCAAAGGAAGAAGAGTATCTGCAAGCCCAAGTCAG |
| | LPLGGPSSGAPPPSG | AAGCAGGAGCAAGCTGGACGCCCCGAAAGTTGACCTCC |
| | GSPAGSPTSTEEGTS | TGGTGAAGAACTGCCTTCTCCCGCTGAGAGAGTACTTCA |
| | ESATPESGPGTSTEP | AGTATTTTTCTCAAAACTCACTTCCTCTTggagggccgagctctgg |
| | SEGSAPGSPAGSPTS | cgcacccccaccaagtggagggtctcctgccgggtccccaacatctactgaagaaggcacc |
| | TEEGTSTEPSEGSAP | agcgaatccgcaacgcccgagtcaggccctggtacctccacagaaccatctgaaggtagtg |
| | GTSTEPSETTQKGQ | cgcctggttccccagctggaagccctacttccaccgaagaaggcacgtcaaccgaaccaagt |
| | KNSRERMKRIEEG | gaaggatctgcccctgggaccagcactgaaccatctgagACCACCCAGAAGGG |
| | IKELGSQILKEHPV | ACAGAAGAACAGCCGCGAGAGAATGAAGCGGATCGAAG |
| | ENTQLQNEKLYLY | AGGGCATCAAAGAGCTGGGCAGCCAGATCCTGAAAGAA |
| | YLQNGRDMYVDQ | CACCCCGTGGAAAACACCCAGCTGCAGAACGAGAAGCT |
| | ELDINRLSDYDVD | GTACCTGTACTACCTGCAGAATGGGCGGGATATGTACGT |
| | AIVPQSFLKDDSID | GGACCAGGAACTGGACATCAACCGGCTGTCCGACTACG |
| | NKVLTRSDKNRGK | ATGTGGACGCTATCGTGCCTCAGAGCTTTCTGAAGGACG |
| | SDNVPSEEVVKKM | ACTCCATCGATAACAAAGTGCTGACTCGGAGCGACAAG |
| | KNYWRQLLNAKLI | AACCGGGGCAAGAGCGACAACGTGCCCTCCGAAGAGGT |
| | TQRKFDNLTKAER | CGTGAAGAAGATGAAGAACTACTGGCGCCAGCTGCTGA |
| | GGLSELDKAGFIK | ATGCCAAGCTGATTACCCAGAGGAAGTTCGACAATCTGA |
| | RQLVETRQITKHV | CCAAGGCCGAGAGAGGCGGCCTGAGCGAACTGGATAAG |
| | AQILDSRMNTKYD | GCCGGCTTCATCAAGAGACAGCTGGTGGAAACCCGGCA |
| | ENDKLIREVKVITL | GATCACAAAGCACGTGGCACAGATCCTGGACTCCCGGAT |
| | KSKLVSDFRKDFQ | GAACACTAAGTACGACGAGAACGACAAACTGATCCGGG |
| | FYKVREINNYHHA | AAGTGAAAGTGATCACCCTGAAGTCCAAGCTGGTGTCCG |
| | HDAYLNAVVGTAL | ATTTCCGGAAGGATTTCCAGTTTTACAAAGTGCGCGAGA |
| | IKKYPKLESEFVY | TCAACAACTACCACCACGCCCACGACGCCTACCTGAACG |
| | GDYKVYDVRKMI | CCGTCGTGGGAACCGCCCTGATCAAAAAGTACCCTAAGC |
| | AKSEQEIGKATAK | TGGAAAGCGAGTTCGTGTACGGCGACTACAAGGTGTAC |
| | YFFYSNIMNFFKTE | GACGTGCGGAAGATGATCGCCAAGAGCGAGCAGGAAAT |
| | ITLANGEIRKRPLI | CGGCAAGGCTACCGCCAAGTACTTCTTCTACAGCAACAT |
| | ETNGETGEIVWDK | CATGAACTTTTTCAAGACCGAGATTACCCTGGCCAACGG |
| | GRDFATVRKVLSM | CGAGATCCGGAAGCGGCCTCTGATCGAGACAAACGGCG |
| | PQVNIVKKTEVQT | AAACAGGCGAGATCGTGTGGGATAAGGGCCGGGACTTT |
| | GGFSKESILPKRNS | GCCACCGTGCGGAAAGTGCTGTCTATGCCCCAAGTGAAT |
| | DKLIARKKDWDPK | ATCGTGAAAAAGACCGAGGTGCAGACAGGCGGCTTCAG |
| | KYGGFDSPTVAYS | CAAAGAGTCTATCCTGCCCAAGAGGAACAGCGACAAGC |
| | VLVVAKVEKGKSK | TGATCGCCAGAAAGAAGGACTGGGACCCTAAGAAGTAC |
| | KLKSVKELLGITI | GGCGGCTTCGACAGCCCCACCGTGGCCTATTCTGTGCTG |
| | MERSSFEKNPIDFL | GTGGTGGCCAAAGTGGAAAAGGGCAAGTCCAAGAAACT |
| | EAKGYKEVKKDLI | GAAGAGTGTGAAAGAGCTGCTGGGGATCACCATCATGG |
| | IKLPKYSLFELENG | AAAGAAGCAGCTTCGAGAAGAATCCCATCGACTTTCTGG |
| | RKRMLASAGELQ | AAGCCAAGGGCTACAAAGAAGTGAAAAAGGACCTGATC |
| | KGNELALPSKYVN | ATCAAGCTGCCTAAGTACTCCCTGTTCGAGCTGGAAAAC |
| | FLYLASHYEKLKG | GGCCGGAAGAGAATGCTGGCCTCTGCCGGCGAACTGCA |
| | SPEDNEQKQLFVE | GAAGGGAAACGAACTGGCCCTGCCCTCCAAATATGTGA |
| | QHKHYLDEIIEQIS | ACTTCCTGTACCTGGCCAGCCACTATGAGAAGCTGAAGG |
| | EFSKRVILADANLD | GCTCCCCCGAGGATAATGAGCAGAAACAGCTGTTTGTGG |
| | KVLSAYNKHRDKP | AACAGCACAAACACTACCTGGACGAGATCATCGAGCAG |
| | IREQAENIIHLFTL | ATCAGCGAGTTCTCCAAGAGAGTGATCCTGGCCGACGCT |
| | TNLGAPAAFKYFD | AATCTGGACAAGGTGCTGAGCGCCTACAACAAGCACAG |
| | TTIDRKRYTSTKE | AGACAAGCCTATCAGAGAGCAGGCCGAGAATATCATCC |
| | VLDATLIHQSITGL | ACCTGTTTACCCTGACCAATCTGGGAGCCCCTGCCGCCT |
| | YETRIDLSQLGGD | TCAAGTACTTTGACACCACCATCGACCGGAAGAGGTACA |
| | YPYDVPDYASLGSG | CCAGCACCAAAGAGGTGCTGGACGCCACCCTGATCCACC |
| | SPKKKRKVEDPKK | AGAGCATCACCGGCCTGTACGAGACACGGATCGACCTGT |
| | KRKVDGSGSETPGT | CTCAGCTGGGAGGCGACTATCCCTATGACGTGCCCGATT |
| | SESATPESRTLVTFK | ATGCCAGCCTGGGCAGCGGCTCCCCCAAGAAAAAACGC |
| | DVFVDFTREEWKL | AAGGTGGAAGATCCTAAGAAAAAGCGGAAAGTGGACGG |
| | LDTAQQIVYRNVML | CagcggcagcgagacccccggtaccagcgagagcgccacccccgagagcCGGAC |
| | ENYKNLVSLGYQLT | ACTGGTGACCTTCAAGGATGTATTTGTGGACTTCACCAG |
| | KPDVILRLEKGEEP | GGAGGAGTGGAAGCTGCTGGACACTGCTCAGCAGATCG |
| | * (SEQ ID NO: 136) | TGTACAGAAATGTGATGCTGGAGAACTATAAGAACCTG |
| | | GTTTCCTTGGGTTATCAGCTTACTAAGCCAGATGTGATCC |
| | | TCCGGTTGGAGAAGGGAGAAGAGCCCTAA (SEQ ID NO: |
| | | 164) |
| dCas9N- | PKKKRKVMDKKYS | CCAAAAAAGAAGAGAAAGGTAATGGACAAGAAGTACA |
| 3- | IGLAIGTNSVGWA | GCATCGGCCTGGCCATCGGCACCAACTCTGTGGGCTGGG |
| DNMT3A- | VITDEYKVPSKKF | CCGTGATCACCGACGAGTACAAGGTGCCCAGCAAGAAA |
| DNMT3L- | KVLGNTDRHSIKK | TTCAAGGTGCTGGGCAACACCGACCGGCACAGCATCAA |
| dCas9C | NLIGALLFDSGETA | GAAGAACCTGATCGGCGCCCTGCTGTTCGACAGCGGAG |
| 3- | EATRLKRTARRRY | AAACAGCCGAGGCCACCCGGCTGAAGAGAACCGCCAGA |
| KRAB | TRRKNRICYLQEIF | AGAAGATACACCAGACGGAAGAACCGGATCTGCTATCT |
| (FIG. | SNEMAKVDDSFFH | GCAAGAGATCTTCAGCAACGAGATGGCCAAGGTGGACG |
| 1B) | RLEESFLVEEDKK | ACAGCTTCTTCCACAGACTGGAAGAGTCCTTCCTGGTGG |
| | HERHPIFGNIVDEV | AAGAGGATAAGAAGCACGAGCGGCACCCCATCTTCGGC |
| | AYHEKYPTIYHLR | AACATCGTGGACGAGGTGGCCTACCACGAGAAGTACCC |
| | KKLVDSTDKADLR | CACCATCTACCACCTGAGAAAGAAACTGGTGGACAGCA |
| | LIYLALAHMIKFR | CCGACAAGGCCGACCTGCGGCTGATCTATCTGGCCCTGG |
| | GHFLIEGDLNPDNS | CCCACATGATCAAGTTCCGGGGCCACTTCCTGATCGAGG |
| | DVDKLFIQLVQTY | GCGACCTGAACCCCGACAACAGCGACGTGGACAAGCTG |
| | NQLFEENPINASGV | TTCATCCAGCTGGTGCAGACCTACAACCAGCTGTTCGAG |
| | DAKAILSARLSKSR | GAAAACCCCATCAACGCCAGCGGCGTGGACGCCAAGGC |
| | RLENLIAQLPGEK | CATCCTGTCTGCCAGACTGAGCAAGAGCAGACGGCTGG |
| | KNGLFGNLIALSL | AAAATCTGATCGCCCAGCTGCCCGGCGAGAAGAAGAAT |
| | GLTPNFKSNFDLA | GGCCTGTTCGGCAACCTGATTGCCCTGAGCCTGGGCCTG |
| | EDAKLQLSKDTYD | ACCCCCAACTTCAAGAGCAACTTCGACCTGGCCGAGGAT |
| | DDLDNLLAQIGDQ | GCCAAACTGCAGCTGAGCAAGGACACCTACGACGACGA |
| | YADLFLAAKNLSD | CCTGGACAACCTGCTGGCCCAGATCGGCGACCAGTACGC |
| | AILLSDILRVNTEIT | CGACCTGTTTCTGGCCGCCAAGAACCTGTCCGACGCCAT |
| | KAPLSASMIKRYD | CCTGCTGAGCGACATCCTGAGAGTGAACACCGAGATCAC |
| | EHHQDLTLLKALV | CAAGGCCCCCCTGAGCGCCTCTATGATCAAGAGATACGA |
| | RQQLPEKYKEIFF | CGAGCACCACCAGGACCTGACCCTGCTGAAAGCTCTCGT |
| | DQSKNGYAGYIDG | GCGGCAGCAGCTGCCTGAGAAGTACAAAGAGATTTTCTT |
| | GASQEEFYKFIKPI | CGACCAGAGCAAGAACGGCTACGCCGGCTACATCGATG |
| | LEKMDGTEELLVK | GCGGAGCCAGCCAGGAAGAGTTCTACAAGTTCATCAAG |
| | LNREDLLRKQRTF | CCCATCCTGGAAAAGATGGACGGCACCGAGGAACTGCT |
| | DNGSIPHQIHLGEL | CGTGAAGCTGAACAGAGAGGACCTGCTGCGGAAGCAGC |
| | HAILRRQEDFYPFL | GGACCTTCGACAACGGCAGCATCCCCCACCAGATCCACC |
| | KDNREKIEKILTFR | TGGGAGAGCTGCACGCCATTCTGCGGCGGCAGGAAGAT |
| | IPYYVGPLARGNS | TTTTACCCATTCCTGAAGGACAACCGGGAAAAGATCGAG |
| | RFAWMTRKSEETI | AAGATCCTGACCTTCCGCATCCCCTACTACGTGGGCCCT |
| | TPWNFEEVVDKGA | CTGGCCAGGGGAAACAGCAGATTCGCCTGGATGACCAG |
| | SAQSFIERMTNFD | AAAGAGCGAGGAAACCATCACCCCCTGGAACTTCGAGG |
| | KNLPNEKVLPKHS | AAGTGGTGGACAAGGGCGCCAGCGCCCAGAGCTTCATC |
| | LLYEYFTVYNELT | GAGCGGATGACCAACTTCGATAAGAACCTGCCCAACGA |
| | KVKYVTEGMRKP | GAAGGTGCTGCCCAAGCACAGCCTGCTGTACGAGTACTT |
| | AFLSGEQKKAIVD | CACCGTGTACAACGAGCTGACCAAAGTGAAATACGTGA |
| | LLFKTNRKVTVKQ | CCGAGGGAATGAGAAAGCCCGCCTTCCTGAGCGGCGAG |
| | LKEDYFKKIECFDS | CAGAAAAAAGCCATCGTGGACCTGCTGTTCAAGACCAA |
| | VEISGVEDRFNASL | CCGGAAAGTGACCGTGAAGCAGCTGAAAGAGGACTACT |
| | GTYHDLLKIIKDK | TCAAGAAAATCGAGTGCTTCGACTCCGTGGAAATCTCCG |
| | DFLDNEENEDILED | GCGTGGAAGATCGGTTCAACGCCTCCCTGGGCACATACC |
| | IVLTLTLFEDREMI | ACGATCTGCTGAAAATTATCAAGGACAAGGACTTCCTGG |
| | EERLKTYAHLFDD | ACAATGAGGAAAACGAGGACATTCTGGAAGATATCGTG |
| | KVMKQLKRRRYT | CTGACCCTGACACTGTTTGAGGACAGAGAGATGATCGAG |
| | GWGRLSRKLINGI | GAACGGCTGAAAACCTATGCCCACCTGTTCGACGACAAA |
| | RDKQSGKTILDFL | GTGATGAAGCAGCTGAAGCGGCGGAGATACACCGGCTG |
| | KSDGFANRNFMQL | GGGCAGGCTGAGCCGGAAGCTGATCAACGGCATCCGGG |
| | IHDDSLTFKEDIQK | ACAAGCAGTCCGGCAAGACAATCCTGGATTTCCTGAAGT |
| | AQVSGQGDSLHEH | CCGACGGCTTCGCCAACAGAAACTTCATGCAGCTGATCC |
| | IANLAGSPAIKKGI | ACGACGACAGCCTGACCTTTAAAGAGGACATCCAGAAA |
| | LQTVKVVDELVKV | GCCCAGGTGTCCGGCCAGGGCGATAGCCTGCACGAGCA |
| | MGRHKPENIVIEM | CATTGCCAATCTGGCCGGCAGCCCCGCCATTAAGAAGGG |
| | ARENQTTQKGQK | CATCCTGCAGACAGTGAAGGTGGTGGACGAGCTCGTGA |
| | NGGPSSGAPPPSGG | AAGTGATGGGCCGGCACAAGCCCGAGAACATCGTGATC |
| | SPAGSPTSTEEGTSE | GAAATGGCCAGAGAGAACCAGACCACCCAGAAGGGACA |
| | SATPESGPGTSTEPS | GAAGAACggagggccgagctctggcgcacccccaccaagtggagggtctcctgccg |
| | EGSAPGSPAGSPTST | ggtccccaacatctactgaagaaggcaccagcgaatccgcaacgcccgagtcaggccctgg |
| | EEGTSTEPSEGSAPG | tacctccacagaaccatctgaaggtagtgcgcctggttccccagctggaagccctacttccac |
| | TSTEPSEMNHDQEF | cgaagaaggcacgtcaaccgaaccaagtgaaggatctgcccctgggaccagcactgaacc |
| | DPPKVYPPVPAEKRK | atctgagATGAACCATGACCAGGAATTTGACCCCCCAAAGG |
| | PIRVLSLFDGIATGLL | TTTACCCACCTGTGCCAGCTGAGAAGAGGAAGCCCATCC |
| | VLKDLGIQVDRYIAS | GCGTGCTGTCTCTCTTTGATGGGATTGCTACAGGGCTCCT |
| | EVCEDSITVGMVRH | GGTGCTGAAGGACCTGGGCATCCAAGTGGACCGCTACAT |
| | QGKIMYVGDVRSVT | TGCCTCCGAGGTGTGTGAGGACTCCATCACGGTGGGCAT |
| | QKHIQEWGPFDLVI | GGTGCGGCACCAGGGAAAGATCATGTACGTCGGGGACG |
| | GGSPCNDLSIVNPAR | TCCGCAGCGTCACACAGAAGCATATCCAGGAGTGGGGC |
| | KGLYEGTGRLFFEFY | CCATTCGACCTGGTGATTGGAGGCAGTCCCTGCAATGAC |
| | RLLHDARPKEGDDR | CTCTCCATTGTCAACCCTGCCCGCAAGGGACTTTATGAG |
| | PFFWLFENVVAMGV | GGTACTGGCCGCCTCTTCTTTGAGTTCTACCGCCTCCTGC |
| | SDKRDISRFLESNPV | ATGATGCGCGGCCCAAGGAGGGAGATGATCGCCCCTTCT |
| | MIDAKEVSAAHRARY | TCTGGCTCTTTGAGAATGTGGTGGCCATGGGCGTTAGTG |
| | FWGNLPGMNRPLAS | ACAAGAGGGACATCTCGCGATTTCTTGAGTCTAACCCCG |
| | TVNDKLELQECLEH | TGATGATTGACGCCAAAGAAGTGTCTGCTGCACACAGGG |
| | GRIAKFSKVRTITTRS | CCCGTTACTTCTGGGGTAACCTTCCTGGCATGAACAGGC |
| | NSIKQGKDQHFPVF | CTTTGGCATCCACTGTGAATGATAAGCTGGAGCTGCAAG |
| | MNEKEDILWCTEME | AGTGTCTGGAGCACGGCAGAATAGCCAAGTTCAGCAAA |
| | RVFGFPVHYTDVSN | GTGAGGACCATTACCACCAGGTCAAACTCTATAAAGCAG |
| | MSRLARQRLLGRS | GGCAAAGACCAGCATTTCCCCGTCTTCATGAACGAGAAG |
| | WSVPVIRHLFAPLK | GAGGACATCCTGTGGTGCACTGAAATGGAAAGGGTGTTT |
| | EYFACVSSGNSNAN | GGCTTCCCCGTCCACTACACAGACGTCTCCAACATGAGC |
| | SRGPSFSSGLVPLSL | CGCTTGGCGAGGCAGAGACTGCTGGGCCGATCGTGGAG |
| | RGSHMGPMEIYKT | CGTGCCGGTCATCCGCCACCTCTTCGCTCCGCTGAAGGA |
| | VSAWKRQPVRVLS | ATATTTTGCTTGTGTGtctagcggcaatagtaacgctaacagccgcgggccga |
| | LFRNIDKVLKSLGF | gcttcagcagcggcctggtgccgttaagcttgcgcggcagccatATGggccctatggaga |
| | LESGSGSGGGTLKY | tatacaagacagtgtctgcatggaagagacagccagtgcgggtactgagcctcttcagaaac |
| | VEDVTNVVRRDVE | atcgacaaggtactaaagagtttgggcttcttGGAAAGCGGTTCTGGTTCTG |
| | KWGPFDLVYGSTQ | GGGGAGGAACGCTGAAGTACGTGGAAGATGTCACAAAT |
| | PLGSSCDRCPGWY | GTCGTGAGGAGAGACGTGGAGAAATGGGGCCCCTTTGA |
| | MFQFHRILQYALPR | CCTGGTGTACGGCTCGACGCAGCCCCTAGGCAGCTCTTG |
| | QESQRPFFWIFMDN | TGATCGCTGTCCCGGCTGGTACATGTTCCAGTTCCACCG |
| | LLLTEDDQETTTRF | GATCCTGCAGTATGCGCTGCCTCGCCAGGAGAGTCAGCG |
| | LQTEAVTLQDVRG | GCCCTTCTTCTGGATATTCATGGACAATCTGCTGCTGACT |
| | RDYQNAMRVWSNI | GAGGATGACCAAGAGACAACTACCCGCTTCCTTCAGACA |
| | PGLKSKHAPLTPKE | GAGGCTGTGACCCTCCAGGATGTCCGTGGCAGAGACTAC |
| | EEYLQAQVRSRSKL | CAGAATGCTATGCGGGTGTGGAGCAACATTCCAGGGCTG |
| | DAPKVDLLVKNCL | AAGAGCAAGCATGCGCCCCTGACCCCAAAGGAAGAAGA |
| | LPLREYFKYFSQNS | GTATCTGCAAGCCCAAGTCAGAAGCAGGAGCAAGCTGG |
| | LPLGGPSSGAPPPSG | ACGCCCCGAAAGTTGACCTCCTGGTGAAGAACTGCCTTC |
| | GSPAGSPTSTEEGTS | TCCCGCTGAGAGAGTACTTCAAGTATTTTTCTCAAAACT |
| | ESATPESGPGTSTEP | CACTTCCTCTTggagggccgagctctggcgcacccccaccaagtggagggtctcc |
| | SEGSAPGSPAGSPTS | tgccgggtccccaacatctactgaagaaggcaccagcgaatccgcaacgcccgagtcaggc |
| | TEEGTSTEPSEGSAP | cctggtacctccacagaaccatctgaaggtagtgcgcctggttccccagctggaagccctact |
| | GTSTEPSESRERMK | tccaccgaagaaggcacgtcaaccgaaccaagtgaaggatctgcccctgggaccagcactg |
| | RIEEGIKELGSQIL | aaccatctgagAGCCGCGAGAGAATGAAGCGGATCGAAGAGG |
| | KEHPVENTQLQNE | GCATCAAAGAGCTGGGCAGCCAGATCCTGAAAGAACAC |
| | KLYLYYLQNGRD | CCCGTGGAAAACACCCAGCTGCAGAACGAGAAGCTGTA |
| | MYVDQELDINRLS | CCTGTACTACCTGCAGAATGGGCGGGATATGTACGTGGA |
| | DYDVDAIVPQSFL | CCAGGAACTGGACATCAACCGGCTGTCCGACTACGATGT |
| | KDDSIDNKVLTRS | GGACGCTATCGTGCCTCAGAGCTTTCTGAAGGACGACTC |
| | DKNRGKSDNVPSE | CATCGATAACAAAGTGCTGACTCGGAGCGACAAGAACC |
| | EVVKKMKNYWRQ | GGGGCAAGAGCGACAACGTGCCCTCCGAAGAGGTCGTG |
| | LLNAKLITQRKFD | AAGAAGATGAAGAACTACTGGCGCCAGCTGCTGAATGC |
| | NLTKAERGGLSEL | CAAGCTGATTACCCAGAGGAAGTTCGACAATCTGACCAA |
| | DKAGFIKRQLVET | GGCCGAGAGAGGCGGCCTGAGCGAACTGGATAAGGCCG |
| | RQITKHVAQILDSR | GCTTCATCAAGAGACAGCTGGTGGAAACCCGGCAGATC |
| | MNTKYDENDKLIR | ACAAAGCACGTGGCACAGATCCTGGACTCCCGGATGAA |
| | EVKVITLKSKLVSD | CACTAAGTACGACGAGAACGACAAACTGATCCGGGAAG |
| | FRKDFQFYKVREI | TGAAAGTGATCACCCTGAAGTCCAAGCTGGTGTCCGATT |
| | NNYHHAHDAYLN | TCCGGAAGGATTTCCAGTTTTACAAAGTGCGCGAGATCA |
| | AVVGTALIKKYPK | ACAACTACCACCACGCCCACGACGCCTACCTGAACGCCG |
| | LESEFVYGDYKVY | TCGTGGGAACCGCCCTGATCAAAAAGTACCCTAAGCTGG |
| | DVRKMIAKSEQEI | AAAGCGAGTTCGTGTACGGCGACTACAAGGTGTACGAC |
| | GKATAKYFFYSNI | GTGCGGAAGATGATCGCCAAGAGCGAGCAGGAAATCGG |
| | MNFFKTEITLANG | CAAGGCTACCGCCAAGTACTTCTTCTACAGCAACATCAT |
| | EIRKRPLIETNGET | GAACTTTTTCAAGACCGAGATTACCCTGGCCAACGGCGA |
| | GEIVWDKGRDFAT | GATCCGGAAGCGGCCTCTGATCGAGACAAACGGCGAAA |
| | VRKVLSMPQVNIV | CAGGCGAGATCGTGTGGGATAAGGGCCGGGACTTTGCC |
| | KKTEVQTGGFSKE | ACCGTGCGGAAAGTGCTGTCTATGCCCCAAGTGAATATC |
| | SILPKRNSDKLIAR | GTGAAAAAGACCGAGGTGCAGACAGGCGGCTTCAGCAA |
| | KKDWDPKKYGGF | AGAGTCTATCCTGCCCAAGAGGAACAGCGACAAGCTGA |
| | DSPTVAYSVLVVA | TCGCCAGAAAGAAGGACTGGGACCCTAAGAAGTACGGC |
| | KVEKGKSKKLKSV | GGCTTCGACAGCCCCACCGTGGCCTATTCTGTGCTGGTG |
| | KELLGITIMERSSF | GTGGCCAAAGTGGAAAAGGGCAAGTCCAAGAAACTGAA |
| | EKNPIDFLEAKGY | GAGTGTGAAAGAGCTGCTGGGGATCACCATCATGGAAA |
| | KEVKKDLIIKLPK | GAAGCAGCTTCGAGAAGAATCCCATCGACTTTCTGGAAG |
| | YSLFELENGRKRM | CCAAGGGCTACAAAGAAGTGAAAAAGGACCTGATCATC |
| | LASAGELQKGNEL | AAGCTGCCTAAGTACTCCCTGTTCGAGCTGGAAAACGGC |
| | ALPSKYVNFLYLA | CGGAAGAGAATGCTGGCCTCTGCCGGCGAACTGCAGAA |
| | SHYEKLKGSPEDN | GGGAAACGAACTGGCCCTGCCCTCCAAATATGTGAACTT |
| | EQKQLFVEQHKH | CCTGTACCTGGCCAGCCACTATGAGAAGCTGAAGGGCTC |
| | YLDEIIEQISEFSKR | CCCCGAGGATAATGAGCAGAAACAGCTGTTTGTGGAAC |
| | VILADANLDKVLS | AGCACAAACACTACCTGGACGAGATCATCGAGCAGATC |
| | AYNKHRDKPIREQ | AGCGAGTTCTCCAAGAGAGTGATCCTGGCCGACGCTAAT |
| | AENIIHLFTLTNLG | CTGGACAAGGTGCTGAGCGCCTACAACAAGCACAGAGA |
| | APAAFKYFDTTIDR | CAAGCCTATCAGAGAGCAGGCCGAGAATATCATCCACCT |
| | KRYTSTKEVLDAT | GTTTACCCTGACCAATCTGGGAGCCCCTGCCGCCTTCAA |
| | LIHQSITGLYETRI | GTACTTTGACACCACCATCGACCGGAAGAGGTACACCAG |
| | DLSQLGGDYPYDV | CACCAAAGAGGTGCTGGACGCCACCCTGATCCACCAGA |
| | PDYASLGSGSPKKK | GCATCACCGGCCTGTACGAGACACGGATCGACCTGTCTC |
| | RKVEDPKKKRKVD | AGCTGGGAGGCGACTATCCCTATGACGTGCCCGATTATG |
| | GSGSETPGTSESATP | CCAGCCTGGGCAGCGGCTCCCCCAAGAAAAAACGCAAG |
| | ESRTLVTFKDVFVD | GTGGAAGATCCTAAGAAAAAGCGGAAAGTGGACGGCagc |
| | FTREEWKLLDTAQ | ggcagcgagacccccggtaccagcgagagcgccacccccgagagcCGGACACT |
| | QIVYRNVMLENYKN | GGTGACCTTCAAGGATGTATTTGTGGACTTCACCAGGGA |
| | LVSLGYQLTKPDVIL | GGAGTGGAAGCTGCTGGACACTGCTCAGCAGATCGTGTA |
| | RLEKGEEP* (SEQ | CAGAAATGTGATGCTGGAGAACTATAAGAACCTGGTTTC |
| | ID NO: 137) | CTTGGGTTATCAGCTTACTAAGCCAGATGTGATCCTCCG |
| | | GTTGGAGAAGGGAGAAGAGCCCTAA (SEQ ID NO: 165) |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| KRAB | QVDRYIASEVCEDSIT | CTGAAGGACCTGGGCATCCAAGTGGACCGCTACATTGCC |
| (FIG. | VGMVRHQGKIMYVG | TCCGAGGTGTGTGAGGACTCCATCACGGTGGGCATGGTG |
| 4B) | DVRSVTQKHIQEWG | CGGCACCAGGGAAAGATCATGTACGTCGGGGACGTCCG |
| | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESRTLVTFKD | CTCAGCTGGGAGGCGACGCCTATCCCTATGACGTGCCCG |
| | VFVDFTREEWKLL | ATTATGCCAGCCTGGGCAGCGGCTCCCCCAAGAAAAAA |
| | DTAQQIVYRNVMLE | CGCAAGGTGGAAGATCCTAAGAAAAAGCGGAAAGTGGA |
| | NYKNLVSLGYQLTK | CGGCagcggcagcgagacccccggtaccagcgagagcgccacccccgagagcCG |
| | PDVILRLEKGEEP | GACACTGGTGACCTTCAAGGATGTATTTGTGGACTTCAC |
| | (SEQ ID NO: 138) | CAGGGAGGAGTGGAAGCTGCTGGACACTGCTCAGCAGA |
| | | TCGTGTACAGAAATGTGATGCTGGAGAACTATAAGAACC |
| | | TGGTTTCCTTGGGTTATCAGCTTACTAAGCCAGATGTGAT |
| | | CCTCCGGTTGGAGAAGGGAGAAGAGCCC (SEQ ID NO: |
| | | 166) |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| EZH2 | QVDRYIASEVCEDSIT | CTGAAGGACCTGGGCATCCAAGTGGACCGCTACATTGCC |
| (FIG. | VGMVRHQGKIMYVG | TCCGAGGTGTGTGAGGACTCCATCACGGTGGGCATGGTG |
| 4B) | DVRSVTQKHIQEWG | CGGCACCAGGGAAAGATCATGTACGTCGGGGACGTCCG |
| | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESMGQTGKK | CTCAGCTGGGAGGCGACGCCTATCCCTATGACGTGCCCG |
| | SEKGPVCWRKRV | ATTATGCCAGCCTGGGCAGCGGCTCCCCCAAGAAAAAA |
| | KSEYMRLRQLKRF | CGCAAGGTGGAAGATCCTAAGAAAAAGCGGAAAGTGGA |
| | RRADEVKSMFSSN | CGGCagcggcagcgagacccccggtaccagcgagagcgccacccccgagagcatgg |
| | RQKILERTEILNQE | gccagactgggaagaaatctgagaagggaccagtttgttggcggaagcgtgtaaaatcaga |
| | WKQRRIQPVHILT | gtacatgcgactgagacagctcaagaggttcagacgagctgatgaagtaaagagtatgtttag |
| | SVSSLRGTRECSVT | ttccaatcgtcagaaaattttggaaagaacggaaatcttaaaccaagaatggaaacagcgaag |
| | SDLDFPTQVIPLKT | gatacagcctgtgcacatcctgacttctgtgagctcattgcgcgggactagggagtgttcggtg |
| | LNAVASVPIMYSW | accagtgacttggattttccaacacaagtcatcccattaaagactctgaatgcagttgcttcagta |
| | SPLQQNFMVEDET | cccataatgtattcttggtctcccctacagcagaattttatggtggaagatgaaactgttttacata |
| | VLHNIPYMGDEVL | acattccttatatgggagatgaagttttagatcaggatggtactttcattgaagaactaataaaaa |
| | DQDGTFIEELIKNY | attatgatgggaaagtacacggggatagagaatgtgggtttataaatgatgaaatttttgtggag |
| | DGKVHGDRECGFI | ttggtgaatgcccttggtcaatataatgatgatgacgatgatgatgatggagacgatcctgaag |
| | NDEIFVELVNALG | aaagagaagaaaagcagaaagatctggaggatcaccgagatgataaagaaagccgcccac |
| | QYNDDDDDDDGD | ctcggaaatttccttctgataaaatttttgaagccatttcctcaatgtttccagataagggcacagc |
| | DPEEREEKQKDLE | agaagaactaaaggaaaaatataaagaactcaccgaacagcagctcccagggcacttcctc |
| | DHRDDKESRPPRK | ctgaatgtacccccaacatagatggaccaaatgctaaatctgttcagagagagcaaagcttac |
| | FPSDKIFEAISSMFP | actcctttcatacgcttttctgtaggcgatgttttaaatatgactgcttcctacatcgtaagtgcaatt |
| | DKGTAEELKEKYK | attcttttcatgcaacacccaacacttataagcggaagaacacagaaacagctctagacaaca |
| | ELTEQQLPGALPP | aaccttgtggaccacagtgttaccagcatttggagggagcaaaggagtttgctgctgctctcac |
| | ECTPNIDGPNAKSV | cgctgagcggataaagaccccaccaaaacgtccaggaggccgcagaagaggacggcttcc |
| | QREQSLHSFHTLF | caataacagtagcaggcccagcacccccaccattaatgtgctggaatcaaaggatacagaca |
| | CRRCFKYDCFLHR | gtgatagggaagcagggactgaaacggggggagagaacaatgataaagaagaagaagag |
| | KCNYSFHATPNTY | aagaaagatgaaacttcgagctcctctgaagcaaattctcggtgtcaaacaccaataaagatg |
| | KRKNTETALDNKP | aagccaaatattgaacctcctgagaatgtggagtggagtggtgctgaagcctcaatgtttagag |
| | CGPQCYQHLEGA | tcctcattggcacttactatgacaatttctgtgccattgctaggttaattgggaccaaaacatgtag |
| | KEFAAALTAERIK | acaggtgtatgagtttagagtcaaagaatctagcatcatagctccagctcccgctgaggatgtg |
| | TPPKRPGGRRRGR | gatactcctccaaggaaaaagaagaggaaacaccggttgtgggctgcacactgcagaaaga |
| | LPNNSSRPSTPTIN | tacagctgaaaaaggacggctcctctaaccatgtttacaactatcaaccctgtgatcatccacg |
| | VLESKDTDSDREA | gcagccttgtgacagttcgtgcccttgtgtgatagcacaaaatttttgtgaaaagttttgtcaatgt |
| | GTETGGENNDKEE | agttcagagtgtcaaaaccgctttccgggatgccgctgcaaagcacagtgcaacaccaagca |
| | EEKKDETSSSSEAN | gtgcccgtgctacctggctgtccgagagtgtgaccctgacctctgtcttacttgtggagccgct |
| | SRCQTPIKMKPNIE | gaccattgggacagtaaaaatgtgtcctgcaagaactgcagtattcagggggctccaaaaa |
| | PPENVEWSGAEAS | gcatctattgctggcaccatctgacgtggcaggctgggggatttttatcaaagatcctgtgcag |
| | MFRVLIGTYYDNF | aaaaatgaattcatctcagaatactgtggagagattatttctcaagatgaagctgacagaagag |
| | CAIARLIGTKTCR | ggaaagtgtatgataaatacatgtgcagctttctgttcaacttgaacaatgattttgtggtggatg |
| | QVYEFRVKESSIIA | caacccgcaagggtaacaaaattcgttttgcaaatcattcggtaaatccaaactgctatgcaaa |
| | PAPAEDVDTPPRK | agttatgatggttaacggtgatcacaggataggtatttttgccaagagagccatccagactggc |
| | KKRKHRLWAAHC | gaagagctgttttttgattacagatacagccaggctgatgccctgaagtatgtcggcatcgaaa |
| | RKIQLKKDGSSNH | gagaaatggaaatcccttga (SEQ ID NO: 167) |
| | VYNYQPCDHPRQP | |
| | CDSSCPCVIAQNFC | |
| | EKFCQCSSECQNR | |
| | FPGCRCKAQCNTK | |
| | QCPCYLAVRECDP | |
| | DLCLTCGAADHW | |
| | DSKNVSCKNCSIQ | |
| | RGSKKHLLLAPSD | |
| | VAGWGIFIKDPVQ | |
| | KNEFISEYCGEIISQ | |
| | DEADRRGKVYDK | |
| | YMCSFLFNLNNDF | |
| | VVDATRKGNKIRF | |
| | ANHSVNPNCYAKV | |
| | MMVNGDHRIGIFA | |
| | KRAIQTGEELFFD | |
| | YRYSQADALKYVG | |
| | IEREMEIP* (SEQ | |
| | ID NO: 139) | |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| G9A | QVDRYIASEVCEDSIT | CTGAAGGACCTGGGCATCCAAGTGGACCGCTACATTGCC |
| (FIG. | VGMVRHQGKIMYVG | TCCGAGGTGTGTGAGGACTCCATCACGGTGGGCATGGTG |
| 4B) | DVRSVTQKHIQEWG | CGGCACCAGGGAAAGATCATGTACGTCGGGGACGTCCG |
| | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESMRGLPRG | CTCAGCTGGGAGGCGACGCCTATCCCTATGACGTGCCCG |
| | RGLMRARGRGRA | ATTATGCCAGCCTGGGCAGCGGCTCCCCCAAGAAAAAA |
| | APPGSRGRGRGGP | CGCAAGGTGGAAGATCCTAAGAAAAAGCGGAAAGTGGA |
| | HRGRGRPRSLLSL | CGGCagcggcagcgagacccccggtaccagcgagagcgccacccccgagagcatgc |
| | PRAQASWTPQLST | ggggtctaccgagagggagggggttgatgcgggcccgggggaggggtcgtgcggcccct |
| | GLTSPPVPCLPSQG | ccgggcagccgaggccgcggaaggggggggccccacagaggaagaggtaggccccgg |
| | EAPAEMGALLLEK | agcctactctctcttcccagggcccaggcatcctggaccccccaactctctactgggctgacc |
| | ETRGATERVHGSL | agccctcctgtcccttgtctcccctcccagggggaggcccccgctgagatgggggcgctgct |
| | GDTPRSEETLPKA | gctggagaaggaaaccagaggagccaccgagagagttcatggctctttgggggacacccct |
| | TPDSLEPAGPSSPA | cgtagtgaagaaaccctgcccaaggccacccccgactccctggagcctgctggcccctcatc |
| | SVTVTVGDEGADT | tccagcctctgtcactgtcactgttggtgatgagggggctgacacccctgtaggggctacacc |
| | PVGATPLIGDESEN | actcattggggatgaatctgagaatcttgagggagatggggacctccgtgggggccggatcc |
| | LEGDGDLRGGRIL | tgctgggccatgccacaaagtcattcccctcttcccccagcaaggggggttcctgtcctagcc |
| | LGHATKSFPSSPSK | gggccaagatgtcaatgacaggggcgggaaaatcacctccatctgtccagagtttggctatg |
| | GGSCPSRAKMSMT | aggctactgagtatgccaggagcccagggagctgcagcagcagggtctgaaccccctcca |
| | GAGKSPPSVQSLA | gccaccacgagcccagagggacagcccaaggtccaccgagcccgcaaaaccatgtccaa |
| | MRLLSMPGAQGA | accaggaaatggacagcccccggtccctgagaagcggccccctgaaatacagcatttccgc |
| | AAAGSEPPPATTSP | atgagtgatgatgtccactcactgggaaaggtgacctcagatctggccaaaaggaggaagct |
| | EGQPKVHRARKT | gaactcaggaggtggcctgtcagaggagttaggttctgcccggcgttcaggagaagtgaccc |
| | MSKPGNGQPPVPE | tgacgaaaggggaccccgggtccctggaggagtgggagacggtggtgggtgatgacttca |
| | KRPPEIQHFRMSD | gtctctactatgattcctactctgtggatgagcgcgtggactccgacagcaagtctgaagttgaa |
| | DVHSLGKVTSDLA | gctctaactgaacaactaagtgaagaggaggaggaggaagaggaggaagaagaagaaga |
| | KRRKLNSGGGLSE | ggaagaggaggaggaagaggaagaagaagaggaagatgaggagtcagggaatcagtca |
| | ELGSARRSGEVTL | gataggagtggttccagtggccggcgcaaggccaagaagaaatggcgaaaagacagccca |
| | TKGDPGSLEEWET | tgggtgaagccgtctcggaaacggcgcaagcgggagcctccgcgggccaaggagccacg |
| | VVGDDFSLYYDSY | aggggtgtccaatgacacatcttcgctggagacagagcgagggtttgaggagttgcccctgt |
| | SVDERVDSDSKSEV | gcagctgccgcatggaggcacccaagattgaccgcatcagcgagagggcggggcacaag |
| | EALTEQLSEEEEEE | tgcatggccactgagagtgtggacggagagctgtcaggctgcaatgccgccatcctcaagc |
| | EEEEEEEEEEEEEE | gggagaccatgaggccatccagccgtgtggccctgatggtgctctgtgagacccaccgcgc |
| | EEEEDEESGNQSD | ccgcatggtcaaacaccactgctgcccgggctgcggctacttctgcacggcgggcaccttcc |
| | RSGSSGRRKAKKK | tggagtgccaccctgacttccgtgtggcccaccgcttccacaaggcctgtgtgtctcagctga |
| | WRKDSPWVKPSR | atgggatggtcttctgtccccactgtggggaggatgcttctgaagctcaagaggtgaccatcc |
| | KRRKREPPRAKEP | cccggggtgacggggtgaccccaccggccggcactgcagctcctgcacccccacccctgt |
| | RGVSNDTSSLETER | cccaggatgtccccgggagagcagacacttctcagcccagtgcccggatgcgagggcatg |
| | GFEELPLCSCRME | gggaaccccggcgcccgccctgcgatcccctggctgacaccattgacagctcagggccctc |
| | APKIDRISERAGHK | cctgaccctgcccaatgggggctgcctttcagccgtggggctgccactggggccaggccgg |
| | CMATESVDGELSG | gaggccctggaaaaggccctggtcatccaggagtcagagaggcggaagaagctccgtttcc |
| | CNAAILKRETMRP | accctcggcagttgtacctgtccgtgaagcagggcgagctgcagaaggtgatcctgatgctgt |
| | SSRVALMVLCETH | tggacaacctggaccccaacttccagagcgaccagcagagcaagcgcacgcccctgcatg |
| | RARMVKHHCCPG | cagccgcccagaagggctccgtggagatctgccatgtgctgctgcaggctggagccaacat |
| | CGYFCTAGTFLEC | aaatgcagtggacaaacagcagcggacgccactgatggaggccgtggtgaacaaccacct |
| | HPDFRVAHRFHKA | ggaggtagcccgttacatggtgcagcgtggtggctgtgtctatagcaaggaggaggacggtt |
| | CVSQLNGMVFCPH | ccacctgcctccaccacgcagccaaaatcgggaacttggagatggtcagcctgctgctgagc |
| | CGEDASEAQEVTIP | acaggacaggtggacgtcaacgcccaggacagtggggggtggacgcccatcatctgggct |
| | RGDGVTPPAGTAA | gcagagcacaagcacatcgaggtgatccgcatgctactgacgcggggcgccgacgtcacc |
| | PAPPPLSQDVPGR | ctcactgacaacgaggagaacatctgcctgcactgggcctccttcacgggcagcgccgccat |
| | ADTSQPSARMRGH | cgccgaagtccttctgaatgcgcgctgtgacctccatgctgtcaactaccatggggacacccc |
| | GEPRRPPCDPLAD | cctgcacatcgcagctcgggagagctaccatgactgcgtgctgttattcctgtcacgtggggc |
| | TIDSSGPSLTLPNG | caaccctgagctgcggaacaaagagggggacacagcatgggacctgactcccgagcgctc |
| | GCLSAVGLPLGPG | cgacgtgtggtttgcgcttcaactcaaccgcaagctccgacttggggtgggaaatcgggccat |
| | REALEKALVIQESE | ccgcacagagaagatcatctgccgggacgtggctcggggctatgagaacgtgcccattccct |
| | RRKKLRFHPRQLY | gtgtcaacggtgtggatggggagccctgccctgaggattacaagtacatctcagagaactgc |
| | LSVKQGELQKVIL | gagacgtccaccatgaacatcgatcgcaacatcacccacctgcagcactgcacgtgtgtgga |
| | MLLDNLDPNFQSD | cgactgctctagctccaactgcctgtgcggccagctcagcatccggtgctggtatgacaagga |
| | QQSKRTPLHAAAQ | tgggcgattgctccaggaatttaacaagattgagcctccgctgattttcgagtgtaaccaggcg |
| | KGSVEICHVLLQA | tgctcatgctggagaaactgcaagaaccgggtcgtacagagtggcatcaaggtgcggctaca |
| | GANINAVDKQQRT | gctctaccgaacagccaagatgggctggggggtccgcgccctgcagaccatcccacaggg |
| | PLMEAVVNNHLEV | gaccttcatctgcgagtatgtcggggagctgatctctgatgctgaggctgatgtgagagagga |
| | ARYMVQRGGCVY | tgattcttacctcttcgacttagacaacaaggatggagaggtgtactgcatagatgcccgttact |
| | SKEEDGSTCLHHA | atggcaacatcagccgcttcatcaaccacctgtgtgaccccaacatcattcccgtccgggtctt |
| | AKIGNLEMVSLLL | catgctgcaccaagacctgcgatttccacgcatcgccttcttcagttcccgagacatccggact |
| | STGQVDVNAQDSG | ggggaggagctagggtttgactatggcgaccgcttctgggacatcaaaagcaaatatttcacc |
| | GWTPIIWAAEHKH | tgccaatgtggctctgagaagtgcaagcactcagccgaagccattgccctggagcagagcc |
| | IEVIRMLLTRGAD | gtctggcccgcctggacccacaccctgagctgctgcccgagctcggctccctgccccctgtc |
| | VTLTDNEENICLH | aacacatga (SEQ ID NO: 168) |
| | WASFTGSAAIAEV | |
| | LLNARCDLHAVNY | |
| | HGDTPLHIAARES | |
| | YHDCVLLFLSRGA | |
| | NPELRNKEGDTA | |
| | WDLTPERSDVWFA | |
| | LQLNRKLRLGVGN | |
| | RAIRTEKIICRDVA | |
| | RGYENVPIPCVNG | |
| | VDGEPCPEDYKYIS | |
| | ENCETSTMNIDRNI | |
| | THLQHCTCVDDCS | |
| | SSNCLCGQLSIRC | |
| | WYDKDGRLLQEF | |
| | NKIEPPLIFECNQA | |
| | CSCWRNCKNRVV | |
| | QSGIKVRLQLYRT | |
| | AKMGWGVRALQT | |
| | IPQGTFICEYVGEL | |
| | ISDAEADVREDDSY | |
| | LFDLDNKDGEVYC | |
| | IDARYYGNISRFIN | |
| | HLCDPNIIPVRVFM | |
| | LHQDLRFPRIAFFS | |
| | SRDIRTGEELGFDY | |
| | GDRFWDIKSKYFT | |
| | CQCGSEKCKHSAE | |
| | AIALEQSRLARLDP | |
| | HPELLPELGSLPPV | |
| | NT* (SEQ ID NO: | |
| | 140) | |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| LSD1 | QVDRYIASEVCEDSIT | CTGAAGGACCTGGGCATCCAAGTGGACCGCTACATTGCC |
| (FIG. | VGMVRHQGKIMYVG | TCCGAGGTGTGTGAGGACTCCATCACGGTGGGCATGGTG |
| 4B) | DVRSVTQKHIQEWG | CGGCACCAGGGAAAGATCATGTACGTCGGGGACGTCCG |
| | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESMLSGKKA | CTCAGCTGGGAGGCGACGCCTATCCCTATGACGTGCCCG |
| | AAAAAAAAAAATG | ATTATGCCAGCCTGGGCAGCGGCTCCCCCAAGAAAAAA |
| | TEAGPGTAGGSEN | CGCAAGGTGGAAGATCCTAAGAAAAAGCGGAAAGTGGA |
| | GSEVAAQPAGLSG | CGGCagcggcagcgagacccccggtaccagcgagagcgccacccccgagagcatgtt |
| | PAEVGPGAVGERT | atctgggaagaaggcggcagccgcggcggcggcggctgcagcggcagcaaccgggacg |
| | PRKKEPPRASPPG | gaggctggccctgggacagcaggcggctccgagaacgggtctgaggtggccgcgcagcc |
| | GLAEPPGSAGPQA | cgcgggcctgtcgggcccagccgaggtcgggccgggggcggtgggggagcgcacaccc |
| | GPTVVPGSATPME | cgcaagaaagagcctccgcgggcctcgccccccgggggcctggcggaaccgccggggtc |
| | TGIAETPEGRRTSR | cgcagggcctcaggccggccctactgtcgtgcctgggtctgcgacccccatggaaactgga |
| | RKRAKVEYREMD | atagcagagactccggaggggcgtcggaccagccggcgcaagcgggcgaaggtagagta |
| | ESLANLSEDEYYSE | cagagagatggatgaaagcttggccaacctctcagaagatgagtattattcagaagaagaga |
| | EERNAKAEKEKKL | gaaatgccaaagcagagaaggaaaagaagcttcccccaccaccccctcaagccccacctg |
| | PPPPPQAPPEEENE | aggaagaaaatgaaagtgagcctgaagaaccatcggggcaagcaggaggacttcaagacg |
| | SEPEEPSGQAGGL | acagttctggagggtatggagacggccaagcatcaggtgtggagggcgcagctttccagag |
| | QDDSSGGYGDGQA | ccgacttcctcatgaccggatgacttctcaagaagcagcctgttttccagatattatcagtggac |
| | SGVEGAAFQSRLP | cacaacagacccagaaggtttttttttcattagaaaccgcacactgcagttgtggttggataat |
| | HDRMTSQEAACFP | ccaaagattcagctgacatttgaggctactctccaacaattagaagcaccttataacagtgatac |
| | DIISGPQQTQKVFL | tgtgcttgtccaccgagttcacagttatttagagcgtcatggtcttatcaacttcggcatctataag |
| | FIRNRTLQLWLDN | aggataaaacccctaccaactaaaaagacaggaaaggtaattattataggctctggggtctca |
| | PKIQLTFEATLQQ | ggcttggcagcagctcgacagttacaaagttttggaatggatgtcacacttttggaagccagg |
| | LEAPYNSDTVLVH | gatcgtgtgggggacgagttgccacatttcgcaaaggaaactatgtagctgatcttggagcc |
| | RVHSYLERHGLIN | atggtggtaacaggtcttggagggaatcctatggctgtggtcagcaaacaagtaaatatggaa |
| | FGIYKRIKPLPTKK | ctggccaagatcaagcaaaaatgcccactttatgaagccaacggacaagctgacactgtcaa |
| | TGKVIIIGSGVSGL | ggttcctaaagagaaagatgaaatggtagagcaagagtttaaccggttgctagaagctacatc |
| | AAARQLQSFGMD | ttaccttagtcatcaactagacttcaatgtcctcaataataagcctgtgtcccttggccaggcatt |
| | VTLLEARDRVGGR | ggaagttgtcattcagttacaagagaagcatgtcaaagatgagcagattgaacattggaagaa |
| | VATFRKGNYVADL | gatagtgaaaactcaggaagaattgaaagaacttcttaataagatggtaaatttgaaagagaaa |
| | GAMVVTGLGGNP | attaaagaactccatcagcaatacaaagaagcatctgaagtaaagccacccagagatattact |
| | MAVVSKQVNMEL | gccgagttcttagtgaaaagcaaacacagggatctgaccgccctatgcaaggaatatgatga |
| | AKIKQKCPLYEAN | attagctgaaacacaaggaaagctagaagaaaaacttcaggagttggaagcgaatcccccaa |
| | GQADTVKVPKEK | gtgatgtatatctctcatcaagagacagacaaatacttgattggcattttgcaaatcttgaatttgc |
| | DEMVEQEFNRLLE | taatgccacacctctctcaactctctcccttaagcactgggatcaggatgatgactttgagttcac |
| | ATSYLSHQLDFNV | tggcagccacctgacagtaaggaatggctactcgtgtgtgcctgtggctttagcagaaggcct |
| | LNNKPVSLGQALE | agacattaaactgaatacagcagtgcgacaggttcgctacacggcttcaggatgtgaagtgat |
| | VVIQLQEKHVKDE | agctgtgaatacccgctccacgagtcaaacctttatttataaatgcgacgcagttctctgtaccct |
| | QIEHWKKIVKTQE | tcccctgggtgtgctgaagcagcagccaccagccgttcagtttgtgccacctctccctgagtg |
| | ELKELLNKMVNL | gaaaacatctgcagtccaaaggatgggatttggcaaccttaacaaggtggtgttgtgttttgatc |
| | KEKIKELHQQYKE | gggtgttctgggatccaagtgtcaatttgttcgggcatgttggcagtacgactgccagcaggg |
| | ASEVKPPRDITAEF | gtgagctcttcctcttctggaacctctataaagctccaatactgttggcactagtggcaggagaa |
| | LVKSKHRDLTALC | gctgctggtatcatggaaaacataagtgacgatgtgattgttggccgatgcctggccattctca |
| | KEYDELAETQGKL | aagggatttttggtagcagtgcagtacctcagcccaaagaaactgtggtgtctcgttggcgtgc |
| | EEKLQELEANPPS | tgatccctgggctcggggctcttattcctatgttgctgcaggatcatctggaaatgactatgattt |
| | DVYLSSRDRQILD | aatggctcagccaatcactcctggcccctcgattccaggtgccccacagccgattccacgact |
| | WHFANLEFANATP | cttctttgcgggagaacatacgatccgtaactacccagccacagtgcatggtgctctgctgagt |
| | LSTLSLKHWDQDD | gggctgcgagaagcgggaagaattgcagaccagtttttgggggccatgtatacgctgcctcg |
| | DFEFTGSHLTVRN | ccaggccacaccaggtgttcctgcacagcagtccccaagcatgtga (SEQ ID NO: |
| | GYSCVPVALAEGL | 169) |
| | DIKLNTAVRQVRY | |
| | TASGCEVIAVNTRS | |
| | TSQTFIYKCDAVL | |
| | CTLPLGVLKQQPP | |
| | AVQFVPPLPEWKT | |
| | SAVQRMGFGNLN | |
| | KVVLCFDRVFWDP | |
| | SVNLFGHVGSTTA | |
| | SRGELFLFWNLYK | |
| | APILLALVAGEAA | |
| | GIMENISDDVIVGR | |
| | CLAILKGIFGSSAV | |
| | PQPKETVVSRWRA | |
| | DPWARGSYSYVAA | |
| | GSSGNDYDLMAQP | |
| | ITPGPSIPGAPQPIP | |
| | RLFFAGEHTIRNYP | |
| | ATVHGALLSGLRE | |
| | AGRIADQFLGAMY | |
| | TLPRQATPGVPAQ | |
| | QSPSM (SEQ ID NO: | |
| | 141) | |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| HP1 | QVDRYIASEVCEDSIT | CTGAAGGACCTGGGCATCCAAGTGGACCGCTACATTGCC |
| (FIG. | VGMVRHQGKIMYVG | TCCGAGGTGTGTGAGGACTCCATCACGGTGGGCATGGTG |
| 4B) | DVRSVTQKHIQEWG | CGGCACCAGGGAAAGATCATGTACGTCGGGGACGTCCG |
| | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESMGKKTKR | CTCAGCTGGGAGGCGACGCCTATCCCTATGACGTGCCCG |
| | TADSSSSEDEEEYV | ATTATGCCAGCCTGGGCAGCGGCTCCCCCAAGAAAAAA |
| | VEKVLDRRVVKG | CGCAAGGTGGAAGATCCTAAGAAAAAGCGGAAAGTGGA |
| | QVEYLLKWKGFSE | CGGCagcggcagcgagacccccggtaccagcgagagcgccacccccgagagcatgg |
| | EHNTWEPEKNLDC | gaaagaaaaccaagcggacagctgacagttcttcttcagaggatgaggaggagtatgttgtg |
| | PELISEFMKKYKK | gagaaggtgctagacaggcgcgtggttaagggacaagtggaatatctactgaagtggaaag |
| | MKEGENNKPREKS | gcttttctgaggagcacaatacttgggaacctgagaaaaacttggattgccctgagctaatttct |
| | ESNKRKSNFSNSAD | gaatttatgaaaaagtataagaagatgaaggagggtgaaaataataaacccagggagaagtc |
| | DIKSKKKREQSNDI | agaaagtaacaagaggaaatccaatttctcaaacagtgccgatgacatcaaatctaaaaaaaa |
| | ARGFERGLEPEKII | gagagagcagagcaatgatatcgctcggggctttgagagaggactggaaccagaaaagatc |
| | GATDSCGDLMFL | attggggcaacagattcctgtggtgatttaatgttcctaatgaaatggaaagacacagatgaag |
| | MKWKDTDEADLV | ctgacctggttcttgcaaaagaagctaatgtgaaatgtccacaaattgtgatagcattttatgaag |
| | LAKEANVKCPQIVI | agagactgacatggcatgcatatcctgaggatgcggaaaacaaagagaaagaaacagcaaa |
| | AFYEERLTWHAYP | gagctaa (SEQ ID NO: 170) |
| | EDAENKEKETAKS | |
| | (SEQ ID NO: 142) | |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| FOG1 | QVDRYIASEVCEDSIT | CTGAAGGACCTGGGCATCCAAGTGGACCGCTACATTGCC |
| (FIG. | VGMVRHQGKIMYVG | TCCGAGGTGTGTGAGGACTCCATCACGGTGGGCATGGTG |
| 4B) | DVRSVTQKHIQEWG | CGGCACCAGGGAAAGATCATGTACGTCGGGGACGTCCG |
| | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccggggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESMDAGPTV | CTCAGCTGGGAGGCGACGCCTATCCCTATGACGTGCCCG |
| | QGGPTRSSRAARA | ATTATGCCAGCCTGGGCAGCGGCTCCCCCAAGAAAAAA |
| | GGGLSPGAGGSR | CGCAAGGTGGAAGATCCTAAGAAAAAGCGGAAAGTGGA |
| | WERDTGSAALLW | CGGCagcggcagcgagacccccggtaccagcgagagcgccacccccgagagcatgg |
| | QEWNPWAWWRP | acgctgggcctacagtacagggagggcccactcgcagctccagggctgccagagcaggtg |
| | RVTAGSLGDMEAR | ggggcctgtcccctggtgctggggggagcaggtgggaaagagacacggggtccgcggcc |
| | EEVQLVGASHME | cttctctggcaagagtggaatccgtgggcgtggtggaggccaagggtcacggcaggttccct |
| | QKATAPEAPSPPSA | cggagacatggaggccagagaggaggtgcagttggtgggtgccagccacatggagcaaa |
| | DVNSPPPLPPPTSP | aggccacggcacctgaagccccgagccctcccagcgcagatgttaactcacccccaccgct |
| | GGPKELEGQEPEP | gccgccccccacatccccaggaggccccaaggagctggaaggacaggaaccagaaccca |
| | RPTEEEPGSPWSG | ggcccacggaggaagagccgggcagtccctggagcgggccagacgagctggagccggt |
| | PDELEPVVQDGQR | ggtgcaggatgggcagaggcgcatacgggcccgactcagcctcgccacgggcctgtcctg |
| | RIRARLSLATGLS | gggcccgttccatgggagtgtccagaccagagcctcatcccccaggcaggcggagccgag |
| | WGPFHGSVQTRAS | cccagccctgaccctgctgctggtggacgaggcctgctggctgaggacgctgccccaggcc |
| | SPRQAEPSPALTLL | ctgactgaggccgaggccaacacagagatccacaggaaggatgacgcactctggtgcagg |
| | LVDEACWLRTLPQ | gtcaccaagccggtgcctgcggggggactcctgagcgtgctcctcacggccgagccccac |
| | ALTEAEANTEIHR | agcacccccggccaccctgtgaagaaggagccagcagagcccacgtgcccggcccctgc |
| | KDDALWCRVTKP | acacgacctccagctcctgccccagcaggccgggatggcctccatccttgccaccgcagtg |
| | VPAGGLLSVLLTA | atcaacaaagacgtcttcccctgcaaggactgtggcatctggtaccgcagcgagcgcaacct |
| | EPHSTPGHPVKKE | gcaggcgcacctgctctactactgcgccagccgccagggcaccggctccccggccgcagc |
| | PAEPTCPAPAHDL | cgccacagacgagaagcccaaagagacctaccccaacgagcgcgtctgccccttccccca |
| | QLLPQQAGMASIL | gtgccgcaagagctgccccagcgccagctccctggagatccacatgcgcagccacagcgg |
| | ATAVINKDVFPCK | agagcggcccttcgtgtgcctgatctgcctgtcggccttcaccaccaaggccaactgcgagc |
| | DCGIWYRSERNLQ | ggcacctcaaggtgcacacggacacgctgagcggtgtctgccacagctgtggcttcatctcc |
| | AHLLYYCASRQGT | accacaagggacatcctctacagccacttggtcaccaaccacatggtctgccagcctggctcc |
| | GSPAAAATDEKPK | aagggtgagatctactcgccaggggccggacacccagcaaccaagctgcccccagacagt |
| | ETYPNERVCPFPQ | ctgggcagcttccagcagcagcacacggccctgcaaggccccctggcctccgcggacctg |
| | CRKSCPSASSLEIH | ggcctggcgcccaccccatcgccaggactggacagaaaggccctggccgaggccaccaa |
| | MRSHSGERPFVCL | cggagaggccagagcggagcctctggcccagaatggaggcagcagcgagcccccggcg |
| | ICLSAFTTKANCER | gcccccaggagcatcaaggtggaggcggtggaggagccggaggcggcccccatcctggg |
| | HLKVHTDTLSGVC | ccccggagagcctgggccccaggccccgtcgcggacgccgtcgccgcgcagccccgccc |
| | HSCGFISTTRDILY | cggccagggtcaaggccgagctgtccagccccacgccgggctccagcccggtgcccggc |
| | SHLVTNHMVCQP | gagctgggcctggccggggccctgttccttccgcagtacgtgttcgggcccgacgcggcgc |
| | GSKGEIYSPGAGH | cccccgcctcggagatcctggccaagatgtccgagctggtgcacagccggctgcagcagg |
| | PATKLPPDSLGSFQ | gcgcgggcgcgggcgccggcggcgcgcagaccgggctcttccccggggcccccaaggg |
| | QQHTALQGPLASA | cgctacgtgcttcgagtgcgagatcaccttcagcaacgtcaacaactactacgtgcacaagcg |
| | DLGLAPTPSPGLD | cctctactgttcaggccgccgtgcgcccgaggacgcgcctgccgcgcgcaggcccaaggc |
| | RKALAEATNGEAR | gccccccggcccggcccgcgcgccccccggccagcccgccgaacccgacgcgccgcgc |
| | AEPLAQNGGSSEP | tcgtccccgggccccggagcgcgcgaggagggggctgggggcgcggccacgcccgag |
| | PAAPRSIKVEAVEE | gacggcgcgggcggccggggcagcgagggcagccagagcccgggtagctccgtggac |
| | PEAAPILGPGEPGP | gacgcggaggacgaccccagccgcacgctgtgcgaggcctgcaacatccgcttcagccgc |
| | QAPSRTPSPRSPAP | cacgagacctacaccgtgcacaagcggtactactgcgcctcgcgccacgacccgccgccg |
| | ARVKAELSSPTPGS | cgccgaccggccgcgcccccgggaccccctgggccggccgcgcccccggccccctctcc |
| | SPVPGELGLAGAL | cgccgcgcctgtgcgcacgcgcagacgccgcaagctctacgagctgcacgcggccggcg |
| | FLPQYVFGPDAAP | ccccgccccccccgccgcccggccacgcccccgcgcccgagtcgccgcggcccggaag |
| | PASEILAKMSELV | cggaagcggaagcggccccggcctcgcccctgcgcgctcgcccggccccgcggccgac |
| | HSRLQQGAGAGA | ggccccatcgacctgagcaagaagccgcggcgcccgctccccggagccccggcaccggc |
| | GGAQTGLFPGAPK | gctggccgactaccacgagtgcacggcctgccgcgtgagcttccacagcctcgaggcctac |
| | GATCFECEITFSNV | ctggcgcacaagaagtactcgtgccccgctgcgccaccgcccggcgcgctcggcctgccc |
| | NNYYVHKRLYCSG | gccgccgcctgcccctactgccccccgaacggcccggtgcgcggggacctgctggagcat |
| | RRAPEDAPAARRP | ttccgcctggcgcacggcctgctgctcggcgcgcccctggccggcccgggggtcgaggcc |
| | KAPPGPARAPPGQ | cggacgccggccgaccgcggcccctcgcccgctcccgcccccgccgcctccccgcagcc |
| | PAEPDAPRSSPGPG | cgggtcccgtggcccccgggacggcctcggcccggagccccaggagccgccgccggc |
| | AREEGAGGAATPE | ccgcccccgtccccggccgccgcgcccgaggccgtgccgcccccgccggcgcccccctc |
| | DGAGGRGSEGSQS | ctactcggacaagggcgtccagactcccagcaagggcacgccggcgccgctgcccaacg |
| | PGSSVDDAEDDPSR | gcaaccaccggtactgccgtctttgcaacatcaagttcagcagcctgtccaccttcatcgccca |
| | TLCEACNIRFSRHE | caagaagtattactgctcctcgcacgccgccgagcacgtgaagtga (SEQ ID NO: |
| | TYTVHKRYYCASR | 171) |
| | HDPPPRRPAAPPG | |
| | PPGPAAPPAPSPAA | |
| | PVRTRRRRKLYEL | |
| | HAAGAPPPPPPGH | |
| | APAPESPRPGSGSG | |
| | SGPGLAPARSPGP | |
| | AADGPIDLSKKPR | |
| | RPLPGAPAPALAD | |
| | YHECTACRVSFHS | |
| | LEAYLAHKKYSCP | |
| | AAPPPGALGLPAA | |
| | ACPYCPPNGPVRG | |
| | DLLEHFRLAHGLL | |
| | LGAPLAGPGVEAR | |
| | TPADRGPSPAPAPA | |
| | ASPQPGSRGPRDG | |
| | LGPEPQEPPPGPPP | |
| | SPAAAPEAVPPPPA | |
| | PPSYSDKGVQTPS | |
| | KGTPAPLPNGNHR | |
| | YCRLCNIKFSSLST | |
| | FIAHKKYYCSSHA | |
| | AEHVK (SEQ ID | |
| | NO: 143) | |
| DNM | MNHDQEFDPPKVYP | ATGAACCATGACCAGGAATTTGACCCCCCAAAGGTTTAC |
| T3A - | PVPAEKRKPIRVLSLF | CCACCTGTGCCAGCTGAGAAGAGGAAGCCCATCCGCGT |
| DNMT3L - | DGIATGLLVLKDLGI | GCTGTCTCTCTTTGATGGGATTGCTACAGGGCTCCTGGTG |
| dCas9- | QVDRYIASEVCEDSIT | CTGAAGGACCTGGGCATCCAAGTGGACCGCTACATTGCC |
| DHAC3 | VGMVRHQGKIMYVG | TCCGAGGTGTGTGAGGACTCCATCACGGTGGGCATGGTG |
| (FIG. | DVRSVTQKHIQEWG | CGGCACCAGGGAAAGATCATGTACGTCGGGGACGTCCG |
| 4B) | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESMAKTVAY | CTCAGCTGGGAGGCGACGCCTATCCCTATGACGTGCCCG |
| | FYDPDVGNFHYGA | ATTATGCCAGCCTGGGCAGCGGCTCCCCCAAGAAAAAA |
| | GHPMKPHRLALT | CGCAAGGTGGAAGATCCTAAGAAAAAGCGGAAAGTGGA |
| | HSLVLHYGLYKK | CGGCagcggcagcgagacccccggtaccagcgagagcgccacccccgagagcatgg |
| | MIVFKPYQASQHD | ccaagaccgtggcctatttctacgaccccgacgtgggcaacttccactacggagctggacac |
| | MCRFHSEDYIDFL | cctatgaagccccatcgcctggcattgacccatagcctggtcctgcattacggtctctataaga |
| | QRVSPTNMQGFTK | agatgatcgtcttcaagccataccaggcctcccaacatgacatgtgccgcttccactccgagg |
| | SLNAFNVGDDCPV | actacattgacttcctgcagagagtcagccccaccaatatgcaaggcttcaccaagagtcttaa |
| | FPGLFEFCSRYTG | tgccttcaacgtaggcgatgactgcccagtgtttcccgggctctttgagttctgctcgcgttaca |
| | ASLQGATQLNNKI | caggcgcatctctgcaaggagcaacccagctgaacaacaagatctgtgatattgccattaact |
| | CDIAINWAGGLHH | gggctggtggtctgcaccatgccaagaagtttgaggcctctggcttctgctatgtcaacgacat |
| | AKKFEASGFCYVN | tgtgattggcatcctggagctgctcaagtaccaccctcgggtgctctacattgacattgacatcc |
| | DIVIGILELLKYHP | accatggtgacggggttcaagaagctttctacctcactgaccgggtcatgacggtgtccttcca |
| | RVLYIDIDIHHGDG | caaatacggaaattacttcttccctggcacaggtgacatgtatgaagtcggggcagagagtgg |
| | VQEAFYLTDRVMT | ccgctactactgtctgaacgtgcccctgcgggatggcattgatgaccagagttacaagcacctt |
| | VSFHKYGNYFFPG | ttccagccggttatcaaccaggtagtggacttctaccaacccacgtgcattgtgctccagtgtg |
| | TGDMYEVGAESG | gagctgactctctgggctgtgatcgattgggctgctttaacctcagcatccgagggcatgggg |
| | RYYCLNVPLRDGI | aatgcgttgaatatgtcaagagcttcaatatccctctactcgtgctgggtggtggtggttatactg |
| | DDQSYKHLFQPVI | tccgaaatgttgcccgctgctggacatatgagacatcgctgctggtagaagaggccattagtg |
| | NQVVDFYQPTCIV | aggagcttccctatagtgaatacttcgagtactttgccccagacttcacacttcatccagatgtca |
| | LQCGADSLGCDRL | gcacccgcatcgagaatcagaactcacgccagtatctggaccagatccgccagacaatcttt |
| | GCFNLSIRGHGEC | gaaaacctgaagatgctgaaccatgcacctagtgtccagattcatgacgtgcctgcagacctc |
| | VEYVKSFNIPLLVL | ctgacctatgacaggactgatgaggctgatgcagaggagaggggtcctgaggagaactata |
| | GGGGYTVRNVAR | gcaggtctgggagcagggacttcagcctactttcaataggcttgctggggaggtttaaaaggg |
| | CWTYETSLLVEEA | aaaaagtaaatgggaaggtattagtttaa (SEQ ID NO: 172) |
| | ISEELPYSEYFEYF | |
| | APDFTLHPDVSTRI | |
| | ENQNSRQYLDQIR | |
| | QTIFENLKMLNHA | |
| | PSVQIHDVPADLLT | |
| | YDRTDEADAEERG | |
| | PEENYSRSGSRDFS | |
| | LLSIGLLGRFKRE | |
| | KVNGKVLV (SEQ | |
| | ID NO: 144) | |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| DQT1L | QVDRYIASEVCEDSIT | CTGAAGGACCTGGGCATCCAAGTGGACCGCTACATTGCC |
| (FIG. | VGMVRHQGKIMYVG | TCCGAGGTGTGTGAGGACTCCATCACGGTGGGCATGGTG |
| 4B) | DVRSVTQKHIQEWG | CGGCACCAGGGAAAGATCATGTACGTCGGGGACGTCCG |
| | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESMGEKLEL | CTCAGCTGGGAGGCGACGCCTATCCCTATGACGTGCCCG |
| | RLKSPVGAEPAVY | ATTATGCCAGCCTGGGCAGCGGCTCCCCCAAGAAAAAA |
| | PWPLPVYDKHHD | CGCAAGGTGGAAGATCCTAAGAAAAAGCGGAAAGTGGA |
| | AAHEIIETIRWVCE | CGGCagcggcagcgagacccccggtaccagcgagagcgccacccccgagagcatgg |
| | EIPDLKLAMENYV | gggagaagctggagctgagactgaagtcgcccgtgggggctgagcccgccgtctacccgt |
| | LIDYDTKSFESMQ | ggccgctgccggtctacgataaacatcacgatgctgctcatgaaatcatcgagaccatccgat |
| | RLCDKYNRAIDSIH | gggtctgtgaagaaatcccggatctcaagctcgctatggagaattacgttttaattgactatgac |
| | QLWKGTTQPMKL | accaaaagcttcgagagcatgcagaggctctgcgacaagtacaaccgtgccatcgacagca |
| | NTRPSTGLLRHILQ | tccaccagctgtggaagggcaccacgcagcccatgaagctgaacacgcggccgtccactg |
| | QVYNHSVTDPEKL | gactcctgcgccatatcctgcagcaggtctacaaccactcggtgaccgaccccgagaagctc |
| | NNYEPFSPEVYGE | aacaactacgagcccttctcccccgaggtgtacggggagacctccttcgacctggtggccca |
| | TSFDLVAQMIDEIK | gatgattgatgagatcaagatgaccgacgacgacctgtttgtggacttggggagcggtgtgg |
| | MTDDDLFVDLGSG | gccaggtcgtgctccaggttgctgctgccaccaactgcaaacatcactatggcgtcgagaaa |
| | VGQVVLQVAAAT | gcagacatcccggccaagtatgcggagaccatggaccgcgagttcaggaagtggatgaaat |
| | NCKHHYGVEKADI | ggtatggaaaaaagcatgcagaatacacattggagagaggcgatttcctctcagaagagtgg |
| | PAKYAETMDREFR | agggagcgaatcgccaacacgagtgttatatttgtgaataattttgcctttggtcctgaggtgga |
| | KWMKWYGKKHA | tcaccagctgaaggagcggtttgcaaacatgaaggaaggtggcagaatcgtgtcctcgaaac |
| | EYTLERGDFLSEE | cctttgcacctctgaacttcagaataaacagtagaaacttgagtgacatcggcaccatcatgcg |
| | WRERIANTSVIFVN | cgtggtggagctctcgcccctgaagggctcggtgtcgtggacggggaagccagtctcctact |
| | NFAFGPEVDHQLK | acctgcacactatcgaccgcaccatacttgaaaactatttttctagtctgaaaaacccaaaactc |
| | ERFANMKEGGRIV | agggaggaacaggaggcagcccggcgccgccagcagcgcgagagcaagagcaacgcg |
| | SSKPFAPLNFRINS | gccacgcccactaagggcccagagggcaaggtggccggccccgccgacgcccccatgga |
| | RNLSDIGTIMRVVE | ctctggtgctgaggaagagaaggcgggagcagccaccgtgaagaagccgtctccctccaa |
| | LSPLKGSVSWTGK | agcccgcaagaagaagctaaacaagaaggggaggaagatggctggccgcaagcgcggg |
| | PVSYYLHTIDRTIL | cgccccaagaagatgaacactgcgaaccccgagcggaagcccaagaagaaccaaactgc |
| | ENYFSSLKNPKLR | actggatgccctgcacgctcagaccgtgtctcagacggcggcctcctcaccccaggatgcct |
| | EEQEAARRRQQRE | acagatcccctcacagcccgttctaccagctacctccgagcgtgcagcggcactcccccaac |
| | SKSNAATPTKGPE | ccgctgctggtggcgcccaccccgcccgcgctgcagaagcttctagagtccttcaagatcca |
| | GKVAGPADAPMDS | gtacctgcagttcctggcatacacaaagaccccccagtacaaggccagcctgcaggagctg |
| | GAEEEKAGAATVK | ctgggccaggagaaggagaagaacgcccagctcctgggtgcggctcagcagctcctcagc |
| | KPSPSKARKKKLN | cactgccaggcccagaaggaggagatcaggaggctgtttcagcaaaaattggatgagctgg |
| | KKGRKMAGRKRG | gtgtgaaggcgctgacctacaacgacctgattcaagcgcagaaggagatctccgcccataac |
| | RPKKMNTANPER | cagcagctgcgggagcagtcggagcagctggagcaggacaaccgcgcgctccgcggcca |
| | KPKKNQTALDALH | gagcttgcagctgctcaaggctcgctgcgaggagctgcagctggactgggccacgctgtcg |
| | AQTVSQTAASSPQ | ctggagaagctgttgaaggagaagcaggccctgaagagccagatctcggagaagcagagg |
| | DAYRSPHSPFYQLP | cactgcctggagctgcagatcagcattgtggagctagagaagagccagcggcagcaggag |
| | PSVQRHSPNPLLV | ctcctgcagctcaagtcctgtgtgccgcctgacgacgccctgtccctgcacctgcgtgggaag |
| | APTPPALQKLLESF | ggcgccctgggccgcgagctggagcctgacgccagccggctgcacctggagctggactgc |
| | KIQYLQFLAYTKT | accaagttctcgctgcctcacttgagcagcatgagcccggagctctccatgaacggccaggc |
| | PQYKASLQELLGQ | tgctggctatgagctctgcggtgtgctgagccggccttcgtcgaagcagaacacgccccagt |
| | EKEKNAQLLGAA | acctggcctcacccctggaccaggaggtggtgccctgtacccctagccacgtcggccggcc |
| | QQLLSHCQAQKEE | gcgcctggagaagctgtctggcctagccgcacccgactacactaggctgtccccggccaag |
| | IRRLFQQKLDELG | attgtgctgaggcggcacctgagccaggaccacacggtgcccggcaggccggctgccagt |
| | VKALTYNDLIQAQ | gagctgcattcgagagctgagcacaccaaggagaacggccttccctaccagagccccagc |
| | KEISAHNQQLREQ | gtgcctggcagcatgaagctgagccctcaggacccgcggcccctgtcccctggggccttgc |
| | SEQLEQDNRALRG | agcttgctggagagaagagcagtgagaagggcctgagagagcgcgcctacggcagcagc |
| | QSLQLLKARCEEL | ggggagctcatcaccagcctgcccatcagcatcccgctcagcaccgtgcagcccaacaagc |
| | QLDWATLSLEKLL | tcccggtcagcattcccctggccagcgtggtgctgcccagccgcgccgagagggcgagga |
| | KEKQALKSQISEK | gcacccccagtcccgtgctgcagccccgtgacccctcgtccacacttgaaaagcagattggt |
| | QRHCLELQISIVEL | gctaatgcccacggtgctgggagcagaagccttgccctggcccccgcaggcttctcctacgc |
| | EKSQRQQELLQLK | tggctcggtggccatcagcggggccttggcgggcagcccggcctctctcacacctggagcc |
| | SCVPPDDALSLHL | gagccggccaccttggatgagtcctccagctctgggagcctttttgccaccgtggggtcccgc |
| | RGKGALGRELEPD | agctccacgccacagcaccccctgctgctggcacagccccggaactcgcttcctgcctctcc |
| | ASRLHLELDCTKF | cgcccaccagctctcctccagtccccggcttggtggggccgcccagggcccgttgcccgag |
| | SLPHLSSMSPELSM | gccagcaagggagacctgccctccgattccggcttctcagatcctgagagtgaagccaaga |
| | NGQAAGYELCGV | ggaggattgtgttcaccatcaccactggtgcgggcagtgccaagcagtcgccctccagcaag |
| | LSRPSSKQNTPQYL | cacagccccctgaccgccagcgcccgtggggactgtgtgccgagccacgggcaggacagt |
| | ASPLDQEVVPCTPS | cgcaggcgcggccggcggaagcgagcatctgcggggacgcccagcttgagcgcaggcgt |
| | HVGRPRLEKLSGL | gtcccccaagcgccgagccctgccgtccgtcgctggccttttcacacagccttcggggtctcc |
| | AAPDYTRLSPAKIV | cctcaacctcaactccatggtcagtaacatcaaccagcccctggagattacagccatctcgtcc |
| | LRRHLSQDHTVPG | ccggagacctccctgaagagctcccctgtgccctaccaggaccacgaccagccccccgtgc |
| | RPAASELHSRAEH | tcaagaaggagcggcctctgagccagaccaatggggcacactactccccactcacctcaga |
| | TKENGLPYQSPSV | cgaggagccaggctctgaggacgagcccagcagtgctcgaattgagagaaaaattgcaaca |
| | PGSMKLSPQDPRP | atctccttagaaagcaaatctcccccgaaaaccttggaaaatggtggtggcttggcgggaagg |
| | LSPGALQLAGEKS | aagcccgcgcccgccggcgagccagtcaatagcagcaagtggaagtccaccttctcgccca |
| | SEKGLRERAYGSS | tctccgacatcggcctggccaagtcggcggacagcccgctgcaggccagctccgccctcag |
| | GELITSLPISIPLST | ccagaactccctgttcacgttccggcccgccctggaggagccctctgccgatgccaagctgg |
| | VQPNKLPVSIPLAS | ccgctcaccccaggaaaggctttcccggctccctgtcgggggctgacggactcagcccggg |
| | VVLPSRAERARSTP | caccaaccctgccaacggctgcaccttcggcgggggcctggccgcggacctgagtttacac |
| | SPVLQPRDPSSTLE | agcttcagtgatggtgcttctcttccccacaagggccccgaggcggccggcctgagctcccc |
| | KQIGANAHGAGSR | gctgagcttcccctcgcagcgcggcaaggagggctcggacgccaaccctttcctgagcaag |
| | SLALAPAGFSYAGS | aggcagctggacggcctggctgggctgaagggcgagggcagccgcggcaaggaggcag |
| | VAISGALAGSPASL | gggagggcggcctaccgctgtgcgggcccacggacaagaccccactgctgagcggcaag |
| | TPGAEPATLDESSS | gccgccaaggcccgggaccgcgaggtcgacctcaagaatggccacaacctcttcatctctg |
| | SGSLFATVGSRSST | cggggccgtgcctcccggaagcctcctcagcggccccggcctggccccggcggcgtcct |
| | PQHPLLLAQPRNS | ccgcaggcggcgcggcgtcctccgcccagacgcaccggtccttcctgggccccttcccgcc |
| | LPASPAHQLSSSPR | gggaccgcagttcgcgctcggccccatgtccctgcaggccaacctcggctccgtggccggc |
| | LGGAAQGPLPEAS | tcctccgtgctgcagtcgctgttcagctctgtgccggccgccgcaggcctggtgcacgtgtcg |
| | KGDLPSDSGFSDPE | tccgctgccaccagactgaccaactcgcacgccatgggcagcttttccggggtggcaggcg |
| | SEAKRRIVFTITTG | gcacagttggaggtgtctttaaccacgcggtgccctccgcctctgctcatccgtttggagcccg |
| | AGSAKQSPSSKHSP | tgtcggccgcggggctgcatgtggcagcgccacactgggcccgagcccgctgcaggcgg |
| | LTASARGDCVPSH | cggccagcgcctcggcctcttcctttcaggccccggcctcggttgagacccggccgccccct |
| | GQDSRRRGRRKR | ccgcctccgcctccgcctcccccgctgcccccgcctgcgcacctgggccggtcccccgcgg |
| | ASAGTPSLSAGVSP | ggccgcccgtcctccacgccccccctccacctaacgccgccttgcctcctcccccaacgctg |
| | KRRALPSVAGLFT | ctggcctctaaccctgagcccgcgcttctgcagagcctcgcgtccctcccgcctaaccaagct |
| | QPSGSPLNLNSMV | ttcttgccccccacctctgctgcctctctgccgcctgctaacgcctctttgtctatcaagctcacct |
| | SNINQPLEITAISSP | ccctcccgcacaagggcgcccgcccctccttcacggtgcaccaccagcccctgccccggct |
| | ETSLKSSPVPYQDH | ggccctggcccaggccgcgcccgggatcccacaggccagcgccacggggccgtccgcg |
| | DQPPVLKKERPLS | gtgtgggtgtccctcggcatgccgcctccctatgccgcgcacctttcgggggttaagccgcga |
| | QTNGAHYSPLTSD | taa (SEQ ID NO: 173) |
| | EEPGSEDEPSSARI | |
| | ERKIATISLESKSPP | |
| | KTLENGGGLAGR | |
| | KPAPAGEPVNSSK | |
| | WKSTFSPISDIGLA | |
| | KSADSPLQASSALS | |
| | QNSLFTFRPALEEP | |
| | SADAKLAAHPRKG | |
| | FPGSLSGADGLSPG | |
| | TNPANGCTFGGGL | |
| | AADLSLHSFSDGAS | |
| | LPHKGPEAAGLSS | |
| | PLSFPSQRGKEGSD | |
| | ANPFLSKRQLDGL | |
| | AGLKGEGSRGKEA | |
| | GEGGLPLCGPTDK | |
| | TPLLSGKAAKARD | |
| | REVDLKNGHNLFI | |
| | SAAAVPPGSLLSGP | |
| | GLAPAASSAGGAA | |
| | SSAQTHRSFLGPFP | |
| | PGPQFALGPMSLQ | |
| | ANLGSVAGSSVLQ | |
| | SLFSSVPAAAGLVH | |
| | VSSAATRLTNSHA | |
| | MGSFSGVAGGTVG | |
| | GVFNHAVPSASAH | |
| | PFGARVGRGAACG | |
| | SATLGPSPLQAAAS | |
| | ASASSFQAPASVET | |
| | RPPPPPPPPPPPLPP | |
| | PAHLGRSPAGPPV | |
| | LHAPPPPNAALPPP | |
| | PTLLASNPEPALLQ | |
| | SLASLPPNQAFLPP | |
| | TSAASLPPANASLS | |
| | IKLTSLPHKGARPS | |
| | FTVHHQPLPRLAL | |
| | AQAAPGIPQASAT | |
| | GPSAVWVSLGMPP | |
| | PYAAHLSGVKPR | |
| | (SEQ ID NO: 145) | |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| KRAB- | QVDRYIASEVCEDSIT | CTGAAGGACCTGGGCATCCAAGTGGACCGCTACATTGCC |
| EZH2 | VGMVRHQGKIMYVG | TCCGAGGTGTGTGAGGACTCCATCACGGTGGGCATGGTG |
| (FIG. | DVRSVTQKHIQEWG | CGGCACCAGGGAAAGATCATGTACGTCGGGGACGTCCG |
| 4B) | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESRTLVTFKD | CTCAGCTGGGAGGCGACGCCTATCCCTATGACGTGCCCG |
| | VFVDFTREEWKLL | ATTATGCCAGCCTGGGCAGCGGCTCCCCCAAGAAAAAA |
| | DTAQQIVYRNVMLE | CGCAAGGTGGAAGATCCTAAGAAAAAGCGGAAAGTGGA |
| | NYKNLVSLGYQLTK | CGGCagcggcagcgagacccccggtaccagcgagagcgccacccccgagagcCG |
| | PDVILRLEKGEEPA | GACACTGGTGACCTTCAAGGATGTATTTGTGGACTTCAC |
| | YPYDVPDYASLGSG | CAGGGAGGAGTGGAAGCTGCTGGACACTGCTCAGCAGA |
| | SPKKKRKVEDPKK | TCGTGTACAGAAATGTGATGCTGGAGAACTATAAGAACC |
| | KRKVDGSGSETPGT | TGGTTTCCTTGGGTTATCAGCTTACTAAGCCAGATGTGAT |
| | SESATPESMGQTG | CCTCCGGTTGGAGAAGGGAGAAGAGCCCagcggcagcgagacc |
| | KKSEKGPVCWRK | cccggtaccagcgagagcgccacccccgagagcatgggccagactgggaagaaatctga |
| | RVKSEYMRLRQL | gaagggaccagtttgttggcggaagcgtgtaaaatcagagtacatgcgactgagacagctca |
| | KRFRRADEVKSMF | agaggttcagacgagctgatgaagtaaagagtatgtttagttccaatcgtcagaaaattttggaa |
| | SSNRQKILERTEIL | agaacggaaatcttaaaccaagaatggaaacagcgaaggatacagcctgtgcacatcctgac |
| | NQEWKQRRIQPV | ttctgtgagctcattgcgcgggactagggagtgttcggtgaccagtgacttggattttccaacac |
| | HILTSVSSLRGTRE | aagtcatcccattaaagactctgaatgcagttgcttcagtacccataatgtattcttggtctcccct |
| | CSVTSDLDFPTQVI | acagcagaattttatggtggaagatgaaactgttttacataacattccttatatgggagatgaagt |
| | PLKTLNAVASVPI | tttagatcaggatggtactttcattgaagaactaataaaaaattatgatgggaaagtacacgggg |
| | MYSWSPLQQNFM | atagagaatgtgggtttataaatgatgaaatttttgtggagttggtgaatgcccttggtcaatataa |
| | VEDETVLHNIPYM | tgatgatgacgatgatgatgatggagacgatcctgaagaaagagaagaaaagcagaaagat |
| | GDEVLDQDGTFIE | ctggaggatcaccgagatgataaagaaagccgcccacctcggaaatttccttctgataaaattt |
| | ELIKNYDGKVHGD | ttgaagccatttcctcaatgtttccagataagggcacagcagaagaactaaaggaaaaatataa |
| | RECGFINDEIFVEL | agaactcaccgaacagcagctcccaggcgcacttcctcctgaatgtacccccaacatagatg |
| | VNALGQYNDDDD | gaccaaatgctaaatctgttcagagagagcaaagcttacactcctttcatacgcttttctgtaggc |
| | DDDGDDPEEREEK | gatgttttaaatatgactgcttcctacatcgtaagtgcaattattcttttcatgcaacacccaacact |
| | QKDLEDHRDDKES | tataagcggaagaacacagaaacagctctagacaacaaaccttgtggaccacagtgttacca |
| | RPPRKFPSDKIFEA | gcatttggagggagcaaaggagtttgctgctgctctcaccgctgagcggataaagaccccac |
| | ISSMFPDKGTAEEL | caaaacgtccaggaggccgcagaagaggacggcttcccaataacagtagcaggcccagca |
| | KEKYKELTEQQLP | cccccaccattaatgtgctggaatcaaaggatacagacagtgatagggaagcagggactgaa |
| | GALPPECTPNIDGP | acggggggagagaacaatgataaagaagaagaagagaagaaagatgaaacttcgagctcc |
| | NAKSVQREQSLHS | tctgaagcaaattctcggtgtcaaacaccaataaagatgaagccaaatattgaacctcctgaga |
| | FHTLFCRRCFKYD | atgtggagtggagtggtgctgaagcctcaatgtttagagtcctcattggcacttactatgacaatt |
| | CFLHRKCNYSFHA | tctgtgccattgctaggttaattgggaccaaaacatgtagacaggtgtatgagtttagagtcaaa |
| | TPNTYKRKNTETA | gaatctagcatcatagctccagctcccgctgaggatgtggatactcctccaaggaaaaagaag |
| | LDNKPCGPQCYQH | aggaaacaccggttgtgggctgcacactgcagaaagatacagctgaaaaaggacggctcct |
| | LEGAKEFAAALTA | ctaaccatgtttacaactatcaaccctgtgatcatccacggcagccttgtgacagttcgtgccctt |
| | ERIKTPPKRPGGR | gtgtgatagcacaaaatttttgtgaaaagttttgtcaatgtagttcagagtgtcaaaaccgctttcc |
| | RRGRLPNNSSRPST | gggatgccgctgcaaagcacagtgcaacaccaagcagtgcccgtgctacctggctgtccga |
| | PTINVLESKDTDSD | gagtgtgaccctgacctctgtcttacttgtggagccgctgaccattgggacagtaaaaatgtgt |
| | REAGTETGGENND | cctgcaagaactgcagtattcagcggggctccaaaaagcatctattgctggcaccatctgacg |
| | KEEEEKKDETSSSS | tggcaggctgggggatttttatcaaagatcctgtgcagaaaaatgaattcatctcagaatactgt |
| | EANSRCQTPIKMK | ggagagattatttctcaagatgaagctgacagaagagggaaagtgtatgataaatacatgtgc |
| | PNIEPPENVEWSG | agctttctgttcaacttgaacaatgattttgtggtggatgcaacccgcaagggtaacaaaattcgt |
| | AEASMFRVLIGTY | tttgcaaatcattcggtaaatccaaactgctatgcaaaagttatgatggttaacggtgatcacag |
| | YDNFCAIARLIGTK | gataggtatttttgccaagagagccatccagactggcgaagagctgttttttgattacagataca |
| | TCRQVYEFRVKES | gccaggctgatgccctgaagtatgtcggcatcgaaagagaaatggaaatcccttga (SEQ |
| | SIIAPAPAEDVDTP | ID NO: 174) |
| | PRKKKRKHRLWA | |
| | AHCRKIQLKKDGS | |
| | SNHVYNYQPCDHP | |
| | RQPCDSSCPCVIAQ | |
| | NFCEKFCQCSSEC | |
| | QNRFPGCRCKAQC | |
| | NTKQCPCYLAVRE | |
| | CDPDLCLTCGAAD | |
| | HWDSKNVSCKNCS | |
| | IQRGSKKHLLLAP | |
| | SDVAGWGIFIKDP | |
| | VQKNEFISEYCGEI | |
| | ISQDEADRRGKVY | |
| | DKYMCSFLFNLNN | |
| | DFVVDATRKGNKI | |
| | RFANHSVNPNCYA | |
| | KVMMVNGDHRIGI | |
| | FAKRAIQTGEELFF | |
| | DYRYSQADALKYV | |
| | GIEREMEIP* (SEQ | |
| | ID NO: 146) | |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| KRAB- | QVDRYIASEVCEDSIT | CTGAAGGACCTGGGCATCCAAGTGGACCGCTACATTGCC |
| G9A | VGMVRHQGKIMYVG | TCCGAGGTGTGTGAGGACTCCATCACGGTGGGCATGGTG |
| (FIG. | DVRSVTQKHIQEWG | CGGCACCAGGGAAAGATCATGTACGTCGGGGACGTCCG |
| 4B) | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLIN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESRTLVTFKD | CTCAGCTGGGAGGCGACGCCTATCCCTATGACGTGCCCG |
| | VFVDFTREEWKLL | ATTATGCCAGCCTGGGCAGCGGCTCCCCCAAGAAAAAA |
| | DTAQQIVYRNVMLE | CGCAAGGTGGAAGATCCTAAGAAAAAGCGGAAAGTGGA |
| | NYKNLVSLGYQLTK | CGGCagcggcagcgagacccccggtaccagcgagagcgccacccccgagagcCG |
| | PDVILRLEKGEEPA | GACACTGGTGACCTTCAAGGATGTATTTGTGGACTTCAC |
| | YPYDVPDYASLGSG | CAGGGAGGAGTGGAAGCTGCTGGACACTGCTCAGCAGA |
| | SPKKKRKVEDPKK | TCGTGTACAGAAATGTGATGCTGGAGAACTATAAGAACC |
| | KRKVDGSGSETPGT | TGGTTTCCTTGGGTTATCAGCTTACTAAGCCAGATGTGAT |
| | SESATPESMRGLPR | CCTCCGGTTGGAGAAGGGAGAAGAGCCCagcggcagcgagacc |
| | GRGLMRARGRGR | cccggtaccagcgagagcgccacccccgagagcatgcggggtctaccgagagggaggg |
| | AAPPGSRGRGRGG | ggttgatgcgggcccgggggaggggtcgtgcggcccctccgggcagccgaggccgcgg |
| | PHRGRGRPRSLLS | aaggggggggccccacagaggaagaggtaggccccggagcctactctctcttcccagggc |
| | LPRAQASWTPQLS | ccaggcatcctggaccccccaactctctactgggctgaccagccctcctgtcccttgtctcccc |
| | TGLTSPPVPCLPSQ | tcccagggggaggcccccgctgagatgggggcgctgctgctggagaaggaaaccagagg |
| | GEAPAEMGALLLE | agccaccgagagagttcatggctctttgggggacacccctcgtagtgaagaaaccctgccca |
| | KETRGATERVHGS | aggccacccccgactccctggagcctgctggcccctcatctccagcctctgtcactgtcactgt |
| | LGDTPRSEETLPK | tggtgatgagggggctgacacccctgtaggggctacaccactcattggggatgaatctgaga |
| | ATPDSLEPAGPSSP | atcttgagggagatggggacctccgtgggggccggatcctgctgggccatgccacaaagtc |
| | ASVTVTVGDEGAD | attcccctcttcccccagcaaggggggttcctgtcctagccgggccaagatgtcaatgacagg |
| | TPVGATPLIGDESE | ggcgggaaaatcacctccatctgtccagagtttggctatgaggctactgagtatgccaggagc |
| | NLEGDGDLRGGRI | ccagggagctgcagcagcagggtctgaaccccctccagccaccacgagcccagagggac |
| | LLGHATKSFPSSPS | agcccaaggtccaccgagcccgcaaaaccatgtccaaaccaggaaatggacagcccccgg |
| | KGGSCPSRAKMS | tccctgagaagcggccccctgaaatacagcatttccgcatgagtgatgatgtccactcactgg |
| | MTGAGKSPPSVQS | gaaaggtgacctcagatctggccaaaaggaggaagctgaactcaggaggtggcctgtcaga |
| | LAMRLLSMPGAQ | ggagttaggttctgcccggcgttcaggagaagtgaccctgacgaaaggggaccccgggtcc |
| | GAAAAGSEPPPAT | ctggaggagtgggagacggtggtgggtgatgacttcagtctctactatgattcctactctgtgg |
| | TSPEGQPKVHRAR | atgagcgcgtggactccgacagcaagtctgaagttgaagctctaactgaacaactaagtgaa |
| | KTMSKPGNGQPPV | gaggaggaggaggaagaggaggaagaagaagaagaggaagaggaggaggaagagga |
| | PEKRPPEIQHFRM | agaagaagaggaagatgaggagtcagggaatcagtcagataggagtggttccagtggccg |
| | SDDVHSLGKVTSD | gcgcaaggccaagaagaaatggcgaaaagacagcccatgggtgaagccgtctcggaaac |
| | LAKRRKLNSGGGL | ggcgcaagcgggagcctccgcgggccaaggagccacgaggggtgtccaatgacacatctt |
| | SEELGSARRSGEV | cgctggagacagagcgagggtttgaggagttgcccctgtgcagctgccgcatggaggcacc |
| | TLTKGDPGSLEEW | caagattgaccgcatcagcgagagggcggggcacaagtgcatggccactgagagtgtgga |
| | ETVVGDDFSLYYD | cggagagctgtcaggctgcaatgccgccatcctcaagcgggagaccatgaggccatccagc |
| | SYSVDERVDSDSKS | cgtgtggccctgatggtgctctgtgagacccaccgcgcccgcatggtcaaacaccactgctg |
| | EVEALTEQLSEEE | cccgggctgcggctacttctgcacggcgggcaccttcctggagtgccaccctgacttccgtgt |
| | EEEEEEEEEEEEEE | ggcccaccgcttccacaaggcctgtgtgtctcagctgaatgggatggtcttctgtccccactgt |
| | EEEEEEEDEESGN | ggggaggatgcttctgaagctcaagaggtgaccatcccccggggtgacggggtgacccca |
| | QSDRSGSSGRRKA | ccggccggcactgcagctcctgcacccccacccctgtcccaggatgtccccgggagagca |
| | KKKWRKDSPWVK | gacacttctcagcccagtgcccggatgcgagggcatggggaaccccggcgcccgccctgc |
| | PSRKRRKREPPRA | gatcccctggctgacaccattgacagctcagggccctccctgaccctgcccaatgggggctg |
| | KEPRGVSNDTSSLE | cctttcagccgtggggctgccactggggccaggccgggaggccctggaaaaggccctggt |
| | TERGFEELPLCSC | catccaggagtcagagaggcggaagaagctccgtttccaccctcggcagttgtacctgtccgt |
| | RMEAPKIDRISERA | gaagcagggcgagctgcagaaggtgatcctgatgctgttggacaacctggaccccaacttcc |
| | GHKCMATESVDG | agagcgaccagcagagcaagcgcacgcccctgcatgcagccgcccagaagggctccgtg |
| | ELSGCNAAILKRE | gagatctgccatgtgctgctgcaggctggagccaacataaatgcagtggacaaacagcagc |
| | TMRPSSRVALMVL | ggacgccactgatggaggccgtggtgaacaaccacctggaggtagcccgttacatggtgca |
| | CETHRARMVKHH | gcgtggtggctgtgtctatagcaaggaggaggacggttccacctgcctccaccacgcagcca |
| | CCPGCGYFCTAGT | aaatcgggaacttggagatggtcagcctgctgctgagcacaggacaggtggacgtcaacgc |
| | FLECHPDFRVAHR | ccaggacagtggggggtggacgcccatcatctgggctgcagagcacaagcacatcgaggt |
| | FHKACVSQLNGM | gatccgcatgctactgacgcggggcgccgacgtcaccctcactgacaacgaggagaacatc |
| | VFCPHCGEDASEA | tgcctgcactgggcctccttcacgggcagcgccgccatcgccgaagtccttctgaatgcgcg |
| | QEVTIPRGDGVTP | ctgtgacctccatgctgtcaactaccatggggacacccccctgcacatcgcagctcgggaga |
| | PAGTAAPAPPPLSQ | gctaccatgactgcgtgctgttattcctgtcacgtggggccaaccctgagctgcggaacaaag |
| | DVPGRADTSQPSA | agggggacacagcatgggacctgactcccgagcgctccgacgtgtggtttgcgcttcaactc |
| | RMRGHGEPRRPPC | aaccgcaagctccgacttggggtgggaaatcgggccatccgcacagagaagatcatctgcc |
| | DPLADTIDSSGPSL | gggacgtggctcggggctatgagaacgtgcccattccctgtgtcaacggtgtggatggggag |
| | TLPNGGCLSAVGL | ccctgccctgaggattacaagtacatctcagagaactgcgagacgtccaccatgaacatcgat |
| | PLGPGREALEKAL | cgcaacatcacccacctgcagcactgcacgtgtgtggacgactgctctagctccaactgcctg |
| | VIQESERRKKLRF | tgcggccagctcagcatccggtgctggtatgacaaggatgggcgattgctccaggaatttaac |
| | HPRQLYLSVKQGE | aagattgagcctccgctgattttcgagtgtaaccaggcgtgctcatgctggagaaactgcaag |
| | LQKVILMLLDNLD | aaccgggtcgtacagagtggcatcaaggtgcggctacagctctaccgaacagccaagatgg |
| | PNFQSDQQSKRTP | gctggggggtccgcgccctgcagaccatcccacaggggaccttcatctgcgagtatgtcgg |
| | LHAAAQKGSVEIC | ggagctgatctctgatgctgaggctgatgtgagagaggatgattcttacctcttcgacttagaca |
| | HVLLQAGANINAV | acaaggatggagaggtgtactgcatagatgcccgttactatggcaacatcagccgcttcatca |
| | DKQQRTPLMEAV | accacctgtgtgaccccaacatcattcccgtccgggtcttcatgctgcaccaagacctgcgatt |
| | VNNHLEVARYMV | tccacgcatcgccttcttcagttcccgagacatccggactggggaggagctagggtttgactat |
| | QRGGCVYSKEEDG | ggcgaccgcttctgggacatcaaaagcaaatatttcacctgccaatgtggctctgagaagtgc |
| | STCLHHAAKIGNL | aagcactcagccgaagccattgccctggagcagagccgtctggcccgcctggacccacacc |
| | EMVSLLLSTGQVD | ctgagctgctgcccgagctcggctccctgccccctgtcaacacatga (SEQ ID NO: |
| | VNAQDSGGWTPII | 175) |
| | WAAEHKHIEVIRM | |
| | LLTRGADVTLTDN | |
| | EENICLHWASFTG | |
| | SAAIAEVLLNARC | |
| | DLHAVNYHGDTPL | |
| | HIAARESYHDCVL | |
| | LFLSRGANPELRN | |
| | KEGDTAWDLTPER | |
| | SDVWFALQLNRKL | |
| | RLGVGNRAIRTEK | |
| | IICRDVARGYENVP | |
| | IPCVNGVDGEPCP | |
| | EDYKYISENCETST | |
| | MNIDRNITHLQHC | |
| | TCVDDCSSSNCLC | |
| | GQLSIRCWYDKDG | |
| | RLLQEFNKIEPPLI | |
| | FECNQACSCWRNC | |
| | KNRVVQSGIKVRL | |
| | QLYRTAKMGWGV | |
| | RALQTIPQGTFICE | |
| | YVGELISDAEADV | |
| | REDDSYLFDLDNK | |
| | DGEVYCIDARYYG | |
| | NISRFINHLCDPNII | |
| | PVRVFMLHQDLRF | |
| | PRIAFFSSRDIRTG | |
| | EELGFDYGDRFWD | |
| | IKSKYFTCQCGSE | |
| | KCKHSAEAIALEQ | |
| | SRLARLDPHPELLP | |
| | ELGSLPPVNT* | |
| | (SEQ ID NO: 147) | |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| KRAB- | QVDRYIASEVCEDSIT | CTGAAGGACCTGGGCATCCAAGTGGACCGCTACATTGCC |
| LSD1 | VGMVRHQGKIMYVG | TCCGAGGTGTGTGAGGACTCCATCACGGTGGGCATGGTG |
| (FIG. | DVRSVTQKHIQEWG | CGGCACCAGGGAAAGATCATGTACGTCGGGGACGTCCG |
| 4B) | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNEDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESRTLVTFKD | CTCAGCTGGGAGGCGACGCCTATCCCTATGACGTGCCCG |
| | VFVDFTREEWKLL | ATTATGCCAGCCTGGGCAGCGGCTCCCCCAAGAAAAAA |
| | DTAQQIVYRNVMLE | CGCAAGGTGGAAGATCCTAAGAAAAAGCGGAAAGTGGA |
| | NYKNLVSLGYQLTK | CGGCagcggcagcgagacccccggtaccagcgagagcgccacccccgagagcCG |
| | PDVILRLEKGEEPA | GACACTGGTGACCTTCAAGGATGTATTTGTGGACTTCAC |
| | YPYDVPDYASLGSG | CAGGGAGGAGTGGAAGCTGCTGGACACTGCTCAGCAGA |
| | SPKKKRKVEDPKK | TCGTGTACAGAAATGTGATGCTGGAGAACTATAAGAACC |
| | KRKVDGSGSETPGT | TGGTTTCCTTGGGTTATCAGCTTACTAAGCCAGATGTGAT |
| | SESATPESMLSGKK | CCTCCGGTTGGAGAAGGGAGAAGAGCCCagcggcagcgagacc |
| | AAAAAAAAAAAAT | cccggtaccagcgagagcgccacccccgagagcatgttatctgggaagaaggcggcagcc |
| | GTEAGPGTAGGSE | gcggcggcggcggctgcagcggcagcaaccgggacggaggctggccctgggacagcag |
| | NGSEVAAQPAGLS | gcggctccgagaacgggtctgaggtggccgcgcagcccgcgggcctgtcgggcccagcc |
| | GPAEVGPGAVGER | gaggtcgggccgggggcggtgggggagcgcacaccccgcaagaaagagcctccgcggg |
| | TPRKKEPPRASPP | cctcgccccccgggggcctggcggaaccgccggggtccgcagggcctcaggccggccct |
| | GGLAEPPGSAGPQ | actgtcgtgcctgggtctgcgacccccatggaaactggaatagcagagactccggaggggc |
| | AGPTVVPGSATPM | gtcggaccagccggcgcaagcgggcgaaggtagagtacagagagatggatgaaagcttg |
| | ETGIAETPEGRRTS | gccaacctctcagaagatgagtattattcagaagaagagagaaatgccaaagcagagaagg |
| | RRKRAKVEYREM | aaaagaagcttcccccaccaccccctcaagccccacctgaggaagaaaatgaaagtgagcc |
| | DESLANLSEDEYYS | tgaagaaccatcggggcaagcaggaggacttcaagacgacagttctggagggtatggagac |
| | EEERNAKAEKEKK | ggccaagcatcaggtgtggagggcgcagctttccagagccgacttcctcatgaccggatgac |
| | LPPPPPQAPPEEEN | ttctcaagaagcagcctgttttccagatattatcagtggaccacaacagacccagaaggtttttct |
| | ESEPEEPSGQAGG | tttcattagaaaccgcacactgcagttgtggttggataatccaaagattcagctgacatttgagg |
| | LQDDSSGGYGDGQ | ctactctccaacaattagaagcaccttataacagtgatactgtgcttgtccaccgagttcacagtt |
| | ASGVEGAAFQSRL | atttagagcgtcatggtcttatcaacttcggcatctataagaggataaaacccctaccaactaaa |
| | PHDRMTSQEAACF | aagacaggaaaggtaattattataggctctggggtctcaggcttggcagcagctcgacagtta |
| | PDIISGPQQTQKVF | caaagttttggaatggatgtcacacttttggaagccagggatcgtgtgggtggacgagttgcca |
| | LFIRNRTLQLWLD | catttcgcaaaggaaactatgtagctgatcttggagccatggtggtaacaggtcttggaggga |
| | NPKIQLTFEATLQ | atcctatggctgtggtcagcaaacaagtaaatatggaactggccaagatcaagcaaaaatgcc |
| | QLEAPYNSDTVLV | cactttatgaagccaacggacaagctgacactgtcaaggttcctaaagagaaagatgaaatgg |
| | HRVHSYLERHGLI | tagagcaagagtttaaccggttgctagaagctacatcttaccttagtcatcaactagacttcaatg |
| | NFGIYKRIKPLPTK | tcctcaataataagcctgtgtcccttggccaggcattggaagttgtcattcagttacaagagaag |
| | KTGKVIIIGSGVSG | catgtcaaagatgagcagattgaacattggaagaagatagtgaaaactcaggaagaattgaa |
| | LAAARQLQSFGMD | agaacttcttaataagatggtaaatttgaaagagaaaattaaagaactccatcagcaatacaaa |
| | VTLLEARDRVGGR | gaagcatctgaagtaaagccacccagagatattactgccgagttcttagtgaaaagcaaacac |
| | VATFRKGNYVADL | agggatctgaccgccctatgcaaggaatatgatgaattagctgaaacacaaggaaagctaga |
| | GAMVVTGLGGNP | agaaaaacttcaggagttggaagcgaatcccccaagtgatgtatatctctcatcaagagacag |
| | MAVVSKQVNMEL | acaaatacttgattggcattttgcaaatcttgaatttgctaatgccacacctctctcaactctctccc |
| | AKIKQKCPLYEAN | ttaagcactgggatcaggatgatgactttgagttcactggcagccacctgacagtaaggaatg |
| | GQADTVKVPKEK | gctactcgtgtgtgcctgtggctttagcagaaggcctagacattaaactgaatacagcagtgcg |
| | DEMVEQEFNRLLE | acaggttcgctacacggcttcaggatgtgaagtgatagctgtgaatacccgctccacgagtca |
| | ATSYLSHQLDFNV | aacctttatttataaatgcgacgcagttctctgtacccttcccctgggtgtgctgaagcagcagc |
| | LNNKPVSLGQALE | caccagccgttcagtttgtgccacctctccctgagtggaaaacatctgcagtccaaaggatgg |
| | VVIQLQEKHVKDE | gatttggcaaccttaacaaggtggtgttgtgttttgatcgggtgttctgggatccaagtgtcaattt |
| | QIEHWKKIVKTQE | gttcgggcatgttggcagtacgactgccagcaggggtgagctcttcctcttctggaacctctat |
| | ELKELLNKMVNL | aaagctccaatactgttggcactagtggcaggagaagctgctggtatcatggaaaacataagt |
| | KEKIKELHQQYKE | gacgatgtgattgttggccgatgcctggccattctcaaagggatttttggtagcagtgcagtacc |
| | ASEVKPPRDITAEF | tcagcccaaagaaactgtggtgtctcgttggcgtgctgatccctgggctcggggctcttattcct |
| | LVKSKHRDLTALC | atgttgctgcaggatcatctggaaatgactatgatttaatggctcagccaatcactcctggcccc |
| | KEYDELAETQGKL | tcgattccaggtgccccacagccgattccacgactcttctttgcgggagaacatacgatccgta |
| | EEKLQELEANPPS | actacccagccacagtgcatggtgctctgctgagtgggctgcgagaagcgggaagaattgc |
| | DVYLSSRDRQILD | agaccagtttttgggggccatgtatacgctgcctcgccaggccacaccaggtgttcctgcaca |
| | WHFANLEFANATP | gcagtccccaagcatgtga (SEQ ID NO: 176) |
| | LSTLSLKHWDQDD | |
| | DFEFTGSHLTVRN | |
| | GYSCVPVALAEGL | |
| | DIKLNTAVRQVRY | |
| | TASGCEVIAVNTRS | |
| | TSQTFIYKCDAVL | |
| | CTLPLGVLKQQPP | |
| | AVQFVPPLPEWKT | |
| | SAVQRMGFGNLN | |
| | KVVLCFDRVFWDP | |
| | SVNLFGHVGSTTA | |
| | SRGELFLFWNLYK | |
| | APILLALVAGEAA | |
| | GIMENISDDVIVGR | |
| | CLAILKGIFGSSAV | |
| | PQPKETVVSRWRA | |
| | DPWARGSYSYVAA | |
| | GSSGNDYDLMAQP | |
| | ITPGPSIPGAPQPIP | |
| | RLFFAGEHTIRNYP | |
| | ATVHGALLSGLRE | |
| | AGRIADQFLGAMY | |
| | TLPRQATPGVPAQ | |
| | QSPSM* (SEQ ID | |
| | NO: 148) | |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| KRAB- | QVDRYIASEVCEDSIT | CTGAAGGACCTGGGCATCCAAGTGGACCGCTACATTGCC |
| DOT1L | VGMVRHQGKIMYVG | TCCGAGGTGTGTGAGGACTCCATCACGGTGGGCATGGTG |
| (FIG. | DVRSVTQKHIQEWG | CGGCACCAGGGAAAGATCATGTACGTCGGGGACGTCCG |
| 4B) | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccggggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctgggcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESRTLVTFKD | CTCAGCTGGGAGGCGACGCCTATCCCTATGACGTGCCCG |
| | VFVDFTREEWKLL | ATTATGCCAGCCTGGGCAGCGGCTCCCCCAAGAAAAAA |
| | DTAQQIVYRNVMLE | CGCAAGGTGGAAGATCCTAAGAAAAAGCGGAAAGTGGA |
| | NYKNLVSLGYQLTK | CGGCagcggcagcgagacccccggtaccagcgagaggccacccccgagagcCG |
| | PDVILRLEKGEEPA | GACACTGGTGACCTTCAAGGATGTATTTGTGGACTTCAC |
| | YPYDVPDYASLGSG | CAGGGAGGAGTGGAAGCTGCTGGACACTGCTCAGCAGA |
| | SPKKKRKVEDPKK | TCGTGTACAGAAATGTGATGCTGGAGAACTATAAGAACC |
| | KRKVDGSGSETPGT | TGGTTTCCTTGGGTTATCAGCTTACTAAGCCAGATGTGAT |
| | SESATPESMAKTVA | CCTCCGGTTGGAGAAGGGAGAAGAGCCCagcggcagcgagacc |
| | YFYDPDVGNFHYG | cccggtaccagcgagagcgccacccccgagagcatgggggagaagctggagctgagact |
| | AGHPMKPHRLAL | gaagtcgcccgtgggggctgagcccgccgtctacccgtggccgctgccggtctacgataaa |
| | THSLVLHYGLYKK | catcacgatgctgctcatgaaatcatcgagaccatccgatgggtctgtgaagaaatcccggat |
| | MIVFKPYQASQHD | ctcaagctcgctatggagaattacgttttaattgactatgacaccaaaagcttcgagagcatgca |
| | MCRFHSEDYIDFL | gaggctctgcgacaagtacaaccgtgccatcgacagcatccaccagctgtggaagggcacc |
| | QRVSPTNMQGFTK | acgcagcccatgaagctgaacacgcggccgtccactggactcctgcgccatatcctgcagc |
| | SLNAFNVGDDCPV | aggtctacaaccactcggtgaccgaccccgagaagctcaacaactacgagcccttctccccc |
| | FPGLFEFCSRYTG | gaggtgtacggggagacctccttcgacctggtggcccagatgattgatgagatcaagatgac |
| | ASLQGATQLNNKI | cgacgacgacctgtttgtggacttggggagcggtgtgggccaggtcgtgctccaggttgctg |
| | CDIAINWAGGLHH | ctgccaccaactgcaaacatcactatggcgtcgagaaagcagacatcccggccaagtatgcg |
| | AKKFEASGFCYVN | gagaccatggaccgcgagttcaggaagtggatgaaatggtatggaaaaaagcatgcagaat |
| | DIVIGILELLKYHP | acacattggagagaggcgatttcctctcagaagagtggagggagcgaatcgccaacacgag |
| | RVLYIDIDIHHGDG | tgttatatttgtgaataattttgcctttggtcctgaggtggatcaccagctgaaggagcggtttgca |
| | VQEAFYLTDRVMT | aacatgaaggaaggtggcagaatcgtgtcctcgaaaccctttgcacctctgaacttcagaata |
| | VSFHKYGNYFFPG | aacagtagaaacttgagtgacatcggcaccatcatgcgcgtggtggagctctcgcccctgaa |
| | TGDMYEVGAESG | gggctcggtgtcgtggacggggaagccagtctcctactacctgcacactatcgaccgcacca |
| | RYYCLNVPLRDGI | tacttgaaaactatttttctagtctgaaaaacccaaaactcagggaggaacaggaggcagccc |
| | DDQSYKHLFQPVI | ggcgccgccagcagcgcgagagcaagagcaacgcggccacgcccactaagggcccaga |
| | NQVVDFYQPTCIV | gggcaaggtggccggccccgccgacgcccccatggactctggtgctgaggaagagaagg |
| | LQCGADSLGCDRL | cgggagcagccaccgtgaagaagccgtctccctccaaagcccgcaagaagaagctaaaca |
| | GCFNLSIRGHGEC | agaaggggaggaagatggctggccgcaagcgcgggcgccccaagaagatgaacactgc |
| | VEYVKSFNIPLLVL | gaaccccgagcggaagcccaagaagaaccaaactgcactggatgccctgcacgctcagac |
| | GGGGYTVRNVAR | cgtgtctcagacggcggcctcctcaccccaggatgcctacagatcccctcacagcccgttcta |
| | CWTYETSLLVEEA | ccagctacctccgagcgtgcagcggcactcccccaacccgctgctggtggcgcccaccccg |
| | ISEELPYSEYFEYF | cccgcgctgcagaagcttctagagtccttcaagatccagtacctgcagttcctggcatacacaa |
| | APDFTLHPDVSTRI | agaccccccagtacaaggccagcctgcaggagctgctgggccaggagaaggagaagaac |
| | ENQNSRQYLDQIR | gcccagctcctgggtgcggctcagcagctcctcagccactgccaggcccagaaggaggag |
| | QTIFENLKMLNHA | atcaggaggctgtttcagcaaaaattggatgagctgggtgtgaaggcgctgacctacaacga |
| | PSVQIHDVPADLLT | cctgattcaagcgcagaaggagatctccgcccataaccagcagctgcgggagcagtcggag |
| | YDRTDEADAEERG | cagctggagcaggacaaccgcgcgctccgcggccagagcttgcagctgctcaaggctcgc |
| | PEENYSRSGSRDFS | tgcgaggagctgcagctggactgggccacgctgtcgctggagaagctgttgaaggagaagc |
| | LLSIGLLGRFKRE | aggccctgaagagccagatctcggagaagcagaggcactgcctggagctgcagatcagca |
| | KVNGKVLV*(SEQ | ttgtggagctagagaagagccagcggcagcaggagctcctgcagctcaagtcctgtgtgcc |
| | ID NO: 149) | gcctgacgacgccctgtccctgcacctgcgtgggaagggcgccctgggccgcgagctgga |
| | | gcctgacgccagccggctgcacctggagctggactgcaccaagttctcgctgcctcacttga |
| | | gcagcatgagcccggagctctccatgaacggccaggctgctggctatgagctctgcggtgtg |
| | | ctgagccggccttcgtcgaagcagaacacgccccagtacctggcctcacccctggaccagg |
| | | aggtggtgccctgtacccctagccacgtcggccggccgcgcctggagaagctgtctggcct |
| | | agccgcacccgactacactaggctgtccccggccaagattgtgctgaggcggcacctgagc |
| | | caggaccacacggtgcccggcaggccggctgccagtgagctgcattcgagagctgagcac |
| | | accaaggagaacggccttccctaccagagccccagcgtgcctggcagcatgaagctgagc |
| | | cctcaggacccgcggcccctgtcccctggggccttgcagcttgctggagagaagagcagtg |
| | | agaagggcctgagagagcgcgcctacggcagcagcggggagctcatcaccagcctgccc |
| | | atcagcatcccgctcagcaccgtgcagcccaacaagctcccggtcagcattcccctggccag |
| | | cgtggtgctgcccagccgcgccgagaggggaggagcacccccagtcccgtgctgcagc |
| | | cccgtgacccctcgtccacacttgaaaagcagattggtgctaatgcccacggtgctgggagc |
| | | agaagccttgccctggcccccgcaggcttctcctacgctggctcggtggccatcagcggggc |
| | | cttgggggcagcccggcctctctcacacctggagccgagccggccaccttggatgagtcct |
| | | ccagctctgggagcctttttgccaccgtggggtcccgcagctccacgccacagcaccccctg |
| | | ctgctggcacagccccggaactcgcttcctgcctctcccgcccaccagctctcctccagtccc |
| | | cggcttggtggggccgcccagggcccgttgcccgaggccagcaagggagacctgccctcc |
| | | gattccggcttctcagatcctgagagtgaagccaagaggaggattgtgttcaccatcaccact |
| | | ggtgcgggcagtgccaagcagtcgccctccagcaagcacagccccctgaccgccagcgcc |
| | | cgtggggactgtgtgccgagccacgggcaggacagtcgcaggcgcggccggcggaagc |
| | | gagcatctgcggggacgcccagcttgagcgcaggcgtgtcccccaagcgccgagccctgc |
| | | cgtccgtcgctggccttttcacacagccttcggggtctcccctcaacctcaactccatggtcagt |
| | | aacatcaaccagcccctggagattacagccatctcgtccccggagacctccctgaagagctc |
| | | ccctgtgccctaccaggaccacgaccagccccccgtgctcaagaaggagcggcctctgagc |
| | | cagaccaatggggcacactactccccactcacctcagacgaggagccaggctctgaggacg |
| | | agcccagcagtgctcgaattgagagaaaaattgcaacaatctccttagaaagcaaatctcccc |
| | | cgaaaaccttggaaaatggtggtggcttggcgggaaggaagcccgcgcccgccggcgagc |
| | | cagtcaatagcagcaagtggaagtccaccttctcgcccatctccgacatcggcctggccaagt |
| | | cggcggacagcccgctgcaggccagctccgccctcagccagaactccctgttcacgttccg |
| | | gcccgccctggaggagccctctgccgatgccaagctggccgctcaccccaggaaaggcttt |
| | | cccggctccctgtcgggggctgacggactcagcccgggcaccaaccctgccaacggctgc |
| | | accttcggcgggggcctggccgcggacctgagtttacacagcttcagtgatggtgcttctcttc |
| | | cccacaagggccccgaggcggccggcctgagctccccgctgagcttcccctcgcagcgcg |
| | | gcaaggagggctcggacgccaaccctttcctgagcaagaggcagctggacggcctggctg |
| | | ggctgaagggcgagggcagccgcggcaaggaggcaggggagggcggcctaccgctgtg |
| | | cgggcccacggacaagaccccactgctgagcggcaaggccgccaaggcccgggaccgc |
| | | gaggtcgacctcaagaatggccacaacctcttcatctctgcggcggccgtgcctcccggaag |
| | | cctcctcagcggccccggcctggccccggcggcgtcctccgcaggcggcgcggcgtcctc |
| | | cgcccagacgcaccggtccttcctgggccccttcccgccgggaccgcagttcgcgctcggc |
| | | cccatgtccctgcaggccaacctcggctccgtggccggctcctccgtgctgcagtcgctgttc |
| | | agctctgtgccggccgccgcaggcctggtgcacgtgtcgtccgctgccaccagactgacca |
| | | actcgcacgccatgggcagcttttccggggtggcaggcggcacagttggaggtgtctttaac |
| | | cacgcggtgccctccgcctctgctcatccgtttggagcccgtgtcggccgcggggctgcatgt |
| | | ggcagcgccacactgggcccgagcccgctgcaggcggcggccagcgcctcggcctcttcc |
| | | tttcaggccccggcctcggttgagacccggccgccccctccgcctccgcctccgcctccccc |
| | | gctgcccccgcctgcgcacctgggccggtcccccgcggggccgcccgtcctccacgcccc |
| | | ccctccacctaacgccgccttgcctcctcccccaacgctgctggcctctaaccctgagcccgc |
| | | gcttctgcagagcctcgcgtccctcccgcctaaccaagctttcttgccccccacctctgctgcct |
| | | ctctgccgcctgctaacgcctctttgtctatcaagctcacctccctcccgcacaagggcgcccg |
| | | cccctccttcacggtgcaccaccagcccctgccccggctggccctggcccaggccgcgccc |
| | | gggatcccacaggccagcgccacggggccgtccgcggtgtgggtgtccctcggcatgccg |
| | | cctccctatgccgcgcacctttcgggggttaagccgcgataa (SEQ ID NO: 177) |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| KRAB- | QVDRYIASEVCEDSIT | CTGAAGGACCTGGGCATCCAAGTGGACCGCTACATTGCC |
| DHAC3 | VGMVRHQGKIMYVG | TCCGAGGTGTGTGAGGACTCCATCACGGTGGGCATGGTG |
| (FIG. | DVRSVTQKHIQEWG | CGGCACCAGGGAAAGATCATGTACGTCGGGGACGTCCG |
| 4B) | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLEDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGSGSETPGTSE | AGAGCATCACCGGCCTGTACGAGACACGGATCGACCTGT |
| | SATPESRTLVTFKD | CTCAGCTGGGAGGCGACGCCTATCCCTATGACGTGCCCG |
| | VFVDFTREEWKLL | ATTATGCCAGCCTGGGCAGCGGCTCCCCCAAGAAAAAA |
| | DTAQQIVYRNVMLE | CGCAAGGTGGAAGATCCTAAGAAAAAGCGGAAAGTGGA |
| | NYKNLVSLGYQLTK | CGGCagcggcagcgagacccccggtaccagcgagagcgccacccccgagagcCG |
| | PDVILRLEKGEEPA | GACACTGGTGACCTTCAAGGATGTATTTGTGGACTTCAC |
| | YPYDVPDYASLGSG | CAGGGAGGAGTGGAAGCTGCTGGACACTGCTCAGCAGA |
| | SPKKKRKVEDPKK | TCGTGTACAGAAATGTGATGCTGGAGAACTATAAGAACC |
| | KRKVDGSGSETPGT | TGGTTTCCTTGGGTTATCAGCTTACTAAGCCAGATGTGAT |
| | SESATPESMAKTVA | CCTCCGGTTGGAGAAGGGAGAAGAGCCCagcggcagcgagacc |
| | YFYDPDVGNFHYG | cccggtaccagcgagagcgccacccccgagagcatggccaagaccgtggcctatttctacg |
| | AGHPMKPHRLAL | accccgacgtgggcaacttccactacggagctggacaccctatgaagccccatcgcctggc |
| | THSLVLHYGLYKK | attgacccatagcctggtcctgcattacggtctctataagaagatgatcgtcttcaagccatacc |
| | MIVFKPYQASQHD | aggcctcccaacatgacatgtgccgcttccactccgaggactacattgacttcctgcagagag |
| | MCRFHSEDYIDFL | tcagccccaccaatatgcaaggcttcaccaagagtcttaatgccttcaacgtaggcgatgactg |
| | QRVSPTNMQGFTK | cccagtgtttcccgggctctttgagttctgctcgcgttacacaggcgcatctctgcaaggagca |
| | SLNAFNVGDDCPV | acccagctgaacaacaagatctgtgatattgccattaactgggctggtggtctgcaccatgcca |
| | FPGLFEFCSRYTG | agaagtttgaggcctctggcttctgctatgtcaacgacattgtgattggcatcctggagctgctc |
| | ASLQGATQLNNKI | aagtaccaccctcgggtgctctacattgacattgacatccaccatggtgacggggttcaagaa |
| | CDIAINWAGGLHH | gctttctacctcactgaccgggtcatgacggtgtccttccacaaatacggaaattacttcttccct |
| | AKKFEASGFCYVN | ggcacaggtgacatgtatgaagtcggggcagagagtggccgctactactgtctgaacgtgcc |
| | DIVIGILELLKYHP | cctgcgggatggcattgatgaccagagttacaagcaccttttccagccggttatcaaccaggta |
| | RVLYIDIDIHHGDG | gtggacttctaccaacccacgtgcattgtgctccagtgtggagctgactctctgggctgtgatc |
| | VQEAFYLTDRVMT | gattgggctgctttaacctcagcatccgagggcatggggaatgcgttgaatatgtcaagagctt |
| | VSFHKYGNYFFPG | caatatccctctactcgtgctgggtggtggtggttatactgtccgaaatgttgcccgctgctgga |
| | TGDMYEVGAESG | catatgagacatcgctgctggtagaagaggccattagtgaggagcttccctatagtgaatactt |
| | RYYCLNVPLRDGI | cgagtactttgccccagacttcacacttcatccagatgtcagcacccgcatcgagaatcagaa |
| | DDQSYKHLFQPVI | ctcacgccagtatctggaccagatccgccagacaatctttgaaaacctgaagatgctgaacca |
| | NQVVDFYQPTCIV | tgcacctagtgtccagattcatgacgtgcctgcagacctcctgacctatgacaggactgatgag |
| | LQCGADSLGCDRL | gctgatgcagaggagaggggtcctgaggagaactatagcaggtctgggagcagggacttca |
| | GCFNLSIRGHGEC | gcctactttcaataggcttgctggggaggtttaaaagggaaaaagtaaatgggaaggtattagt |
| | VEYVKSFNIPLLVL | ttaa (SEQ ID NO: 178) |
| | GGGGYTVRNVAR | |
| | CWTYETSLLVEEA | |
| | ISEELPYSEYFEYF | |
| | APDFTLHPDVSTRI | |
| | ENQNSRQYLDQIR | |
| | QTIFENLKMLNHA | |
| | PSVQIHDVPADLLT | |
| | YDRTDEADAEERG | |
| | PEENYSRSGSRDFS | |
| | LLSIGLLGRFKRE | |
| | KVNGKVLV* (SEQ | |
| | ID NO: 150) | |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9- | DGIATGLLVLKDLGI | GCTGTCTCTCTTTGATGGGATTGCTACAGGGCTCCTGGTG |
| 10X | QVDRYIASEVCEDSIT | CTGAAGGACCTGGGCATCCAAGTGGACCGCTACATTGCC |
| GCN4 | VGMVRHQGKIMYVG | TCCGAGGTGTGTGAGGACTCCATCACGGTGGGCATGGTG |
| (FIG. | DVRSVTQKHIQEWG | CGGCACCAGGGAAAGATCATGTACGTCGGGGACGTCCG |
| 2B) | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLEDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGDAYP | ACCTGTTTACCCTGACCAATCTGGGAGCCCCTGCCGCCT |
| | YDVPDYASLGSGSP | TCAAGTACTTTGACACCACCATCGACCGGAAGAGGTACA |
| | KKKRKVEDPKKKR | CCAGCACCAAAGAGGTGCTGGACGCCACCCTGATCCACC |
| | KVDGIGSGSNGSSG | AGAGCATCACCGGCCTGTACGAGACACGGATCGACCTGT |
| | EELLSKNYHLENE | CTCAGCTGGGAGGCGACGCCTATCCCTATGACGTGCCCG |
| | VARLKKGSGSGGS | ATTATGCCAGCCTGGGCAGCGGCTCCCCCAAGAAAAAA |
| | GSGGSGSGSGGSG | CGCAAGGTGGAAGATCCTAAGAAAAAGCGGAAAGTGGA |
| | SGGSGSGEELLSK | CGGCATTGGTAGTGGGAGCAACGGCAGCAGCGGAGAGG |
| | NYHLENEVARLKK | AACTGCTGAGCAAGAACTACCACCTGGAAAACGAGGTG |
| | GSGSGGSGSGGSG | GCCAGACTGAAGAAAGGCTCTGGCTCTGGCGGAAGCGG |
| | SGSGGSGSGGSGS | TTCTGGCGGATCAGGATCTGGAAGTGGCGGCTCTGGAAG |
| | GEELLSKNYHLEN | CGGAGGTTCTGGATCTGGCGAAGAACTGCTGTCTAAGAA |
| | EVARLKKGSGSGG | TTATCACCTCGAGAACGAAGTGGCTCGGCTCAAGAAAG |
| | SGSGGSGSGSGGS | GCAGTGGCAGCGGAGGAAGTGGATCCGGCGGTAGTGGT |
| | GSGGSGSGEELLS | AGTGGAAGCGGCGGATCCGGCTCAGGCGGATCTGGTTC |
| | KNYHLENEVARLK | AGGGGAAGAACTCCTCTCCAAAAACTACCATCTCGAGA |
| | KGSGSGGSGSGGS | ATGAGGTCGCCCGCCTGAAAAAAGGATCAGGTTCCGGT |
| | GSGSGGSGSGGSG | GGTTCTGGCAGCGGTGGTTCAGGCTCAGGCAGCGGCGGT |
| | SGEELLSKNYHLE | AGCGGTAGCGGTGGAAGCGGAAGCGGAGAAGAACTTCT |
| | NEVARLKKGSGSG | CAGCAAAAATTACCACTTGGAGAATGAAGTTGCAAGAC |
| | GSGSGGSGSGSGG | TCAAAAAAGGTTCCGGCAGTGGCGGCAGCGGCAGCGGC |
| | SGSGGSGSGEELLS | GGATCTGGTAGTGGATCTGGTGGCAGTGGTTCAGGCGGA |
| | KNYHLENEVARLK | AGTGGTAGCGGAGAGGAATTGCTCTCAAAGAACTATCAT |
| | KGSGSGGSGSGGS | TTGGAGAACGAGGTTGCACGCTTGAAGAAAGGCAGCGG |
| | GSGSGGSGSGGSG | ATCAGGCGGATCTGGCAGCGGTGGATCTGGTTCTGGATC |
| | SGEELLSKNYHLE | CGGCGGCTCCGGTAGTGGTGGAAGTGGCTCTGGGGAAG |
| | NEVARLKKGSGSG | AATTGCTTAGCAAGAATTATCATCTTGAAAATGAGGTTG |
| | GSGSGGSGSGSGG | CCAGGCTTAAAAAAGGCAGTGGCTCCGGCGGATCCGGA |
| | SGSGGSGSGEELLS | AGCGGAGGCAGCGGATCTGGATCTGGTGGTTCAGGATCT |
| | KNYHLENEVARLK | GGCGGTTCTGGTAGCGGGGAAGAACTGTTGAGTAAAAA |
| | KGSGSGGSGSGGS | CTATCACCTTGAGAACGAGGTCGCAAGGTTGAAAAAAG |
| | GSGSGGSGSGGSG | GATCCGGCTCTGGCGGCTCCGGAAGTGGCGGATCTGGCT |
| | SGEELLSKNYHLE | CCGGTAGCGGAGGATCAGGATCCGGCGGAAGCGGATCA |
| | NEVARLKKGSGSG | GGCGAGGAACTGCTTTCCAAAAATTACCACCTTGAAAAC |
| | GSGSGGSGSGSGG | GAAGTCGCCCGCCTCAAGAAAGGTTCTGGCAGCGGAGG |
| | SGSGGSGSGEELLS | CTCTGGCAGTGGTGGTAGCGGAAGTGGAAGTGGTGGCA |
| | KNYHLENEVARLK | GTGGTAGCGGTGGATCTGGAAGCGGCGAGGAACTCCTG |
| | KGSGSGQRPQGGG | TCAAAGAATTACCATCTCGAAAACGAGGTCGCAAGGCTC |
| | GPKKKRKV (SEQ ID | AAGAAAGGCTCAGGATCAGGCGGCTCTGGATCCGGCGG |
| | NO: 151) | TTCTGGTTCCGGCTCAGGTGGAAGTGGATCTGGCGGCTC |
| | | AGGTTCCGGCGAAGAATTGCTTTCCAAGAACTACCATTT |
| | | GGAAAATGAAGTCGCTCGTTTGAAGAAAGGTTCAGGCTC |
| | | CGGCcagcggccgcaaGGTGGAGGTGGACCCAAGAAGAAGCG |
| | | CAAGGTG (SEQ ID NO: 179) |
| SCFV- | MGPDIVMTQSPSS | ATGGGCCCCGACATCGTGATGACCCAGAGCCCCAGCAG |
| KRAB | LSASVGDRVTITCR | CCTGAGCGCCAGCGTGGGCGACCGCGTGACCATCACCTG |
| (FIG. | SSTGAVTTSNYAS | CCGCAGCAGCACCGGCGCCGTGACCACCAGCAACTACG |
| 2B) | WVQEKPGKLFKG | CCAGCTGGGTGCAGGAGAAGCCCGGCAAGCTGTTCAAG |
| | LIGGTNNRAPGVP | GGCCTGATCGGCGGCACCAACAACCGCGCCCCCGGCGT |
| | SRFSGSLIGDKATL | GCCCAGCCGCTTCAGCGGCAGCCTGATCGGCGACAAGG |
| | TISSLQPEDFATYF | CCACCCTGACCATCAGCAGCCTGCAGCCCGAGGACTTCG |
| | CALWYSNHWVFG | CCACCTACTTCTGCGCCCTGTGGTACAGCAACCACTGGG |
| | QGTKVELKRGGG | TGTTCGGCCAGGGCACCAAGGTGGAGCTGAAGCGCGGC |
| | GSGGGGSGGGGSS | GGCGGCGGCAGCGGCGGCGGCGGCAGCGGCGGCGGCGG |
| | GGGSEVKLLESGG | CAGCAGCGGCGGCGGCAGCGAGGTGAAGCTGCTGGAGA |
| | GLVQPGGSLKLSC | GCGGCGGCGGCCTGGTGCAGCCCGGCGGCAGCCTGAAG |
| | AVSGFSLTDYGVN | CTGAGCTGCGCCGTGAGCGGCTTCAGCCTGACCGACTAC |
| | WVRQAPGRGLEW | GGCGTGAACTGGGTGCGCCAGGCCCCCGGCCGCGGCCT |
| | IGVIWGDGITDYNS | GGAGTGGATCGGCGTGATCTGGGGCGACGGCATCACCG |
| | ALKDRFIISKDNGK | ACTACAACAGCGCCCTGAAGGACCGCTTCATCATCAGCA |
| | NTVYLQMSKVRSD | AGGACAACGGCAAGAACACCGTGTACCTGCAGATGAGC |
| | DTALYYCVTGLFD | AAGGTGCGCAGCGACGACACCGCCCTGTACTACTGCGTG |
| | YWGQGTLVTVSSY | ACCGGCCTGTTCGACTACTGGGGCCAGGGCACCCTGGTG |
| | PYDVPDYAGGGG | ACCGTGAGCAGCTACCCATACGATGTTCCAGATTACGCT |
| | GSGGGGSGGGGS | GGTGGAGGCGGAGGTTCTGGGGGAGGAGGTAGTGGCGG |
| | GGGGSLDPGGGGS | TGGTGGTTCAGGAGGCGGCGGAAGCTTGGATCCAGGTG |
| | GRTLVTFKDVFVD | GAGGTGGAAGCGGTCGGACACTGGTGACCTTCAAGGAT |
| | FTREEWKLLDTAQ | GTATTTGTGGACTTCACCAGGGAGGAGTGGAAGCTGCTG |
| | QIVYRNVMLENYK | GACACTGCTCAGCAGATCGTGTACAGAAATGTGATGCTG |
| | NLVSLGYQLTKPD | GAGAACTATAAGAACCTGGTTTCCTTGGGTTATCAGCTT |
| | VILRLEKGEEPGSG | ACTAAGCCAGATGTGATCCTCCGGTTGGAGAAGGGAGA |
| | KRTADGSEFESPKK | AGAGCCCGGAAGCGGAaagcgcaccgccgatggttccgagttcgaaagccc |
| | KRK (SEQ ID NO: | caaaaaaaagcgcaaggt (SEQ ID NO: 180) |
| | 152) | |
| dCas2- | MDKKYSIGLAIGT | CCAAAAAAGAAGAGAAAGGTAATGGACAAGAAGTACA |
| 10X | NSVGWAVITDEYK | GCATCGGCCTGGCCATCGGCACCAACTCTGTGGGCTGGG |
| GCN4 | VPSKKFKVLGNTD | CCGTGATCACCGACGAGTACAAGGTGCCCAGCAAGAAA |
| (FIG. | RHSIKKNLIGALLF | TTCAAGGTGCTGGGCAACACCGACCGGCACAGCATCAA |
| 2B) | DSGETAEATRLKR | GAAGAACCTGATCGGCGCCCTGCTGTTCGACAGCGGAG |
| | TARRRYTRRKNRI | AAACAGCCGAGGCCACCCGGCTGAAGAGAACCGCCAGA |
| | CYLQEIFSNEMAK | AGAAGATACACCAGACGGAAGAACCGGATCTGCTATCT |
| | VDDSFFHRLEESFL | GCAAGAGATCTTCAGCAACGAGATGGCCAAGGTGGACG |
| | VEEDKKHERHPIF | ACAGCTTCTTCCACAGACTGGAAGAGTCCTTCCTGGTGG |
| | GNIVDEVAYHEKY | AAGAGGATAAGAAGCACGAGCGGCACCCCATCTTCGGC |
| | PTIYHLRKKLVDS | AACATCGTGGACGAGGTGGCCTACCACGAGAAGTACCC |
| | TDKADLRLIYLAL | CACCATCTACCACCTGAGAAAGAAACTGGTGGACAGCA |
| | AHMIKFRGHFLIE | CCGACAAGGCCGACCTGCGGCTGATCTATCTGGCCCTGG |
| | GDLNPDNSDVDKL | CCCACATGATCAAGTTCCGGGGCCACTTCCTGATCGAGG |
| | FIQLVQTYNQLFE | GCGACCTGAACCCCGACAACAGCGACGTGGACAAGCTG |
| | ENPINASGVDAKAI | TTCATCCAGCTGGTGCAGACCTACAACCAGCTGTTCGAG |
| | LSARLSKSRRLENL | GAAAACCCCATCAACGCCAGCGGCGTGGACGCCAAGGC |
| | IAQLPGEKKNGLF | CATCCTGTCTGCCAGACTGAGCAAGAGCAGACGGCTGG |
| | GNLIALSLGLTPNF | AAAATCTGATCGCCCAGCTGCCCGGCGAGAAGAAGAAT |
| | KSNFDLAEDAKLQ | GGCCTGTTCGGCAACCTGATTGCCCTGAGCCTGGGCCTG |
| | LSKDTYDDDLDNL | ACCCCCAACTTCAAGAGCAACTTCGACCTGGCCGAGGAT |
| | LAQIGDQYADLFL | GCCAAACTGCAGCTGAGCAAGGACACCTACGACGACGA |
| | AAKNLSDAILLSDI | CCTGGACAACCTGCTGGCCCAGATCGGCGACCAGTACGC |
| | LRVNTEITKAPLSA | CGACCTGTTTCTGGCCGCCAAGAACCTGTCCGACGCCAT |
| | SMIKRYDEHHQDL | CCTGCTGAGCGACATCCTGAGAGTGAACACCGAGATCAC |
| | TLLKALVRQQLPE | CAAGGCCCCCCTGAGCGCCTCTATGATCAAGAGATACGA |
| | KYKEIFFDQSKNG | CGAGCACCACCAGGACCTGACCCTGCTGAAAGCTCTCGT |
| | YAGYIDGGASQEE | GCGGCAGCAGCTGCCTGAGAAGTACAAAGAGATTTTCTT |
| | FYKFIKPILEKMD | CGACCAGAGCAAGAACGGCTACGCCGGCTACATCGATG |
| | GTEELLVKLNRED | GCGGAGCCAGCCAGGAAGAGTTCTACAAGTTCATCAAG |
| | LLRKQRTFDNGSIP | CCCATCCTGGAAAAGATGGACGGCACCGAGGAACTGCT |
| | HQIHLGELHAILR | CGTGAAGCTGAACAGAGAGGACCTGCTGCGGAAGCAGC |
| | RQEDFYPFLKDNR | GGACCTTCGACAACGGCAGCATCCCCCACCAGATCCACC |
| | EKIEKILTFRIPYY | TGGGAGAGCTGCACGCCATTCTGCGGCGGCAGGAAGAT |
| | VGPLARGNSRFAW | TTTTACCCATTCCTGAAGGACAACCGGGAAAAGATCGAG |
| | MTRKSEETITPWN | AAGATCCTGACCTTCCGCATCCCCTACTACGTGGGCCCT |
| | FEEVVDKGASAQS | CTGGCCAGGGGAAACAGCAGATTCGCCTGGATGACCAG |
| | FIERMTNFDKNLP | AAAGAGCGAGGAAACCATCACCCCCTGGAACTTCGAGG |
| | NEKVLPKHSLLYE | AAGTGGTGGACAAGGGCGCCAGCGCCCAGAGCTTCATC |
| | YFTVYNELTKVKY | GAGCGGATGACCAACTTCGATAAGAACCTGCCCAACGA |
| | VTEGMRKPAFLSG | GAAGGTGCTGCCCAAGCACAGCCTGCTGTACGAGTACTT |
| | EQKKAIVDLLFKT | CACCGTGTACAACGAGCTGACCAAAGTGAAATACGTGA |
| | NRKVTVKQLKED | CCGAGGGAATGAGAAAGCCCGCCTTCCTGAGCGGCGAG |
| | YFKKIECFDSVEIS | CAGAAAAAAGCCATCGTGGACCTGCTGTTCAAGACCAA |
| | GVEDRFNASLGTY | CCGGAAAGTGACCGTGAAGCAGCTGAAAGAGGACTACT |
| | HDLLKIIKDKDFLD | TCAAGAAAATCGAGTGCTTCGACTCCGTGGAAATCTCCG |
| | NEENEDILEDIVLT | GCGTGGAAGATCGGTTCAACGCCTCCCTGGGCACATACC |
| | LTLFEDREMIEER | ACGATCTGCTGAAAATTATCAAGGACAAGGACTTCCTGG |
| | LKTYAHLFDDKV | ACAATGAGGAAAACGAGGACATTCTGGAAGATATCGTG |
| | MKQLKRRRYTGW | CTGACCCTGACACTGTTTGAGGACAGAGAGATGATCGAG |
| | GRLSRKLINGIRDK | GAACGGCTGAAAACCTATGCCCACCTGTTCGACGACAAA |
| | QSGKTILDFLKSD | GTGATGAAGCAGCTGAAGCGGCGGAGATACACCGGCTG |
| | GFANRNFMQLIHD | GGGCAGGCTGAGCCGGAAGCTGATCAACGGCATCCGGG |
| | DSLTFKEDIQKAQ | ACAAGCAGTCCGGCAAGACAATCCTGGATTTCCTGAAGT |
| | VSGQGDSLHEHIA | CCGACGGCTTCGCCAACAGAAACTTCATGCAGCTGATCC |
| | NLAGSPAIKKGILQ | ACGACGACAGCCTGACCTTTAAAGAGGACATCCAGAAA |
| | TVKVVDELVKVM | GCCCAGGTGTCCGGCCAGGGCGATAGCCTGCACGAGCA |
| | GRHKPENIVIEMA | CATTGCCAATCTGGCCGGCAGCCCCGCCATTAAGAAGGG |
| | RENQTTQKGQKNS | CATCCTGCAGACAGTGAAGGTGGTGGACGAGCTCGTGA |
| | RERMKRIEEGIKE | AAGTGATGGGCCGGCACAAGCCCGAGAACATCGTGATC |
| | LGSQILKEHPVEN | GAAATGGCCAGAGAGAACCAGACCACCCAGAAGGGACA |
| | TQLQNEKLYLYYL | GAAGAACAGCCGCGAGAGAATGAAGCGGATCGAAGAG |
| | QNGRDMYVDQEL | GGCATCAAAGAGCTGGGCAGCCAGATCCTGAAAGAACA |
| | DINRLSDYDVDAIV | CCCCGTGGAAAACACCCAGCTGCAGAACGAGAAGCTGT |
| | PQSFLKDDSIDNKV | ACCTGTACTACCTGCAGAATGGGCGGGATATGTACGTGG |
| | LTRSDKNRGKSDN | ACCAGGAACTGGACATCAACCGGCTGTCCGACTACGATG |
| | VPSEEVVKKMKNY | TGGACGCTATCGTGCCTCAGAGCTTTCTGAAGGACGACT |
| | WRQLLNAKLITQR | CCATCGATAACAAAGTGCTGACTCGGAGCGACAAGAAC |
| | KFDNLTKAERGGL | CGGGGCAAGAGCGACAACGTGCCCTCCGAAGAGGTCGT |
| | SELDKAGFIKRQL | GAAGAAGATGAAGAACTACTGGCGCCAGCTGCTGAATG |
| | VETRQITKHVAQI | CCAAGCTGATTACCCAGAGGAAGTTCGACAATCTGACCA |
| | LDSRMNTKYDEND | AGGCCGAGAGAGGCGGCCTGAGCGAACTGGATAAGGCC |
| | KLIREVKVITLKSK | GGCTTCATCAAGAGACAGCTGGTGGAAACCCGGCAGAT |
| | LVSDFRKDFQFYK | CACAAAGCACGTGGCACAGATCCTGGACTCCCGGATGA |
| | VREINNYHHAHDA | ACACTAAGTACGACGAGAACGACAAACTGATCCGGGAA |
| | YLNAVVGTALIKK | GTGAAAGTGATCACCCTGAAGTCCAAGCTGGTGTCCGAT |
| | YPKLESEFVYGDY | TTCCGGAAGGATTTCCAGTTTTACAAAGTGCGCGAGATC |
| | KVYDVRKMIAKSE | AACAACTACCACCACGCCCACGACGCCTACCTGAACGCC |
| | QEIGKATAKYFFY | GTCGTGGGAACCGCCCTGATCAAAAAGTACCCTAAGCTG |
| | SNIMNFFKTEITLA | GAAAGCGAGTTCGTGTACGGCGACTACAAGGTGTACGA |
| | NGEIRKRPLIETNG | CGTGCGGAAGATGATCGCCAAGAGCGAGCAGGAAATCG |
| | ETGEIVWDKGRDF | GCAAGGCTACCGCCAAGTACTTCTTCTACAGCAACATCA |
| | ATVRKVLSMPQVN | TGAACTTTTTCAAGACCGAGATTACCCTGGCCAACGGCG |
| | IVKKTEVQTGGFS | AGATCCGGAAGCGGCCTCTGATCGAGACAAACGGCGAA |
| | KESILPKRNSDKLI | ACAGGCGAGATCGTGTGGGATAAGGGCCGGGACTTTGC |
| | ARKKDWDPKKYG | CACCGTGCGGAAAGTGCTGTCTATGCCCCAAGTGAATAT |
| | GFDSPTVAYSVLV | CGTGAAAAAGACCGAGGTGCAGACAGGCGGCTTCAGCA |
| | VAKVEKGKSKKL | AAGAGTCTATCCTGCCCAAGAGGAACAGCGACAAGCTG |
| | KSVKELLGITIMER | ATCGCCAGAAAGAAGGACTGGGACCCTAAGAAGTACGG |
| | SSFEKNPIDFLEAK | CGGCTTCGACAGCCCCACCGTGGCCTATTCTGTGCTGGT |
| | GYKEVKKDLIIKL | GGTGGCCAAAGTGGAAAAGGGCAAGTCCAAGAAACTGA |
| | PKYSLFELENGRK | AGAGTGTGAAAGAGCTGCTGGGGATCACCATCATGGAA |
| | RMLASAGELQKG | AGAAGCAGCTTCGAGAAGAATCCCATCGACTTTCTGGAA |
| | NELALPSKYVNFL | GCCAAGGGCTACAAAGAAGTGAAAAAGGACCTGATCAT |
| | YLASHYEKLKGSP | CAAGCTGCCTAAGTACTCCCTGTTCGAGCTGGAAAACGG |
| | EDNEQKQLFVEQH | CCGGAAGAGAATGCTGGCCTCTGCCGGCGAACTGCAGA |
| | KHYLDEIIEQISEFS | AGGGAAACGAACTGGCCCTGCCCTCCAAATATGTGAACT |
| | KRVILADANLDKV | TCCTGTACCTGGCCAGCCACTATGAGAAGCTGAAGGGCT |
| | LSAYNKHRDKPIR | CCCCCGAGGATAATGAGCAGAAACAGCTGTTTGTGGAA |
| | EQAENIIHLFTLTN | CAGCACAAACACTACCTGGACGAGATCATCGAGCAGAT |
| | LGAPAAFKYFDTTI | CAGCGAGTTCTCCAAGAGAGTGATCCTGGCCGACGCTAA |
| | DRKRYTSTKEVLD | TCTGGACAAGGTGCTGAGCGCCTACAACAAGCACAGAG |
| | ATLIHQSITGLYET | ACAAGCCTATCAGAGAGCAGGCCGAGAATATCATCCAC |
| | RIDLSQLGGDAYP | CTGTTTACCCTGACCAATCTGGGAGCCCCTGCCGCCTTC |
| | YDVPDYASLGSGSP | AAGTACTTTGACACCACCATCGACCGGAAGAGGTACACC |
| | KKKRKVEDPKKKR | AGCACCAAAGAGGTGCTGGACGCCACCCTGATCCACCA |
| | KVDGIGSGSNGSSG | GAGCATCACCGGCCTGTACGAGACACGGATCGACCTGTC |
| | EELLSKNYHLENE | TCAGCTGGGAGGCGACGCCTATCCCTATGACGTGCCCGA |
| | VARLKKGSGSGGS | TTATGCCAGCCTGGGCAGCGGCTCCCCCAAGAAAAAAC |
| | GSGGSGSGSGGSG | GCAAGGTGGAAGATCCTAAGAAAAAGCGGAAAGTGGAC |
| | SGGSGSGEELLSK | GGCATTGGTAGTGGGAGCAACGGCAGCAGCGGAGAGGA |
| | NYHLENEVARLKK | ACTGCTGAGCAAGAACTACCACCTGGAAAACGAGGTGG |
| | GSGSGGSGSGGSG | CCAGACTGAAGAAAGGCTCTGGCTCTGGCGGAAGCGGT |
| | SGSGGSGSGGSGS | TCTGGCGGATCAGGATCTGGAAGTGGCGGCTCTGGAAGC |
| | GEELLSKNYHLEN | GGAGGTTCTGGATCTGGCGAAGAACTGCTGTCTAAGAAT |
| | EVARLKKGSGSGG | TATCACCTCGAGAACGAAGTGGCTCGGCTCAAGAAAGG |
| | SGSGGSGSGSGGS | CAGTGGCAGCGGAGGAAGTGGATCCGGCGGTAGTGGTA |
| | GSGGSGSGEELLS | GTGGAAGCGGCGGATCCGGCTCAGGCGGATCTGGTTCA |
| | KNYHLENEVARLK | GGGGAAGAACTCCTCTCCAAAAACTACCATCTCGAGAAT |
| | KGSGSGGSGSGGS | GAGGTCGCCCGCCTGAAAAAAGGATCAGGTTCCGGTGG |
| | GSGSGGSGSGGSG | TTCTGGCAGCGGTGGTTCAGGCTCAGGCAGCGGCGGTAG |
| | SGEELLSKNYHLE | CGGTAGCGGTGGAAGCGGAAGCGGAGAAGAACTTCTCA |
| | NEVARLKKGSGSG | GCAAAAATTACCACTTGGAGAATGAAGTTGCAAGACTC |
| | GSGSGGSGSGSGG | AAAAAAGGTTCCGGCAGTGGCGGCAGCGGCAGCGGCGG |
| | SGSGGSGSGEELLS | ATCTGGTAGTGGATCTGGTGGCAGTGGTTCAGGCGGAAG |
| | KNYHLENEVARLK | TGGTAGCGGAGAGGAATTGCTCTCAAAGAACTATCATTT |
| | KGSGSGGSGSGGS | GGAGAACGAGGTTGCACGCTTGAAGAAAGGCAGCGGAT |
| | GSGSGGSGSGGSG | CAGGCGGATCTGGCAGCGGTGGATCTGGTTCTGGATCCG |
| | SGEELLSKNYHLE | GCGGCTCCGGTAGTGGTGGAAGTGGCTCTGGGGAAGAA |
| | NEVARLKKGSGSG | TTGCTTAGCAAGAATTATCATCTTGAAAATGAGGTTGCC |
| | GSGSGGSGSGSGG | AGGCTTAAAAAAGGCAGTGGCTCCGGCGGATCCGGAAG |
| | SGSGGSGSGEELLS | CGGAGGCAGCGGATCTGGATCTGGTGGTTCAGGATCTGG |
| | KNYHLENEVARLK | CGGTTCTGGTAGCGGGGAAGAACTGTTGAGTAAAAACT |
| | KGSGSGGSGSGGS | ATCACCTTGAGAACGAGGTCGCAAGGTTGAAAAAAGGA |
| | GSGSGGSGSGGSG | TCCGGCTCTGGCGGCTCCGGAAGTGGCGGATCTGGCTCC |
| | SGEELLSKNYHLE | GGTAGCGGAGGATCAGGATCCGGCGGAAGCGGATCAGG |
| | NEVARLKKGSGSG | CGAGGAACTGCTTTCCAAAAATTACCACCTTGAAAACGA |
| | GSGSGGSGSGSGG | AGTCGCCCGCCTCAAGAAAGGTTCTGGCAGCGGAGGCTC |
| | SGSGGSGSGEELLS | TGGCAGTGGTGGTAGCGGAAGTGGAAGTGGTGGCAGTG |
| | KNYHLENEVARLK | GTAGCGGTGGATCTGGAAGCGGCGAGGAACTCCTGTCA |
| | KGSGSGQRPQGGG | AAGAATTACCATCTCGAAAACGAGGTCGCAAGGCTCAA |
| | GPKKKRKV (SEQ ID | GAAAGGCTCAGGATCAGGCGGCTCTGGATCCGGCGGTTC |
| | NO: 153) | TGGTTCCGGCTCAGGTGGAAGTGGATCTGGCGGCTCAGG |
| | | TTCCGGCGAAGAATTGCTTTCCAAGAACTACCATTTGGA |
| | | AAATGAAGTCGCTCGTTTGAAGAAAGGTTCAGGCTCCGG |
| | | CcagcggccgcaaGGTGGAGGTGGACCCAAGAAGAAGCGCAA |
| | | GGTG (SEQ ID NO: 181) |
| SCFV- | MGPDIVMTQSPSS | ATGGGCCCCGACATCGTGATGACCCAGAGCCCCAGCAG |
| DNMT3A- | LSASVGDRVTITCR | CCTGAGCGCCAGCGTGGGCGACCGCGTGACCATCACCTG |
| DNMT3L- | SSTGAVTTSNYAS | CCGCAGCAGCACCGGCGCCGTGACCACCAGCAACTACG |
| KRAB | WVQEKPGKLFKG | CCAGCTGGGTGCAGGAGAAGCCCGGCAAGCTGTTCAAG |
| (FIG. | LIGGTNNRAPGVP | GGCCTGATCGGCGGCACCAACAACCGCGCCCCCGGCGT |
| 2B) | SRFSGSLIGDKATL | GCCCAGCCGCTTCAGCGGCAGCCTGATCGGCGACAAGG |
| | TISSLQPEDFATYF | CCACCCTGACCATCAGCAGCCTGCAGCCCGAGGACTTCG |
| | CALWYSNHWVFG | CCACCTACTTCTGCGCCCTGTGGTACAGCAACCACTGGG |
| | QGTKVELKRGGG | TGTTCGGCCAGGGCACCAAGGTGGAGCTGAAGCGCGGC |
| | GSGGGGSGGGGSS | GGCGGCGGCAGCGGCGGCGGCGGCAGCGGCGGCGGCGG |
| | GGGSEVKLLESGG | CAGCAGCGGCGGCGGCAGCGAGGTGAAGCTGCTGGAGA |
| | GLVQPGGSLKLSC | GCGGCGGCGGCCTGGTGCAGCCCGGCGGCAGCCTGAAG |
| | AVSGFSLTDYGVN | CTGAGCTGCGCCGTGAGCGGCTTCAGCCTGACCGACTAC |
| | WVRQAPGRGLEW | GGCGTGAACTGGGTGCGCCAGGCCCCCGGCCGCGGCCT |
| | IGVIWGDGITDYNS | GGAGTGGATCGGCGTGATCTGGGGCGACGGCATCACCG |
| | ALKDRFIISKDNGK | ACTACAACAGCGCCCTGAAGGACCGCTTCATCATCAGCA |
| | NTVYLQMSKVRSD | AGGACAACGGCAAGAACACCGTGTACCTGCAGATGAGC |
| | DTALYYCVTGLFD | AAGGTGCGCAGCGACGACACCGCCCTGTACTACTGCGTG |
| | YWGQGTLVTVSSY | ACCGGCCTGTTCGACTACTGGGGCCAGGGCACCCTGGTG |
| | PYDVPDYAGGGG | ACCGTGAGCAGCTACCCATACGATGTTCCAGATTACGCT |
| | GSGGGGSGGGGS | GGTGGAGGCGGAGGTTCTGGGGGAGGAGGTAGTGGCGG |
| | GGGGSLDPGGGGS | TGGTGGTTCAGGAGGCGGCGGAAGCTTGGATCCAGGTG |
| | GMNHDQEFDPPKVY | GAGGTGGAAGCGGTATGAACCATGACCAGGAATTTGAC |
| | PPVPAEKRKPIRVLSL | CCCCCAAAGGTTTACCCACCTGTGCCAGCTGAGAAGAGG |
| | FDGIATGLLVLKDLG | AAGCCCATCCGCGTGCTGTCTCTCTTTGATGGGATTGCTA |
| | IQVDRYIASEVCEDSI | CAGGGCTCCTGGTGCTGAAGGACCTGGGCATCCAAGTGG |
| | TVGMVRHQGKIMYV | ACCGCTACATTGCCTCCGAGGTGTGTGAGGACTCCATCA |
| | GDVRSVTQKHIQEW | CGGTGGGCATGGTGCGGCACCAGGGAAAGATCATGTAC |
| | GPFDLVIGGSPCNDL | GTCGGGGACGTCCGCAGCGTCACACAGAAGCATATCCA |
| | SIVNPARKGLYEGTG | GGAGTGGGGCCCATTCGACCTGGTGATTGGAGGCAGTCC |
| | RLFFEFYRLLHDARP | CTGCAATGACCTCTCCATTGTCAACCCTGCCCGCAAGGG |
| | KEGDDRPFFWLFEN | ACTTTATGAGGGTACTGGCCGCCTCTTCTTTGAGTTCTAC |
| | VVAMGVSDKRDISRF | CGCCTCCTGCATGATGCGCGGCCCAAGGAGGGAGATGA |
| | LESNPVMIDAKEVSA | TCGCCCCTTCTTCTGGCTCTTTGAGAATGTGGTGGCCATG |
| | AHRARYFWGNLPGM | GGCGTTAGTGACAAGAGGGACATCTCGCGATTTCTTGAG |
| | NRPLASTVNDKLELQ | TCTAACCCCGTGATGATTGACGCCAAAGAAGTGTCTGCT |
| | ECLEHGRIAKFSKVR | GCACACAGGGCCCGTTACTTCTGGGGTAACCTTCCTGGC |
| | TITTRSNSIKQGKDQ | ATGAACAGGCCTTTGGCATCCACTGTGAATGATAAGCTG |
| | HFPVFMNEKEDILW | GAGCTGCAAGAGTGTCTGGAGCACGGCAGAATAGCCAA |
| | CTEMESSGNSNANS | GTTCAGCAAAGTGAGGACCATTACCACCAGGTCAAACTC |
| | RGPSFSSGLVPLSLR | TATAAAGCAGGGCAAAGACCAGCATTTCCCCGTCTTCAT |
| | GSHMGPMEIYKTVS | GAACGAGAAGGAGGACATCCTGTGGTGCACTGAAATGG |
| | AWKRQPVRVLSLF | AAAGGGTGTTTGGCTTCCCCGTCCACTACACAGACGTCT |
| | RNIDKVLKSLGFLE | CCAACATGAGCCGCTTGGCGAGGCAGAGACTGCTGGGC |
| | SGSGSGGGTLKYVE | CGATCGTGGAGCGTGCCGGTCATCCGCCACCTCTTCGCT |
| | DVTNVVRRDVEKW | CCGCTGAAGGAATATTTTGCTTGTGTGtctagggcaatagtaacgc |
| | GPFDLVYGSTQPLG | taacagccgcgggccgagcttcagcagcggcctggtgccgttaagcttgcgcggcagccat |
| | SSCDRCPGWYMFQ | ATGggccctatggagatatacaagacagtgtctgcatggaagagacagccagtgcgggta |
| | FHRILQYALPRQES | ctgagcctcttcagaaacatcgacaaggtactaaagagtttgggcttcttGGAAAGCG |
| | QRPFFWIFMDNLLL | GTTCTGGTTCTGGGGGAGGAACGCTGAAGTACGTGGAA |
| | TEDDQETTTRFLQT | GATGTCACAAATGTCGTGAGGAGAGACGTGGAGAAATG |
| | EAVTLQDVRGRDY | GGGCCCCTTTGACCTGGTGTACGGCTCGACGCAGCCCCT |
| | QNAMRVWSNIPGL | AGGCAGCTCTTGTGATCGCTGTCCCGGCTGGTACATGTT |
| | KSKHAPLTPKEEEY | CCAGTTCCACCGGATCCTGCAGTATGCGCTGCCTCGCCA |
| | LQAQVRSRSKLDAP | GGAGAGTCAGCGGCCCTTCTTCTGGATATTCATGGACAA |
| | KVDLLVKNCLLPLR | TCTGCTGCTGACTGAGGATGACCAAGAGACAACTACCCG |
| | EYFKYFSQNSLPLR | CTTCCTTCAGACAGAGGCTGTGACCCTCCAGGATGTCCG |
| | VFGFPVHYTDVSN | TGGCAGAGACTACCAGAATGCTATGCGGGTGTGGAGCA |
| | MSRLARQRLLGRS | ACATTCCAGGGCTGAAGAGCAAGCATGCGCCCCTGACCC |
| | WSVPVIRHLFAPLK | CAAAGGAAGAAGAGTATCTGCAAGCCCAAGTCAGAAGC |
| | EYFACVGGPSSGAP | AGGAGCAAGCTGGACGCCCCGAAAGTTGACCTCCTGGT |
| | PPSGGSPAGSPTSTE | GAAGAACTGCCTTCTCCCGCTGAGAGAGTACTTCAAGTA |
| | EGTSESATPESGPGT | TTTTTCTCAAAACTCACTTCCTCTTggagggccgagctctggcgcacc |
| | STEPSEGSAPGSPAG | cccaccaagtggagggtctcctgccgggtccccaacatctactgaagaaggcaccagcgaa |
| | SPTSTEEGTSTEPSE | tccgcaacgcccgagtcaggccctggtacctccacagaaccatctgaaggtagtgcgcctgg |
| | GSAPGTSTEPSEPKK | ttccccagctggaagccctacttccaccgaagaaggcacgtcaaccgaaccaagtgaaggat |
| | KRKVRTLVTFKDVF | ctgcccctgggaccagcactgaaccatctgagCCAAAAAAGAAGAGAAAG |
| | VDFTREEWKLLDT | GTACGGACACTGGTGACCTTCAAGGATGTATTTGTGGAC |
| | AQQIVYRNVMLENY | TTCACCAGGGAGGAGTGGAAGCTGCTGGACACTGCTCA |
| | KNLVSLGYQLTKPD | GCAGATCGTGTACAGAAATGTGATGCTGGAGAACTATA |
| | VILRLEKGEEPGSG | AGAACCTGGTTTCCTTGGGTTATCAGCTTACTAAGCCAG |
| | KRTADGSEFESPKK | ATGTGATCCTCCGGTTGGAGAAGGGAGAAGAGCCCGGA |
| | KRK (SEQ ID NO: | AGCGGAaagcgcaccgccgatggttccgagttcgaaagccccaaaaaaaagcgcaag |
| | 154) | gtc (SEQ ID NO: 182) |
| SCFV - | MGPDIVMTQSPSS | ATGGGCCCCGACATCGTGATGACCCAGAGCCCCAGCAG |
| DNMT3L- | LSASVGDRVTITCR | CCTGAGCGCCAGCGTGGGCGACCGCGTGACCATCACCTG |
| DNMT3A - | SSTGAVTTSNYAS | CCGCAGCAGCACCGGCGCCGTGACCACCAGCAACTACG |
| KRAB | WVQEKPGKLFKG | CCAGCTGGGTGCAGGAGAAGCCCGGCAAGCTGTTCAAG |
| (FIG. | LIGGTNNRAPGVP | GGCCTGATCGGCGGCACCAACAACCGCGCCCCCGGCGT |
| 2B) | SRFSGSLIGDKATL | GCCCAGCCGCTTCAGCGGCAGCCTGATCGGCGACAAGG |
| | TISSLQPEDFATYF | CCACCCTGACCATCAGCAGCCTGCAGCCCGAGGACTTCG |
| | CALWYSNHWVFG | CCACCTACTTCTGCGCCCTGTGGTACAGCAACCACTGGG |
| | QGTKVELKRGGG | TGTTCGGCCAGGGCACCAAGGTGGAGCTGAAGCGCGGC |
| | GSGGGGSGGGGSS | GGCGGCGGCAGCGGCGGCGGCGGCAGCGGCGGCGGCGG |
| | GGGSEVKLLESGG | CAGCAGCGGCGGCGGCAGCGAGGTGAAGCTGCTGGAGA |
| | GLVQPGGSLKLSC | GCGGCGGCGGCCTGGTGCAGCCCGGCGGCAGCCTGAAG |
| | AVSGFSLTDYGVN | CTGAGCTGCGCCGTGAGCGGCTTCAGCCTGACCGACTAC |
| | WVRQAPGRGLEW | GGCGTGAACTGGGTGCGCCAGGCCCCCGGCCGCGGCCT |
| | IGVIWGDGITDYNS | GGAGTGGATCGGCGTGATCTGGGGCGACGGCATCACCG |
| | ALKDRFIISKDNGK | ACTACAACAGCGCCCTGAAGGACCGCTTCATCATCAGCA |
| | NTVYLQMSKVRSD | AGGACAACGGCAAGAACACCGTGTACCTGCAGATGAGC |
| | DTALYYCVTGLFD | AAGGTGCGCAGCGACGACACCGCCCTGTACTACTGCGTG |
| | YWGQGTLVTVSSY | ACCGGCCTGTTCGACTACTGGGGCCAGGGCACCCTGGTG |
| | PYDVPDYAGGGG | ACCGTGAGCAGCTACCCATACGATGTTCCAGATTACGCT |
| | GSGGGGSGGGGS | GGTGGAGGCGGAGGTTCTGGGGGAGGAGGTAGTGGCGG |
| | GGGGSLDPGGGGS | TGGTGGTTCAGGAGGCGGCGGAAGCTTGGATCCAGGTG |
| | GMGPMEIYKTVSA | GAGGTGGAAGCGGTATGggccctatggagatatacaagacagtgtctgcatg |
| | WKRQPVRVLSLFR | gaagagacagccagtgcgggtactgagcctcttcagaaacatcgacaaggtactaaagagtt |
| | NIDKVLKSLGFLES | tgggcttcttGGAAAGCGGTTCTGGTTCTGGGGGAGGAACGCT |
| | GSGSGGGTLKYVE | GAAGTACGTGGAAGATGTCACAAATGTCGTGAGGAGAG |
| | DVTNVVRRDVEKW | ACGTGGAGAAATGGGGCCCCTTTGACCTGGTGTACGGCT |
| | GPFDLVYGSTQPLG | CGACGCAGCCCCTAGGCAGCTCTTGTGATCGCTGTCCCG |
| | SSCDRCPGWYMFQ | GCTGGTACATGTTCCAGTTCCACCGGATCCTGCAGTATG |
| | FHRILQYALPRQES | CGCTGCCTCGCCAGGAGAGTCAGCGGCCCTTCTTCTGGA |
| | QRPFFWIFMDNLLL | TATTCATGGACAATCTGCTGCTGACTGAGGATGACCAAG |
| | TEDDQETTTRFLQT | AGACAACTACCCGCTTCCTTCAGACAGAGGCTGTGACCC |
| | EAVTLQDVRGRDY | TCCAGGATGTCCGTGGCAGAGACTACCAGAATGCTATGC |
| | QNAMRVWSNIPGL | GGGTGTGGAGCAACATTCCAGGGCTGAAGAGCAAGCAT |
| | KSKHAPLTPKEEEY | GCGCCCCTGACCCCAAAGGAAGAAGAGTATCTGCAAGC |
| | LQAQVRSRSKLDAP | CCAAGTCAGAAGCAGGAGCAAGCTGGACGCCCCGAAAG |
| | KVDLLVKNCLLPLR | TTGACCTCCTGGTGAAGAACTGCCTTCTCCCGCTGAGAG |
| | EYFKYFSQNSLPLSS | AGTACTTCAAGTATTTTTCTCAAAACTCACTTCCTCTTtcta |
| | GNSNANSRGPSFSS | gcggcaatagtaacgctaacagccgcgggccgagcttcagcagcggcctggtgccgttaag |
| | GLVPLSLRGSHMNH | cttgcgcggcagccatATGAACCATGACCAGGAATTTGACCCCCC |
| | DQEFDPPKVYPPVP | AAAGGTTTACCCACCTGTGCCAGCTGAGAAGAGGAAGC |
| | AEKRKPIRVLSLFDGI | CCATCCGCGTGCTGTCTCTCTTTGATGGGATTGCTACAGG |
| | ATGLLVLKDLGIQVD | GCTCCTGGTGCTGAAGGACCTGGGCATCCAAGTGGACCG |
| | RYIASEVCEDSITVGM | CTACATTGCCTCCGAGGTGTGTGAGGACTCCATCACGGT |
| | VRHQGKIMYVGDVR | GGGCATGGTGCGGCACCAGGGAAAGATCATGTACGTCG |
| | SVTQKHIQEWGPFD | GGGACGTCCGCAGCGTCACACAGAAGCATATCCAGGAG |
| | LVIGGSPCNDLSIVNP | TGGGGCCCATTCGACCTGGTGATTGGAGGCAGTCCCTGC |
| | ARKGLYEGTGRLFFE | AATGACCTCTCCATTGTCAACCCTGCCCGCAAGGGACTT |
| | FYRLLHDARPKEGD | TATGAGGGTACTGGCCGCCTCTTCTTTGAGTTCTACCGCC |
| | DRPFFWLFENVVAM | TCCTGCATGATGCGCGGCCCAAGGAGGGAGATGATCGC |
| | GVSDKRDISRFLESN | CCCTTCTTCTGGCTCTTTGAGAATGTGGTGGCCATGGGC |
| | PVMIDAKEVSAAHRA | GTTAGTGACAAGAGGGACATCTCGCGATTTCTTGAGTCT |
| | RYFWGNLPGMNRPL | AACCCCGTGATGATTGACGCCAAAGAAGTGTCTGCTGCA |
| | ASTVNDKLELQECLE | CACAGGGCCCGTTACTTCTGGGGTAACCTTCCTGGCATG |
| | HGRIAKFSKVRTITTR | AACAGGCCTTTGGCATCCACTGTGAATGATAAGCTGGAG |
| | SNSIKQGKDQHFPVF | CTGCAAGAGTGTCTGGAGCACGGCAGAATAGCCAAGTT |
| | MNEKEDILWCTEME | CAGCAAAGTGAGGACCATTACCACCAGGTCAAACTCTAT |
| | RVFGFPVHYTDVSN | AAAGCAGGGCAAAGACCAGCATTTCCCCGTCTTCATGAA |
| | MSRLARQRLLGRS | CGAGAAGGAGGACATCCTGTGGTGCACTGAAATGGAAA |
| | WSVPVIRHLFAPLK | GGGTGTTTGGCTTCCCCGTCCACTACACAGACGTCTCCA |
| | EYFACVGGPSSGAP | ACATGAGCCGCTTGGCGAGGCAGAGACTGCTGGGCCGA |
| | PPSGGSPAGSPTSTE | TCGTGGAGCGTGCCGGTCATCCGCCACCTCTTCGCTCCG |
| | EGTSESATPESGPGT | CTGAAGGAATATTTTGCTTGTGTGggagggccgagctctggcgcacc |
| | STEPSEGSAPGSPAG | cccaccaagtggagggtctcctgccgggtccccaacatctactgaagaaggcaccagcgaa |
| | SPTSTEEGTSTEPSE | tccgcaacgcccgagtcaggccctggtacctccacagaaccatctgaaggtagtgcgcctgg |
| | GSAPGTSTEPSEPKK | ttccccagctggaagccctacttccaccgaagaaggcacgtcaaccgaaccaagtgaaggat |
| | KRKVRTLVTFKDVF | ctgcccctgggaccagcactgaaccatctgagCCAAAAAAGAAGAGAAAG |
| | VDFTREEWKLLDT | GTACGGACACTGGTGACCTTCAAGGATGTATTTGTGGAC |
| | AQQIVYRNVMLENY | TTCACCAGGGAGGAGTGGAAGCTGCTGGACACTGCTCA |
| | KNLVSLGYQLTKPD | GCAGATCGTGTACAGAAATGTGATGCTGGAGAACTATA |
| | VILRLEKGEEPGSG | AGAACCTGGTTTCCTTGGGTTATCAGCTTACTAAGCCAG |
| | KRTADGSEFESPKK | ATGTGATCCTCCGGTTGGAGAAGGGAGAAGAGCCCGGA |
| | KRK (SEQ ID NO: | AGCGGAaagcgcaccgccgatggttccgagttcgaaagccccaaaaaaaagcgcaag |
| | 155) | gtc (SEQ ID NO: 183) |
| SCFV - | MGPDIVMTQSPSSLSA | ATGGGCCCCGACATCGTGATGACCCAGAGCCCCAGCAGCCTGAGCGC |
| DNMT3A- | SVGDRVTITCRSSTGAV | CAGCGTGGGCGACCGCGTGACCATCACCTGCCGCAGCAGCACCGGCG |
| DNMT3L | TTSNYASWVQEKPGKL | CCGTGACCACCAGCAACTACGCCAGCTGGGTGCAGGAGAAGCCCGGC |
| (FIG. | FKGLIGGTNNRAPGVPS | AAGCTGTTCAAGGGCCTGATCGGCGGCACCAACAACCGCGCCCCCGG |
| 2B) | RFSGSLIGDKATLTISSL | CGTGCCCAGCCGCTTCAGCGGCAGCCTGATCGGCGACAAGGCCACCC |
| | QPEDFATYFCALWYSN | TGACCATCAGCAGCCTGCAGCCCGAGGACTTCGCCACCTACTTCTGCG |
| | HWVFGQGTKVELKRG | CCCTGTGGTACAGCAACCACTGGGTGTTCGGCCAGGGCACCAAGGTG |
| | GGGSGGGGSGGGGSS | GAGCTGAAGCGCGGCGGCGGCGGCAGCGGCGGCGGCGGCAGCGGC |
| | GGGSEVKLLESGGGLV | GGCGGCGGCAGCAGCGGCGGCGGCAGCGAGGTGAAGCTGCTGGAG |
| | QPGGSLKLSCAVSGFSL | AGCGGCGGCGGCCTGGTGCAGCCCGGCGGCAGCCTGAAGCTGAGCT |
| | TDYGVNWVRQAPGRG | GCGCCGTGAGCGGCTTCAGCCTGACCGACTACGGCGTGAACTGGGTG |
| | LEWIGVIWGDGITDYN | CGCCAGGCCCCCGGCCGCGGCCTGGAGTGGATCGGCGTGATCTGGG |
| | SALKDRFIISKDNGKNT | GCGACGGCATCACCGACTACAACAGCGCCCTGAAGGACCGCTTCATC |
| | VYLQMSKVRSDDTALY | ATCAGCAAGGACAACGGCAAGAACACCGTGTACCTGCAGATGAGCAA |
| | YCVTGLFDYWGQGTLV | GGTGCGCAGCGACGACACCGCCCTGTACTACTGCGTGACCGGCCTGT |
| | TVSSYPYDVPDYAGGG | TCGACTACTGGGGCCAGGGCACCCTGGTGACCGTGAGCAGCTACCCA |
| | GGSGGGGGGGGSGG | TACGATGTTCCAGATTACGCTGGTGGAGGCGGAGGTTCTGGGGGAG |
| | GGSMNHDQEFDPPKVY | GAGGTAGTGGCGGTGGTGGTTCAGGAGGCGGCGGAAGCATGAACCA |
| | PPVPAEKRKPIRVLSLFD | TGACCAGGAATTTGACCCCCCAAAGGTTTACCCACCTGTGCCAGCTGA |
| | GIATGLLVLKDLGIQVDR | GAAGAGGAAGCCCATCCGCGTGCTGTCTCTCTTTGATGGGATTGCTAC |
| | YIASEVCEDSITVGMVR | AGGGCTCCTGGTGCTGAAGGACCTGGGCATCCAAGTGGACCGCTACA |
| | HQGKIMYVGDVRSVTQ | TTGCCTCCGAGGTGTGTGAGGACTCCATCACGGTGGGCATGGTGCGG |
| | KHIQEWGPFDLVIGGSP | CACCAGGGAAAGATCATGTACGTCGGGGACGTCCGCAGCGTCACACA |
| | CNDLSIVNPARKGLYEG | GAAGCATATCCAGGAGTGGGGCCCATTCGACCTGGTGATTGGAGGCA |
| | TGRLFFEFYRLLHDARPK | GTCCCTGCAATGACCTCTCCATTGTCAACCCTGCCCGCAAGGGACTTT |
| | EGDDRPFFWLFENVVA | ATGAGGGTACTGGCCGCCTCTTCTTTGAGTTCTACCGCCTCCTGCATG |
| | MGVSDKRDISRFLESNP | ATGCGCGGCCCAAGGAGGGAGATGATCGCCCCTTCTTCTGGCTCTTTG |
| | VMIDAKEVSAAHRARYF | AGAATGTGGTGGCCATGGGCGTTAGTGACAAGAGGGACATCTCGCG |
| | WGNLPGMNRPLASTV | ATTTCTTGAGTCTAACCCCGTGATGATTGACGCCAAAGAAGTGTCTGC |
| | NDKLELQECLEHGRIAKF | TGCACACAGGGCCCGTTACTTCTGGGGTAACCTTCCTGGCATGAACAG |
| | SKVRTITTRSNSIKQGKD | GCCTTTGGCATCCACTGTGAATGATAAGCTGGAGCTGCAAGAGTGTC |
| | QHFPVFMNEKEDILWC | TGGAGCACGGCAGAATAGCCAAGTTCAGCAAAGTGAGGACCATTACC |
| | TEMERVFGFPVHYTDVS | ACCAGGTCAAACTCTATAAAGCAGGGCAAAGACCAGCATTTCCCCGTC |
| | NMSRLARQRLLGRSWS | TTCATGAACGAGAAGGAGGACATCCTGTGGTGCACTGAAATGGAAAG |
| | VPVIRHLFAPLKEYFACV | GGTGTTTGGCTTCCCCGTCCACTACACAGACGTCTCCAACATGAGCCG |
| | SSGNSNANSRGPSFSSG | CTTGGCGAGGCAGAGACTGCTGGGCCGATCGTGGAGCGTGCCGGTC |
| | LVPLSLRGSHMGPMEIY | ATCCGCCACCTCTTCGCTCCGCTGAAGGAATATTTTGCTTGTGTGTctag |
| | KTVSAWKRQPVRVLSLF | cggcaatagtaacgctaacagccgcgggccgagcttcagcagcggcctggtgccgttaagc |
| | RNIDKVLKSLGFLESGSG | ttgcgcggcagccatATGggccctatggagatatacaagacagtgtctgcatggaagaga |
| | SGGGTLKYVEDVTNVVR | cagccagtgcgggtactgagcctcttcagaaacatcgacaaggtactaaagagtttgggctt |
| | RDVEKWGPFDLVYGST | cttGGAAAGCGGTTCTGGTTCTGGGGGAGGAACGCTGAAGTACGTGG |
| | QPLGSSCDRCPGWYMF | AAGATGTCACAAATGTCGTGAGGAGAGACGTGGAGAAATGGGGCCC |
| | QFHRILQYALPRQESQR | CTTTGACCTGGTGTACGGCTCGACGCAGCCCCTAGGCAGCTCTTGTGA |
| | PFFWIFMDNLLLTEDDQ | TCGCTGTCCCGGCTGGTACATGTTCCAGTTCCACCGGATCCTGCAGTA |
| | ETTTRFLQTEAVTLQDV | TGCGCTGCCTCGCCAGGAGAGTCAGCGGCCCTTCTTCTGGATATTCAT |
| | RGRDYQNAMRVWSNI | GGACAATCTGCTGCTGACTGAGGATGACCAAGAGACAACTACCCGCT |
| | PGLKSKHAPLTPKEEEYL | TCCTTCAGACAGAGGCTGTGACCCTCCAGGATGTCCGTGGCAGAGAC |
| | QAQVRSRSKLDAPKVDL | TACCAGAATGCTATGCGGGTGTGGAGCAACATTCCAGGGCTGAAGAG |
| | LVKNCLLPLREYFKYFSQ | CAAGCATGCGCCCCTGACCCCAAAGGAAGAAGAGTATCTGCAAGCCC |
| | NSLPLQGGGGPKKKRKV | AAGTCAGAAGCAGGAGCAAGCTGGACGCCCCGAAAGTTGACCTCCTG |
| | (SEQ ID NO: 200) | GTGAAGAACTGCCTTCTCCCGCTGAGAGAGTACTTCAAGTATTTTTCT |
| | | CAAAACTCACTTCCTCTTcaaGGTGGAGGTGGACCCAAGAAGAAGCGC |
| | | AAGGTG (SEQ ID NO: 201) |
| SCFV - | MGPDIVMTQSPSSLSA | ATGGGCCCCGACATCGTGATGACCCAGAGCCCCAGCAGCCTGAGCGC |
| KRAB | SVGDRVTITCRSSTGAV | CAGCGTGGGCGACCGCGTGACCATCACCTGCCGCAGCAGCACCGGCG |
| (FIG. | TTSNYASWVQEKPGKL | CCGTGACCACCAGCAACTACGCCAGCTGGGTGCAGGAGAAGCCCGGC |
| 2B | FKGLIGGTNNRAPGVPS | AAGCTGTTCAAGGGCCTGATCGGCGGCACCAACAACCGCGCCCCCGG |
| | RFSGSLIGDKATLTISSL | CGTGCCCAGCCGCTTCAGCGGCAGCCTGATCGGCGACAAGGCCACCC |
| | QPEDFATYFCALWYSN | TGACCATCAGCAGCCTGCAGCCCGAGGACTTCGCCACCTACTTCTGCG |
| | HWVFGQGTKVELKRG | CCCTGTGGTACAGCAACCACTGGGTGTTCGGCCAGGGCACCAAGGTG |
| | GGGSGGGGSGGGGSS | GAGCTGAAGCGCGGCGGCGGCGGCAGCGGCGGCGGCGGCAGCGGC |
| | GGGSEVKLLESGGGLV | GGCGGCGGCAGCAGCGGCGGCGGCAGCGAGGTGAAGCTGCTGGAG |
| | QPGGSLKLSCAVSGFSL | AGCGGCGGCGGCCTGGTGCAGCCCGGCGGCAGCCTGAAGCTGAGCT |
| | TDYGVNWVRQAPGRG | GCGCCGTGAGCGGCTTCAGCCTGACCGACTACGGCGTGAACTGGGTG |
| | LEWIGVIWGDGITDYN | CGCCAGGCCCCCGGCCGCGGCCTGGAGTGGATCGGCGTGATCTGGG |
| | SALKDRFIISKDNGKNT | GCGACGGCATCACCGACTACAACAGCGCCCTGAAGGACCGCTTCATC |
| | VYLQMSKVRSDDTALY | ATCAGCAAGGACAACGGCAAGAACACCGTGTACCTGCAGATGAGCAA |
| | YCVTGLFDYWGQGTLV | GGTGCGCAGCGACGACACCGCCCTGTACTACTGCGTGACCGGCCTGT |
| | TVSSYPYDVPDYAGGG | TCGACTACTGGGGCCAGGGCACCCTGGTGACCGTGAGCAGCTACCCA |
| | GGSGGGGSGGGGSGG | TACGATGTTCCAGATTACGCTGGTGGAGGCGGAGGTTCTGGGGGAG |
| | GGSLDPGGGGSGRTLV | GAGGTAGTGGCGGTGGTGGTTCAGGAGGCGGCGGAAGCTTGGATCC |
| | TFKDVFVDFTREEWKLL | AGGTGGAGGTGGAAGCGGTCGGACACTGGTGACCTTCAAGGATGTA |
| | DTAQQIVYRNVMLENY | TTTGTGGACTTCACCAGGGAGGAGTGGAAGCTGCTGGACACTGCTCA |
| | KNLVSLGYQLTKPDVILR | GCAGATCGTGTACAGAAATGTGATGCTGGAGAACTATAAGAACCTGG |
| | LEKGEEPGSGKRTADGS | TTTCCTTGGGTTATCAGCTTACTAAGCCAGATGTGATCCTCCGGTTGG |
| | EFESPKKKRKV (SEQ ID | AGAAGGGAGAAGAGCCCGGAAGCGGAaagcgcaccgccgatggttccgagtt |
| | NO: 202) | cgaaagccccaaaaaaaagcgcaaggtc (SEQ ID NO: 203) |
| DNMT3A - | MNHDQEFDPPKVYP | ATGAACCATGACCAGGAATTTGACCCCCCAAAGGTTTAC |
| DNMT3L - | PVPAEKRKPIRVLSLF | CCACCTGTGCCAGCTGAGAAGAGGAAGCCCATCCGCGT |
| dCas9 | DGIATGLLVLKDLGI | GCTGTCTCTCTTTGATGGGATTGCTACAGGGCTCCTGGTG |
| (FIG. | QVDRYIASEVCEDSIT | CTGAAGGACCTGGGCATCCAAGTGGACCGCTACATTGCC |
| 3B) | VGMVRHQGKIMYVG | TCCGAGGTGTGTGAGGACTCCATCACGGTGGGCATGGTG |
| | DVRSVTQKHIQEWG | CGGCACCAGGGAAAGATCATGTACGTCGGGGACGTCCG |
| | PFDLVIGGSPCNDLS | CAGCGTCACACAGAAGCATATCCAGGAGTGGGGCCCAT |
| | IVNPARKGLYEGTGR | TCGACCTGGTGATTGGAGGCAGTCCCTGCAATGACCTCT |
| | LFFEFYRLLHDARPK | CCATTGTCAACCCTGCCCGCAAGGGACTTTATGAGGGTA |
| | EGDDRPFFWLFENV | CTGGCCGCCTCTTCTTTGAGTTCTACCGCCTCCTGCATGA |
| | VAMGVSDKRDISRFL | TGCGCGGCCCAAGGAGGGAGATGATCGCCCCTTCTTCTG |
| | ESNPVMIDAKEVSAA | GCTCTTTGAGAATGTGGTGGCCATGGGCGTTAGTGACAA |
| | HRARYFWGNLPGMN | GAGGGACATCTCGCGATTTCTTGAGTCTAACCCCGTGAT |
| | RPLASTVNDKLELQE | GATTGACGCCAAAGAAGTGTCTGCTGCACACAGGGCCC |
| | CLEHGRIAKFSKVRTI | GTTACTTCTGGGGTAACCTTCCTGGCATGAACAGGCCTT |
| | TTRSNSIKQGKDQHF | TGGCATCCACTGTGAATGATAAGCTGGAGCTGCAAGAGT |
| | PVFMNEKEDILWCT | GTCTGGAGCACGGCAGAATAGCCAAGTTCAGCAAAGTG |
| | EMESSGNSNANSRG | AGGACCATTACCACCAGGTCAAACTCTATAAAGCAGGG |
| | PSFSSGLVPLSLRGS | CAAAGACCAGCATTTCCCCGTCTTCATGAACGAGAAGGA |
| | HMGPMEIYKTVSA | GGACATCCTGTGGTGCACTGAAATGGAAAGGGTGTTTGG |
| | WKRQPVRVLSLFR | CTTCCCCGTCCACTACACAGACGTCTCCAACATGAGCCG |
| | NIDKVLKSLGFLES | CTTGGCGAGGCAGAGACTGCTGGGCCGATCGTGGAGCG |
| | GSGSGGGTLKYVE | TGCCGGTCATCCGCCACCTCTTCGCTCCGCTGAAGGAAT |
| | DVTNVVRRDVEKW | ATTTTGCTTGTGTGtctagcggcaatagtaacgctaacagccgcgggccgagct |
| | GPFDLVYGSTQPLG | tcagcagcggcctggtgccgttaagcttgcgcggcagccatATGggccctatggagatat |
| | SSCDRCPGWYMFQ | acaagacagtgtctgcatggaagagacagccagtgcgggtactgagcctcttcagaaacatc |
| | FHRILQYALPRQES | gacaaggtactaaagagtttgggcttcttGGAAAGCGGTTCTGGTTCTGG |
| | QRPFFWIFMDNLLL | GGGAGGAACGCTGAAGTACGTGGAAGATGTCACAAATG |
| | TEDDQETTTRFLQT | TCGTGAGGAGAGACGTGGAGAAATGGGGCCCCTTTGAC |
| | EAVTLQDVRGRDY | CTGGTGTACGGCTCGACGCAGCCCCTAGGCAGCTCTTGT |
| | QNAMRVWSNIPGL | GATCGCTGTCCCGGCTGGTACATGTTCCAGTTCCACCGG |
| | KSKHAPLTPKEEEY | ATCCTGCAGTATGCGCTGCCTCGCCAGGAGAGTCAGCGG |
| | LQAQVRSRSKLDAP | CCCTTCTTCTGGATATTCATGGACAATCTGCTGCTGACTG |
| | KVDLLVKNCLLPLR | AGGATGACCAAGAGACAACTACCCGCTTCCTTCAGACAG |
| | EYFKYFSQNSLPLR | AGGCTGTGACCCTCCAGGATGTCCGTGGCAGAGACTACC |
| | VFGFPVHYTDVSN | AGAATGCTATGCGGGTGTGGAGCAACATTCCAGGGCTG |
| | MSRLARQRLLGRS | AAGAGCAAGCATGCGCCCCTGACCCCAAAGGAAGAAGA |
| | WSVPVIRHLFAPLK | GTATCTGCAAGCCCAAGTCAGAAGCAGGAGCAAGCTGG |
| | EYFACVGGPSSGAP | ACGCCCCGAAAGTTGACCTCCTGGTGAAGAACTGCCTTC |
| | PPSGGSPAGSPTSTE | TCCCGCTGAGAGAGTACTTCAAGTATTTTTCTCAAAACT |
| | EGTSESATPESGPGT | CACTTCCTCTTggagggccgagctctggcgcacccccaccaagtggagggtctcc |
| | STEPSEGSAPGSPAG | tgccgggtccccaacatctactgaagaaggcaccagcgaatccgcaacgcccgagtcaggc |
| | SPTSTEEGTSTEPSE | cctggtacctccacagaaccatctgaaggtagtgcgcctggttccccagctggaagccctact |
| | GSAPGTSTEPSEPKK | tccaccgaagaaggcacgtcaaccgaaccaagtgaaggatctgcccctgggaccagcactg |
| | KRKVMDKKYSIGL | aaccatctgagCCAAAAAAGAAGAGAAAGGTAATGGACAAGA |
| | AIGTNSVGWAVIT | AGTACAGCATCGGCCTGGCCATCGGCACCAACTCTGTGG |
| | DEYKVPSKKFKVL | GCTGGGCCGTGATCACCGACGAGTACAAGGTGCCCAGC |
| | GNTDRHSIKKNLI | AAGAAATTCAAGGTGCTGGGCAACACCGACCGGCACAG |
| | GALLFDSGETAEA | CATCAAGAAGAACCTGATCGGCGCCCTGCTGTTCGACAG |
| | TRLKRTARRRYTR | CGGAGAAACAGCCGAGGCCACCCGGCTGAAGAGAACCG |
| | RKNRICYLQEIFSN | CCAGAAGAAGATACACCAGACGGAAGAACCGGATCTGC |
| | EMAKVDDSFFHRL | TATCTGCAAGAGATCTTCAGCAACGAGATGGCCAAGGTG |
| | EESFLVEEDKKHE | GACGACAGCTTCTTCCACAGACTGGAAGAGTCCTTCCTG |
| | RHPIFGNIVDEVAY | GTGGAAGAGGATAAGAAGCACGAGCGGCACCCCATCTT |
| | HEKYPTIYHLRKK | CGGCAACATCGTGGACGAGGTGGCCTACCACGAGAAGT |
| | LVDSTDKADLRLI | ACCCCACCATCTACCACCTGAGAAAGAAACTGGTGGAC |
| | YLALAHMIKFRGH | AGCACCGACAAGGCCGACCTGCGGCTGATCTATCTGGCC |
| | FLIEGDLNPDNSDV | CTGGCCCACATGATCAAGTTCCGGGGCCACTTCCTGATC |
| | DKLFIQLVQTYNQ | GAGGGCGACCTGAACCCCGACAACAGCGACGTGGACAA |
| | LFEENPINASGVDA | GCTGTTCATCCAGCTGGTGCAGACCTACAACCAGCTGTT |
| | KAILSARLSKSRRL | CGAGGAAAACCCCATCAACGCCAGCGGCGTGGACGCCA |
| | ENLIAQLPGEKKN | AGGCCATCCTGTCTGCCAGACTGAGCAAGAGCAGACGG |
| | GLFGNLIALSLGLT | CTGGAAAATCTGATCGCCCAGCTGCCCGGCGAGAAGAA |
| | PNFKSNFDLAEDA | GAATGGCCTGTTCGGCAACCTGATTGCCCTGAGCCTGGG |
| | KLQLSKDTYDDDL | CCTGACCCCCAACTTCAAGAGCAACTTCGACCTGGCCGA |
| | DNLLAQIGDQYAD | GGATGCCAAACTGCAGCTGAGCAAGGACACCTACGACG |
| | LFLAAKNLSDAILL | ACGACCTGGACAACCTGCTGGCCCAGATCGGCGACCAGT |
| | SDILRVNTEITKAP | ACGCCGACCTGTTTCTGGCCGCCAAGAACCTGTCCGACG |
| | LSASMIKRYDEHH | CCATCCTGCTGAGCGACATCCTGAGAGTGAACACCGAGA |
| | QDLTLLKALVRQQ | TCACCAAGGCCCCCCTGAGCGCCTCTATGATCAAGAGAT |
| | LPEKYKEIFFDQSK | ACGACGAGCACCACCAGGACCTGACCCTGCTGAAAGCT |
| | NGYAGYIDGGASQ | CTCGTGCGGCAGCAGCTGCCTGAGAAGTACAAAGAGAT |
| | EEFYKFIKPILEKM | TTTCTTCGACCAGAGCAAGAACGGCTACGCCGGCTACAT |
| | DGTEELLVKLNRE | CGATGGCGGAGCCAGCCAGGAAGAGTTCTACAAGTTCA |
| | DLLRKQRTFDNGS | TCAAGCCCATCCTGGAAAAGATGGACGGCACCGAGGAA |
| | IPHQIHLGELHAIL | CTGCTCGTGAAGCTGAACAGAGAGGACCTGCTGCGGAA |
| | RRQEDFYPFLKDN | GCAGCGGACCTTCGACAACGGCAGCATCCCCCACCAGAT |
| | REKIEKILTFRIPY | CCACCTGGGAGAGCTGCACGCCATTCTGCGGCGGCAGG |
| | YVGPLARGNSRFA | AAGATTTTTACCCATTCCTGAAGGACAACCGGGAAAAGA |
| | WMTRKSEETITPW | TCGAGAAGATCCTGACCTTCCGCATCCCCTACTACGTGG |
| | NFEEVVDKGASAQ | GCCCTCTGGCCAGGGGAAACAGCAGATTCGCCTGGATG |
| | SFIERMTNFDKNLP | ACCAGAAAGAGCGAGGAAACCATCACCCCCTGGAACTT |
| | NEKVLPKHSLLYE | CGAGGAAGTGGTGGACAAGGGCGCCAGCGCCCAGAGCT |
| | YFTVYNELTKVKY | TCATCGAGCGGATGACCAACTTCGATAAGAACCTGCCCA |
| | VTEGMRKPAFLSG | ACGAGAAGGTGCTGCCCAAGCACAGCCTGCTGTACGAG |
| | EQKKAIVDLLFKT | TACTTCACCGTGTACAACGAGCTGACCAAAGTGAAATAC |
| | NRKVTVKQLKED | GTGACCGAGGGAATGAGAAAGCCCGCCTTCCTGAGCGG |
| | YFKKIECFDSVEIS | CGAGCAGAAAAAAGCCATCGTGGACCTGCTGTTCAAGA |
| | GVEDRFNASLGTY | CCAACCGGAAAGTGACCGTGAAGCAGCTGAAAGAGGAC |
| | HDLLKIIKDKDFLD | TACTTCAAGAAAATCGAGTGCTTCGACTCCGTGGAAATC |
| | NEENEDILEDIVLT | TCCGGCGTGGAAGATCGGTTCAACGCCTCCCTGGGCACA |
| | LTLFEDREMIEER | TACCACGATCTGCTGAAAATTATCAAGGACAAGGACTTC |
| | LKTYAHLFDDKV | CTGGACAATGAGGAAAACGAGGACATTCTGGAAGATAT |
| | MKQLKRRRYTGW | CGTGCTGACCCTGACACTGTTTGAGGACAGAGAGATGAT |
| | GRLSRKLINGIRDK | CGAGGAACGGCTGAAAACCTATGCCCACCTGTTCGACGA |
| | QSGKTILDFLKSD | CAAAGTGATGAAGCAGCTGAAGCGGCGGAGATACACCG |
| | GFANRNFMQLIHD | GCTGGGGCAGGCTGAGCCGGAAGCTGATCAACGGCATC |
| | DSLTFKEDIQKAQ | CGGGACAAGCAGTCCGGCAAGACAATCCTGGATTTCCTG |
| | VSGQGDSLHEHIA | AAGTCCGACGGCTTCGCCAACAGAAACTTCATGCAGCTG |
| | NLAGSPAIKKGILQ | ATCCACGACGACAGCCTGACCTTTAAAGAGGACATCCAG |
| | TVKVVDELVKVM | AAAGCCCAGGTGTCCGGCCAGGGCGATAGCCTGCACGA |
| | GRHKPENIVIEMA | GCACATTGCCAATCTGGCCGGCAGCCCCGCCATTAAGAA |
| | RENQTTQKGQKNS | GGGCATCCTGCAGACAGTGAAGGTGGTGGACGAGCTCG |
| | RERMKRIEEGIKE | TGAAAGTGATGGGCCGGCACAAGCCCGAGAACATCGTG |
| | LGSQILKEHPVEN | ATCGAAATGGCCAGAGAGAACCAGACCACCCAGAAGGG |
| | TQLQNEKLYLYYL | ACAGAAGAACAGCCGCGAGAGAATGAAGCGGATCGAAG |
| | QNGRDMYVDQEL | AGGGCATCAAAGAGCTGGGCAGCCAGATCCTGAAAGAA |
| | DINRLSDYDVDAIV | CACCCCGTGGAAAACACCCAGCTGCAGAACGAGAAGCT |
| | PQSFLKDDSIDNKV | GTACCTGTACTACCTGCAGAATGGGCGGGATATGTACGT |
| | LTRSDKNRGKSDN | GGACCAGGAACTGGACATCAACCGGCTGTCCGACTACG |
| | VPSEEVVKKMKNY | ATGTGGACGCTATCGTGCCTCAGAGCTTTCTGAAGGACG |
| | WRQLLNAKLITQR | ACTCCATCGATAACAAAGTGCTGACTCGGAGCGACAAG |
| | KFDNLTKAERGGL | AACCGGGGCAAGAGCGACAACGTGCCCTCCGAAGAGGT |
| | SELDKAGFIKRQL | CGTGAAGAAGATGAAGAACTACTGGCGCCAGCTGCTGA |
| | VETRQITKHVAQI | ATGCCAAGCTGATTACCCAGAGGAAGTTCGACAATCTGA |
| | LDSRMNTKYDEND | CCAAGGCCGAGAGAGGCGGCCTGAGCGAACTGGATAAG |
| | KLIREVKVITLKSK | GCCGGCTTCATCAAGAGACAGCTGGTGGAAACCCGGCA |
| | LVSDFRKDFQFYK | GATCACAAAGCACGTGGCACAGATCCTGGACTCCCGGAT |
| | VREINNYHHAHDA | GAACACTAAGTACGACGAGAACGACAAACTGATCCGGG |
| | YLNAVVGTALIKK | AAGTGAAAGTGATCACCCTGAAGTCCAAGCTGGTGTCCG |
| | YPKLESEFVYGDY | ATTTCCGGAAGGATTTCCAGTTTTACAAAGTGCGCGAGA |
| | KVYDVRKMIAKSE | TCAACAACTACCACCACGCCCACGACGCCTACCTGAACG |
| | QEIGKATAKYFFY | CCGTCGTGGGAACCGCCCTGATCAAAAAGTACCCTAAGC |
| | SNIMNFFKTEITLA | TGGAAAGCGAGTTCGTGTACGGCGACTACAAGGTGTAC |
| | NGEIRKRPLIETNG | GACGTGCGGAAGATGATCGCCAAGAGCGAGCAGGAAAT |
| | ETGEIVWDKGRDF | CGGCAAGGCTACCGCCAAGTACTTCTTCTACAGCAACAT |
| | ATVRKVLSMPQVN | CATGAACTTTTTCAAGACCGAGATTACCCTGGCCAACGG |
| | IVKKTEVQTGGFS | CGAGATCCGGAAGCGGCCTCTGATCGAGACAAACGGCG |
| | KESILPKRNSDKLI | AAACAGGCGAGATCGTGTGGGATAAGGGCCGGGACTTT |
| | ARKKDWDPKKYG | GCCACCGTGCGGAAAGTGCTGTCTATGCCCCAAGTGAAT |
| | GFDSPTVAYSVLV | ATCGTGAAAAAGACCGAGGTGCAGACAGGCGGCTTCAG |
| | VAKVEKGKSKKL | CAAAGAGTCTATCCTGCCCAAGAGGAACAGCGACAAGC |
| | KSVKELLGITIMER | TGATCGCCAGAAAGAAGGACTGGGACCCTAAGAAGTAC |
| | SSFEKNPIDFLEAK | GGCGGCTTCGACAGCCCCACCGTGGCCTATTCTGTGCTG |
| | GYKEVKKDLIIKL | GTGGTGGCCAAAGTGGAAAAGGGCAAGTCCAAGAAACT |
| | PKYSLFELENGRK | GAAGAGTGTGAAAGAGCTGCTGGGGATCACCATCATGG |
| | RMLASAGELQKG | AAAGAAGCAGCTTCGAGAAGAATCCCATCGACTTTCTGG |
| | NELALPSKYVNFL | AAGCCAAGGGCTACAAAGAAGTGAAAAAGGACCTGATC |
| | YLASHYEKLKGSP | ATCAAGCTGCCTAAGTACTCCCTGTTCGAGCTGGAAAAC |
| | EDNEQKQLFVEQH | GGCCGGAAGAGAATGCTGGCCTCTGCCGGCGAACTGCA |
| | KHYLDEIIEQISEFS | GAAGGGAAACGAACTGGCCCTGCCCTCCAAATATGTGA |
| | KRVILADANLDKV | ACTTCCTGTACCTGGCCAGCCACTATGAGAAGCTGAAGG |
| | LSAYNKHRDKPIR | GCTCCCCCGAGGATAATGAGCAGAAACAGCTGTTTGTGG |
| | EQAENIIHLFTLTN | AACAGCACAAACACTACCTGGACGAGATCATCGAGCAG |
| | LGAPAAFKYFDTTI | ATCAGCGAGTTCTCCAAGAGAGTGATCCTGGCCGACGCT |
| | DRKRYTSTKEVLD | AATCTGGACAAGGTGCTGAGCGCCTACAACAAGCACAG |
| | ATLIHQSITGLYET | AGACAAGCCTATCAGAGAGCAGGCCGAGAATATCATCC |
| | RIDLSQLGGD (SEQ | ACCTGTTTACCCTGACCAATCTGGGAGCCCCTGCCGCCT |
| | ID NO: 156) | TCAAGTACTTTGACACCACCATCGACCGGAAGAGGTACA |
| | | CCAGCACCAAAGAGGTGCTGGACGCCACCCTGATCCACC |
| | | AGAGCATCACCGGCCTGTACGAGACACGGATCGACCTGT |
| | | CTCAGCTGGGAGGCGAC (SEQ ID NO: 184) |
| MCP - | MASNFTQFVLVDN | atggcttctaactttactcagttcgttctcgtcgacaatggcggaactggcgacgtgactgtcgc |
| KRAB | GGTGDVTVAPSNF | cccaagcaacttcgctaacgggatcgctgaatggatcagctctaactcgcgttcacaggctta |
| (FIG. | ANGIAEWISSNSRS | caaagtaacctgtagcgttcgtcagagctctgcgcagaatcgcaaatacaccatcaaagtcga |
| 3B) | QAYKVTCSVRQSS | ggtgcctaaaggcgcctggcgttcgtacttaaatatggaactaaccattccaattttcgccacga |
| | AQNRKYTIKVEVP | attccgactgcgagcttattgttaaggcaatgcaaggtctcctaaaagatggaaacccgattcc |
| | KGAWRSYLNMEL | ctcagcaatcgcagcaaactccggcatctacTTGGATCCAGGTGGAGGTG |
| | TIPIFATNSDCELIV | GAAGCGGTCGGACACTGGTGACCTTCAAGGATGTATTTG |
| | KAMQGLLKDGNPI | TGGACTTCACCAGGGAGGAGTGGAAGCTGCTGGACACT |
| | PSAIAANSGIYLDP | GCTCAGCAGATCGTGTACAGAAATGTGATGCTGGAGAA |
| | GGGGSGRTLVTFK | CTATAAGAACCTGGTTTCCTTGGGTTATCAGCTTACTAA |
| | DVFVDFTREEWKL | GCCAGATGTGATCCTCCGGTTGGAGAAGGGAGAAGAGC |
| | LDTAQQIVYRNVML | CCGGAAGCGGAaagcgcaccgccgatggttccgagttcgaaagccccaaaaaaa |
| | ENYKNLVSLGYQLT | agcgcaaggtc (SEQ ID NO: 185) |
| | KPDVILRLEKGEEP | |
| | GSGKRTADGSEFES | |
| | PKKKRK (SEQ ID | |
| | NO: 157) | |
| MCP - | MASNFTQFVLVDN | atggcttctaactttactcagttcgttctcgtcgacaatggcggaactggcgacgtgactgtcgc |
| KRAB- | GGTGDVTVAPSNF | cccaagcaacttcgctaacgggatcgctgaatggatcagctctaactcgcgttcacaggctta |
| DNMT3A- | ANGIAEWISSNSRS | caaagtaacctgtagcgttcgtcagagctctgcgcagaatcgcaaatacaccatcaaagtcga |
| DNMT3L | QAYKVTCSVRQSS | ggtgcctaaaggcgcctggcgttcgtacttaaatatggaactaaccattccaattttcgccacga |
| (FIG. | AQNRKYTIKVEVP | attccgactgcgagcttattgttaaggcaatgcaaggtctcctaaaagatggaaacccgattcc |
| 3B) | KGAWRSYLNMEL | ctcagcaatcgcagcaaactccggcatctacggagggccgagctctggcgcacccccacca |
| | TIPIFATNSDCELIV | agtggagggtctcctgccgggtccccaacatctactgaagaaggcaccagcgaatccgcaa |
| | KAMQGLLKDGNPI | cgcccgagtcaggccctggtacctccacagaaccatctgaaggtagtgcgcctggttcccca |
| | PSAIAANSGIYRVF | gctggaagccctacttccaccgaagaaggcacgtcaaccgaaccaagtgaaggatctgccc |
| | GFPVHYTDVSNMS | ctgggaccagcactgaaccatctgagCCAAAAAAGAAGAGAAAGGTA |
| | RLARQRLLGRSWS | CGGACACTGGTGACCTTCAAGGATGTATTTGTGGACTTC |
| | VPVIRHLFAPLKEYF | ACCAGGGAGGAGTGGAAGCTGCTGGACACTGCTCAGCA |
| | ACVGGPSSGAPPPS | GATCGTGTACAGAAATGTGATGCTGGAGAACTATAAGA |
| | GGSPAGSPTSTEEG | ACCTGGTTTCCTTGGGTTATCAGCTTACTAAGCCAGATGT |
| | TSESATPESGPGTST | GATCCTCCGGTTGGAGAAGGGAGAAGAGCCCGGAAGCG |
| | EPSEGSAPGSPAGSP | GATTGGATCCAGGTGGAGGTGGAAGCGGTATGAACCAT |
| | TSTEEGTSTEPSEGS | GACCAGGAATTTGACCCCCCAAAGGTTTACCCACCTGTG |
| | APGTSTEPSEPKKK | CCAGCTGAGAAGAGGAAGCCCATCCGCGTGCTGTCTCTC |
| | RKVRTLVTFKDVFV | TTTGATGGGATTGCTACAGGGCTCCTGGTGCTGAAGGAC |
| | DFTREEWKLLDTA | CTGGGCATCCAAGTGGACCGCTACATTGCCTCCGAGGTG |
| | QQIVYRNVMLENYK | TGTGAGGACTCCATCACGGTGGGCATGGTGCGGCACCAG |
| | NLVSLGYQLTKPDV | GGAAAGATCATGTACGTCGGGGACGTCCGCAGCGTCAC |
| | ILRLEKGEEPLDPG | ACAGAAGCATATCCAGGAGTGGGGCCCATTCGACCTGGT |
| | GGGSGMNHDQEFD | GATTGGAGGCAGTCCCTGCAATGACCTCTCCATTGTCAA |
| | PPKVYPPVPAEKRKP | CCCTGCCCGCAAGGGACTTTATGAGGGTACTGGCCGCCT |
| | IRVLSLFDGIATGLLV | CTTCTTTGAGTTCTACCGCCTCCTGCATGATGCGCGGCCC |
| | LKDLGIQVDRYIASE | AAGGAGGGAGATGATCGCCCCTTCTTCTGGCTCTTTGAG |
| | VCEDSITVGMVRHQ | AATGTGGTGGCCATGGGCGTTAGTGACAAGAGGGACAT |
| | GKIMYVGDVRSVTQ | CTCGCGATTTCTTGAGTCTAACCCCGTGATGATTGACGC |
| | KHIQEWGPFDLVIG | CAAAGAAGTGTCTGCTGCACACAGGGCCCGTTACTTCTG |
| | GSPCNDLSIVNPARK | GGGTAACCTTCCTGGCATGAACAGGCCTTTGGCATCCAC |
| | GLYEGTGRLFFEFYR | TGTGAATGATAAGCTGGAGCTGCAAGAGTGTCTGGAGC |
| | LLHDARPKEGDDRP | ACGGCAGAATAGCCAAGTTCAGCAAAGTGAGGACCATT |
| | FFWLFENVVAMGVS | ACCACCAGGTCAAACTCTATAAAGCAGGGCAAAGACCA |
| | DKRDISRFLESNPVM | GCATTTCCCCGTCTTCATGAACGAGAAGGAGGACATCCT |
| | IDAKEVSAAHRARYF | GTGGTGCACTGAAATGGAAAGGGTGTTTGGCTTCCCCGT |
| | WGNLPGMNRPLAST | CCACTACACAGACGTCTCCAACATGAGCCGCTTGGCGAG |
| | VNDKLELQECLEHG | GCAGAGACTGCTGGGCCGATCGTGGAGCGTGCCGGTCAT |
| | RIAKFSKVRTITTRSN | CCGCCACCTCTTCGCTCCGCTGAAGGAATATTTTGCTTGT |
| | SIKQGKDQHFPVFM | GTGtctagcggcaatagtaacgctaacagccgcgggccgagcttcagcagcggcctggt |
| | NEKEDILWCTEMESS | gccgttaagcttgcgcggcagccatATGggccctatggagatatacaagacagtgtctgca |
| | GNSNANSRGPSFSS | tggaagagacagccagtgcgggtactgagcctcttcagaaacatcgacaaggtactaaaga |
| | GLVPLSLRGSHMGP | gtttgggcttcttGGAAAGCGGTTCTGGTTCTGGGGGAGGAACG |
| | MEIYKTVSAWKRQ | CTGAAGTACGTGGAAGATGTCACAAATGTCGTGAGGAG |
| | PVRVLSLFRNIDKV | AGACGTGGAGAAATGGGGCCCCTTTGACCTGGTGTACGG |
| | LKSLGFLESGSGSG | CTCGACGCAGCCCCTAGGCAGCTCTTGTGATCGCTGTCC |
| | GGTLKYVEDVTNV | CGGCTGGTACATGTTCCAGTTCCACCGGATCCTGCAGTA |
| | VRRDVEKWGPFDL | TGCGCTGCCTCGCCAGGAGAGTCAGCGGCCCTTCTTCTG |
| | VYGSTQPLGSSCDR | GATATTCATGGACAATCTGCTGCTGACTGAGGATGACCA |
| | CPGWYMFQFHRILQ | AGAGACAACTACCCGCTTCCTTCAGACAGAGGCTGTGAC |
| | YALPRQESQRPFFW | CCTCCAGGATGTCCGTGGCAGAGACTACCAGAATGCTAT |
| | IFMDNLLLTEDDQE | GCGGGTGTGGAGCAACATTCCAGGGCTGAAGAGCAAGC |
| | TTTRFLQTEAVTLQ | ATGCGCCCCTGACCCCAAAGGAAGAAGAGTATCTGCAA |
| | DVRGRDYQNAMRV | GCCCAAGTCAGAAGCAGGAGCAAGCTGGACGCCCCGAA |
| | WSNIPGLKSKHAPL | AGTTGACCTCCTGGTGAAGAACTGCCTTCTCCCGCTGAG |
| | TPKEEEYLQAQVRS | AGAGTACTTCAAGTATTTTTCTCAAAACTCACTTCCTCTT |
| | RSKLDAPKVDLLVK | aagcgcaccgccgatggttccgagttcgaaagccccaaaaaaaagcgcaaggtc (SEQ |
| | NCLLPLREYFKYFS | ID NO: 186) |
| | QNSLPLGSGKRTAD | |
| | GSEFESPKKKRK | |
| | (SEQ ID NO: 158) | |
| MCP - | MASNFTQFVLVDN | atggcttctaactttactcagttcgttctcgtcgacaatggcggaactggcgacgtgactgtcgc |
| KRAB- | GGTGDVTVAPSNF | cccaagcaacttcgctaacgggatcgctgaatggatcagctctaactcgcgttcacaggctta |
| DNMT3L- | ANGIAEWISSNSRS | caaagtaacctgtagcgttcgtcagagctctgcgcagaatcgcaaatacaccatcaaagtcga |
| DNMT3A | QAYKVTCSVRQSS | ggtgcctaaaggcgcctggcgttcgtacttaaatatggaactaaccattccaattttcgccacga |
| (FIG. | AQNRKYTIKVEVP | attccgactgcgagcttattgttaaggcaatgcaaggtctcctaaaagatggaaacccgattcc |
| 3B) | KGAWRSYLNMEL | ctcagcaatcgcagcaaactccggcatctacggagggccgagctctggcgcacccccacca |
| | TIPIFATNSDCELIV | agtggagggtctcctgccgggtccccaacatctactgaagaaggcaccagcgaatccgcaa |
| | KAMQGLLKDGNPI | cgcccgagtcaggccctggtacctccacagaaccatctgaaggtagtgcgcctggttcccca |
| | PSAIAANSGIYRVF | gctggaagccctacttccaccgaagaaggcacgtcaaccgaaccaagtgaaggatctgccc |
| | GFPVHYTDVSNMS | ctgggaccagcactgaaccatctgagCCAAAAAAGAAGAGAAAGGTA |
| | RLARQRLLGRSWS | CGGACACTGGTGACCTTCAAGGATGTATTTGTGGACTTC |
| | VPVIRHLFAPLKEYF | ACCAGGGAGGAGTGGAAGCTGCTGGACACTGCTCAGCA |
| | ACVGGPSSGAPPPS | GATCGTGTACAGAAATGTGATGCTGGAGAACTATAAGA |
| | GGSPAGSPTSTEEG | ACCTGGTTTCCTTGGGTTATCAGCTTACTAAGCCAGATGT |
| | TSESATPESGPGTST | GATCCTCCGGTTGGAGAAGGGAGAAGAGCCCGGAAGCG |
| | EPSEGSAPGSPAGSP | GATTGGATCCAGGTGGAGGTGGAAGCGGTATGggccctatgg |
| | TSTEEGTSTEPSEGS | agatatacaagacagtgtctgcatggaagagacagccagtgcgggtactgagcctcttcaga |
| | APGTSTEPSEPKKK | aacatcgacaaggtactaaagagtttgggcttcttGGAAAGCGGTTCTGGTTC |
| | RKVRTLVTFKDVFV | TGGGGGAGGAACGCTGAAGTACGTGGAAGATGTCACAA |
| | DFTREEWKLLDTA | ATGTCGTGAGGAGAGACGTGGAGAAATGGGGCCCCTTT |
| | QQIVYRNVMLENYK | GACCTGGTGTACGGCTCGACGCAGCCCCTAGGCAGCTCT |
| | NLVSLGYQLTKPDV | TGTGATCGCTGTCCCGGCTGGTACATGTTCCAGTTCCACC |
| | ILRLEKGEEPGSGL | GGATCCTGCAGTATGCGCTGCCTCGCCAGGAGAGTCAGC |
| | DPGGGGSGMGPME | GGCCCTTCTTCTGGATATTCATGGACAATCTGCTGCTGAC |
| | IYKTVSAWKRQPVR | TGAGGATGACCAAGAGACAACTACCCGCTTCCTTCAGAC |
| | VLSLFRNIDKVLKS | AGAGGCTGTGACCCTCCAGGATGTCCGTGGCAGAGACTA |
| | LGFLESGSGSGGGT | CCAGAATGCTATGCGGGTGTGGAGCAACATTCCAGGGCT |
| | LKYVEDVTNVVRR | GAAGAGCAAGCATGCGCCCCTGACCCCAAAGGAAGAAG |
| | DVEKWGPFDLVYG | AGTATCTGCAAGCCCAAGTCAGAAGCAGGAGCAAGCTG |
| | STQPLGSSCDRCPG | GACGCCCCGAAAGTTGACCTCCTGGTGAAGAACTGCCTT |
| | WYMFQFHRILQYA | CTCCCGCTGAGAGAGTACTTCAAGTATTTTTCTCAAAAC |
| | LPRQESQRPFFWIF | TCACTTCCTCTTtctagcggcaatagtaacgctaacagccgcgggccgagcttcag |
| | MDNLLLTEDDQETT | cagcggcctggtgccgttaagcttgcgcggcagccatATGAACCATGACCAG |
| | TRFLQTEAVTLQDV | GAATTTGACCCCCCAAAGGTTTACCCACCTGTGCCAGCT |
| | RGRDYQNAMRVW | GAGAAGAGGAAGCCCATCCGCGTGCTGTCTCTCTTTGAT |
| | SNIPGLKSKHAPLTP | GGGATTGCTACAGGGCTCCTGGTGCTGAAGGACCTGGGC |
| | KEEEYLQAQVRSRS | ATCCAAGTGGACCGCTACATTGCCTCCGAGGTGTGTGAG |
| | KLDAPKVDLLVKN | GACTCCATCACGGTGGGCATGGTGCGGCACCAGGGAAA |
| | CLLPLREYFKYFSQ | GATCATGTACGTCGGGGACGTCCGCAGCGTCACACAGA |
| | NSLPLSSGNSNANS | AGCATATCCAGGAGTGGGGCCCATTCGACCTGGTGATTG |
| | RGPSFSSGLVPLSLR | GAGGCAGTCCCTGCAATGACCTCTCCATTGTCAACCCTG |
| | GSHMNHDQEFDPP | CCCGCAAGGGACTTTATGAGGGTACTGGCCGCCTCTTCT |
| | KVYPPVPAEKRKPIR | TTGAGTTCTACCGCCTCCTGCATGATGCGCGGCCCAAGG |
| | VLSLFDGIATGLLVL | AGGGAGATGATCGCCCCTTCTTCTGGCTCTTTGAGAATG |
| | KDLGIQVDRYIASEV | TGGTGGCCATGGGCGTTAGTGACAAGAGGGACATCTCGC |
| | CEDSITVGMVRHQG | GATTTCTTGAGTCTAACCCCGTGATGATTGACGCCAAAG |
| | KIMYVGDVRSVTQK | AAGTGTCTGCTGCACACAGGGCCCGTTACTTCTGGGGTA |
| | HIQEWGPFDLVIGGS | ACCTTCCTGGCATGAACAGGCCTTTGGCATCCACTGTGA |
| | PCNDLSIVNPARKGL | ATGATAAGCTGGAGCTGCAAGAGTGTCTGGAGCACGGC |
| | YEGTGRLFFEFYRLL | AGAATAGCCAAGTTCAGCAAAGTGAGGACCATTACCAC |
| | HDARPKEGDDRPFF | CAGGTCAAACTCTATAAAGCAGGGCAAAGACCAGCATT |
| | WLFENVVAMGVSDK | TCCCCGTCTTCATGAACGAGAAGGAGGACATCCTGTGGT |
| | RDISRFLESNPVMID | GCACTGAAATGGAAAGGGTGTTTGGCTTCCCCGTCCACT |
| | AKEVSAAHRARYFW | ACACAGACGTCTCCAACATGAGCCGCTTGGCGAGGCAG |
| | GNLPGMNRPLASTV | AGACTGCTGGGCCGATCGTGGAGCGTGCCGGTCATCCGC |
| | NDKLELQECLEHGRI | CACCTCTTCGCTCCGCTGAAGGAATATTTTGCTTGTGTGa |
| | AKFSKVRTITTRSNSI | agcgcaccgccgatggttccgagttcgaaagccccaaaaaaaagcgcaaggtc SEQ ID |
| | KQGKDQHFPVFMN | NO: 187) |
| | EKEDILWCTEMEKR | |
| | TADGSEFESPKKKR | |
| | K (SEQ ID NO: 159) | |
| EPIC | MGPMEIYKTVSAWK | ATGggccctatggagatatacaagacagtgtctgcatggaagagacagccagtgcgggta |
| AS- | RQPVRVLSLFRNIDK | ctgagcctcttcagaaacatcgacaaggtactaaagagtttgggcttcttGGAAAGCG |
| V2 | VLKSLGFLESGSGSG | GTTCTGGTTCTGGGGGAGGAACGCTGAAGTACGTGGAA |
| DNMT3A- | GGTLKYVEDVTNVVR | GATGTCACAAATGTCGTGAGGAGAGACGTGGAGAAATG |
| DNMT3L- | RDVEKWGPFDLVYG | GGGCCCCTTTGACCTGGTGTACGGCTCGACGCAGCCCCT |
| dCas9- | STQPLGSSCDRCPG | AGGCAGCTCTTGTGATCGCTGTCCCGGCTGGTACATGTT |
| ZIM3 | WYMFQFHRILQYAL | CCAGTTCCACCGGATCCTGCAGTATGCGCTGCCTCGCCA |
| Krab | PRQESQRPFFWIFM | GGAGAGTCAGCGGCCCTTCTTCTGGATATTCATGGACAA |
| (FIG. | DNLLLTEDDQETTTR | TCTGCTGCTGACTGAGGATGACCAAGAGACAACTACCCG |
| 6B) | FLQTEAVTLQDVRGR | CTTCCTTCAGACAGAGGCTGTGACCCTCCAGGATGTCCG |
| | DYQNAMRVWSNIPG | TGGCAGAGACTACCAGAATGCTATGCGGGTGTGGAGCA |
| | LKSKHAPLTPKEEEY | ACATTCCAGGGCTGAAGAGCAAGCATGCGCCCCTGACCC |
| | LQAQVRSRSKLDAPK | CAAAGGAAGAAGAGTATCTGCAAGCCCAAGTCAGAAGC |
| | VDLLVKNCLLPLREY | AGGAGCAAGCTGGACGCCCCGAAAGTTGACCTCCTGGT |
| | FKYFSQNSLPLSSGN | GAAGAACTGCCTTCTCCCGCTGAGAGAGTACTTCAAGTA |
| | SNANSRGPSFSSGL | TTTTTCTCAAAACTCACTTCCTCTTtctagcggcaatagtaacgctaaca |
| | VPLSLRGSHMNHD | gccgcgggccgagcttcagcagcggcctggtgccgttaagcttgcgcggcagccatATG |
| | QEFDPPKVYPPVPA | AACCATGACCAGGAATTTGACCCCCCAAAGGTTTACCCA |
| | EKRKPIRVLSLFDGI | CCTGTGCCAGCTGAGAAGAGGAAGCCCATCCGCGTGCTG |
| | ATGLLVLKDLGIQV | TCTCTCTTTGATGGGATTGCTACAGGGCTCCTGGTGCTGA |
| | DRYIASEVCEDSITV | AGGACCTGGGCATCCAAGTGGACCGCTACATTGCCTCCG |
| | GMVRHQGKIMYVG | AGGTGTGTGAGGACTCCATCACGGTGGGCATGGTGCGGC |
| | DVRSVTQKHIQEW | ACCAGGGAAAGATCATGTACGTCGGGGACGTCCGCAGC |
| | GPFDLVIGGSPCND | GTCACACAGAAGCATATCCAGGAGTGGGGCCCATTCGA |
| | LSIVNPARKGLYEG | CCTGGTGATTGGAGGCAGTCCCTGCAATGACCTCTCCAT |
| | TGRLFFEFYRLLHD | TGTCAACCCTGCCCGCAAGGGACTTTATGAGGGTACTGG |
| | ARPKEGDDRPFFWL | CCGCCTCTTCTTTGAGTTCTACCGCCTCCTGCATGATGCG |
| | FENVVAMGVSDKR | CGGCCCAAGGAGGGAGATGATCGCCCCTTCTTCTGGCTC |
| | DISRFLESNPVMIDA | TTTGAGAATGTGGTGGCCATGGGCGTTAGTGACAAGAGG |
| | KEVSAAHRARYFW | GACATCTCGCGATTTCTTGAGTCTAACCCCGTGATGATT |
| | GNLPGMNRPLASTV | GACGCCAAAGAAGTGTCTGCTGCACACAGGGCCCGTTAC |
| | NDKLELQECLEHGR | TTCTGGGGTAACCTTCCTGGCATGAACAGGCCTTTGGCA |
| | IAKFSKVRTITTRSN | TCCACTGTGAATGATAAGCTGGAGCTGCAAGAGTGTCTG |
| | SIKQGKDQHFPVFM | GAGCACGGCAGAATAGCCAAGTTCAGCAAAGTGAGGAC |
| | NEKEDILWCTEMER | CATTACCACCAGGTCAAACTCTATAAAGCAGGGCAAAG |
| | VFGFPVHYTDVSN | ACCAGCATTTCCCCGTCTTCATGAACGAGAAGGAGGACA |
| | MSRLARQRLLGRS | TCCTGTGGTGCACTGAAATGGAAAGGGTGTTTGGCTTCC |
| | WSVPVIRHLFAPLK | CCGTCCACTACACAGACGTCTCCAACATGAGCCGCTTGG |
| | EYFACVGGPSSGAP | CGAGGCAGAGACTGCTGGGCCGATCGTGGAGCGTGCCG |
| | PPSGGSPAGSPTSTE | GTCATCCGCCACCTCTTCGCTCCGCTGAAGGAATATTTTG |
| | EGTSESATPESGPGT | CTTGTGTGggagggccgagctctggcgcacccccaccaagtggagggtctcctgcc |
| | STEPSEGSAPGSPAG | gggtccccaacatctactgaagaaggcaccagcgaatccgcaacgcccgagtcaggccctg |
| | SPTSTEEGTSTEPSE | gtacctccacagaaccatctgaaggtagtgcgcctggttccccagctggaagccctacttcca |
| | GSAPGTSTEPSEPKK | ccgaagaaggcacgtcaaccgaaccaagtgaaggatctgcccctgggaccagcactgaac |
| | KRKVMDKKYSIGL | catctgagCCAAAAAAGAAGAGAAAGGTAATGGACAAGAAG |
| | AIGTNSVGWAVIT | TACAGCATCGGCCTGGCCATCGGCACCAACTCTGTGGGC |
| | DEYKVPSKKFKVL | TGGGCCGTGATCACCGACGAGTACAAGGTGCCCAGCAA |
| | GNTDRHSIKKNLI | GAAATTCAAGGTGCTGGGCAACACCGACCGGCACAGCA |
| | GALLFDSGETAEA | TCAAGAAGAACCTGATCGGCGCCCTGCTGTTCGACAGCG |
| | TRLKRTARRRYTR | GAGAAACAGCCGAGGCCACCCGGCTGAAGAGAACCGCC |
| | RKNRICYLQEIFSN | AGAAGAAGATACACCAGACGGAAGAACCGGATCTGCTA |
| | EMAKVDDSFFHRL | TCTGCAAGAGATCTTCAGCAACGAGATGGCCAAGGTGG |
| | EESFLVEEDKKHE | ACGACAGCTTCTTCCACAGACTGGAAGAGTCCTTCCTGG |
| | RHPIFGNIVDEVAY | TGGAAGAGGATAAGAAGCACGAGCGGCACCCCATCTTC |
| | HEKYPTIYHLRKK | GGCAACATCGTGGACGAGGTGGCCTACCACGAGAAGTA |
| | LVDSTDKADLRLI | CCCCACCATCTACCACCTGAGAAAGAAACTGGTGGACA |
| | YLALAHMIKFRGH | GCACCGACAAGGCCGACCTGCGGCTGATCTATCTGGCCC |
| | FLIEGDLNPDNSDV | TGGCCCACATGATCAAGTTCCGGGGCCACTTCCTGATCG |
| | DKLFIQLVQTYNQ | AGGGCGACCTGAACCCCGACAACAGCGACGTGGACAAG |
| | LFEENPINASGVDA | CTGTTCATCCAGCTGGTGCAGACCTACAACCAGCTGTTC |
| | KAILSARLSKSRRL | GAGGAAAACCCCATCAACGCCAGCGGCGTGGACGCCAA |
| | ENLIAQLPGEKKN | GGCCATCCTGTCTGCCAGACTGAGCAAGAGCAGACGGCT |
| | GLFGNLIALSLGLT | GGAAAATCTGATCGCCCAGCTGCCCGGCGAGAAGAAGA |
| | PNFKSNFDLAEDA | ATGGCCTGTTCGGCAACCTGATTGCCCTGAGCCTGGGCC |
| | KLQLSKDTYDDDL | TGACCCCCAACTTCAAGAGCAACTTCGACCTGGCCGAGG |
| | DNLLAQIGDQYAD | ATGCCAAACTGCAGCTGAGCAAGGACACCTACGACGAC |
| | LFLAAKNLSDAILL | GACCTGGACAACCTGCTGGCCCAGATCGGCGACCAGTAC |
| | SDILRVNTEITKAP | GCCGACCTGTTTCTGGCCGCCAAGAACCTGTCCGACGCC |
| | LSASMIKRYDEHH | ATCCTGCTGAGCGACATCCTGAGAGTGAACACCGAGATC |
| | QDLTLLKALVRQQ | ACCAAGGCCCCCCTGAGCGCCTCTATGATCAAGAGATAC |
| | LPEKYKEIFFDQSK | GACGAGCACCACCAGGACCTGACCCTGCTGAAAGCTCTC |
| | NGYAGYIDGGASQ | GTGCGGCAGCAGCTGCCTGAGAAGTACAAAGAGATTTTC |
| | EEFYKFIKPILEKM | TTCGACCAGAGCAAGAACGGCTACGCCGGCTACATCGAT |
| | DGTEELLVKLNRE | GGCGGAGCCAGCCAGGAAGAGTTCTACAAGTTCATCAA |
| | DLLRKQRTFDNGS | GCCCATCCTGGAAAAGATGGACGGCACCGAGGAACTGC |
| | IPHQIHLGELHAIL | TCGTGAAGCTGAACAGAGAGGACCTGCTGCGGAAGCAG |
| | RRQEDFYPFLKDN | CGGACCTTCGACAACGGCAGCATCCCCCACCAGATCCAC |
| | REKIEKILTFRIPY | CTGGGAGAGCTGCACGCCATTCTGCGGCGGCAGGAAGA |
| | YVGPLARGNSRFA | TTTTTACCCATTCCTGAAGGACAACCGGGAAAAGATCGA |
| | WMTRKSEETITPW | GAAGATCCTGACCTTCCGCATCCCCTACTACGTGGGCCC |
| | NFEEVVDKGASAQ | TCTGGCCAGGGGAAACAGCAGATTCGCCTGGATGACCA |
| | SFIERMTNFDKNLP | GAAAGAGCGAGGAAACCATCACCCCCTGGAACTTCGAG |
| | NEKVLPKHSLLYE | GAAGTGGTGGACAAGGGCGCCAGCGCCCAGAGCTTCAT |
| | YFTVYNELTKVKY | CGAGCGGATGACCAACTTCGATAAGAACCTGCCCAACG |
| | VTEGMRKPAFLSG | AGAAGGTGCTGCCCAAGCACAGCCTGCTGTACGAGTACT |
| | EQKKAIVDLLFKT | TCACCGTGTACAACGAGCTGACCAAAGTGAAATACGTG |
| | NRKVTVKQLKED | ACCGAGGGAATGAGAAAGCCCGCCTTCCTGAGCGGCGA |
| | YFKKIECFDSVEIS | GCAGAAAAAAGCCATCGTGGACCTGCTGTTCAAGACCA |
| | GVEDRFNASLGTY | ACCGGAAAGTGACCGTGAAGCAGCTGAAAGAGGACTAC |
| | HDLLKIIKDKDFLD | TTCAAGAAAATCGAGTGCTTCGACTCCGTGGAAATCTCC |
| | NEENEDILEDIVLT | GGCGTGGAAGATCGGTTCAACGCCTCCCTGGGCACATAC |
| | LTLFEDREMIEER | CACGATCTGCTGAAAATTATCAAGGACAAGGACTTCCTG |
| | LKTYAHLFDDKV | GACAATGAGGAAAACGAGGACATTCTGGAAGATATCGT |
| | MKQLKRRRYTGW | GCTGACCCTGACACTGTTTGAGGACAGAGAGATGATCGA |
| | GRLSRKLINGIRDK | GGAACGGCTGAAAACCTATGCCCACCTGTTCGACGACAA |
| | QSGKTILDFLKSD | AGTGATGAAGCAGCTGAAGCGGCGGAGATACACCGGCT |
| | GFANRNFMQLIHD | GGGGCAGGCTGAGCCGGAAGCTGATCAACGGCATCCGG |
| | DSLTFKEDIQKAQ | GACAAGCAGTCCGGCAAGACAATCCTGGATTTCCTGAAG |
| | VSGQGDSLHEHIA | TCCGACGGCTTCGCCAACAGAAACTTCATGCAGCTGATC |
| | NLAGSPAIKKGILQ | CACGACGACAGCCTGACCTTTAAAGAGGACATCCAGAA |
| | TVKVVDELVKVM | AGCCCAGGTGTCCGGCCAGGGCGATAGCCTGCACGAGC |
| | GRHKPENIVIEMA | ACATTGCCAATCTGGCCGGCAGCCCCGCCATTAAGAAGG |
| | RENQTTQKGQKNS | GCATCCTGCAGACAGTGAAGGTGGTGGACGAGCTCGTG |
| | RERMKRIEEGIKE | AAAGTGATGGGCCGGCACAAGCCCGAGAACATCGTGAT |
| | LGSQILKEHPVEN | CGAAATGGCCAGAGAGAACCAGACCACCCAGAAGGGAC |
| | TQLQNEKLYLYYL | AGAAGAACAGCCGCGAGAGAATGAAGCGGATCGAAGA |
| | QNGRDMYVDQEL | GGGCATCAAAGAGCTGGGCAGCCAGATCCTGAAAGAAC |
| | DINRLSDYDVDAIV | ACCCCGTGGAAAACACCCAGCTGCAGAACGAGAAGCTG |
| | PQSFLKDDSIDNKV | TACCTGTACTACCTGCAGAATGGGCGGGATATGTACGTG |
| | LTRSDKNRGKSDN | GACCAGGAACTGGACATCAACCGGCTGTCCGACTACGAT |
| | VPSEEVVKKMKNY | GTGGACGCTATCGTGCCTCAGAGCTTTCTGAAGGACGAC |
| | WRQLLNAKLITQR | TCCATCGATAACAAAGTGCTGACTCGGAGCGACAAGAA |
| | KFDNLTKAERGGL | CCGGGGCAAGAGCGACAACGTGCCCTCCGAAGAGGTCG |
| | SELDKAGFIKRQL | TGAAGAAGATGAAGAACTACTGGCGCCAGCTGCTGAAT |
| | VETRQITKHVAQI | GCCAAGCTGATTACCCAGAGGAAGTTCGACAATCTGACC |
| | LDSRMNTKYDEND | AAGGCCGAGAGAGGCGGCCTGAGCGAACTGGATAAGGC |
| | KLIREVKVITLKSK | CGGCTTCATCAAGAGACAGCTGGTGGAAACCCGGCAGA |
| | LVSDFRKDFQFYK | TCACAAAGCACGTGGCACAGATCCTGGACTCCCGGATGA |
| | VREINNYHHAHDA | ACACTAAGTACGACGAGAACGACAAACTGATCCGGGAA |
| | YLNAVVGTALIKK | GTGAAAGTGATCACCCTGAAGTCCAAGCTGGTGTCCGAT |
| | YPKLESEFVYGDY | TTCCGGAAGGATTTCCAGTTTTACAAAGTGCGCGAGATC |
| | KVYDVRKMIAKSE | AACAACTACCACCACGCCCACGACGCCTACCTGAACGCC |
| | QEIGKATAKYFFY | GTCGTGGGAACCGCCCTGATCAAAAAGTACCCTAAGCTG |
| | SNIMNFFKTEITLA | GAAAGCGAGTTCGTGTACGGCGACTACAAGGTGTACGA |
| | NGEIRKRPLIETNG | CGTGCGGAAGATGATCGCCAAGAGCGAGCAGGAAATCG |
| | ETGEIVWDKGRDF | GCAAGGCTACCGCCAAGTACTTCTTCTACAGCAACATCA |
| | ATVRKVLSMPQVN | TGAACTTTTTCAAGACCGAGATTACCCTGGCCAACGGCG |
| | IVKKTEVQTGGFS | AGATCCGGAAGCGGCCTCTGATCGAGACAAACGGCGAA |
| | KESILPKRNSDKLI | ACAGGCGAGATCGTGTGGGATAAGGGCCGGGACTTTGC |
| | ARKKDWDPKKYG | CACCGTGCGGAAAGTGCTGTCTATGCCCCAAGTGAATAT |
| | GFDSPTVAYSVLV | CGTGAAAAAGACCGAGGTGCAGACAGGCGGCTTCAGCA |
| | VAKVEKGKSKKL | AAGAGTCTATCCTGCCCAAGAGGAACAGCGACAAGCTG |
| | KSVKELLGITIMER | ATCGCCAGAAAGAAGGACTGGGACCCTAAGAAGTACGG |
| | SSFEKNPIDFLEAK | CGGCTTCGACAGCCCCACCGTGGCCTATTCTGTGCTGGT |
| | GYKEVKKDLIIKL | GGTGGCCAAAGTGGAAAAGGGCAAGTCCAAGAAACTGA |
| | PKYSLFELENGRK | AGAGTGTGAAAGAGCTGCTGGGGATCACCATCATGGAA |
| | RMLASAGELQKG | AGAAGCAGCTTCGAGAAGAATCCCATCGACTTTCTGGAA |
| | NELALPSKYVNFL | GCCAAGGGCTACAAAGAAGTGAAAAAGGACCTGATCAT |
| | YLASHYEKLKGSP | CAAGCTGCCTAAGTACTCCCTGTTCGAGCTGGAAAACGG |
| | EDNEQKQLFVEQH | CCGGAAGAGAATGCTGGCCTCTGCCGGCGAACTGCAGA |
| | KHYLDEIIEQISEFS | AGGGAAACGAACTGGCCCTGCCCTCCAAATATGTGAACT |
| | KRVILADANLDKV | TCCTGTACCTGGCCAGCCACTATGAGAAGCTGAAGGGCT |
| | LSAYNKHRDKPIR | CCCCCGAGGATAATGAGCAGAAACAGCTGTTTGTGGAA |
| | EQAENIIHLFTLTN | CAGCACAAACACTACCTGGACGAGATCATCGAGCAGAT |
| | LGAPAAFKYFDTTI | CAGCGAGTTCTCCAAGAGAGTGATCCTGGCCGACGCTAA |
| | DRKRYTSTKEVLD | TCTGGACAAGGTGCTGAGCGCCTACAACAAGCACAGAG |
| | ATLIHQSITGLYET | ACAAGCCTATCAGAGAGCAGGCCGAGAATATCATCCAC |
| | RIDLSQLGGDAYP | CTGTTTACCCTGACCAATCTGGGAGCCCCTGCCGCCTTC |
| | YDVPDYASLGSGSP | AAGTACTTTGACACCACCATCGACCGGAAGAGGTACACC |
| | KKKRKVEDPKKKR | AGCACCAAAGAGGTGCTGGACGCCACCCTGATCCACCA |
| | KVDGSGSETPGTSE | GAGCATCACCGGCCTGTACGAGACACGGATCGACCTGTC |
| | SATPESMNNSQGRV | TCAGCTGGGAGGCGACGCCTATCCCTATGACGTGCCCGA |
| | TFEDVTVNFTQGE | TTATGCCAGCCTGGGCAGCGGCTCCCCCAAGAAAAAAC |
| | WQRLNPEQRNLYR | GCAAGGTGGAAGATCCTAAGAAAAAGCGGAAAGTGGAC |
| | DVMLENYSNLVSVG | GGCagcggcagcgagacccccggtaccagcgagagcgccacccccgagagcatgaac |
| | QGETTKPDVILRLE | aattcccagggaagagtgaccttcgaggatgtcactgtgaacttcacccagggggagtggca |
| | QGKEPWLEEEEVL | gcggctgaatcccgaacagagaaacttgtacagggatgtgatgctggagaattacagcaacc |
| | GSGRAEKNGDIGGQ | ttgtctctgtgggacaaggggaaaccaccaaacccgatgtgatcttgaggttggaacaagga |
| | IWKPKDVKESL | aaggagccatggttggaggaagaggaagtgctgggaagtggccgtgcagaaaaaaatggg |
| | (SEQ ID NO: 160) | gacattggagggcagatttggaagccaaaggatgtgaaagagagtctc SEQ ID NO: |
| | | 188) |
| EPICAS- | MNHDQEFDPPKVY | ATGAACCATGACCAGGAATTTGACCCCCCAAAGGTTTAC |
| V3 | PPVPAEKRKPIRVLS | CCACCTGTGCCAGCTGAGAAGAGGAAGCCCATCCGCGT |
| DNMT3L- | LFDGIATGLLVLKD | GCTGTCTCTCTTTGATGGGATTGCTACAGGGCTCCTGGTG |
| DNMT3A- | LGIQVDRYIASEVC | CTGAAGGACCTGGGCATCCAAGTGGACCGCTACATTGCC |
| dCas9- | EDSITVGMVRHQG | TCCGAGGTGTGTGAGGACTCCATCACGGTGGGCATGGTG |
| ZIM3 | KIMYVGDVRSVTQ | CGGCACCAGGGAAAGATCATGTACGTCGGGGACGTCCG |
| Krab | KHIQEWGPFDLVIG | CAGCGTCACACAGAAGCATATCCAGGAGTGGGGCCCAT |
| (FIG. | GSPCNDLSIVNPAR | TCGACCTGGTGATTGGAGGCAGTCCCTGCAATGACCTCT |
| 6B) | KGLYEGTGRLFFEF | CCATTGTCAACCCTGCCCGCAAGGGACTTTATGAGGGTA |
| | YRLLHDARPKEGD | CTGGCCGCCTCTTCTTTGAGTTCTACCGCCTCCTGCATGA |
| | DRPFFWLFENVVA | TGCGCGGCCCAAGGAGGGAGATGATCGCCCCTTCTTCTG |
| | MGVSDKRDISRFLE | GCTCTTTGAGAATGTGGTGGCCATGGGCGTTAGTGACAA |
| | SNPVMIDAKEVSAA | GAGGGACATCTCGCGATTTCTTGAGTCTAACCCCGTGAT |
| | HRARYFWGNLPGM | GATTGACGCCAAAGAAGTGTCTGCTGCACACAGGGCCC |
| | NRPLASTVNDKLEL | GTTACTTCTGGGGTAACCTTCCTGGCATGAACAGGCCTT |
| | QECLEHGRIAKFSK | TGGCATCCACTGTGAATGATAAGCTGGAGCTGCAAGAGT |
| | VRTITTRSNSIKQGK | GTCTGGAGCACGGCAGAATAGCCAAGTTCAGCAAAGTG |
| | DQHFPVFMNEKEDI | AGGACCATTACCACCAGGTCAAACTCTATAAAGCAGGG |
| | LWCTEMESSGNSN | CAAAGACCAGCATTTCCCCGTCTTCATGAACGAGAAGGA |
| | ANSRGPSFSSGLVPL | GGACATCCTGTGGTGCACTGAAATGGAAAGGGTGTTTGG |
| | SLRGSHMGPMEIYK | CTTCCCCGTCCACTACACAGACGTCTCCAACATGAGCCG |
| | TVSAWKRQPVRVLSL | CTTGGCGAGGCAGAGACTGCTGGGCCGATCGTGGAGCG |
| | FRNIDKVLKSLGFLE | TGCCGGTCATCCGCCACCTCTTCGCTCCGCTGAAGGAAT |
| | SGSGSGGGTLKYVED | ATTTTGCTTGTGTGtctagcggcaatagtaacgctaacagccgcgggccgagct |
| | VTNVVRRDVEKWGP | tcagcagcggcctggtgccgttaagcttgcgcggcagccatATGggccctatggagatat |
| | FDLVYGSTQPLGSSC | acaagacagtgtctgcatggaagagacagccagtgcgggtactgagcctcttcagaaacatc |
| | DRCPGWYMFQFHRI | gacaaggtactaaagagtttgggcttcttGGAAAGCGGTTCTGGTTCTGG |
| | LQYALPRQESQRPFF | GGGAGGAACGCTGAAGTACGTGGAAGATGTCACAAATG |
| | WIFMDNLLLTEDDQ | TCGTGAGGAGAGACGTGGAGAAATGGGGCCCCTTTGAC |
| | ETTTRFLQTEAVTLQ | CTGGTGTACGGCTCGACGCAGCCCCTAGGCAGCTCTTGT |
| | DVRGRDYQNAMRV | GATCGCTGTCCCGGCTGGTACATGTTCCAGTTCCACCGG |
| | WSNIPGLKSKHAPLT | ATCCTGCAGTATGCGCTGCCTCGCCAGGAGAGTCAGCGG |
| | PKEEEYLQAQVRSRS | CCCTTCTTCTGGATATTCATGGACAATCTGCTGCTGACTG |
| | KLDAPKVDLLVKNC | AGGATGACCAAGAGACAACTACCCGCTTCCTTCAGACAG |
| | LLPLREYFKYFSQNS | AGGCTGTGACCCTCCAGGATGTCCGTGGCAGAGACTACC |
| | LPLRVFGFPVHYTD | AGAATGCTATGCGGGTGTGGAGCAACATTCCAGGGCTG |
| | VSNMSRLARQRLL | AAGAGCAAGCATGCGCCCCTGACCCCAAAGGAAGAAGA |
| | GRSWSVPVIRHLFA | GTATCTGCAAGCCCAAGTCAGAAGCAGGAGCAAGCTGG |
| | PLKEYFACVGGPSS | ACGCCCCGAAAGTTGACCTCCTGGTGAAGAACTGCCTTC |
| | GAPPPSGGSPAGSPT | TCCCGCTGAGAGAGTACTTCAAGTATTTTTCTCAAAACT |
| | STEEGTSESATPESG | CACTTCCTCTTggagggccgagctctggcgcacccccaccaagtggagggtctcc |
| | PGTSTEPSEGSAPGS | tgccgggtccccaacatctactgaagaaggcaccagcgaatccgcaacgcccgagtcaggc |
| | PAGSPTSTEEGTSTE | cctggtacctccacagaaccatctgaaggtagtgcgcctggttccccagctggaagccctact |
| | PSEGSAPGTSTEPSE | tccaccgaagaaggcacgtcaaccgaaccaagtgaaggatctgcccctgggaccagcactg |
| | PKKKRKVMDKKYS | aaccatctgagCCAAAAAAGAAGAGAAAGGTAATGGACAAGA |
| | IGLAIGTNSVGWA | AGTACAGCATCGGCCTGGCCATCGGCACCAACTCTGTGG |
| | VITDEYKVPSKKF | GCTGGGCCGTGATCACCGACGAGTACAAGGTGCCCAGC |
| | KVLGNTDRHSIKK | AAGAAATTCAAGGTGCTGGGCAACACCGACCGGCACAG |
| | NLIGALLFDSGETA | CATCAAGAAGAACCTGATCGGCGCCCTGCTGTTCGACAG |
| | EATRLKRTARRRY | CGGAGAAACAGCCGAGGCCACCCGGCTGAAGAGAACCG |
| | TRRKNRICYLQEIF | CCAGAAGAAGATACACCAGACGGAAGAACCGGATCTGC |
| | SNEMAKVDDSFFH | TATCTGCAAGAGATCTTCAGCAACGAGATGGCCAAGGTG |
| | RLEESFLVEEDKK | GACGACAGCTTCTTCCACAGACTGGAAGAGTCCTTCCTG |
| | HERHPIFGNIVDEV | GTGGAAGAGGATAAGAAGCACGAGCGGCACCCCATCTT |
| | AYHEKYPTIYHLR | CGGCAACATCGTGGACGAGGTGGCCTACCACGAGAAGT |
| | KKLVDSTDKADLR | ACCCCACCATCTACCACCTGAGAAAGAAACTGGTGGAC |
| | LIYLALAHMIKFR | AGCACCGACAAGGCCGACCTGCGGCTGATCTATCTGGCC |
| | GHFLIEGDLNPDNS | CTGGCCCACATGATCAAGTTCCGGGGCCACTTCCTGATC |
| | DVDKLFIQLVQTY | GAGGGCGACCTGAACCCCGACAACAGCGACGTGGACAA |
| | NQLFEENPINASGV | GCTGTTCATCCAGCTGGTGCAGACCTACAACCAGCTGTT |
| | DAKAILSARLSKSR | CGAGGAAAACCCCATCAACGCCAGCGGCGTGGACGCCA |
| | RLENLIAQLPGEK | AGGCCATCCTGTCTGCCAGACTGAGCAAGAGCAGACGG |
| | KNGLFGNLIALSL | CTGGAAAATCTGATCGCCCAGCTGCCCGGCGAGAAGAA |
| | GLTPNFKSNFDLA | GAATGGCCTGTTCGGCAACCTGATTGCCCTGAGCCTGGG |
| | EDAKLQLSKDTYD | CCTGACCCCCAACTTCAAGAGCAACTTCGACCTGGCCGA |
| | DDLDNLLAQIGDQ | GGATGCCAAACTGCAGCTGAGCAAGGACACCTACGACG |
| | YADLFLAAKNLSD | ACGACCTGGACAACCTGCTGGCCCAGATCGGCGACCAGT |
| | AILLSDILRVNTEIT | ACGCCGACCTGTTTCTGGCCGCCAAGAACCTGTCCGACG |
| | KAPLSASMIKRYD | CCATCCTGCTGAGCGACATCCTGAGAGTGAACACCGAGA |
| | EHHQDLTLLKALV | TCACCAAGGCCCCCCTGAGCGCCTCTATGATCAAGAGAT |
| | RQQLPEKYKEIFF | ACGACGAGCACCACCAGGACCTGACCCTGCTGAAAGCT |
| | DQSKNGYAGYIDG | CTCGTGCGGCAGCAGCTGCCTGAGAAGTACAAAGAGAT |
| | GASQEEFYKFIKPI | TTTCTTCGACCAGAGCAAGAACGGCTACGCCGGCTACAT |
| | LEKMDGTEELLVK | CGATGGCGGAGCCAGCCAGGAAGAGTTCTACAAGTTCA |
| | LNREDLLRKQRTF | TCAAGCCCATCCTGGAAAAGATGGACGGCACCGAGGAA |
| | DNGSIPHQIHLGEL | CTGCTCGTGAAGCTGAACAGAGAGGACCTGCTGCGGAA |
| | HAILRRQEDFYPFL | GCAGCGGACCTTCGACAACGGCAGCATCCCCCACCAGAT |
| | KDNREKIEKILTFR | CCACCTGGGAGAGCTGCACGCCATTCTGCGGCGGCAGG |
| | IPYYVGPLARGNS | AAGATTTTTACCCATTCCTGAAGGACAACCGGGAAAAGA |
| | RFAWMTRKSEETI | TCGAGAAGATCCTGACCTTCCGCATCCCCTACTACGTGG |
| | TPWNFEEVVDKGA | GCCCTCTGGCCAGGGGAAACAGCAGATTCGCCTGGATG |
| | SAQSFIERMTNFD | ACCAGAAAGAGCGAGGAAACCATCACCCCCTGGAACTT |
| | KNLPNEKVLPKHS | CGAGGAAGTGGTGGACAAGGGCGCCAGCGCCCAGAGCT |
| | LLYEYFTVYNELT | TCATCGAGCGGATGACCAACTTCGATAAGAACCTGCCCA |
| | KVKYVTEGMRKP | ACGAGAAGGTGCTGCCCAAGCACAGCCTGCTGTACGAG |
| | AFLSGEQKKAIVD | TACTTCACCGTGTACAACGAGCTGACCAAAGTGAAATAC |
| | LLFKTNRKVTVKQ | GTGACCGAGGGAATGAGAAAGCCCGCCTTCCTGAGCGG |
| | LKEDYFKKIECFDS | CGAGCAGAAAAAAGCCATCGTGGACCTGCTGTTCAAGA |
| | VEISGVEDRFNASL | CCAACCGGAAAGTGACCGTGAAGCAGCTGAAAGAGGAC |
| | GTYHDLLKIIKDK | TACTTCAAGAAAATCGAGTGCTTCGACTCCGTGGAAATC |
| | DFLDNEENEDILED | TCCGGCGTGGAAGATCGGTTCAACGCCTCCCTGGGCACA |
| | IVLTLTLFEDREMI | TACCACGATCTGCTGAAAATTATCAAGGACAAGGACTTC |
| | EERLKTYAHLFDD | CTGGACAATGAGGAAAACGAGGACATTCTGGAAGATAT |
| | KVMKQLKRRRYT | CGTGCTGACCCTGACACTGTTTGAGGACAGAGAGATGAT |
| | GWGRLSRKLINGI | CGAGGAACGGCTGAAAACCTATGCCCACCTGTTCGACGA |
| | RDKQSGKTILDFL | CAAAGTGATGAAGCAGCTGAAGCGGCGGAGATACACCG |
| | KSDGFANRNFMQL | GCTGGGGCAGGCTGAGCCGGAAGCTGATCAACGGCATC |
| | IHDDSLTFKEDIQK | CGGGACAAGCAGTCCGGCAAGACAATCCTGGATTTCCTG |
| | AQVSGQGDSLHEH | AAGTCCGACGGCTTCGCCAACAGAAACTTCATGCAGCTG |
| | IANLAGSPAIKKGI | ATCCACGACGACAGCCTGACCTTTAAAGAGGACATCCAG |
| | LQTVKVVDELVKV | AAAGCCCAGGTGTCCGGCCAGGGCGATAGCCTGCACGA |
| | MGRHKPENIVIEM | GCACATTGCCAATCTGGCCGGCAGCCCCGCCATTAAGAA |
| | ARENQTTQKGQK | GGGCATCCTGCAGACAGTGAAGGTGGTGGACGAGCTCG |
| | NSRERMKRIEEGI | TGAAAGTGATGGGCCGGCACAAGCCCGAGAACATCGTG |
| | KELGSQILKEHPV | ATCGAAATGGCCAGAGAGAACCAGACCACCCAGAAGGG |
| | ENTQLQNEKLYLY | ACAGAAGAACAGCCGCGAGAGAATGAAGCGGATCGAAG |
| | YLQNGRDMYVDQ | AGGGCATCAAAGAGCTGGGCAGCCAGATCCTGAAAGAA |
| | ELDINRLSDYDVD | CACCCCGTGGAAAACACCCAGCTGCAGAACGAGAAGCT |
| | AIVPQSFLKDDSID | GTACCTGTACTACCTGCAGAATGGGCGGGATATGTACGT |
| | NKVLTRSDKNRGK | GGACCAGGAACTGGACATCAACCGGCTGTCCGACTACG |
| | SDNVPSEEVVKKM | ATGTGGACGCTATCGTGCCTCAGAGCTTTCTGAAGGACG |
| | KNYWRQLLNAKLI | ACTCCATCGATAACAAAGTGCTGACTCGGAGCGACAAG |
| | TQRKFDNLTKAER | AACCGGGGCAAGAGCGACAACGTGCCCTCCGAAGAGGT |
| | GGLSELDKAGFIK | CGTGAAGAAGATGAAGAACTACTGGCGCCAGCTGCTGA |
| | RQLVETRQITKHV | ATGCCAAGCTGATTACCCAGAGGAAGTTCGACAATCTGA |
| | AQILDSRMNTKYD | CCAAGGCCGAGAGAGGCGGCCTGAGCGAACTGGATAAG |
| | ENDKLIREVKVITL | GCCGGCTTCATCAAGAGACAGCTGGTGGAAACCCGGCA |
| | KSKLVSDFRKDFQ | GATCACAAAGCACGTGGCACAGATCCTGGACTCCCGGAT |
| | FYKVREINNYHHA | GAACACTAAGTACGACGAGAACGACAAACTGATCCGGG |
| | HDAYLNAVVGTAL | AAGTGAAAGTGATCACCCTGAAGTCCAAGCTGGTGTCCG |
| | IKKYPKLESEFVY | ATTTCCGGAAGGATTTCCAGTTTTACAAAGTGCGCGAGA |
| | GDYKVYDVRKMI | TCAACAACTACCACCACGCCCACGACGCCTACCTGAACG |
| | AKSEQEIGKATAK | CCGTCGTGGGAACCGCCCTGATCAAAAAGTACCCTAAGC |
| | YFFYSNIMNFFKTE | TGGAAAGCGAGTTCGTGTACGGCGACTACAAGGTGTAC |
| | ITLANGEIRKRPLI | GACGTGCGGAAGATGATCGCCAAGAGCGAGCAGGAAAT |
| | ETNGETGEIVWDK | CGGCAAGGCTACCGCCAAGTACTTCTTCTACAGCAACAT |
| | GRDFATVRKVLSM | CATGAACTTTTTCAAGACCGAGATTACCCTGGCCAACGG |
| | PQVNIVKKTEVQT | CGAGATCCGGAAGCGGCCTCTGATCGAGACAAACGGCG |
| | GGFSKESILPKRNS | AAACAGGCGAGATCGTGTGGGATAAGGGCCGGGACTTT |
| | DKLIARKKDWDPK | GCCACCGTGCGGAAAGTGCTGTCTATGCCCCAAGTGAAT |
| | KYGGFDSPTVAYS | ATCGTGAAAAAGACCGAGGTGCAGACAGGCGGCTTCAG |
| | VLVVAKVEKGKSK | CAAAGAGTCTATCCTGCCCAAGAGGAACAGCGACAAGC |
| | KLKSVKELLGITI | TGATCGCCAGAAAGAAGGACTGGGACCCTAAGAAGTAC |
| | MERSSFEKNPIDFL | GGCGGCTTCGACAGCCCCACCGTGGCCTATTCTGTGCTG |
| | EAKGYKEVKKDLI | GTGGTGGCCAAAGTGGAAAAGGGCAAGTCCAAGAAACT |
| | IKLPKYSLFELENG | GAAGAGTGTGAAAGAGCTGCTGGGGATCACCATCATGG |
| | RKRMLASAGELQ | AAAGAAGCAGCTTCGAGAAGAATCCCATCGACTTTCTGG |
| | KGNELALPSKYVN | AAGCCAAGGGCTACAAAGAAGTGAAAAAGGACCTGATC |
| | FLYLASHYEKLKG | ATCAAGCTGCCTAAGTACTCCCTGTTCGAGCTGGAAAAC |
| | SPEDNEQKQLFVE | GGCCGGAAGAGAATGCTGGCCTCTGCCGGCGAACTGCA |
| | QHKHYLDEIIEQIS | GAAGGGAAACGAACTGGCCCTGCCCTCCAAATATGTGA |
| | EFSKRVILADANLD | ACTTCCTGTACCTGGCCAGCCACTATGAGAAGCTGAAGG |
| | KVLSAYNKHRDKP | GCTCCCCCGAGGATAATGAGCAGAAACAGCTGTTTGTGG |
| | IREQAENIIHLFTL | AACAGCACAAACACTACCTGGACGAGATCATCGAGCAG |
| | TNLGAPAAFKYFD | ATCAGCGAGTTCTCCAAGAGAGTGATCCTGGCCGACGCT |
| | TTIDRKRYTSTKE | AATCTGGACAAGGTGCTGAGCGCCTACAACAAGCACAG |
| | VLDATLIHQSITGL | AGACAAGCCTATCAGAGAGCAGGCCGAGAATATCATCC |
| | YETRIDLSQLGGD | ACCTGTTTACCCTGACCAATCTGGGAGCCCCTGCCGCCT |
| | AYPYDVPDYASLGS | TCAAGTACTTTGACACCACCATCGACCGGAAGAGGTACA |
| | GSPKKKRKVEDPK | CCAGCACCAAAGAGGTGCTGGACGCCACCCTGATCCACC |
| | KKRKVDGSGSETPG | AGAGCATCACCGGCCTGTACGAGACACGGATCGACCTGT |
| | TSESATPESMNNSQ | CTCAGCTGGGAGGCGACGCCTATCCCTATGACGTGCCCG |
| | GRVTFEDVTVNFTQ | ATTATGCCAGCCTGGGCAGCGGCTCCCCCAAGAAAAAA |
| | GEWQRLNPEQRNL | CGCAAGGTGGAAGATCCTAAGAAAAAGCGGAAAGTGGA |
| | YRDVMLENYSNLVS | CGGCagcggcagcgagacccccggtaccagcgagagcgccacccccgagagcatga |
| | VGQGETTKPDVILR | acaattcccagggaagagtgaccttcgaggatgtcactgtgaacttcacccagggggagtgg |
| | LEQGKEPWLEEEE | cagcggctgaatcccgaacagagaaacttgtacagggatgtgatgctggagaattacagcaa |
| | VLGSGRAEKNGDIG | ccttgtctctgtgggacaaggggaaaccaccaaacccgatgtgatcttgaggttggaacaag |
| | GQIWKPKDVKESL | gaaaggagccatggttggaggaagaggaagtgctgggaagtggccgtgcagaaaaaaatg |
| | (SEQ ID NO: 161) | gggacattggagggcagatttggaagccaaaggatgtgaaagagagtctc SEQ ID |
| | | NO: 189) |

The present disclosure provides the constructs comprising Formula: 5′-(A-B)-CasN-CasC-Kᵣ-D_{q}-3′, wherein: r is an integer selected from 0 to 5; q is an integer selected from 0 to 5; and at least one of q and r is not 0.

In some embodiments, the construct comprises the following amino acid sequence (KRAB3 shown underlined):

(1) DNMT3A-DNMT3L-dCas9-KRAB(KOX1)
(SEQ ID NO: 138)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSKVRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELL VKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESA TPES RTLVTFKDVFVDFTR EEWKLLDTAQQIVY RNVMLENYKNLVSL
GYQLTKPDVILRLEK GEEP,

wherein the nucleic acid sequence of KRAB(KOX1) is:

(SEQ ID NO: 52)
CGGACACTGGTGACCTTCAAGGATGTATTTGTGGACTTCACCAGG
GAGGAGTGGAAGCTGCTGGACACTGCTCAGCAGATCGTGTACAGA
AATGTGATGCTGGAGAACTATAAGAACCTGGTTTCCTTGGGTTAT
CAGCTTACTAAGCCAGATGTGATCCTCCGGTTGGAGAAGGGAGAA
GAGCCC.
(2) DNMT3A-DNMT3L-dCas9-KRAB(ZIM3)
(SEQ ID NO: 204)
MNHDQEFDPPKVYPPVPAEKRKPIRVLSLFDGIATGLLVLKDLGI
QVDRYIASEVCEDSITVGMVRHQGKIMYVGDVRSVTQKHIQEWGP
FDLVIGGSPCNDLSIVNPARKGLYEGTGRLFFEFYRLLHDARPKE
GDDRPFFWLFENVVAMGVSDKRDISRFLESNPVMIDAKEVSAAHR
ARYFWGNLPGMNRPLASTVNDKLELQECLEHGRIAKFSKVRTITT
RSNSIKQGKDQHFPVFMNEKEDILWCTEMESSGNSNANSRGPSFS
SGLVPLSLRGSHMGPMEIYKTVSAWKRQPVRVLSLFRNIDKVLKS
LGFLESGSGSGGGTLKYVEDVTNVVRRDVEKWGPFDLVYGSTQPL
GSSCDRCPGWYMFQFHRILQYALPRQESQRPFFWIFMDNLLLTED
DQETTTRFLQTEAVTLQDVRGRDYQNAMRVWSNIPGLKSKHAPLT
PKEEEYLQAQVRSRSKLDAPKVDLLVKNCLLPLREYFKYFSQNSL
PLRVFGFPVHYTDVSNMSRLARQRLLGRSWSVPVIRHLFAPLKEY
FACVGGPSSGAPPPSGGSPAGSPTSTEEGTSESATPESGPGTSTE
PSEGSAPGSPAGSPTSTEEGTSTEPSEGSAPGTSTEPSEPKKKRK
VMDKKYSIGLAIGTNSVGWAVITDEYKVPSKKFKVLGNTDRHSIK
KNLIGALLFDSGETAEATRLKRTARRRYTRRKNRICYLQEIFSNE
MAKVDDSFFHRLEESFLVEEDKKHERHPIFGNIVDEVAYHEKYPT
IYHLRKKLVDSTDKADLRLIYLALAHMIKFRGHFLIEGDLNPDNS
DVDKLFIQLVQTYNQLFEENPINASGVDAKAILSARLSKSRRLEN
LIAQLPGEKKNGLFGNLIALSLGLTPNFKSNFDLAEDAKLQLSKD
TYDDDLDNLLAQIGDQYADLFLAAKNLSDAILLSDILRVNTEITK
APLSASMIKRYDEHHQDLTLLKALVRQQLPEKYKEIFFDQSKNGY
AGYIDGGASQEEFYKFIKPILEKMDGTEELLVKLNREDLLRKQRT
FDNGSIPHQIHLGELHAILRRQEDFYPFLKDNREKIEKILTFRIP
YYVGPLARGNSRFAWMTRKSEETITPWNFEEVVDKGASAQSFIER
MTNFDKNLPNEKVLPKHSLLYEYFTVYNELTKVKYVTEGMRKPAF
LSGEQKKAIVDLLFKTNRKVTVKQLKEDYFKKIECFDSVEISGVE
DRFNASLGTYHDLLKIIKDKDFLDNEENEDILEDIVLTLTLFEDR
EMIEERLKTYAHLFDDKVMKQLKRRRYTGWGRLSRKLINGIRDKQ
SGKTILDFLKSDGFANRNFMQLIHDDSLTFKEDIQKAQVSGQGDS
LHEHIANLAGSPAIKKGILQTVKVVDELVKVMGRHKPENIVIEMA
RENQTTQKGQKNSRERMKRIEEGIKELGSQILKEHPVENTQLQNE
KLYLYYLQNGRDMYVDQELDINRLSDYDVDAIVPQSFLKDDSIDN
KVLTRSDKNRGKSDNVPSEEVVKKMKNYWRQLLNAKLITQRKFDN
LTKAERGGLSELDKAGFIKRQLVETRQITKHVAQILDSRMNTKYD
ENDKLIREVKVITLKSKLVSDFRKDFQFYKVREINNYHHAHDAYL
NAVVGTALIKKYPKLESEFVYGDYKVYDVRKMIAKSEQEIGKATA
KYFFYSNIMNFFKTEITLANGEIRKRPLIETNGETGEIVWDKGRD
FATVRKVLSMPQVNIVKKTEVQTGGFSKESILPKRNSDKLIARKK
DWDPKKYGGFDSPTVAYSVLVVAKVEKGKSKKLKSVKELLGITIM
ERSSFEKNPIDFLEAKGYKEVKKDLIIKLPKYSLFELENGRKRML
ASAGELQKGNELALPSKYVNFLYLASHYEKLKGSPEDNEQKQLFV
EQHKHYLDEIIEQISEFSKRVILADANLDKVLSAYNKHRDKPIRE
QAENIIHLFTLTNLGAPAAFKYFDTTIDRKRYTSTKEVLDATLIH
QSITGLYETRIDLSQLGGDAYPYDVPDYASLGSGSPKKKRKVEDP
KKKRKVDGSGSETPGTSESATPESRTLVTFEDVTVNFTQGEWQRL
NPEQRNLYRDVMLENYSNLVSVGQGETTKPDVILRLEQGKEPWL.

wherein the nucleic acid sequence of KRAB(ZIM3) is: CGGACACTGGTGACCTTCGAGGACGTGACCGTGAACTTCACCCAAGGCGAGTGGCA GAGACTGAACCCCGAGCAGAGAAACCTGTACCGGGACGTGATGCTGGAAAACTACA GCAACCTGGTGTCCGTCGGCCAGGGCGAGACAACAAAGCCTGACGTGATCCTGCGG CTGGAACAGGGCAAAGAACCTTGGCTG(SEQ ID NO: 54).

(SEQ ID NO: 54)
CGGACACTGGTGACCTTCGAGGACGTGACCGTGAACTTCACCCAA
GGCGAGTGGCAGAGACTGAACCCCGAGCAGAGAAACCTGTACCGG
GACGTGATGCTGGAAAACTACAGCAACCTGGTGTCCGTCGGCCAG
GGCGAGACAACAAAGCCTGACGTGATCCTGCGGCTGGAACAGGGC
AAAGAACCTTGGCTG.
(3) DNMT3A-DNMT3L-dCas9-KRAB(ZNF680)
(SEQ ID NO: 205)
MNHDQEFDPPKVYPPVPAEKRKPIRVLSLFDGIATGLLVLKDLGI
QVDRYIASEVCEDSITVGMVRHQGKIMYVGDVRSVTQKHIQEWGP
FDLVIGGSPCNDLSIVNPARKGLYEGTGRLFFEFYRLLHDARPKE
GDDRPFFWLFENVVAMGVSDKRDISRFLESNPVMIDAKEVSAAHR
ARYFWGNLPGMNRPLASTVNDKLELQECLEHGRIAKFSKVRTITT
RSNSIKQGKDQHFPVFMNEKEDILWCTEMESSGNSNANSRGPSFS
SGLVPLSLRGSHMGPMEIYKTVSAWKRQPVRVLSLFRNIDKVLKS
LGFLESGSGSGGGTLKYVEDVTNVVRRDVEKWGPFDLVYGSTQPL
GSSCDRCPGWYMFQFHRILQYALPRQESQRPFFWIFMDNLLLTED
DQETTTRFLQTEAVTLQDVRGRDYQNAMRVWSNIPGLKSKHAPLT
PKEEEYLQAQVRSRSKLDAPKVDLLVKNCLLPLREYFKYFSQNSL
PLRVFGFPVHYTDVSNMSRLARQRLLGRSWSVPVIRHLFAPLKEY
FACVGGPSSGAPPPSGGSPAGSPTSTEEGTSESATPESGPGTSTE
PSEGSAPGSPAGSPTSTEEGTSTEPSEGSAPGTSTEPSEPKKKRK
VMDKKYSIGLAIGTNSVGWAVITDEYKVPSKKFKVLGNTDRHSIK
KNLIGALLFDSGETAEATRLKRTARRRYTRRKNRICYLQEIFSNE
MAKVDDSFFHRLEESFLVEEDKKHERHPIFGNIVDEVAYHEKYPT
IYHLRKKLVDSTDKADLRLIYLALAHMIKFRGHFLIEGDLNPDNS
DVDKLFIQLVQTYNQLFEENPINASGVDAKAILSARLSKSRRLEN
LIAQLPGEKKNGLFGNLIALSLGLTPNFKSNFDLAEDAKLQLSKD
TYDDDLDNLLAQIGDQYADLFLAAKNLSDAILLSDILRVNTEITK
APLSASMIKRYDEHHQDLTLLKALVRQQLPEKYKEIFFDQSKNGY
AGYIDGGASQEEFYKFIKPILEKMDGTEELLVKLNREDLLRKQRT
FDNGSIPHQIHLGELHAILRRQEDFYPFLKDNREKIEKILTFRIP
YYVGPLARGNSRFAWMTRKSEETITPWNFEEVVDKGASAQSFIER
MTNFDKNLPNEKVLPKHSLLYEYFTVYNELTKVKYVTEGMRKPAF
LSGEQKKAIVDLLFKTNRKVTVKQLKEDYFKKIECFDSVEISGVE
DRFNASLGTYHDLLKIIKDKDFLDNEENEDILEDIVLTLTLFEDR
EMIEERLKTYAHLFDDKVMKQLKRRRYTGWGRLSRKLINGIRDKQ
SGKTILDFLKSDGFANRNFMQLIHDDSLTFKEDIQKAQVSGQGDS
LHEHIANLAGSPAIKKGILQTVKVVDELVKVMGRHKPENIVIEMA
RENQTTQKGQKNSRERMKRIEEGIKELGSQILKEHPVENTQLQNE
KLYLYYLQNGRDMYVDQELDINRLSDYDVDAIVPQSFLKDDSIDN
KVLTRSDKNRGKSDNVPSEEVVKKMKNYWRQLLNAKLITQRKFDN
LTKAERGGLSELDKAGFIKRQLVETRQITKHVAQILDSRMNTKYD
ENDKLIREVKVITLKSKLVSDFRKDFQFYKVREINNYHHAHDAYL
NAVVGTALIKKYPKLESEFVYGDYKVYDVRKMIAKSEQEIGKATA
KYFFYSNIMNFFKTEITLANGEIRKRPLIETNGETGEIVWDKGRD
FATVRKVLSMPQVNIVKKTEVQTGGFSKESILPKRNSDKLIARKK
DWDPKKYGGFDSPTVAYSVLVVAKVEKGKSKKLKSVKELLGITIM
ERSSFEKNPIDFLEAKGYKEVKKDLIIKLPKYSLFELENGRKRML
ASAGELQKGNELALPSKYVNFLYLASHYEKLKGSPEDNEQKQLFV
EQHKHYLDEIIEQISEFSKRVILADANLDKVLSAYNKHRDKPIRE
QAENIIHLFTLTNLGAPAAFKYFDTTIDRKRYTSTKEVLDATLIH
QSITGLYETRIDLSQLGGDAYPYDVPDYASLGSGSPKKKRKVEDP
KKKRKVDGSGSETPGTSESATPESRTLLTFRDVAIEFSLEEWQCL
DTAQRNLYRKVMFENYRNLVFLGIAVSKPHLITCLEQGKEPWN,

Wherein the nucleic acid sequence of KRAB(ZNF680) is:

(SEQ ID NO: 206)
CGGACACTGCTGACCTTCAGAGATGTGGCCATCGAGTTCAGCCTGGAAGAGTGGCA
GTGCCTGGACACAGCCCAGCGGAACCTGTACAGAAAAGTGATGTTCGAGAACTACC
GGAACCTCGTGTTCCTGGGAATCGCCGTGTCTAAGCCCCACCTGATCACCTGTCTCG
AGCAAGGGAAAGAACCCTGGAAC.
(4) DNMT3A-DNMT3L-dCas9-KRAB(ZNF554)
(SEQ ID NO: 207)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSKVRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELLVKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESATPESRTLVTFEDVSMDFSQEEWELLEPAQKNLYREVMLENYRNVVSLEA
LKNQCTDVGIKEGPLSPAQT,

wherein the nucleic acid sequence of KRAB(ZNF554) is:

(SEQ ID NO: 208)
CGGACACTGGTGACATTTGAGGATGTCTCCATGGACTTCAGCCAAGAGGAATGGGA
GCTGCTGGAACCCGCTCAGAAGAACCTGTATAGGGAAGTCATGCTCGAAAATTACC
GGAATGTGGTGTCCCTGGAAGCCCTGAAGAACCAGTGTACCGACGTGGGCATCAAA
GAGGGCCCACTGTCTCCAGCTCAGACC.
(5) DNMT3A-DNMT3L-dCas9-KRAB(ZNF264)
(SEQ ID NO: 209)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSKVRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELLVKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESATPESRTLVTFDDVAVTFTKEEWGQLDLAQRTLYQEVMLENCGLLVSLGC
PVPKAELICHLEHGQEPWT,

wherein the nucleic acid sequence of KRAB(ZNF264) is:

(SEQ ID NO: 210)
CGGACACTGGTGACTTTCGACGATGTGGCCGTGACCTTTACCAAAGAAGAGTGGGG
CCAGCTGGATCTGGCCCAGAGAACACTGTACCAAGAAGTTATGCTGGAGAACTGCG
GCCTGCTGGTGTCTCTGGGATGTCCTGTGCCTAAGGCCGAGCTGATCTGCCACCTGG
AACACGGACAAGAGCCTTGGACC.
(6) DNMT3A-DNMT3L-dCas9-KRAB(ZNF582)
(SEQ ID NO: 211)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSKVRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELLVKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESATPESRTLELFRDVAIVFSQEEWQWLAPAQRDLYRDVMLETYSNLVSLGL
AVSKPDVISFLEQGKEPWM,

wherein the nucleic acid sequence of KRAB(ZNF582) is:

(SEQ ID NO: 212)
CGGACACTGGAGCTGTTCCGCGACGTGGCCATTGTGTTCTCTCAAGAGGAATGGCAG
TGGCTGGCCCCTGCTCAGAGAGATCTCTACCGCGACGTTATGCTGGAGACATACTCC
AATCTCGTGTCCCTCGGCCTGGCCGTGTCCAAACCTGATGTGATCAGCTTCCTCGAG
CAGGGAAAAGAACCTTGGATG.
(7) DNMT3A-DNMT3L-dCas9-KRAB(ZNF324)
(SEQ ID NO: 213)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSKVRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELLVKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESATPESRTLMAFEDVAVYFSQEEWGLLDTAQRALYRRVMLDNFALVASLG
LSTSRPRVVIQLERGEEPWV,

wherein the nucleic acid sequence of KRAB(ZNF324) is:

(SEQ ID NO: 214)
CGGACACTGATGGCCTTTGAGGACGTCGCCGTGTATTTTTCCCAAGAAGAATGGGGA
CTGCTCGACACTGCCCAGAGAGCCCTGTATCGCAGAGTCATGCTGGACAACTTCGCC
CTGGTGGCCTCCCTGGGCCTGTCTACAAGCAGACCCAGAGTGGTCATTCAGCTGGAA
AGAGGCGAGGAACCCTGGGTC.
(8) DNMT3A-DNMT3L-dCas9-KRAB(ZNF669)
(SEQ ID NO: 215)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSKVRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELLVKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESATPESRTLVAFEDVAVNFTQEEWALLDSSQKNLYREVMQETCRNLASVGS
QWKDQNIEDHFEKPGKDIRN,

wherein the nucleic acid sequence of KRAB(ZNF669) is:

(SEQ ID NO: 216)
CGGACACTGGTCGCCTTTGAAGATGTGGCTGTGAATTTCACCCAAGAAGAGTGGGCT
CTGCTGGACAGCAGCCAGAAGAATCTCTACCGCGAAGTGATGCAAGAGACATGCCG
GAACCTGGCCTCTGTGGGCTCTCAGTGGAAGGACCAGAACATCGAGGACCACTTCG
AGAAGCCCGGCAAGGACATCAGAAAC.
(9) DNMT3A-DNMT3L-dCas9-KRAB(ZNF354A)
(SEQ ID NO: 217)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSKVRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELLVKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESATPESRTLLTFEDVAVLFTRDEWRKLAPSQRNLYRDVMLENYRNLVSLGL
PFTKPKVISLLQQGEDPWE,

wherein the nucleic acid sequence of KRAB(ZNF354A) is:

(SEQ ID NO: 218)
CGGACACTGCTGACATTCGAAGATGTCGCAGTGCTGTTCACCCGGGACGAGTGGAG
GAAACTGGCTCCCAGCCAGCGCAATCTGTATAGAGATGTGATGCTGGAGAATTACA
GAAATCTCGTCAGCCTGGGGCTGCCCTTCACCAAGCCTAAAGTGATCAGCCTGCTGC
AACAAGGCGAGGATCCTTGGGAA.
(10) DNMT3A-DNMT3L-dCas9-KRAB(ZNF82)
(SEQ ID NO: 219)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSKVRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELLVKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESATPESRTLVMFSDVSIDESPEEWEYLDLEQKDLYRDVMLENYSNLVSLGCF
ISKPDVISSLEQGKEPWK,

wherein the nucleic acid sequence of KRAB ZNF82 is:

(SEQ ID NO: 220)
CGGACACTGGTTATGTTCTCCGACGTGTCCATCGACTTTAGCCCCGAAGAGTGGGAG
TATCTGGACCTGGAACAGAAGGATCTGTATCGCGACGTGATGCTCGAGAATTACTCT
AACCTGGTGAGCCTGGGCTGCTTCATCAGCAAGCCCGATGTGATCTCTAGCCTGGAG
CAAGGCAAAGAGCCATGGAAA.
(11) DNMT3A-DNMT3L-dCas9-KRAB(ZNF595)
(SEQ ID NO: 221)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSKVRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELLVKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESATPESRTLVTFRDVAIEFSPEEWKCLDPAQQNLYRDVMLENYRNLVSLGFV
ISNPDLVTCLEQIKEPCN,

wherein the nucleic acid sequence of KRAB(ZNF595) is:

(SEQ ID NO: 222)
CGGACACTGGTCACCTTCCGGGACGTCGCAATCGAATTCAGCCCCGAGGAATGGAA
ATGTCTGGACCCCGCACAGCAAAACCTCTACAGAGATGTCATGCTCGAAAACTATA
GGAACCTGGTCTCCCTGGGCTTCGTGATCAGCAACCCTGATCTCGTGACCTGCCTCG
AACAGATCAAAGAGCCCTGCAAC.
(12) DNMT3A-DNMT3L-dCas9-KRAB(ZNF419)
(SEQ ID NO: 223)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSKVRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELLVKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESATPESRTLVTFEDVAVYFSQEEWRLLDDAQRLLYRNVMLENFTLLASLGL
ASSKTHEITQLESWEEPFM,

wherein the nucleic acid sequence of KRAB(ZNF419) is:

(SEQ ID NO: 224)
CGGACACTGGTTACCTTCGAAGATGTTGCCGTGTACTTCAGCCAAGAAGAGTGGCGG
CTGCTGGATGACGCCCAGAGACTGCTGTATCGGAATGTTATGCTCGAAAACTTCACC
CTGCTGGCTTCCCTGGGACTCGCCAGCTCTAAGACCCACGAGATTACCCAGCTGGAA
TCCTGGGAAGAACCCTTCATG.
(13) DNMT3A-DNMT3L-dCas9-KRAB(ZNF566)
(SEQ ID NO: 225)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSKVRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELLVKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESATPESRTLVMFSDVSVDFSQEEWECLNDDQRDLYRDVMLENYSNLVSMG
HSISKPNVISYLEQGKEPWL,

wherein the nucleic acid sequence of KRAB(ZNF566) is:

(SEQ ID NO: 226)
CGGACACTGGTCATGTTCAGCGACGTGTCCGTGGACTTTAGCCAAGAGGAATGGGA
ATGCCTGAACGACGACCAGCGGGACCTCTATAGGGATGTCATGCTCGAGAACTACA
GCAATCTCGTTTCCATGGGCCACAGCATCTCCAAGCCAAACGTCATCAGCTATCTCG
AACAAGGCAAAGAGCCCTGGCTG.
(14) DNMT3A-DNMT3L-dCas9-KRAB(ZIM2)
(SEQ ID NO: 227)
MNHDQEFDPPKVYP PVP AEKRKPIRVLSLF DGIATGLLVLKDLGIQ
VDRYIASEVCEDSITV GMVRHQGKIMYVGD VRSVTQKHIQEWGPF
DLVIGGSPCNDLSIVN P ARKGLYEGTGRLFF EFYRLLHDARPKEGD
DRPFFWLFENVVAM GVSDKRDISRFLESNP VMIDAKEVSAAHRAR
YFWGNLPGMNRPLAS TVNDKLELQECLEHG RIAKFSK VRTITTRSNS
IKQGKDQHFPVFMN EKEDILWCTEMESSG NSNANSRGPSFSSGL VPLSLRGSHMGPME
IYKTVSAWKRQPVR VLSLFRNIDKVLKSL GFLESGSGSGGGTL KYVEDVTNVVRRD
VEKWGPFDLVYGST QPLGSSCDRCPGWY MFQFHRILQYALPR QESQRPFFWIFMDN
LLLTEDDQETTTRFL QTEA VTLQDVRGRD YQNAMRVWSNIPGL
KSKHAPLTPKEEEYL QAQVRSRSKLDAPK VDLLVKNCLLPLRE YFKYFSQNSLPLRVF
GFPVHYTDVSNMSRLARQRLLGRSWSVP VIRHLFAPLKEYFAC VGGPSSGAPPPSGGS
PAGSPTSTEEGTSES A TPESGPGTSTEPSE GSAPGSPAGSPTSTE EGTSTEPSEGSAPGT
STEPSEPKKKRKVM DKKYSIGLAIGTNS VGWAVITDEYKVP SKKFKVLGNTDRH
SIKKNLIGALLFDS GETAEATRLKRTA RRRYTRRKNRICY LQEIFSNEMAKVDD
SFFHRLEESFL VEE DKKHERHPIFGNIV DEVAYHEKYPTIYH LRKKLVDSTDKAD
LRLIYLALAHMIKF RGHFLIEGDLNPDN SDVDKLFIQLVQTY NQLFEENPINASGV
DAKAILSARLSKSR RLENLIAQLPGEKK NGLFGNLIALSLGL TPNFKSNFDLAEDA
KLQLSKDTYDDDL DNLLAQIGDQYAD LFLAAKNLSDAILL SDILRVNTEITKAPL
SASMIKRYDEHHQ DLTLLKALVRQQL PEKYKEIFFDQSKN GYAGYIDGGASQE
EFYKFIKPILEKMD GTEELLVKLNRED LLRKQRTFDNGSIP HQIHLGELHAILRR
QEDFYPFLKDNRE KIEKILTFRIPYYVG PLARGNSRFAWMT RKSEETITPWNFEE
VVDKGASAQSFIER MTNFDKNLPNEKVLPKHSLLYEYFTVY NELTKVKYVTEGM
RKPAFLSGEQKKAI VDLLFKTNRKVTV KQLKEDYFKKIECF DSVEISGVEDRFNA
SLGTYHDLLKIIKD KDFLDNEENEDILE DIVLTLTLFEDREM IEERLKTYAHLFDD
KVMKQLKRRRYT GWGRLSRKLINGIR DKQSGKTILDFLKS DGFANRNFMQLIH
DDSLTFKEDIQKAQ VSGQGDSLHEHIAN LAGSPAIKKGILQT VKVVDELVKVMGR
HKPENIVIEMAREN QTTQKGQKNSRER MKRIEEGIKELGSQ ILKEHPVENTQLQN
EKLYLYYLQNGRD MYVDQELDINRLS DYDVDAIVPQSFLK DDSIDNKVLTRSDK
NRGKSDNVPSEEVV KKMKNYWRQLLN AKLITQRKFDNLTK AERGGLSELDKAG
FIKRQLVETRQITK HV AQILDSRMNTK YDENDKLIREVKVI TLKSKLVSDFRKDF
QFYKVREINNYHH AHDAYLNAVVGTA LIKKYPKLESEFVY GDYKVYDVRKMIA
KSEQEIGKATAKYF FYSNIMNFFKTEIT LANGEIRKRPLIET NGETGEIVWDKGR
DFATVRKVLSMPQ VNIVKKTEVQTGG FSKESILPKRNSDKLIARKKDWDPKKY
GGFDSPTVAYSVLV V AKVEKGKSKKLK SVKELLGITIMERS SFEKNPIDFLEAKG
YKEVKKDLIIKLPK YSLFELENGRKRM LASAGELQKGNEL ALPSKYVNFLYLAS
HYEKLKGSPEDNE QKQLFVEQHKHYL DEIIEQISEFSKRVI LADANLDKVLSAY
NKHRDKPIREQAE NIIHLFTLTNLGAP AAFKYFDTTIDRKR YTSTKEVLDATLIH
QSITGLYETRIDLS QLGGDAYPYDVPD Y ASLGSGSPKKKRK VEDPKKKRKVDGSG
SETPGTSESATPESRTLVTFEDVLVDFSPEELSSLSAAQRNLYREVMLENYRNLVSLGHQ
FSKPDIISRLEEEESYAM,

wherein the nucleic acid sequence of KRAB(ZIM2) is:

(SEQ ID NO: 228)
CGGACACTGGTCACCTTCGAGGATGTGCTGGTGGATTTCAGCCCTGAGG
AACTGAGCAGCCTGTCCGCCGCACAGAGAAATCTCTATCGGGAAGTGAT
GCTGGAGAACTATCGGAATCTGGTGTCTCTGGGCCACCAGTTCAGCAAG
CCTGACATCATCAGCAGACTGGAAGAAGAGGAATCCTACGCCATG

The constructs may be DNA or RNA. In some embodiments, the construct is mRNA. In some embodiments, the construct is a double-stranded DNA. In some embodiments, the construct is a double-stranded RNA. In some embodiments, the construct is a single-stranded DNA. In some embodiments, the construct is a single-stranded RNA.

### CRISPR-Cas Systems

The present disclosure provides CRISPR/Cas9-based engineered systems for use in genome editing and treating genetic diseases. The CRISPR/Cas9-based engineered systems may be designed to target any gene, including genes involved in angiogenesis, such as VEGFA. The present disclosure provides a CRISPR-Cas system comprising genetically engineered Cas proteins and/or guide RNAs with desired specificity and activity (e.g. reducing or eliminating expression of VEGFA gene product). The CRISPR/Cas9-based systems may include a Cas9 protein, a mutated Cas9 protein or Cas9 fusion protein (e.g. DNMT3A-DNMT3L(3A3L)-dCas9-KRAB fusion molecule) and at least one sgRNA (e.g. VEGFA sgRNA). The Cas9 fusion protein may, for example, include a domain that has a different activity from what is endogenous to Cas9 (e.g. DNMT3A, DNMT3L or KRAB).

The Cas9 protein may be split into an N-terminal portion (CasN) and a C-terminal portion (CasC).

In general, a Cas protein (used interchangeably herein with CRISPR protein, CRISPR enzyme, CRISPR-Cas protein, CRISPR-Cas enzyme, Cas, CRISPR effector, or Cas effector protein) and/or a guide sequence is a component of a CRISPR-Cas system. A CRISPR-Cas system or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, e.g. CRISPR RNA and transactivating (tracr) RNA or a single guide RNA (aka sgRNA; chimeric RNA) or other sequences and transcripts from a CRISPR locus.

In general, a CRISPR system is characterized by elements that promote the formation of a CRISPR complex at the site of a target sequence (also referred to as a protospacer in the context of an endogenous CRISPR system). In an engineered system of the disclosure, the direct repeat may encompass naturally occurring sequences or non-naturally occurring sequences. The direct repeat of the disclosure is not limited to naturally occurring lengths and sequences. Furthermore, a direct repeat of the disclosure may include insertions of nucleotides such as an aptamer or sequences that bind to an adapter protein (for association with functional domains). In certain embodiments, one end of a direct repeat containing such as an insertion is roughly the first half of a short DR and the end is roughly the second half of the short DR.

In the context of formation of a CRISPR complex, “target sequence” or “target polynucleotides” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell.

In general, a guide sequence (or spacer sequence) may be any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 70%, 75%, 80%, 85%, 90%, 91%, 92%, 93%, 94%, 95%, 96%, 97%, 98%, 99%, or more.

In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more central (i.e. not 3′ or 5′) for instance, a double mismatch is, the more cleavage efficiency is affected. Accordingly, by choosing mismatch positions along the spacer, cleavage efficiency can be modulated. By means of example, if less than 100% cleavage of targets is desired (e.g. in a cell population), 1 or more, such as preferably 2 mismatches between spacer and target sequence may be introduced in the spacer sequences. The more central along the spacer of the mismatch position, the lower the cleavage percentage.

A CRISPR-Cas system or components thereof may be used for introducing one or more mutations in a target locus or nucleic acid sequence. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s).

Typically, in the context of an endogenous CRISPR-Cas system, formation of a CRISPR complex (comprising a guide sequence hybridized to a target sequence and complexed with one or more Cas proteins) results in cleavage in or near (e.g. within 1, 2, 3, 4, 5, 6, 7, 8, 9, 10, 20, 50, or more base pairs from) the target sequence, but may depend on for instance secondary structure, in particular in the case of RNA targets. In some cases, in the context of an endogenous CRISPR system, formation of a CRISPR complex (comprising a guide sequence hybridized to a target sequence and complexed with one or more Cas proteins) results in cleavage of one or both strands (if applicable) in or near (e.g. within 1, 2, 3, 4, 5, 6, 7, 8, 9, 10, 20, 50, or more base pairs from) the target sequence.

In some embodiments, the guide RNA (capable of guiding Cas to a target locus) may comprise (1) a guide sequence capable of hybridizing to a target locus (a polynucleotide target locus, such as an RNA target locus) in the eukaryotic cell; (2) a direct repeat (DR) sequence, which reside in a single RNA, i.e. an sgRNA (arranged in a 5′ to 3′ orientation) or crRNA.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant disclosure, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publication Nos. US 2014-0310830, US 2014-0287938 A1, US 2014-0273234 A1, US 2014-0273232 A1, US 2014-0273231 A1, US 2014-0256046 A1, US 2014-0248702 A1, US 2014-0242700 A1, US 2014-0242699 A1, US 2014-0242664 A1, US 2014-0234972 A1, US 2014-0227787 A1, US 2014-0189896 A1, US 2014-0186958, US 2014-0186919 A1, US 2014-0186843 A1, US 2014-0179770 A1 and US 2014-0179006 A1, US 2014-0170753; European Patents EP 2784162 B1 and EP 2771468 B1; European Patent Applications EP 2771468, EP 2764103, and EP 2784162; and PCT Patent Publications WO 2021/183807A1 (PCT/US2021/021973), WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), each of which are incorporated herein by reference in their entirety.

### Cas Proteins

The Cas protein (e.g., engineered Cas protein) may have a nuclease activity that is substantially the same (e.g., between 80% and 100%, between 90% and 100%, between 95% and 100%, between 98% and 100%, between 99% and 100%, between 99.9% and 100%, or about 100%) as a wildtype counterpart Cas protein. In certain cases, the engineered Cas protein has a nuclease activity that is higher than (e.g., at least 5%, at least 10%, at least 20%, at least 30%, at least 40%, at least 50%, at least 60%, at least 70%, at least 80%, or at least 90% higher than) a wildtype counterpart Cas protein.

Alternatively or additionally, the Cas protein (e.g., engineered Cas protein) may have a specificity at least 5%, at least 10%, at least 20%, at least 30%, at least 40%, at least 50%, at least 60%, at least 70%, at least 80%, or at least 90% higher than the wildtype counterpart Cas protein. In a particular example, the Cas protein (e.g., engineered Cas protein) may have a specificity at least 30% higher than the wildtype counterpart Cas protein. As used herein, the term “specificity” of a Cas may correspond to the number or percentage of on-target polynucleotide cleavage events relative to the number or percentage of all polynucleotide cleavage events, including on-target and off-target events. The activity and specificity of a Cas protein are consistent with those described in Hsu P D et al., DNA targeting specificity of RNA-guided Cas9 nucleases, Nat Biotechnol. 2013 September; 31(9): 827-832; and Slaymaker I M, et al., Rationally engineered Cas9 nucleases with improved specificity, Science. 2016 Jan. 1; 351(6268): 84-88, which also describe examples of methods for detecting the activity and specificity of Cas proteins, and are incorporated herein by reference in their entireties, and are detailed elsewhere herein.

In some embodiments, the Cas protein (e.g., its RuvC domain) may slide one base upstream (with respect to the PAM), and produce a staggered cut, which may be filled and lead to duplication of a single base (i.e., +1 insertion). An example of a +1 insertion position is described in Zuo, Z., and Liu, J. (2016). Cas9-catalyzed DNA Cleavage Generates Staggered Ends: Evidence from Molecular Dynamics Simulations. Scientific Reports 6, 37584. In some embodiments, the engineered Cas protein has a +1 insertion frequency different from the wildtype counterpart Cas protein. For example, the +1 insertion frequency when a guanine is present in the −2 position with respect a PAM is higher than the +1 insertion frequency when a thymidine, a cytidine, or a adenine is present in the −2 position with respect the PAM. In some cases, the +1 insertions depend on host machinery in human cells. In some examples, the Cas protein may generate a staggered cut. The staggered cut may be a 1-bp or 1-nucleotide 5′ overhang. The staggered cut may be a 1-bp or 1-nucleotide 3′ overhang.

The nucleic acid molecule encoding a Cas may be codon optimized. An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a Cas is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In some embodiments, the Cas proteins may have nucleic acid cleavage activity. The Cas proteins may have RNA binding and DNA cleaving function. In some embodiments, Cas may direct cleavage of one or two nucleic acid strands at the location of or near a target sequence, such as within the target sequence and/or within the complement of the target sequence or at sequences associated with the target sequence, e.g., within about 1, 2, 3, 4, 5, 6, 7, 8, 9, 10, 15, 20, 25, 50, 100, 200, 500, or more base pairs from the first or last nucleotide of a target sequence. In some embodiments, the Cas protein may direct more than one cleavage (such as one, two three, four, five, or more cleavages) of one or two strands within the target sequence and/or within the complement of the target sequence or at sequences associated with the target sequence and/or within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt, i.e., generating blunt ends. In some embodiments, the cleavage may be staggered, i.e., generating sticky ends.

In some embodiments, a vector encodes a nucleic acid-targeting Cas protein that may be mutated with respect to a corresponding wild-type enzyme such that the mutated nucleic acid-targeting Cas protein lacks the ability to cleave one or two strands of a target polynucleotide containing a target sequence, e.g., alteration or mutation in a HNH domain to produce a mutated Cas substantially lacking all DNA cleavage activity, e.g., the DNA cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. As used herein, the term “derived” with reference to an enzyme means that the derived enzyme is largely based, in the sense of having a high degree of sequence homology with, a wildtype enzyme, but that it has been mutated (modified) in some way as known in the art or as described herein.

Typically, in the context of an endogenous nucleic acid-targeting system, formation of a nucleic acid-targeting complex (comprising a guide RNA or crRNA hybridized to a target sequence and complexed with one or more nucleic acid-targeting effector proteins) results in cleavage of DNA strand(s) in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest).

It will be appreciated that the effector protein is based on or derived from an enzyme, so the term “effector protein” certainly includes “enzyme” in some embodiments. However, it will also be appreciated that the effector protein may, as required in some embodiments, have DNA or RNA binding, but not necessarily cutting or nicking, activity, including a dead-Cas protein function.

In some embodiments, a Cas protein may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the CRISPR effector protein may be a part of a Light Inducible Transcriptional Effector (LITE) to direct changes in transcriptional activity in a sequence-specific manner. The components of a light may include a CRISPR effector protein, a light-responsive cytochrome heterodimer (e.g. from Arabidopsis thaliana), and a transcriptional activation/repression domain. Further examples of inducible DNA binding proteins and methods for their use are provided in U.S. 61/736,465 and U.S. 61/721,283, and WO 2014018423 A2 which are hereby incorporated by reference in their entirety.

In some embodiments, a mutated Cas may have one or more mutations resulting in reduced off-target effects, e.g., improved CRISPR enzymes for use in effecting modifications to target loci but which reduce or eliminate activity towards off-targets, such as when complexed to guide RNAs, as well as improved CRISPR enzymes for increasing the activity of CRISPR enzymes, such as when complexed with guide RNAs. It is to be understood that mutated enzymes as described herein below may be used in any of the methods according to the disclosure as described herein elsewhere. Any of the methods, products, compositions and uses as described herein elsewhere are equally applicable with the mutated CRISPR enzymes as further detailed below.

The methods and mutations which can be employed in various combinations to increase or decrease activity and/or specificity of on-target vs. off-target activity, or increase or decrease binding and/or specificity of on-target vs. off-target binding, can be used to compensate or enhance mutations or modifications made to promote other effects. Such mutations or modifications made to promote other effects in include mutations or modification to the Cas and or mutation or modification made to a guide RNA. The methods and mutations of the disclosure are used to modulate Cas nuclease activity and/or binding with chemically modified guide RNAs.

In certain embodiments, the catalytic activity of the Cas protein of the disclosure is altered or modified. It is to be understood that mutated Cas has an altered or modified catalytic activity if the catalytic activity is different than the catalytic activity of the corresponding wild type Cas protein (e.g., unmutated Cas protein). Catalytic activity can be determined by means known in the art. By means of example, and without limitation, catalytic activity can be determined in vitro or in vivo by determination of indel percentage (for instance after a given time, or at a given dose). In certain embodiments, catalytic activity is increased. In certain embodiments, catalytic activity is increased by at least 5%, at least 10%, at least 20%, at least 30%, at least 40%, at least 50%, at least 60%, at least 70%, at least 80%, at least 90%, or at least 100%. In certain embodiments, catalytic activity is decreased. In certain embodiments, catalytic activity is decreased by at least 5%, preferably at least 10%, more preferably at least 20%, such as at least 30%, at least 40%, at least 50%, at least 60%, at least 70%, at least 80%, at least 90%, or (substantially) 100%. The one or more mutations herein may inactivate the catalytic activity, which may substantially decrease all catalytic activity, decrease activity to below detectable levels, or decrease to no measurable catalytic activity.

One or more characteristics of the engineered Cas protein may be different from a corresponding wildtype Cas protein. Examples of such characteristics include catalytic activity, gRNA binding, specificity of the Cas protein (e.g., specificity of editing a defined target), stability of the Cas protein, off-target binding, target binding, protease activity, nickase activity, PFS recognition. In some examples, a engineered Cas protein may comprise one or more mutations of the corresponding wild type Cas protein. In some embodiments, the catalytic activity of the engineered Cas protein is increased as compared to a corresponding wildtype Cas protein. In some embodiments, the catalytic activity of the engineered Cas protein is decreased as compared to a corresponding wildtype Cas protein. In some embodiments, the gRNA binding of the engineered Cas protein is increased as compared to a corresponding wildtype Cas protein. In some embodiments, the gRNA binding of the engineered Cas protein is decreased as compared to a corresponding wildtype Cas protein. In some embodiments, the specificity of the Cas protein is increased as compared to a corresponding wildtype Cas protein. In some embodiments, the specificity of the Cas protein is decreased as compared to a corresponding wildtype Cas protein. In some embodiments, the stability of the Cas protein is increased as compared to a corresponding wildtype Cas protein. In some embodiments, the stability of the Cas protein is decreased as compared to a corresponding wildtype Cas protein. In some embodiments, the engineered Cas protein further comprises one or more mutations which inactivate catalytic activity. In some embodiments, the off-target binding of the Cas protein is increased as compared to a corresponding wildtype Cas protein. In some embodiments, the off-target binding of the Cas protein is decreased as compared to a corresponding wildtype Cas protein. In some embodiments, the target binding of the Cas protein is increased as compared to a corresponding wildtype Cas protein. In some embodiments, the target binding of the Cas protein is decreased as compared to a corresponding wildtype Cas protein. In some embodiments, the engineered Cas protein has a higher protease activity or polynucleotide-binding capability compared with a corresponding wildtype Cas protein. In some embodiments, the PFS recognition is altered as compared to a corresponding wildtype Cas protein.

### Examples of Cas Proteins

Examples of Cas proteins include those of Class I (e.g., Type I, Type III, and Type IV) and Class 2 (e.g., Type II, Type V, and Type VI) Cas proteins, e.g., Cas9, Cas12 (e.g., Cas12a, Cas12b, Cas12c, Cas12d), Cas13 (e.g., Cas13a, Cas13b, Cas13c, Cas13d,), CasX, CasY, Cas14, variants thereof (e.g., mutated forms, truncated forms), homologs thereof, and orthologs thereof. The terms “ortholog” and “homolog” are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

### Class 2 Cas Proteins

In some embodiments, the Cas protein is a class 2 Cas protein, i.e., a Cas protein of a class 2 CRISPR-Cas system. A class 2 CRISPR-Cas system may be of a subtype, e.g., Type II-A, Type II-B, Type II-C, Type V-A, Type V-B, Type V-C, or Type V-U. In some embodiments, the Cas protein is Cas9, Cas12a, Cas12b, Cas12c, or Cas12d. In some embodiments, Cas9 may be SpCas9, SaCas9, StCas9 and other Cas9 orthologs. Cas12 may be Cas12a, Cas12b, and Cas12c, including FnCas12a, or homology or orthologs thereof. The definition and exemplary members of the CRISPR-Cas system include those described in Kira S. Makarova and Eugene V. Koonin, Annotation and Classification of CRISPR-Cas systems, Methods Mol Biol. 2015; 1311: 47-75; and Sergey Shmakov et al., Diversity and evolution of class 2 CRISPR-Cas systems, Nat Rev Microbial. 2017 March; 15(3): 169-182.

### Cas Protein Linkers

In some examples, the Cas protein comprises at least one RuvC domain and at least one HNH domain. The Cas protein may further comprise a first and a second linker domain connecting the RuvC domain and the HNH domain. The first linker (L1) and second linker (L2) connecting the HNH and RuvC domains in Cas9 are described in studies by Nishimasu, H. et al. “Crystal structure of Cas9 in complex with guide RNA and target RNA” Cell 156 (Feb. 27, 2014): 935-949 and Ribeiro, L. et al. (2018) “Protein engineering strategies to expand CRISPR-Cas9 applications” International Journal of Genomics Volume 2018, Article ID 1652567 (doi.org/10.1155/2018/1652567). FIG. 1 of Ribeiro shows the overall organization, structure and function of Cas9, incorporated specifically herein by reference. Specifically, FIG. 1A shows a schematic representation of the domain organization of SpCas9 indicating the genetic architecture of the HNH and RuvC domains including the linkers L1 (spanning amino acids 765-780) and L2 (spanning amino acids 906-918) as described herein.

Similarly, the domain organization of Staphylococcus aureus Cas9 (SaCas9) can be utilized when referencing the first and second linker domains. In an aspect, the Linker 1 domain region spans residues 481-519, and connects the RuvC-II domain to the HNH domain in SaCas9. In some embodiments, Linker 2 region spans residues 629-649, and connects the RuvC-III domain and the HNH domain of SaCas9. Accordingly, the first and/or second linker domain may be mutated in a Cas9 ortholog, and reference may be made to amino acid residues corresponding to the amino acids of a wild-type SaCas9. See, Nishimasu, Cell. 2015 Aug. 27; 162(5): 1113-1126; doi: 10.1016/j.cell.2015.08.007, incorporated by reference herein. In particular, FIG. 1, S1-S3 of Nishimasu detail domain organization of Cas9 proteins, and are incorporated specifically by reference herein for their teachings.

The first and second linker may comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or more amino acids. The first and second linker may correspond to wild-type linkers. In some aspects, the first and second linkers may comprise one or more mutations in the first and/or second linker. In an aspect the first and/or second linker comprise one or more mutations that improve specificity of the Cas9 protein.

In some embodiments, the linkers, L1 and L2, connecting the HNH and RuvC domains of Cas9 contain the wild-type amino acid sequences. In some embodiments, the linkers connecting the HNH and RuvC domains contain mutations in one or more amino acids. In an example embodiment, the first linker (L1) contains the mutation corresponding to amino acid T769I of SpCas9 and/or the second linker (L2) contains the mutation corresponding to amino acid G915M of SpCas9. In an example embodiment, one or more linker mutations, e.g., T769I and G915M, confer improved specificity upon the Cas9 protein.

In one embodiment, one or mutations in the first and second linker may be combined with one or more mutations in other portions of the Cas9 protein for further improved specificity and/or retention of activity that is substantially equivalent to a wild-type Cas9 protein, as described herein. In one embodiment, mutations in the linker and/or additional mutations within the Cas protein can be identified utilizing the methods detailed herein that enhance/improve specificity and substantially retain wild-type activity to the wild-type Cas9.

Class 2, Type II Cas Proteins (e.g. Cas9)

In some embodiments, the Cas protein may be a Cas protein of a Class 2, Type II CRISPR-Cas system (a Type II Cas protein). In some embodiments, the Cas protein may be a class 2 Type II Cas protein, e.g., Cas9. In some embodiments, the CRISPR/Cas9-based system may include a Cas9 protein or a fragment thereof, a Cas9 fusion protein, a nucleic acid encoding a Cas9 protein or a fragment thereof, or a nucleic acid encoding a Cas9 fusion protein. By “Cas9 (CRISPR associated protein 9)” is meant a polypeptide or fragment thereof having at least about 85% amino acid identity to NCBI Accession No. NP_269215 and having RNA binding activity, DNA binding activity, and/or DNA cleavage activity (e.g., endonuclease or nickase activity). “Cas9 function” can be defined by any of a number of assays including, but not limited to, fluorescence polarization-based nucleic acid bind assays, fluorescence polarization-based strand invasion assays, transcription assays, EGFP disruption assays, DNA cleavage assays, and/or Surveyor assays, for example, as described herein. By “Cas 9 nucleic acid molecule” is meant a polynucleotide encoding a Cas9 polypeptide or fragment thereof. An exemplary Cas9 nucleic acid molecule sequence is provided at genome sequence No. NC_002737. In some embodiments, disclosed herein are inhibitors of Cas9, e.g., naturally occurring Cas9 in S. pyogenes (SpCas9) or S. aureus (SaCas9), or variants thereof. Cas9 recognizes foreign DNA using Protospacer Adjacent Motif (PAM) sequence and the base pairing of the target DNA by the guide RNA (gRNA). The relative ease of inducing targeted strand breaks at any genomic loci by Cas9 has enabled efficient genome editing in multiple cell types and organisms. Cas9 derivatives can also be used as transcriptional activators/repressors.

In some cases, the CRISPR-Cas protein is Cas9 or a variant thereof. In some examples, Cas9 may be wildtype Cas9 including any naturally occurring bacterial Cas9. Cas9 orthologs typically share the general organization of 3-4 RuvC domains and a HNH domain. The 5′ most RuvC domain cleaves the non-complementary strand, and the HNH domain cleaves the complementary strand. All notations are in reference to the guide sequence. The catalytic residue in the 5′ RuvC domain is identified through homology comparison of the Cas9 of interest with other Cas9 orthologs (from S. pyogenes type II CRISPR locus, S. thermophilus CRISPR locus 1, S. thermophilus CRISPR locus 3, and Franciscilla novicida type II CRISPR locus), and the conserved Asp residue (D10) is mutated to alanine to convert Cas9 into a complementary-strand nicking enzyme. Accordingly, the Cas enzyme can be wildtype Cas9 including any naturally occurring bacterial Cas9. The CRISPR, Cas or Cas9 enzyme can be codon optimized, or a modified version, including any chimaeras, mutants, homologs or orthologs. In an additional aspect of the disclosure, a Cas9 enzyme may comprise one or more mutations and may be used as a generic DNA binding protein with or without fusion to a functional domain.

The mutations may be artificially introduced mutations or gain-of-function or loss-of-function mutations. In some embodiments, the transcriptional activation domain may be VP64. In some embodiments, the transcriptional repressor domain may be KRAB or SID4×. Other aspects of the disclosure relate to the mutated Cas9 enzyme being fused to domains which include but are not limited to a nuclease, a transcriptional activator, repressor, a recombinase, a transposase, a histone remodeler, a demethylase, a DNA methyltransferase, a cryptochrome, a light inducible/controllable domain or a chemically inducible/controllable domain. The disclosure can involve sgRNAs or tracrRNAs or guide or chimeric guide sequences that allow for enhancing performance of these RNAs in cells. This type II CRISPR enzyme may be any Cas enzyme. In some cases, the Cas9 enzyme is from, or is derived from, SpCas9 or SaCas9. As used herein, the term “derived” with reference to an enzyme means that the derived enzyme is largely based, in the sense of having a high degree of sequence homology with, a wildtype enzyme, but that it has been mutated (modified) in some way as known in the art or as described herein. In an example the mutation may comprise one or more mutations in a first linker domain, a second linker domain, and/or other portions of the protein. The high degree of sequence homology may comprise at least 80%, 85%, 86%, 87%, 88%, 89%, 90%, 91%, 92%, 93%, 94%, 95%, 96%, 97%, 98%, 99% or more relative to a wildtype enzyme.

A Cas enzyme may be identified Cas9 as this can refer to the general class of enzymes that share homology to the biggest nuclease with multiple nuclease domains from the type II CRISPR system. In some cases, the Cas9 enzyme is from, or is derived from, SpCas9 (S. pyogenes Cas9) or saCas9 (S. aureus Cas9). “StCas9” refers to wildtype Cas9 from S. thermophilus (UniProt ID: G3ECR1). Similarly, “SpCas9” refers to wildtype Cas9 from S. pyogenes (UniProt ID: Q99ZW2). As used herein, the term “derived” with reference to an enzyme means that the derived enzyme is largely based, in the sense of having a high degree of sequence homology with, a wildtype enzyme, but that it has been mutated (modified) in some way as known in the art or as described herein. It will be appreciated that the terms Cas and CRISPR enzyme are generally used herein interchangeably, unless otherwise apparent. As mentioned above, many of the residue numberings used herein refer to the Cas9 enzyme from the type II CRISPR locus in Streptococcus pyogenes.

In particular embodiments, the effector protein is a Cas9 effector protein from or originated from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacte, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus, Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, or Campylobacter.

In some embodiments, the Cas9 protein is from or originated from an organism selected from S. mutans, S. agalactiae, S. equisimilis, S. sanguinis, S. pneumonia, C. jejuni, C. coli; N salsuginis, N tergarcus; S. auricularis, S. carnosus; N meningitides, N gonorrhoeae, L. monocytogenes, L. ivanovii; C. botulinum, C. difficile, C. tetani, or C. sordellii, Francisella tularensis 1, Francisella tularensis subsp. novicida, Prevotella albensis, Lachnospiraceae bacterium MC2017 1, Butyrivibrio proteoclasticus, Peregrinibacteria bacterium GW2011 GWA2_33_10, Parcubacteria bacterium GW2011 GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. In some embodiments, Cas9 effector protein from an organism from or originated from Streptococcus pyogenes, Staphylococcus aureus, or Streptococcus thermophilus Cas9.

In a more preferred embodiment, the Cas9 protein is derived from a bacterial species selected from Streptococcus pyogenes, Staphylococcus aureus, or Streptococcus thermophilus Cas9. In certain embodiments, the Cas9 is derived from a bacterial species selected from Francisella tularensis 1, Prevotella albensis, Lachnospiraceae bacterium MC20171, Butyrivibrio proteoclasticus, Peregrinibacteria bacterium GW2011 GWA2 33 JO, Parcubacteria bacterium GW2011 GWC2_44_17, Smithella sp. SCADC, Acidaminococcus sp. BV3L6, Lachnospiraceae bacterium MA2020, Candidatus methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cas9 protein is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida.

Cas9 enzymes include but are not limited to S. pyogenes serotype M1 (UniProt ID: Q99ZW2), S. aureus Cas9 (UniProt ID: J7RUA5), Eubacterium ventriosum Cas9 (UniProt ID: A5Z395), Azospirillum (strain B510) Cas9 (UniProt ID: D3NTO9), Gluconacetobacter diazotrophicus (strain ATCC 49037) Cas9 (UnitProt ID: A9HKP2), Nisseria cinerea Cas9 (UniProt ID: DOW2Z9), Roseburia intestinalis Cas9 (UniProt ID: C7G697), Parvibaculum lavamentivorans (strain DS-1) Cas9 (UniProt ID: A7HP89), Nitratifractor salsuginis (strain DSM 16511) Cas9 (UniProt ID: E6WZS9), Campylobacter lari Cas9 (UniProt ID: G1UFN3).

Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence (examples include NGG/NRG or a PAM that can be determined as described herein) following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defense in bacteria and archaea, Mole Cell 2010, January 15; 37(1): 7. The type II CRISPR locus from Streptococcus pyogenes SF370, which contains a cluster of four genes Cas9, Cas1, Cas2, and Csnl, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. A pre-crRNA array consisting of a single spacer flanked by two direct repeats (DRs) is also encompassed by the term “tracr-mate sequences”). In certain embodiments, Cas9 may be constitutively present or inducibly present or conditionally present or administered or delivered. Cas9 optimization may be used to enhance function or to develop new functions. One can generate chimeric Cas9 proteins and Cas9 may be used as a generic DNA binding protein. The structural information provided for Cas9 may be used to further engineer and optimize the CRISPR-Cas system and this may be extrapolated to interrogate structure-function relationships in other CRISPR enzyme systems as well, particularly structure-function relationships in other Type II CRISPR enzymes or Cas9 orthologs. The crystal structure information (described in U.S. provisional applications 61/915,251 filed Dec. 12, 2013, 61/930,214 filed on Jan. 22, 2014, 61/980,012 filed Apr. 15, 2014; and Nishimasu et al, “Crystal Structure of Cas9 in Complex with Guide RNA and Target DNA,” Cell 156(5):935-949, DOI: http://dx.doi.org/10.1016/j.cell.2014.02.001 (2014), each and all of which are incorporated herein by reference) provides structural information to truncate and create modular or multi-part CRISPR enzymes which may be incorporated into inducible CRISPR-Cas systems. In particular, structural information is provided for S. pyogenes Cas9 (SpCas9) and this may be extrapolated to other Cas9 orthologs or other Type II CRISPR enzymes. The Cas9 gene is found in several diverse bacterial genomes, typically in the same locus with casl, cas2, and cas4 genes and a CRISPR cassette. Furthermore, the Cas9 protein contains a readily identifiable C-terminal region that is homologous to the transposon ORF-B and includes an active RuvC-like nuclease, an arginine-rich region.

dCas9

The Cas9 protein may be mutated so that the nuclease activity is inactivated. An inactivated Cas9 protein from S. pyogenes (iCas9, also referred to as “dCas9”) with no endonuclease activity has been recently targeted to genes in bacteria, yeast, and human cells by gRNA to silence gene expression through steric hindrance. As used herein, a “dCas molecule” may refer to a dCas protein, or a fragment thereof. As used herein, a “dCas9 molecule” may refer to a dCas9 protein, or a fragment thereof. As used herein, the terms “iCas” and “dCas” are used interchangeably and refer to a catalytically inactive CRISPR associated protein. In one embodiment, the dCas molecule comprises one or more mutations in a DNA-cleavage domain. In one embodiment, the dCas molecule comprises one or more mutations in the RuvC or HNH domain. In one embodiment, the dCas molecule comprises one or more mutations in both the RuvC and HNH domain. In one embodiment, the dCas molecule is a fragment of a wild-type Cas molecule. In one embodiment, the dCas molecule comprises a functional domain from a wild-type Cas molecule, wherein the functional domain is chosen from a Reel domain, a bridge helix domain, or a PAM interacting domain. In one embodiment, the nuclease activity of the dCas molecule is reduced by at least 40%, 45%, 50%, 55%, 60%, 65%, 70%, 75%, 80%, 85%, 90%, 95%, or 99% compared to that of a corresponding wild type Cas molecule. An exemplary amino acid sequence of a wildtype dCas9 protein is set forth in SEQ ID NO: 1. An exemplary nucleic acid sequence encoding a wildtype dCas9 is set forth in SEQ ID NO: 26.

Suitable dCas molecule can be derived from a wild type Cas molecule. The Cas molecule can be from a type I, type II, or type III CRISPR-Cas systems. In one embodiment, suitable dCas molecules can be derived from a Cas1, Cas2, Cas3, Cas4, Cas5, Cas6, Cas7, Cas8, Cas9, or Cas10 molecule. In one embodiment, the dCas molecule is derived from a Cas9 molecule. The dCas9 molecule can be obtained, for example, by introducing point mutations (e.g., substitutions, deletions, or additions) in the Cas9 molecule at the DNA-cleavage domain, e.g., the nuclease domain, e.g., the RuvC and/or HNH domain. See, e.g., Jinek et al., Science (2012) 337:816-21, incorporated by reference herein in its entirety. For example, introducing two point mutations in the RuvC and HNH domains reduces the Cas9 nuclease activity while retaining the Cas9 sgRNA and DNA binding activity. In one embodiment, the two point mutations within the RuvC and HNH active sites are D10A and H840A mutations of the S. pyogenes Cas9 molecule. Alternatively, D10 and H840 of the S. pyogenes Cas9 molecule can be deleted to abolish the Cas9 nuclease activity while retaining its sgRNA and DNA binding activity. In one embodiment, the two point mutations within the RuvC and HNH active sites are D10A and N580A mutations of the S. pyogenes Cas9 molecule.

In one embodiment, the dCas molecule is an S. aureus dCas9 molecule comprising a mutation at D10 and/or N580, numbered according to SEQ ID NO: 1. In one embodiment, the dCas molecule is an S. aureus dCas9 molecule comprising D10A and/or N580A mutations, numbered according to SEQ ID NO: 1.

In one embodiment, the dCas9 molecule is an S. aureus dCas9 molecule comprising the amino acid sequence of SEQ ID NO: 1, a sequence substantially identical (e.g., at least 80%, 85%, 90%, 91%, 92%, 93%, 94%, 95%, 96%, 97%, 98%, 99% or higher in sequence identity) to SEQ ID NO: 1, or a sequence having one, two, three, four, five or more changes, e.g., amino acid substitutions, insertions, or deletions, relative to SEQ ID NO: 1, or any fragment thereof.

Similar mutations can also apply to any other naturally-occurring Cas9 (e.g., Cas9 from other species) or engineered Cas9 molecules. In certain embodiments, the dCas9 molecule comprises a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B 510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 1651 1) dCas9 molecule, a Campylobacter lari (strain CF89-12) dCas9 molecule, a Streptococcus thermophilus (strain LMD-9) dCas9 molecule, or fragment thereof.

In certain embodiments, the present disclosure provides a vector comprising a nucleotide encoding a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 1651 1) dCas9 molecule, a Campylobacter lari (strain CF89-12) dCas9 molecule, a Streptococcus thermophilus (strain LMD-9) dCas9 molecule, or fragment thereof.

In some embodiments, the dCas9 protein comprises the sequence set forth in any one of SEQ ID NOs: 106-122.

The dCas9 molecule in a construct provided herein may be continuous or split into an N-terminal portion (dCas9N) and a C-terminal portion (dCas9C). In some embodiments, the N-terminal and C-terminal portions are separated by DNMT3A and/or DNMT3L. Thus, provided herein is a construct comprising, in N-terminal to C-terminal order: dCas9N, DNMT3A, DNMT3L, and dCas9C.

A person of skill in the art will appreciate that any dCas9 protein may be split at various points in the protein sequence, so long as the fusion protein is able to refold into a functional dCas9 molecule. Table 2 sets out illustrative sequences of dCas9-N and dCas9-C sequences. N-terminal and C-terminal sequences are preferably paired according to their number, e.g., dCas9N-1 is used with dCas9C-1, dCas9N-2 is used with dCas9C-2, and so forth. In some embodiments, a construct provided herein comprises a dCas9N comprising the sequence set forth in SEQ ID NO: 2 and a dCas9C comprising the sequence set forth in SEQ ID NO: 3. In some embodiments, a construct provided herein comprises a dCas9N comprising the sequence set forth in SEQ ID NO: 4 and a dCas9C comprising the sequence set forth in SEQ ID NO: 5. In some embodiments, a construct provided herein comprises a dCas9N comprising the sequence set forth in SEQ ID NO: 6 and a dCas9C comprising the sequence set forth in SEQ ID NO: 7. In some embodiments, a construct provided herein comprises a dCas9N comprising the sequence set forth in SEQ ID NO: 8 and a dCas9C comprising the sequence set forth in SEQ ID NO: 9. In some embodiments, a construct provided herein comprises a dCas9N comprising the sequence set forth in SEQ ID NO: 10 and a dCas9C comprising the sequence set forth in SEQ ID NO: 11. In some embodiments, a construct provided herein comprises a dCas9N comprising the sequence set forth in SEQ ID NO: 12 and a dCas9C comprising the sequence set forth in SEQ ID NO: 13. In some embodiments, a construct provided herein comprises a dCas9N comprising the sequence set forth in SEQ ID NO: 14 and a dCas9C comprising the sequence set forth in SEQ ID NO: 15. In some embodiments, a construct provided herein comprises a dCas9N comprising the sequence set forth in SEQ ID NO: 16 and a dCas9C comprising the sequence set forth in SEQ ID NO: 17. In some embodiments, a construct provided herein comprises a dCas9N comprising the sequence set forth in SEQ ID NO: 18 and a dCas9C comprising the sequence set forth in SEQ ID NO: 19. In some embodiments, a construct provided herein comprises a dCas9N comprising the sequence set forth in SEQ ID NO: 20 and a dCas9C comprising the sequence set forth in SEQ ID NO: 21. In some embodiments, a construct provided herein comprises a dCas9N comprising the sequence set forth in SEQ ID NO: 22 and a dCas9C comprising the sequence set forth in SEQ ID NO: 23. In some embodiments, a construct provided herein comprises a dCas9N comprising the sequence set forth in SEQ ID NO: 24 and a dCas9C comprising the sequence set forth in SEQ ID NO: 25.

In some embodiments, a dCas9N is encoded by a polynucleotide comprising the nucleic acid sequence in SEQ ID NO: 27, 29, 31, 33, 35, 37, 39, 41, 43, 45, 47, or 49. In some embodiments, a dCas9C is encoded by a polynucleotide comprising the nucleic acid sequence set forth in SEQ ID NO: 28, 30, 32, 34, 36, 38, 40, 42, 44, 46, 48, or 50.

TABLE 2
Exemplary dCas9 and Split dCas9 Sequences

| Cas9 | Sequence |
|---|---|
| wt | MDKKYSIGLAIGTNSVGWAVITDEYKVPSKKFKVLGNTDRHSIKKNLIGALLFD |
| | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQTTQKGQKNSRERMKRIEEGIKELGSQILKEHPVENTQLQN |
| | EKLYLYYLQNGRDMYVDQELDINRLSDYDVDAIVPQSFLKDDSIDNKVLTRSD |
| | KNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFR |
| | KDFQFYKVREINNYHHAHDAYLNAVVGTALIKKYPKLESEFVYGDYKVYDVR |
| | KMIAKSEQEIGKATAKYFFYSNIMNFFKTEITLANGEIRKRPLIETNGETGEIVW |
| | DKGRDFATVRKVLSMPQVNIVKKTEVQTGGFSKESILPKRNSDKLIARKKDWD |
| | PKKYGGFDSPTVAYSVLVVAKVEKGKSKKLKSVKELLGITIMERSSFEKNPIDF |
| | LEAKGYKEVKKDLIIKLPKYSLFELENGRKRMLASAGELQKGNELALPSKYVN |
| | FLYLASHYEKLKGSPEDNEQKQLFVEQHKHYLDEIIEQISEFSKRVILADANLDK |
| | VLSAYNKHRDKPIREQAENIIHLFTLTNLGAPAAFKYFDTTIDRKRYTSTKEVLD |
| | ATLIHQSITGLYETRIDLSQLGGD (SEQ ID NO: 1) |
| dCas9N-1 | MDKKYSIGLAIGTNSVGWAVITDEYKVPSKKFKVLGNTDRHSIKKNLIGALLFD |
| (531aa) | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVT (SEQ ID |
| | NO: 2) |
| dCas9C-1 | EGMRKPAFLSGEQKKAIVDLLFKTNRKVTVKQLKEDYFKKIECFDSVEISGVED |
| (531aa) | RFNASLGTYHDLLKIIKDKDFLDNEENEDILEDIVLTLTLFEDREMIEERLKTYA |
| | HLFDDKVMKQLKRRRYTGWGRLSRKLINGIRDKQSGKTILDFLKSDGFANRNF |
| | MQLIHDDSLTFKEDIQKAQVSGQGDSLHEHIANLAGSPAIKKGILQTVKVVDEL |
| | VKVMGRHKPENIVIEMARENQTTQKGQKNSRERMKRIEEGIKELGSQILKEHP |
| | VENTQLQNEKLYLYYLQNGRDMYVDQELDINRLSDYDVDAIVPQSFLKDDSID |
| | NKVLTRSDKNRGKSDNVPSEEVVKKMKNYWRQLLNAKLITQRKFDNLTKAER |
| | GGLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVYGD |
| | YKVYDVRKMIAKSEQEIGKATAKYFFYSNIMNFFKTEITLANGEIRKRPLIETNG |
| | ETGEIVWDKGRDFATVRKVLSMPQVNIVKKTEVQTGGFSKESILPKRNSDKLIA |
| | RKKDWDPKKYGGFDSPTVAYSVLVVAKVEKGKSKKLKSVKELLGITIMERSSF |
| | EKNPIDFLEAKGYKEVKKDLIIKLPKYSLFELENGRKRMLASAGELQKGNELAL |
| | PSKYVNFLYLASHYEKLKGSPEDNEQKQLFVEQHKHYLDEIIEQISEFSKRVILA |
| | DANLDKVLSAYNKHRDKPIREQAENIIHLFTLTNLGAPAAFKYFDTTIDRKRYT |
| | STKEVLDATLIHQSITGLYETRIDLSQLGGD (SEQ ID NO: 3) |
| dCas9N-2 | MDKKYSIGLAIGTNSVGWAVITDEYKVPSKKFKVLGNTDRHSIKKNLIGALLFD |
| (768aa) | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQ (SEQ ID NO: 4) |
| d Cas9C- | TTQKGQKNSRERMKRIEEGIKELGSQILKEHPVENTQLQNEKLYLYYLQNGRD |
| 2(768aa) | MYVDQELDINRLSDYDVDAIVPQSFLKDDSIDNKVLTRSDKNRGKSDNVPSEE |
| | VVKKMKNYWRQLLNAKLITQRKFDNLTKAERGGLSELDKAGFIKRQLVETRQ |
| | ITKHVAQILDSRMNTKYDENDKLIREVKVITLKSKLVSDFRKDFQFYKVREINN |
| | YHHAHDAYLNAVVGTALIKKYPKLESEFVYGDYKVYDVRKMIAKSEQEIGKA |
| | TAKYFFYSNIMNFFKTEITLANGEIRKRPLIETNGETGEIVWDKGRDFATVRKVL |
| | SMPQVNIVKKTEVQTGGFSKESILPKRNSDKLIARKKDWDPKKYGGFDSPTVA |
| | YSVLVVAKVEKGKSKKLKSVKELLGITIMERSSFEKNPIDFLEAKGYKEVKKDL |
| | IIKLPKYSLFELENGRKRMLASAGELQKGNELALPSKYVNFLYLASHYEKLKGS |
| | PEDNEQKQLFVEQHKHYLDEIIEQISEFSKRVILADANLDKVLSAYNKHRDKPIR |
| | EQAENIIHLFTLTNLGAPAAFKYFDTTIDRKRYTSTKEVLDATLIHQSITGLYETR |
| | IDLSQLGGD (SEQ ID NO: 5) |
| d Cas9N- | MDKKYSIGLAIGTNSVGWAVITDEYKVPSKKFKVLGNTDRHSIKKNLIGALLFD |
| 3(776aa) | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQTTQKGQKN (SEQ ID NO: 6) |
| dCas9C-3 | SRERMKRIEEGIKELGSQILKEHPVENTQLQNEKLYLYYLQNGRDMYVDQELD |
| (776aa) | INRLSDYDVDAIVPQSFLKDDSIDNKVLTRSDKNRGKSDNVPSEEVVKKMKNY |
| | WRQLLNAKLITQRKFDNLTKAERGGLSELDKAGFIKRQLVETRQITKHVAQILD |
| | SRMNTKYDENDKLIREVKVITLKSKLVSDFRKDFQFYKVREINNYHHAHDAYL |
| | NAVVGTALIKKYPKLESEFVYGDYKVYDVRKMIAKSEQEIGKATAKYFFYSNI |
| | MNFFKTEITLANGEIRKRPLIETNGETGEIVWDKGRDFATVRKVLSMPQVNIVK |
| | KTEVQTGGFSKESILPKRNSDKLIARKKDWDPKKYGGFDSPTVAYSVLVVAKV |
| | EKGKSKKLKSVKELLGITIMERSSFEKNPIDFLEAKGYKEVKKDLIIKLPKYSLF |
| | ELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHL |
| | FTLTNLGAPAAFKYFDTTIDRKRYTSTKEVLDATLIHQSITGLYETRIDLSQLGG |
| | D (SEQ ID NO: 7) |
| dCas9N- | MDKKYSIGLAIGTNSVGWAVITDEYKVPSKKFKVLGNTDRHSIKKNLIGALLFD |
| 4(1009aa) | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQTTQKGQKNSRERMKRIEEGIKELGSQILKEHPVENTQLQN |
| | EKLYLYYLQNGRDMYVDQELDINRLSDYDVDAIVPQSFLKDDSIDNKVLTRSD |
| | KNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFR |
| | KDFQFYKVREINNYHHAHDAYLNAVVGTALIKKYPKLESEFV (SEQ ID NO: 8) |
| dCas9C- | YGDYKVYDVRKMIAKSEQEIGKATAKYFFYSNIMNFFKTEITLANGEIRKRPLI |
| 4(1009aa) | ETNGETGEIVWDKGRDFATVRKVLSMPQVNIVKKTEVQTGGFSKESILPKRNS |
| | DKLIARKKDWDPKKYGGFDSPTVAYSVLVVAKVEKGKSKKLKSVKELLGITI |
| | MERSSFEKNPIDFLEAKGYKEVKKDLIIKLPKYSLFELENGRKRMLASAGELQK |
| | GNELALPSKYVNFLYLASHYEKLKGSPEDNEQKQLFVEQHKHYLDEIIEQISEFS |
| | KRVILADANLDKVLSAYNKHRDKPIREQAENIIHLFTLTNLGAPAAFKYFDTTI |
| | DRKRYTSTKEVLDATLIHQSITGLYETRIDLSQLGGD (SEQ ID NO: 9) |
| dCas9N- | MDKKYSIGLAIGTNSVGWAVITDEYKVPSKKFKVLGNTDRHSIKKNLIGALLFD |
| 5(1048- | SGETAEATRLKRTARRRYTRRKNRICYLQEIFSNEMAKVDDSFFHRLEESFLVE |
| 1063aa) | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQTTQKGQKNSRERMKRIEEGIKELGSQILKEHPVENTQLQN |
| | EKLYLYYLQNGRDMYVDQELDINRLSDYDVDAIVPQSFLKDDSIDNKVLTRSD |
| | KNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFR |
| | KDFQFYKVREINNYHHAHDAYLNAVVGTALIKKYPKLESEFVYGDYKVYDVR |
| | KMIAKSEQEIGKATAKYFFYSNIMNFFK (SEQ ID NO: 10) |
| dCas9C- | ETNGETGEIVWDKGRDFATVRKVLSMPQVNIVKKTEVQTGGFSKESILPKRNS |
| 5(1048- | DKLIARKKDWDPKKYGGFDSPTVAYSVLVVAKVEKGKSKKLKSVKELLGITI |
| 1063aa) | MERSSFEKNPIDFLEAKGYKEVKKDLIIKLPKYSLFELENGRKRMLASAGELQK |
| | GNELALPSKYVNFLYLASHYEKLKGSPEDNEQKQLFVEQHKHYLDEIIEQISEFS |
| | KRVILADANLDKVLSAYNKHRDKPIREQAENIIHLFTLTNLGAPAAFKYFDTTI |
| | DRKRYTSTKEVLDATLIHQSITGLYETRIDLSQLGGD (SEQ ID NO: 11) |
| dCas9N- | MDKKYSIGLAIGTNSVGWAVITDEYKVPSKKFKVLGNTDRHSIKKNLIGALLFD |
| 6(1072aa) | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQTTQKGQKNSRERMKRIEEGIKELGSQILKEHPVENTQLQN |
| | EKLYLYYLQNGRDMYVDQELDINRLSDYDVDAIVPQSFLKDDSIDNKVLTRSD |
| | KNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFR |
| | KDFQFYKVREINNYHHAHDAYLNAVVGTALIKKYPKLESEFVYGDYKVYDVR |
| | KMIAKSEQEIGKATAKYFFYSNIMNFFKTEITLANGEIRKRPLIETNGETGEIM |
| | (SEQ ID NO: 12) |
| dCas9C- | VWDKGRDFATVRKVLSMPQVNIVKKTEVQTGGFSKESILPKRNSDKLIARKKD |
| 6(1072aa) | WDPKKYGGFDSPTVAYSVLVVAKVEKGKSKKLKSVKELLGITIMERSSFEKNPI |
| | DFLEAKGYKEVKKDLIIKLPKYSLFELENGRKRMLASAGELQKGNELALPSKY |
| | VNFLYLASHYEKLKGSPEDNEQKQLFVEQHKHYLDEIIEQISEFSKRVILADANL |
| | DKVLSAYNKHRDKPIREQAENIIHLFTLTNLGAPAAFKYFDTTIDRKRYTSTKE |
| | VLDATLIHQSITGLYETRIDLSQLGGD (SEQ ID NO: 13) |
| dCas9N- | MDKKYSIGLAIGTNSVGWAVITDEYKVPSKKFKVLGNTDRHSIKKNLIGALLFD |
| 7(1246aa) | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQTTQKGQKNSRERMKRIEEGIKELGSQILKEHPVENTQLQN |
| | EKLYLYYLQNGRDMYVDQELDINRLSDYDVDAIVPQSFLKDDSIDNKVLTRSD |
| | KNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFR |
| | KDFQFYKVREINNYHHAHDAYLNAVVGTALIKKYPKLESEFVYGDYKVYDVR |
| | KMIAKSEQEIGKATAKYFFYSNIMNFFKTEITLANGEIRKRPLIETNGETGEIVW |
| | DKGRDFATVRKVLSMPQVNIVKKTEVQTGGFSKESILPKRNSDKLIARKKDWD |
| | PKKYGGFDSPTVAYSVLVVAKVEKGKSKKLKSVKELLGITIMERSSFEKNPIDF |
| | LEAKGYKEVKKDLIIKLPKYSLFELENGRKRMLASAGELQKGNELALPSKYVN |
| | FLYLASHYEKLK (SEQ ID NO: 14) |
| dCas9C-7 | GSPEDNEQKQLFVEQHKHYLDEIIEQISEFSKRVILADANLDKVLSAYNKHRDK |
| (1246aa) | PIREQAENIIHLFTLTNLGAPAAFKYFDTTIDRKRYTSTKEVLDATLIHQSITGLY |
| | ETRIDLSQLGGD (SEQ ID NO: 15) |
| dCas9N-8 | MDKKYSIGLAIGTNSVGWAVITDEYKVPSKKFKVLGNTDRHSIKKNLIGALLFD |
| (1248aa) | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQTTQKGQKNSRERMKRIEEGIKELGSQILKEHPVENTQLQN |
| | EKLYLYYLQNGRDMYVDQELDINRLSDYDVDAIVPQSFLKDDSIDNKVLTRSD |
| | KNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFR |
| | KDFQFYKVREINNYHHAHDAYLNAVVGTALIKKYPKLESEFVYGDYKVYDVR |
| | KMIAKSEQEIGKATAKYFFYSNIMNFFKTEITLANGEIRKRPLIETNGETGEIVW |
| | DKGRDFATVRKVLSMPQVNIVKKTEVQTGGFSKESILPKRNSDKLIARKKDWD |
| | PKKYGGFDSPTVAYSVLVVAKVEKGKSKKLKSVKELLGITIMERSSFEKNPIDF |
| | LEAKGYKEVKKDLIIKLPKYSLFELENGRKRMLASAGELQKGNELALPSKYVN |
| | FLYLASHYEKLKGS (SEQ ID NO: 16) |
| dCas9C-8 | PEDNEQKQLFVEQHKHYLDEIIEQISEFSKRVILADANLDKVLSAYNKHRDKPIR |
| (1248aa) | EQAENIIHLFTLTNLGAPAAFKYFDTTIDRKRYTSTKEVLDATLIHQSITGLYETR |
| | IDLSQLGGD (SEQ ID NO: 17) |
| dCas9N-9 | MDKKYSIGLAIGTNSVGWAVITDEYKVPSKKFKVLGNTDRHSIKKNLIGALLFD |
| (1253aa) | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQTTQKGQKNSRERMKRIEEGIKELGSQILKEHPVENTQLQN |
| | EKLYLYYLQNGRDMYVDQELDINRLSDYDVDAIVPQSFLKDDSIDNKVLTRSD |
| | KNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFR |
| | KDFQFYKVREINNYHHAHDAYLNAVVGTALIKKYPKLESEFVYGDYKVYDVR |
| | KMIAKSEQEIGKATAKYFFYSNIMNFFKTEITLANGEIRKRPLIETNGETGEIVW |
| | DKGRDFATVRKVLSMPQVNIVKKTEVQTGGFSKESILPKRNSDKLIARKKDWD |
| | PKKYGGFDSPTVAYSVLVVAKVEKGKSKKLKSVKELLGITIMERSSFEKNPIDF |
| | LEAKGYKEVKKDLIIKLPKYSLFELENGRKRMLASAGELQKGNELALPSKYVN |
| | FLYLASHYEKLKGSPEDNE (SEQ ID NO: 18) |
| dCas9C-9 | QKQLFVEQHKHYLDEIIEQISEFSKRVILADANLDKVLSAYNKHRDKPIREQAE |
| (1253aa) | NIIHLFTLTNLGAPAAFKYFDTTIDRKRYTSTKEVLDATLIHQSITGLYETRIDLS |
| | QLGGD (SEQ ID NO: 19) |
| dCas9N-10 | MDKKYSIGLAIGTNSVGWAVITDEYKVPSKKFKVLGNTDRHSIKKNLIGALLFD |
| (1260aa) | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQTTQKGQKNSRERMKRIEEGIKELGSQILKEHPVENTQLQN |
| | EKLYLYYLQNGRDMYVDQELDINRLSDYDVDAIVPQSFLKDDSIDNKVLTRSD |
| | KNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFR |
| | KDFQFYKVREINNYHHAHDAYLNAVVGTALIKKYPKLESEFVYGDYKVYDVR |
| | KMIAKSEQEIGKATAKYFFYSNIMNFFKTEITLANGEIRKRPLIETNGETGEIVW |
| | DKGRDFATVRKVLSMPQVNIVKKTEVQTGGFSKESILPKRNSDKLIARKKDWD |
| | PKKYGGFDSPTVAYSVLVVAKVEKGKSKKLKSVKELLGITIMERSSFEKNPIDF |
| | LEAKGYKEVKKDLIIKLPKYSLFELENGRKRMLASAGELQKGNELALPSKYVN |
| | FLYLASHYEKLKGSPEDNEQKQLFVE (SEQ ID NO: 20) |
| dCas9C-10 | QHKHYLDEIIEQISEFSKRVILADANLDKVLSAYNKHRDKPIREQAENIIHLFTLT |
| (1260aa) | NLGAPAAFKYFDTTIDRKRYTSTKEVLDATLIHQSITGLYETRIDLSQLGGD |
| | (SEQ ID NO: 21) |
| dCas9N-11 | MDKKYSIGLAIGTNSVGWAVITDEYKVPSKKFKVLGNTDRHSIKKNLIGALLFD |
| (1263aa) | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQTTQKGQKNSRERMKRIEEGIKELGSQILKEHPVENTQLQN |
| | EKLYLYYLQNGRDMYVDQELDINRLSDYDVDAIVPQSFLKDDSIDNKVLTRSD |
| | KNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFR |
| | KDFQFYKVREINNYHHAHDAYLNAVVGTALIKKYPKLESEFVYGDYKVYDVR |
| | KMIAKSEQEIGKATAKYFFYSNIMNFFKTEITLANGEIRKRPLIETNGETGEIVW |
| | DKGRDFATVRKVLSMPQVNIVKKTEVQTGGFSKESILPKRNSDKLIARKKDWD |
| | PKKYGGFDSPTVAYSVLVVAKVEKGKSKKLKSVKELLGITIMERSSFEKNPIDF |
| | LEAKGYKEVKKDLIIKLPKYSLFELENGRKRMLASAGELQKGNELALPSKYVN |
| | FLYLASHYEKLKGSPEDNEQKQLFVEQHK (SEQ ID NO: 22) |
| dCas9C-11 | HYLDEIIEQISEFSKRVILADANLDKVLSAYNKHRDKPIREQAENIIHLFTLTNLG |
| (1263aa) | APAAFKYFDTTIDRKRYTSTKEVLDATLIHQSITGLYETRIDLSQLGGD (SEQ |
| | ID NO: 23) |
| dCas9N-12 | MDKKYSIGLAIGTNSVGWAVITDEYKVPSKKFKVLGNTDRHSIKKNLIGALLFD |
| (1276aa) | SGETAEATRLKRTARRRYTRRKNRICYLQEIFSNEMAKVDDSFFHRLEESFLVE |
| | EDKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARLS |
| | KSRRLENLIAQLPGEKKNGLFGNLIALSLGLTPNFKSNFDLAEDAKLQLSKDTY |
| | DDDLDNLLAQIGDQYADLFLAAKNLSDAILLSDILRVNTEITKAPLSASMIKRY |
| | DEHHQDLTLLKALVRQQLPEKYKEIFFDQSKNGYAGYIDGGASQEEFYKFIKPI |
| | LEKMDGTEELLVKLNREDLLRKQRTFDNGSIPHQIHLGELHAILRRQEDFYPFL |
| | KDNREKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRH |
| | KPENIVIEMARENQTTQKGQKNSRERMKRIEEGIKELGSQILKEHPVENTQLQN |
| | EKLYLYYLQNGRDMYVDQELDINRLSDYDVDAIVPQSFLKDDSIDNKVLTRSD |
| | KNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFR |
| | KDFQFYKVREINNYHHAHDAYLNAVVGTALIKKYPKLESEFVYGDYKVYDVR |
| | KMIAKSEQEIGKATAKYFFYSNIMNFFKTEITLANGEIRKRPLIETNGETGEIVW |
| | DKGRDFATVRKVLSMPQVNIVKKTEVQTGGFSKESILPKRNSDKLIARKKDWD |
| | PKKYGGFDSPTVAYSVLVVAKVEKGKSKKLKSVKELLGITIMERSSFEKNPIDF |
| | LEAKGYKEVKKDLIIKLPKYSLFELENGRKRMLASAGELQKGNELALPSKYVNFL |
| | YLASHYEKLKGSPEDNEQKQLFVEQHKHYLDEIIEQISEF (SEQ ID NO: 24) |
| dCas9C-12 | SKRVILADANLDKVLSAYNKHRDKPIREQAENIIHLFTLTNLGAPAAFKYFDTTI |
| (1276aa) | DRKRYTSTKEVLDATLIHQSITGLYETRIDLSQLGGD (SEQ ID NO: 25) |

### Cas9 Fusion Proteins

The CRISPR/Cas9-based system may include a fusion molecule (e.g., DNMT3A-DNMT3L(3A3L)-dCas9-KRAB). In some embodiments, the fusion molecule comprises dCas9, KRAB, DNMT3A, and DNMT3L,

In some embodiments, the methods and compositions disclosed herein include a fusion molecule comprising a dCas9 molecule fused to DNMT3A or fragment thereof. In some embodiments, the methods and compositions disclosed herein include a fusion molecule comprising a dCas9 molecule fused to DNMT3L or fragment thereof. In some embodiments, the methods and compositions disclosed herein include a fusion molecule comprising a dCas9 molecule fused to DNMT3L and DNMT3L or fragments thereof. In some embodiments, the methods and compositions disclosed herein include a fusion molecule comprising a dCas9 molecule fused to DNMT3A-DNMT3L fusion peptide.

DNMT3A
(SEQ ID NO: 69)
MNHDQEFDPPKVYPPVPAEKRKPIRVLSLFDGIATGLLVLKDLGIQVDRYIASEVCEDSITVGMVRHQGK
IMYVGDVRSVTQKHIQEWGPFDLVIGGSPCNDLSIVNPARKGLYEGTGRLFFEFYRLLHDARPKEGDDRP
FFWLFENVVAMGVSDKRDISRFLESNPVMIDAKEVSAAHRARYFWGNLPGMNRPLASTVNDKLELQECLE
HGRIAKFSKVRTITTRSNSIKQGKDQHFPVEMNEKEDILWCTEMERVFGFPVHYTDVSNMSRLARQRLLG
RSWSVPVIRHLFAPLKEYFACV
DNMT3L
(SEQ ID NO: 70)
MGPMEIYKTVSAWKRQPVRVLSLFRNIDKVLKSLGFLESGSGSGGGTLKYVEDVTNVVRRDVEKWGPFDL
VYGSTQPLGSSCDRCPGWYMFQFHRILQYALPRQESQRPFFWIFMDNLLLTEDDQETTTRFLQTEAVTLQ
DVRGRDYQNAMRVWSNIPGLKSKHAPLTPKEEEYLQAQVRSRSKLDAPKVDLLVKNCLLPLREYFKYFSQ
NSLPL
DNMT3A-DNMT3L fusion peptide
(SEQ ID NO: 71)
MNHDQEFDPPKVYPPVPAEKRKPIRVLSLFDGIATGLLVLKDLGIQVDRYIASEVCEDSITVGMVRHQGK
IMYVGDVRSVTQKHIQEWGPEDLVIGGSPCNDLSIVNPARKGLYEGTGRLFFEFYRLLHDARPKEGDDRP
FFWLFENVVAMGVSDKRDISRFLESNPVMIDAKEVSAAHRARYFWGNLPGMNRPLASTVNDKLELQECLE
HGRIAKFSKVRTITTRSNSIKQGKDOHFPVEMNEKEDILWCTEMERVFGFPVHYTDVSNMSRLARQRLLG
RSWSVPVIRHLFAPLKEYFACVSSGNSNANSRGPSFSSGLVPLSLRGSHMGPMEIYKTVSAWKRQPVRVL
SLFRNIDKVLKSLGFLESGSGSGGGTLKYVEDVTNVVRRDVEKWGPFDLVYGSTQPLGSSCDRCPGWYMF
QFHRILQYALPRQESQRPFFWIFMDNLLLTEDDQETTTRFLQTEAVTLQDVRGRDYQNAMRVWSNIPGLK
SKHAPLTPKEEEYLQAQVRSRSKLDAPKVDLLVKNCLLPLREYFKYFSQNSLPL

In some embodiments, the DNMT3 is a human DNMT. In some embodiments, the DNMT3A is a non-human DNMT3A, for example, a rodent DNMT3A. In some embodiments, the DNMT3A is selected from Mus musculus DNMT3A, Mus caroli DNMT3A, Mus pahari DNMT3A, Rattus norvegicus DNMT3A, Rattus Rattus DNMT3A, Arvicanthis niloticus DNMT3A, Grammomys surdaster DNMT3A, Mastomys coucha DNTM3A.

In some embodiments, the DNMT3L is a human DNMT3L (SEQ ID NO: 74). In some embodiments, the DNMT3L is a non-human DNMT3L, for example, a rodent DNMT3L. In some embodiments, the DNMT3L is selected from Mus musculus DNMT3L (SEQ ID NO: 75), Mus caroli DNMT3L (SEQ ID NO: 76), Mus pahari DNMT3L (SEQ ID NO: 77), Rattus norvegicus DNMT3L (SEQ ID NO: 78), Rattus Rattus DNMT3L (SEQ ID NO: 79), Arvicanthis niloticus DNMT3L (SEQ ID NO: 80), Grammomys surdaster DNMT3L (SEQ ID NO: 81), and Mastomys coucha DNTM3L (SEQ ID NO: 82).

In some embodiments, the DNMT3L is encoded by a polynucleotide comprising the nucleic acid sequence set forth in one of SEQ ID NOs: 83-92).

In one embodiment, the Cas9 fusion protein also comprises a nuclear localization sequence (NLS), e.g., n NLS fused to the N-terminus and/or C-terminus of Cas9.

Nuclear localization sequences are known in the art. In one embodiment, the NLS comprises the amino acid sequence of SEQ ID NO: 72, 73, 125 or 126, a sequence substantially identical (e.g., at least 80%, 85%, 90%, 92%, 95%, 97%, 98%, 99% or higher identical) to SEQ ID NO: 72, 73, 125 or 126, or a sequence having one, two, three, four, five or more changes, e.g., amino acid substitutions, insertions, or deletions, relative to SEQ ID NO: 72 or 73, or any fragment thereof.

SEQ ID NO: 72 (exemplary nuclear localization
sequence):
APKKKRKVGIHGVPAA
SEQ ID NO: 73 (exemplary nuclear localization
sequence):
KRPAATKKAGQAKKKK
SEQ ID NO: 125 (BPNLS):
KRTADGSEFESPKKKRKV
SEQ ID NO: 126 (SV40 NLS):
PKKKRKV

The construct may further comprises modulator of gene expression. Different modulators of gene expression are known in the art, see, e.g., Thakore et al., Nat Methods. 2016; 13: 127-37, incorporated by reference herein in its entirety. Non-limiting examples of modulators of gene expression include a Kruppel-associated suppression box (KRAB), a Enhancer of zeste homolog 2 (EZH2), a G9A, a Lysine-Specific histone Demethylase 1A (LSD1), a Heterochromatin Protein 1 (HP1), a Friend of GATA protein 1 (FOG1), a Histone Deacetylase (HDAC3) and a DOT1L, each of which is described below in more detail.

In some embodiments, the modulator of epigenetic modification may have DNA methylase activity. For example, the modulator of epigenetic modification may have methylase activity which involves transferring a methyl group to DNA, RNA, proteins, small molecules, cytosine or adenine.

In some embodiments, the CRISPR/Cas9-based system may include a dCas9 molecule and a modulator of gene expression, or a nucleic acid encoding a dCas9 molecule and a modulator of gene expression. In one embodiment, the dCas9 molecule and the modulator of gene expression are linked covalently. In one embodiment, the modulator of gene expression is covalently fused to the dCas9 molecule directly. In one embodiment, the modulator of gene expression is covalently fused to the dCas9 molecule indirectly, e.g., via a non-modulator or linker, or via a second modulator. In one embodiment, the modulator of gene expression is at the N-terminus and/or C-terminus of the dCas9 molecule. In one embodiment, the dCas9 molecule and the modulator of gene expression are linked non-covalently. Exemplary sequences include but are not limited to those listed in Table 3. In some embodiments, the linker between the dCas9 and the at least one modulator of gene expression comprises an amino acid sequence corresponding to a linker listed in Table 3.

TABLE 3
Exemplary Linker Sequences

| Description | Sequence | SEQ ID NO: |
|---|---|---|
| linker | SSGNSNANSRGPSFSSGLVPLSLRGSH | SEQ ID NO: 93 |
| XTEN80 | GGPSSGAPPPSGGSPAGSPTSTEEGTSESATPESGPGTST | SEQ ID NO: 94 |
| linker | EPSEGSAPGSPAGSPTSTEEGTSTEPSEGSAPGTSTEPSE | |
| XTEN16 | SGSETPGTSESATPES | SEQ ID NO: 95 |
| linker | | |
| Link-1 | VRHKLKRVGS | SEQ ID NO: 127 |
| Link-2 | MKIIEQLPSA | SEQ ID NO: 128 |
| Link-3 | VPFLLEPDNINGKTC | SEQ ID NO: 129 |
| Link-4 | GHGTGSTGSGSS | SEQ ID NO: 130 |
| Link-5 | MSRPDPA | SEQ ID NO: 131 |
| Link-6 | SGSETPGTSESATPES | SEQ ID NO: 132 |
| Link-7 | SGSETPGTSESATPEGGSGGS | SEQ ID NO: 133 |
| Link-8 | RVFGFPVHYTDVSNMSRLARQRLLGRSWSVPVIRHLFAP | SEQ ID NO: 123 |
| | LKEYFACVGGPSSGAPPPSGGSPAGSPTSTEEGTSESATPE | |
| | SGPGTSTEPSEGSAPGSPAGSPTSTEEGTSTEPSEGSAPGT | |
| | STEPSEPKKKRKV | |
| Link-9 | AYPYDVPDYASLGSGSPKKKRKVEDPKKKRKVDGSGSE | SEQ ID NO: 124 |
| | TPGTSESATPES | |

In one embodiment, the dCas9 molecule is fused to a first tag, e.g., a first peptide tag. In one embodiment, the modulator of gene expression is fused to a second tag, e.g., a second peptide tag. In one embodiment, the first and second tag, e.g., the first peptide tag and the second peptide tag, non-covalently interact with each other, thereby brining the dCas9 molecule and the modulator of gene expression into close proximity.

In one embodiment, the CRISPR/Cas9-based system includes a fusion molecule or a nucleic acid encoding a fusion molecule. In one embodiment, the fusion molecule comprises a sequence comprising a dCas9 fused to a modulator of gene expression. In one embodiment, the dCas9 molecule comprises a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 16511) dCas9 molecule, a Campylobacter lari (strain CF89-12) dCas9 molecule, a Streptococcus thermophilus (strain LMD-9) dCas9 molecule, or fragment thereof.

### Modulators of Gene Expression

In some embodiments, the constructs provided herein comprise one or more modulators of gene expression. A modulator of gene expression may be a repressor of gene expression or an activator of gene expression. The repressor may be any known repressor of gene expression, for example, a repressor chosen from Kruppel associated box (KRAB) domain, mSin3 interaction domain (SID), MAX-interacting protein 1 (MXI1), a chromo shadow domain, an EAR-repression domain (SRDX), eukaryotic release factor 1 (ERFI), eukaryotic release factor 3 (ERF3), tetracycline repressor, the lad repressor, Catharanthus roseus G-box binding factors 1 and 2, Drosophila Groucho, Tripartite motif-containing 28 (TRTM28), Nuclear receptor co-repressor 1, Nuclear receptor co-repressor 2, or fragment or fusion thereof. The activator may be any known activator of gene expression, for example, a VP16 activation domain, a VP64 activation domain, a p65 activation domain, an Epstein-Barr virus R transactivator Rta molecule, or fragment thereof. Activations that can be used with a dCas9 molecule are known in the art. See, e.g., Chavez et al., Nat Methods. (2016) 13. 563-67, incorporated by reference herein in its entirety.

In particular embodiments, the methods and compositions disclosed herein include a fusion molecule comprising a dCas9 molecule fused to a modulator of gene expression. In some embodiments, the modulator of gene expression comprises a modulator of epigenetic modification. In one embodiment, the fusion molecule modulates target gene expression via epigenetic modification, e.g., via histone acetylation or methylation, or DNA methylation, at a regulatory element of target gene, e.g., a promoter, enhancer or transcription start site. The modulator may be any known modulator of epigenetic modification, e.g., a histone acetyltransferase (e.g., p300 catalytic domain), a histone deacetylase, a histone methyltransferase (e.g., SUV39H1 or G9a (EHMT2)), a histone demethylase (e.g., LSD1), a DNA methyltransferase (e.g., DNMT3a or DNMT3a-DNMT3L), a DNA demethylase (e.g., TET1 catalytic domain or TDG), or fragment thereof.

### Kruppel Associated Box (KRAB)

The KRAB domain is a type of transcriptional repression domains present in the N-terminal part of many zinc finger protein-based transcription factors. The KRAB domain functions as a transcriptional repressor when tethered to a target DNA by a DNA-binding domain. The KRAB domain is enriched in charged amino acids and can be divided into sub-domains A and B. The KRAB A and B sub-domains can be separated by variable spacer segments and many KRAB proteins contain only the A sub-domain. A sequence of 45 amino acids in the KRAB A sub-domain has been shown to be important for transcriptional repression. The B sub-domain does not repress transcription by itself but does potentiate the repression exerted by the KRAB A sub-domain. The KRAB domain recruits corepressors KAP1 (KRAB-associated protein-1, also known as transcription intermediary factor 1 beta, KRAB-A interacting protein and tripartite motif protein 28) and heterochromatin protein 1 (HP1), as well as other chromatin modulating proteins, leading to transcriptional repression through heterochromatin formation. In one embodiment, the methods and compositions disclosed herein include a fusion molecule comprising a dCas9 molecule fused to a KRAB domain or fragment thereof. In one embodiment, the KRAB domain or fragment thereof is fused to the N-terminus of the dCas9 molecule. In one embodiment, the KRAB domain or fragment thereof is fused to the C-terminus of the dCas9 molecule. In one embodiment, the KRAB domain or fragment thereof is fused to both the N-terminus and the C-terminus of the dCas9 molecule. In one embodiment, the fusion molecule comprises a KRAB domain comprising the sequence of SEQ ID NO: 51, 53 or 230-241, a sequence substantially identical (e.g., at least 80%, 85%, 90%, 92%, 95%, 97%, 98%, 99% or higher identical) to SEQ ID NO: 51, 53 or 230-241 or a sequence having one, two, three, four, five or more changes, e.g., amino acid substitutions, insertions, or deletions, relative to SEQ ID NO: 51, 53 or 230-241, or any fragment thereof.

### Exemplary KRAB Domain Sequence

Exemplary KRAB domain sequence
(SEQ ID NO: 51)
RTLVTFKDVFVDFTREEWKLLDTAQQIVYRNVMLENYKNLVSLGYQLTK
PDVILRLEKGEEP.

In one embodiment, the fusion molecule is a DNMT3A-DNMT3L(3A3L)-dCas9-KRAB fusion molecule comprising from the N-terminus to the C-terminus: a DNMT3A-DNMT3L fusion peptide (3A3L), a dCas9 peptide, and a KRAB peptide domain, fused directly or indirectly (e.g., via a linker).

In one embodiment, the fusion molecule is a DNMT3A-DNMT3L(3A3L)-dCas9-KRAB fusion molecule comprising from the N-terminus to the C-terminus: a DNMT3A-DNMT3L fusion peptide (3A3L), a dCas9 peptide, and a KRAB peptide domain, fused directly or indirectly (e.g., via a linker).

In one embodiment, the fusion molecule comprises the fusion molecule comprises the amino acid sequence of SEQ ID NO: 96, a sequence substantially identical (e.g., at least 80%, 85%, 90%, 91%, 92%, 93%, 94%, 95%, 96%, 97%, 98%, 99% or higher in sequence identity) to SEQ ID NO: 97, or a sequence having one, two, three, four, five or more changes, e.g., substitutions, insertions, or deletions, relative to SEQ ID NO: 96, or any fragment thereof.

DNMT3A-DNMT3L(3A3L)-dCas9-KRAB
(SEQ ID NO: 96)
MNHDQEFDPPKVYPPVPAEKRKPIRVLSLFDGIATGLLVLKDLGIQVDRYIASEVCEDSITVGM
VRHQGKIMYVGDVRSVTQKHIQEWGPFDLVIGGSPCNDLSIVNPARKGLYEGTGRLFFEFYRLL
HDARPKEGDDRPFFWLFENVVAMGVSDKRDISRFLESNPVMIDAKEVSAAHRARYFWGNLPGMN
RPLASTVNDKLELQECLEHGRIAKFSKVRTITTRSNSIKOGKDOHFPVFMNEKEDILWCTEMER
VFGFPVHYTDVSNMSRLARORLLGRSWSVPVIRHLFAPLKEYFACVSSGNSNANSRGPSFSSGL
VPLSLRGSHMGPMEIYKTVSAWKRQPVRVLSLERNIDKVLKSLGFLESGSGSGGGTLKYVEDVT
NVVRRDVEKWGPFDLVYGSTQPLGSSCDRCPGWYMFQFHRILQYALPRQESQRPFFWIEMDNLL
LTEDDQETTTRFLQTEAVTLQDVRGRDYQNAMRVWSNIPGLKSKHAPLTPKEEEYLQAQVRSRS
KLDAPKVDLLVKNCLLPLREYFKYFSQNSLPLGGPSSGAPPPSGGSPAGSPTSTEEGTSESATP
ESGPGTSTEPSEGSAPGSPAGSPTSTEEGTSTEPSEGSAPGTSTEPSEPKKKRKVMDKKYSIGL
AIGTNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYPTIYH
LRKKLVDSTDKADLRLIYLALAHMIKFRGHFLIEGDLNPDNSDVDKLFIQLVQTYNOLFEENPI
NASGVDAKAILSARLSKSRRLENLIAQLPGEKKNGLFGNLIALSLGLTPNFKSNFDLAEDAKLQ
LSKDTYDDDLDNLLAQIGDQYADLFLAAKNLSDAILLSDILRVNTEITKAPLSASMIKRYDEHH
QDLTLLKALVRQQLPEKYKEIFFDQSKNGYAGYIDGGASQEEFYKFIKPILEKMDGTEELLVKL
NREDLLRKORTFDNGSIPHQIHLGELHAILRRQEDFYPFLKDNREKIEKILTFRIPYYVGPLAR
GNSRFAWMTRKSEETITPWNFEEVVDKGASAQSFIERMTNFDKNLPNEKVLPKHSLLYEYFTVY
NELTKVKYVTEGMRKPAFLSGEQKKAIVDLLEKTNRKVTVKOLKEDYFKKIECFDSVEISGVED
RFNASLGTYHDLLKIIKDKDELDNEENEDILEDIVLTLTLFEDREMIEERLKTYAHLEDDKVMK
QLKRRRYTGWGRLSRKLINGIRDKQSGKTILDFLKSDGFANRNFMOLIHDDSLTFKEDIQKAQV
SGQGDSLHEHIANLAGSPAIKKGILQTVKVVDELVKVMGRHKPENIVIEMARENOTTOKGQKNS
RERMKRIEEGIKELGSQILKEHPVENTQLQNEKLYLYYLONGRDMYVDQELDINRLSDYDVDAI
VPQSFLKDDSIDNKVLTRSDKNRGKSDNVPSEEVVKKMKNYWRQLLNAKLITQRKEDNLTKAER
GGLSELDKAGFIKRQLVETROITKHVAQILDSRMNTKYDENDKLIREVKVITLKSKLVSDFRKD
FQFYKVREINNYHHAHDAYLNAVVGTALIKKYPKLESEFVYGDYKVYDVRKMIAKSEQEIGKAT
AKYFFYSNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNE
LALPSKYVNFLYLASHYEKLKGSPEDNEQKOLFVEQHKHYLDEIIEQISEFSKRVILADANLDK
VLSAYNKHRDKPIREQAENIIHLFTLTNLGAPAAFKYFDTTIDRKRYTSTKEVLDATLIHQSIT
GLYETRIDLSQLGGDAYPYDVPDYASLGSGSPKKKRKVEDPKKKRKVDGSGSETPGTSESATPE
SRTLVTFKDVFVDFTREEWKLLDTAQQIVYRNVMLENYKNLVSLGYQLTKPDVILRLEKGEEP

In some embodiments, the KRAB domain is a Zinc Finger Imprinted 3 (ZIM3) KRAB domain. The ZIM3 KRAB domain is a potent repressor of gene expression. In some embodiments, the ZIM3 KRAB domain the sequence of SEQ ID NO: 53, a sequence substantially identical (e.g., at least 80%, 85%, 90%, 92%, 95%, 97%, 98%, 99% or higher identical) to SEQ ID NO: 53, or a sequence having one, two, three, four, five or more changes, e.g., amino acid substitutions, insertions, or deletions, relative to SEQ ID NO: 53, or any fragment thereof. In some embodiments, the ZIM3 KRAB domain is encoded by the sequence set forth in SEQ ID NO: 54.

### Enhancer of Zeste Homolog 2 (EZH2)

In one embodiment a construct provided herein comprises an Enhancer of Zeste Homolg 2 (EZH2) domain. EZH2 is a histone methyl transferase that regulates several aspects of cell cycle progression. removes methyl groups from mono- and dimethylated lysine 4 and/or lysine 9 on histone H3 (H3K4me1/2 and H3K9me1/2),

In some embodiments, an EZH2 domain comprises the sequence of SEQ ID NO: 55, a sequence substantially identical (e.g., at least 80%, 85%, 90%, 92%, 95%, 97%, 98%, 99% or higher identical) to SEQ ID NO: 55, or a sequence having one, two, three, four, five or more changes, e.g., amino acid substitutions, insertions, or deletions, relative to SEQ ID NO: 55, or any fragment thereof. In some embodiments, an EZH2 domain is encoded by the sequence set forth in SEQ ID NO: 56.

### G9a (Euchromatic Histone Lysine Methyltransferase 2, EHMT2),

In some embodiments, a construct provided herein comprises an G9A or EhMT2 domain. EHMT2 is a methyltransferase that methylates lysine residues of histone H3. In some embodiments, the EHMT2 domain is fused to the C-terminus of a dCas9 protein. In some embodiments, the EHMT2 domain is fused to the C-terminus of a KRAB domain.

In some embodiments, an EHMT2 domain comprises the sequence of SEQ ID NO: 57, a sequence substantially identical (e.g., at least 80%, 85%, 90%, 92%, 95%, 97%, 98%, 99% or higher identical) to SEQ ID NO: 57, or a sequence having one, two, three, four, five or more changes, e.g., amino acid substitutions, insertions, or deletions, relative to SEQ ID NO: 57, or any fragment thereof. In some embodiments, an EZH2 domain is encoded by the sequence set forth in SEQ ID NO: 58.

### Lysine-Specific Histone Demethylase 1A (LSD1),

In some embodiments, a construct provided herein comprises an LSD1 domain. LSD1 is a histone demethylase that removes methyl groups from mono- and dimethylated lysine 4 and/or lysine 9 on histone H3 (H3K4me1/2 and H3K9me1/2) In some embodiments, the LSD1 domain is fused to the C-terminus of a dCas9 protein. In some embodiments, the LSD1 domain is fused to the C-terminus of a KRAB domain.

In some embodiments, an LSD1 domain comprises the sequence of SEQ ID NO: 59, a sequence substantially identical (e.g., at least 80%, 85%, 90%, 92%, 95%, 97%, 98%, 99% or higher identical) to SEQ ID NO: 59, or a sequence having one, two, three, four, five or more changes, e.g., amino acid substitutions, insertions, or deletions, relative to SEQ ID NO: 59, or any fragment thereof. In some embodiments, an LSD1 domain is encoded by the sequence set forth in SEQ ID NO: 60.

### Heterochromatin Protein 1 (HP1)

In some embodiments, a construct provided herein comprises an HP1 domain. HP1 contributes to the formation of beterochromatic structures. In some embodiments, the HP1 domain is fused to the C-terminus of a dCas9 protein. In some embodiments, the HP1 domain is fused to the C-terminus of a KRAB domain.

In some embodiments, an HP1 domain comprises the sequence of SEQ ID NO: 61, a sequence substantially identical (e.g., at least 80%, 85%, 90%, 92%, 95%, 97%, 98%, 99% or higher identical) to SEQ ID NO: 61, or a sequence having one, two, three, four, five or more changes, e.g., amino acid substitutions, insertions, or deletions, relative to SEQ ID NO: 61, or any fragment thereof. In some embodiments, an LSD1 domain is encoded by the sequence set forth in SEQ ID NO: 62.

Friend of GATA protein 1 (FOG1)

In some embodiments, a construct provided herein comprises Friend of GATA protein 1 (FOG1). FOG1 is a co-factor of the GATA1 transcription factor and regulates cell differentiation. In some embodiments, the FOG1 domain is fused to the C-terminus of a dCas9 protein. In some embodiments, the FOG1 domain is fused to the C-terminus of a KRAB domain.

In some embodiments, an FOG1 domain comprises the sequence of SEQ ID NO: 63, a sequence substantially identical (e.g., at least 80%, 85%, 90%, 92%, 95%, 97%, 98%, 99% or higher identical) to SEQ ID NO: 63, or a sequence having one, two, three, four, five or more changes, e.g., amino acid substitutions, insertions, or deletions, relative to SEQ ID NO: 63, or any fragment thereof. In some embodiments, an FOG1 domain is encoded by the sequence set forth in SEQ ID NO: 64.

### Histone Deacetylase (HDAC3)

In some embodiments, a construct provided herein comprises a HDAC3. HDAC3 contributes to the deacetylation of lysine residues on the N-terminal part of the core histones (H2A, H2B, H3 and H4). In some embodiments, the HDAC3 domain is fused to the C-terminus of a dCas9 protein. In some embodiments, the HDAC3 domain is fused to the C-terminus of a KRAB domain.

In some embodiments, an HDAC3 domain comprises the sequence of SEQ ID NO: 65, a sequence substantially identical (e.g., at least 80%, 85%, 90%, 92%, 95%, 97%, 98%, 99% or higher identical) to SEQ ID NO: 65, or a sequence having one, two, three, four, five or more changes, e.g., amino acid substitutions, insertions, or deletions, relative to SEQ ID NO: 65, or any fragment thereof. In some embodiments, an HDAC3 domain is encoded by the sequence set forth in SEQ ID NO: 66.

### DOT1 Like Histone Lysine Methyltransferase (DOT1L)

In some embodiments, a construct provided herein comprises a DOT1L. DOT1L is a histone methyltransferase that methylates lysine-79 of histone H3. In some embodiments, the DOT1L domain is fused to the C-terminus of a dCas9 protein. In some embodiments, the DOT1L domain is fused to the C-terminus of a KRAB domain.

In some embodiments, an DOT1L domain comprises the sequence of SEQ ID NO: 67, a sequence substantially identical (e.g., at least 80%, 85%, 90%, 92%, 95%, 97%, 98%, 99% or higher identical) to SEQ ID NO: 67, or a sequence having one, two, three, four, five or more changes, e.g., amino acid substitutions, insertions, or deletions, relative to SEQ ID NO: 67, or any fragment thereof. In some embodiments, an DOT1L domain is encoded by the sequence set forth in SEQ ID NO: 68.

### Histone Modification Activity

In some embodiments, the modulator of epigenetic modification may have histone modification activity. Histone modification activity may include but are not limited to histone deacetylase, histone acetyltransferase, histone demethylase, or histone methyltransferase activity.

In some embodiments, the modulator of epigenetic modification may have histone acetyltransferase activity. The histone acetyltransferase may be p300 or CREB-binding protein (CBP) protein, or fragments thereof. In some embodiments, the methods and compositions disclosed herein include a fusion molecule comprising a dCas9 molecule fused to acetyltransferase p300 or fragment thereof, e.g., the catalytic core of p300. In some embodiments, the methods and compositions disclosed herein include a fusion molecule comprising a dCas9 molecule fused to CREB-binding protein (CBP) protein or fragment thereof.

In some embodiments, the modulator of epigenetic modification may have histone demethylase activity. For example, the modulator of epigenetic modification may include an enzyme that removes methyl (CH3-) groups from nucleic acids or proteins (e.g., histones). In some embodiments, the methods and compositions disclosed herein include a fusion molecule comprising a dCas9 molecule fused to Lys-specific histone demethylase 1 (LSD1) or fragment thereof.

In some embodiments, the modulator of epigenetic modification may have histone methyltransferase activity. In some embodiments, the methods and compositions disclosed herein include a fusion molecule comprising a dCas9 molecule fused to SUV39H1 or fragment thereof. In some embodiments, the methods and compositions disclosed herein include a fusion molecule comprising a dCas9 molecule fused to G9a (EHMT2) or fragment thereof.

### DNA Demethylase Activity

In some embodiments, the modulator of epigenetic modification may have DNA demethylase activity. For example, the modulator of epigenetic modification may covert the methyl group to hydroxymethylcytosine as a mechanism for demethylating DNA. In some embodiments, the methods and compositions disclosed herein include a fusion molecule comprising a dCas9 molecule fused to Ten-eleven translocation methylcytosine dioxygenase 1 (TET1) or fragment thereof. In some embodiments, the methods and compositions disclosed herein include a fusion molecule comprising a dCas9 molecule fused to thymine DNA glycosylase (TDG) or fragment thereof.

gRNA

As used herein, the term “guide sequence” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequence may form a duplex with a target sequence. The duplex may be a DNA duplex, an RNA duplex, or a RNA/DNA duplex. The terms “guide molecule” and “guide RNA” and “single guide RNA” are used interchangeably herein to refer to RNA-based molecules that are capable of forming a complex with a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA specifically encompasses RNA-based molecules having one or more chemically modifications (e.g., by chemical linking two ribonucleotides or by replacement of one or more ribonucleotides with one or more deoxyribonucleotides), as described herein.

The guide molecule or guide RNA of a CRISPR-Cas protein may comprise a tracr-mate sequence (encompassing a “direct repeat” in the context of an endogenous CRISPR system) and a guide sequence (also referred to as a “spacer” in the context of an endogenous CRISPR system).

In some embodiments, the CRISPR-Cas system or complex as described herein does not comprise and/or does not rely on the presence of a tracr sequence. In certain embodiments, the guide molecule may comprise, consist essentially of, or consist of a direct repeat sequence fused or linked to a guide sequence or spacer sequence.

In general, a CRISPR-Cas system is characterized by elements that promote the formation of a CRISPR complex at the site of a target sequence. In the context of formation of a CRISPR complex, “target sequence” refers to a sequence to which a guide sequence is designed to have complementarity, where hybridization between a target DNA sequence and a guide sequence promotes the formation of a CRISPR complex.

In certain embodiments, the guide sequence or spacer length of the guide molecules is 15 to 50 nucleotides in length. In certain embodiments, the spacer length of the guide RNA is at least 15 nucleotides in length. In certain embodiments, the spacer length is from 15 to 17 nucleotides in length, from 17 to 20 nucleotides in length, from 20 to 24 nucleotides in length, from 23 to 25 nucleotides in length, from 24 to 27 nucleotides in length, from 27-30 nucleotides in length, from 30-35 nucleotides in length, or greater than 35 nucleotides in length.

In some embodiments, the guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

As described above, the CRISPR/Cas9 system utilizes gRNA that provides the targeting of the CRISPR/Cas9-based system. The gRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA: tracrRNA duplex involved in the Type II Effector system. This duplex, which may include, for example, a 42-nucleotide crRNA and a 75-nucleotide tracrRNA, acts as a guide for the Cas9 to cleave the target nucleic acid.

The term “target region”, “target sequence” or “protospacer” as used interchangeably herein refers to the region of the target gene to which the CRISPR/Cas9-based system targets. The CRISPR/Cas9-based system may include at least one gRNA, wherein the gRNAs target different DNA sequences. The target DNA sequences may be overlapping. The target sequence or protospacer is followed by a PAM sequence at the 3′ end of the protospacer. Different Type II systems have differing PAM requirements. For example, the S. pyogenes Type II system uses an “NGG” sequence, where “N” can be any nucleotide.

In some embodiments, the number of gRNA administered to the cell may be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 19 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs.

In some embodiments, the number of gRNAs administered to the cell may be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

In some embodiments, the gRNA is selected to increase or decrease transcription of a target gene. In some embodiment, the gRNA targets a region upstream of the transcription start site (TSS) of a target gene (e.g. VEGFA), e.g., between 0-1000 bp upstream of the transcription start site of a target gene. In some embodiments, the gRNA targets a region between 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp upstream of the transcription start site of the target gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the transcription start site of the target gene. In one embodiment, the gRNA targets a region 0-300 bp upstream of the TSS of the target gene.

In some embodiments, the gRNA targets a region downstream of the transcription start site of a target gene, e.g., between 0-1000 bp downstream of the transcription start site of a target gene. In some embodiments, the gRNA targets a region between 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp downstream of the transcription start site of the target gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the transcription start site of the target gene. In one embodiment, the gRNA targets a region 0-300 bp downstream of the TSS of the target gene.

The present disclosure provides sgRNA sequences that target a human VEGFA target gene as well as sgRNA sequences that target a mouse VEGFA target gene. The sequence of human VEGFA is provided in NCBI Reference Sequence: NG_008732.1. The sequence of mouse VEGFA is provided in NCBI Reference Sequence: NC_000083.7. The sequence of rhesus monkey VEGFA is provided in NCBI Reference Sequence: NC_041757.1. The present disclosure provides sgRNA sequences that also target CD151, CD81 and PCSK9 target genes. Exemplary sgRNAs include but are not limited to those listed in Table 4.

TABLE 4
Exemplary sgRNAs

| | Gene | sgRNA sequence |
|---|---|---|
| sgRNA-1 | VEGFA | gctcggaggtcgtggcgctg |
| | (Human) | (SEQ ID NO: 190) |
| sgRNA-2 | VEGFA | ggctagcaccagcgctctgt |
| | (Human) | (SEQ ID NO: 191) |
| sgRNA-3 | CD151 | CCGGACTCGGACGCGTGGT |
| | | (SEQ ID NO: 192) |
| sgRNA-4 | CD151 | TGTCCAGGGACAATGAGCA |
| | | (SEQ ID NO: 193) |
| sgRNA-5 | CD151 | GACAATGAGCAGGGTGTCC |
| | | (SEQ ID NO: 194) |
| sgRNA-6 | CD81 | GAGAGCGAGCGCGCAACGG |
| | | (SEQ ID NO: 195) |
| sgRNA-7 | CD81 | GGCCTGGCAGGATGCGCGG |
| | | (SEQ ID NO: 196) |
| sgRNA-8 | CD81 | GCCGAAACGCGCCAAGTTGG |
| | | (SEQ ID NO: 197) |
| sgRNA-9 | PCSK9 | aacctgatcctttagtaccg |
| | | (SEQ ID NO: 198) |
| sgRNA-10 | PCSK9 | Ggatcttccgatggggctcg |
| | | (SEQ ID NO: 199) |
| sgRNA-11 | — | CTGAAGGTGTCTGGCAGAGC |
| (Nt sgRNA) | | (SEQ ID NO: 229) |

In one embodiment, the gRNA targets a promoter region of a target gene. In one embodiment, the gRNA targets an enhancer region of a target gene. gRNA can be divided into a target binding region, a Cas9 binding region, and a transcription termination region. The target binding region hybridizes with a target region in a target gene. Methods for designing such target binding regions are known in the art, see, e.g., Doench et al., Nat Biotechnol. (2014) 32: 1262-7; and Doench et al., Nat Biotechnol. (2016) 34: 184-91, incorporated by reference herein in their entirety. Design tools are available at, e.g., Feng Zhang lab's target Finder, Michael Boutros lab's Target Finder (E-CRISP), RGEN Tools (Cas-OF Finder), CasFinder, and CRISPR Optimal Target Finder. In certain embodiments, the target binding region can be between about 15 and about 50 nucleotides in length (about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 nucleotides in length). In certain embodiments, the target binding region can be between about 19 and about 21 nucleotides in length. In one embodiment, the target binding region is 15, 16, 17, 18, 19, 20, 21, 22, 23, 24, or 25 nucleotides in length.

In one embodiment, the target binding region is complementary, e.g., completely complementary, to the target region in the target gene. In one embodiment, the target binding region is substantially complementary to the target region in the target gene. In one embodiment, the target binding region comprises no more than 1, 2, 3, 4, 5, 6, 7, 8, 9, or 10 nucleotides that are not complementary to the target region in the target gene.

In one embodiment, the target binding region is engineered to improve stability or extend half-life, e.g., by incorporating a non-natural nucleotide or a modified nucleotide in the target binding region, by removing or modifying an RNA destabilizing sequence element, by adding an RNA stabilizing sequence element, or by increasing the stability of the Cas9/gRNA complex. In one embodiment, the target binding region is engineered to enhance its transcription. In one embodiment, the target binding region is engineered to reduce secondary structure formation. In one embodiment, the Cas9 binding region of gRNA is modified to enhance the transcription of the gRNA. In one embodiment, the Cas9 binding region of gRNA is modified to improve stability or assembly of the Cas9/gRNA complex.

### Delivery Systems

The present disclosure also provides delivery systems for introducing components of the systems and compositions herein to cells, tissues, organs, or organisms. A delivery system may comprise one or more delivery vehicles and/or cargos.

### Cargos

The delivery systems may comprise one or more cargos. The cargos may comprise one or more components of the systems and compositions herein. A cargo may comprise one or more of the following: i) a plasmid encoding one or more Cas proteins; ii) a plasmid encoding one or more guide RNAs, iii) mRNA of one or more Cas proteins; iv) one or more guide RNAs; v) one or more Cas proteins; vi) any combination thereof. In some examples, a cargo may comprise a plasmid encoding one or more Cas protein and one or more (e.g., a plurality of) guide RNAs. In some embodiments, a cargo may comprise mRNA encoding one or more Cas proteins and one or more guide RNAs.

In some examples, a cargo may comprise one or more Cas proteins and one or more guide RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516.

### Physical Delivery

In some embodiments, the cargos may be introduced to cells by physical delivery methods. Examples of physical methods include microinjection, electroporation, and hydrodynamic delivery.

### Microinjection

Microinjection of the cargo directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for Cas proteins and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas-encoding mRNA to the cytoplasm, e.g., facilitating translation and shuttling of Cas to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present disclosure may a greatest dimension (e.g. diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

### Vectors

The systems, compositions, and/or delivery systems may comprise one or more vectors. The present disclosure also include vector systems. A vector system may comprise one or more vectors. In some embodiments, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells.

Common expression vectors of utility in recombinant DNA techniques are often in the form of plasmids.

Examples of vectors include pGEX, pMAL, pRIT5, E. coli expression vectors (e.g., pTrc, pET11d, yeast expression vectors (e.g., pYepSecl, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC.

A vector may comprise i) Cas encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively or additionally, in a single vector, there may be a promoter controlling (e.g., driving transcription and/or expression) multiple RNA encoding sequences.

### Regulatory Elements

A vector may comprise one or more regulatory elements. The regulatory element(s) may be operably linked to coding sequences of Cas proteins, accessary proteins, guide RNAs (e.g., a single guide RNA, crRNA, and/or tracrRNA), or combination thereof. The term “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). In certain examples, a vector may comprise: a first regulatory element operably linked to a nucleotide sequence encoding a Cas protein, and a second regulatory element operably linked to a nucleotide sequence encoding a guide RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and HI promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the -actin promoter, the phosphoglycerol kinase (PGK) promoter, and the EF1a promoter.

### Viral Vectors

The cargos may be delivered by viruses. In some embodiments, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno-Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In some embodiments, AAV may provide a persistent source of the provided DNA, as AAV delivered genomic material can exist indefinitely in cells, e.g., either as exogenous DNA or, with some modification, be directly integrated into the host DNA In some embodiments, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82: 5887-5911 (2008)) and WO 2021/183807A1, which are incorporated by reference herein in their entirety.

CRISPR-Cas AAV particles may be created in HEK 293 T cells. Once particles with specific tropism have been created, they are used to infect the target cell line much in the same way that native viral particles do. This may allow for persistent presence of CRISPR-Cas components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of Cas and gRNA may be packaged directly onto one DNA plasmid vector and delivered via one AAV particle. In some examples, AAVs may be used to deliver gRNAs into cells that have been previously engineered to express Cas. In some examples, coding sequences of Cas and gRNA may be made into two separate AAV particles, which are used for co-transfection of target cells. In some examples, markers, tags, and other sequences may be packaged in the same AAV particles as coding sequences of Cas and/or gRNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In certain embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In some embodiments, adenoviruses do not integrate into the genome of host cells, which may be used for limiting off-target effects of CRISPR-Cas systems in gene editing applications.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin 0, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

In some embodiments, LNPs are used for delivering an mRNA and gRNAs (e.g. mRNA fusion molecule comprising DNMT3A-DNMT3L(3A-3L)-dCas9-KRAB and at least one sgRNA targeting VEGFA.

Components of LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Conway et al, Molecular Therapy, vol. 27, no. 4, pages 866-877, April 2019 and Rosin et al, Molecular Therapy, vol. 19, no. 12, pages 1286-2200, December 2011.

In some embodiments, LNPs may comprise ionizable lipids. In some embodiments, ionizable lipids include but are not limited to pH-responsive ionizable lipids, thermal-responsive ionizable lipids and light-responsive ionizable lipids. In some embodiments, ionizable lipids include cationic lipids and anionic lipids that are ionized under the certain conditions, such as, but not limited to pH, temperature or light. In some embodiments, the molar ratio of ionizable lipids of the LNP is 20% to about 70% (e.g., about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 30% to about 70%, about 30% to about 65%, about 30% to about 60%, about 30% to about 55%, about 30% to about 50%, about 30% to about 45%, about 30% to about 40%, about 30% to about 35%, about 40% to about 70%, about 40% to about 65%, about 40% to about 60%, about 40% to about 55%, about 40% to about 50%, about 40% to about 45%, about 50% to about 70%, about 50% to about 65%, about 50% to about 60%, about 50% to about 55%, about 60% to about 70%, or about 60% to about 65%)

In some embodiments, LNPs may comprise PEGylated lipids. In some embodiments, the molar ratio of PEGylated lipids of the LNP is 0% to about 30% (e.g., about 0% to about 30%, about 0% to about 25%, about 0% to about 20%, about 0% to about 15%, about 0% to about 10%, about 10% to about 30%, about 10% to about 25%, about 10% to about 20%, about 10% to about 15%, about 20% to about 30%, or about 20% to about 25%).

In some embodiments, LNPs may comprise supporting lipids. In some embodiments, the molar ratio of supporting lipids of the LNP is 30% to about 50% (e.g. about 30% to about 50%, about 30% to about 45%, about 30% to about 40%, about 30% to about 35%, about 40% to about 50%, or about 40% to about 45%)

In some embodiments, LNPs may comprise cholesterol. In some embodiments, the molar ratio of cholesterol of the LNP is 10% to about 50% (e.g., about 10% to about 50%, about 10% to about 45%, about 10% to about 40%, about 10% to about 35%, about 10% to about 30%, about 10% to about 25%, about 10% to about 20%, about 10% to about 15%, about 20% to about 50%, about 20% to about 45%, about 20% to about 40%, about 20% to about 35%, about 20% to about 30%, about 20% to about 25%, about 30% to about 50%, about 30% to about 45%, about 30% to about 40%, about 30% to about 35%, about 40% to about 50% or about 40% to about 45%).

In some embodiments, LNPs may comprise a mixture of ionizable lipids (20%-70%, molar ratio), PEGylated lipids (0%-30%, molar ratio), supporting lipids (30%-50%, molar ratio), and cholesterol (10%-50%, molar ratio).

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMA)

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

Lipoplexes and/or Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca²+ microcomplexes), polyethenimine (PEI) (e.g., branched PEI), and poly(L-lysine) (PLL).

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus I (HIV-I). Examples of CPPs include to Penetratin, Tat (48-60), Transportan, and (R-AhX-R4) (Ahx refers to aminohexanoyl). Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

### DNA Nanoclews

In some embodiments, the delivery vehicles comprise DNA nanoclews. A DNA nanoclew refers to a sphere-like structure of DNA (e.g., with a shape of a ball of yam). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Gold Nanoparticles

In some embodiments, the delivery vehicles comprise gold nanoparticles (also referred to AuNPs or colloidal gold). Gold nanoparticles may form complex with cargos, e.g., Cas:gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10. 1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi:10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci US A 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. AMEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Ace Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates Kand Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Methods of Use

The compositions and systems herein may be used for a variety of applications, including modifying non-animal organisms such as plants and fungi, and modifying animals, treating and diagnosing diseases in plants, animals, and humans. In general, the compositions and systems may be introduced to cells, tissues, organs, or organisms, where they modify the expression and/or activity of one or more genes.

### Cells and Organisms

The present disclosure provides cells, tissues, organisms comprising the engineered Cas protein, the CRISPR-Cas systems, the constructs, the polynucleotides encoding one or more components of the CRISPR-Cas systems, and/or vectors comprising the polynucleotides. The disclosure also provides for the nucleotide sequence encoding the effector protein being codon optimized for expression in a eukaryote or eukaryotic cell in any of the herein described methods or compositions. In an embodiment of the disclosure, the codon optimized effector protein is any Cas protein discussed herein and is codon optimized for operability in a eukaryotic cell or organism, e.g., such cell or organism as elsewhere herein mentioned, for instance, without limitation, a yeast cell, or a mammalian cell or organism, including a mouse cell, a rat cell, and a human cell or non-human eukaryote organism, e.g., plant.

In certain embodiments, the modification of the target locus of interest may result in: the eukaryotic cell comprising altered expression of at least one gene product; the eukaryotic cell comprising altered expression of at least one gene product, wherein the expression of the at least one gene product is increased; the eukaryotic cell comprising altered expression of at least one gene product, wherein the expression of the at least one gene product is decreased; or the eukaryotic cell comprising an edited genome.

In certain embodiments, the eukaryotic cell may be a mammalian cell or a human cell.

In further embodiments, the non-naturally occurring or engineered compositions, the vector systems, or the delivery systems as described in the present specification may be used for: site-specific gene knockout; site-specific genome editing; RNA sequence-specific interference; or multiplexed genome engineering.

Also provided is a gene product from the cell, the cell line, or the organism as described herein. In certain embodiments, the amount of gene product expressed may be greater than or less than the amount of gene product from a cell that does not have altered expression or edited genome.

In certain embodiments, the gene product may be altered in comparison with the gene product from a cell that does not have altered expression or edited genome.

Also provided herein are compositions comprising the cells provided herein. In some embodiments, provided herein is a pharmaceutical composition comprising a cell provided herein and a pharmaceutically acceptable carrier.

### Methods of Modifying Gene Expression

In another aspect, provided herein are methods of modifying gene expression in a cell or in a subject in vivo. In particular, the methods provided herein may be used to modify the expression of a gene product in a cell while minimizing off-target modifications.

In some embodiments, provided herein is a method of modifying the expression of a gene product and minimizing off-target modifications in a population of cells or in a subject in vivo, the method comprising the step of introducing into the population of cells or into the cells of a subject (i) a construct described herein or a polypeptide(s) expressed by the construct; and (ii) at least one sgRNA, wherein the KRAB and/or modulator of gene expression provides a modification of at least one nucleotide near the gene and/or within a regulatory element of the gene, thereby modifying the expression of the gene product.

In some embodiments, the construct of step (i) has the structure of A construct of Formula I: 5′-(Am1-Bm2)n1-CasN-(Am3-Bm4)n2-CasC-Tp-E-3′ (I), wherein one of A and B is a polynucleotide encoding DNMT3A or a portion thereof and the other of A and B is a polynucleotide encoding DNMT3L or a portion thereof, CasN is a polynucleotide encoding a N-terminal portion of dCas9; CasC is a polynucleotide encoding a C-terminal portion of dCas9; E is 5′-(Am5-Bm6)n3-Kr-Dq-3′ or 5′-Kr-Dq-(Am5-Bm6)n3-3′; K is a polynucleotide encoding KRAB or a portion thereof, D is a polynucleotide encoding a modulator of gene expression; T comprises a polynucleotide encoding (i) an epitope capable of binding to an antibody or an antigen binding fragment thereof or (ii) a polypeptide sequence capable of binding to a nucleic acid structural element; m1, m2, m3, m4, m5, and m6 are each independently an integer selected from 0 to 3; n1, n2, and n3 are each independently an integer selected from 0 to 2; p is an integer selected from 0 to 20; q is an integer selected from 0 to 5; r is an integer selected from 0 to 5; and wherein when p is 0, at least one of m1, m2, m3, and m4 is not 0, at least one of n1 and n2 is not 0, and at least one of q and r is not 0.

In some embodiments, the construct has the structure of Formula II: 5′-CasN-(Am3-Bm4)n2-CasC-Kr-Dq-3′ (II), wherein n2 is an integer selected from 1 and 2; r is an integer selected from 1 to 5; q is an integer selected from 0 to 5; and at least one of m3 and m4 is not 0.

In some embodiments, the construct has the structure of Formula IIa: 5′-CasN-(A-B)-CasC-Kr-Dq-3′ (IIa). In some embodiments, the construct has the structure of Formula III: 5′-(Am1-Bm2)n1-CasN-(Am3-Bm4)n2-CasC-Tp-E-3′ (III), wherein p is an integer selected from 1 to 20.

In some embodiments, the construct has the structure of Formula IIIa: 5′-CasN-CasC-Tp-3′ (IIIa). In some embodiments, the construct has the structure of Formula IIIb: 5′-(Am1-Bm2)-CasN-CasC-Tp-E-3′ (IIIb), wherein at least one of m1 and m2 is not 0. In some embodiments, the construct has the structure of Formula IIIb-1: 5′-(A-B)-CasN-CasC-Tp-Kr-Dq-3′ (IIIb-1), wherein: r is an integer selected from 1 to 5; and q is an integer selected from 0 to 5.

In some embodiments, the construct has the structure of Formula IIIc: 5′-CasN-CasC-Tp-E-3′ (IIIc), wherein: n3 is an integer selected from 1 and 2; r is an integer selected from 1 to 5; and q is an integer selected from 0 to 5; and at least one of m5 and m6 is not 0. In some embodiments, the construct has the structure of Formula IIIc-1: 5′-CasN-CasC-Tp-(Am5-Bm6)n3-Kr-Dq-3′ (IIIc-1). In some embodiments, the construct has the structure of Formula IIIc-2: 5′-CasN-CasC-Tp-Kr-Dq-(Am5-Bm6)n3-3′ (IIIc-2).

Without wishing to be bound by theory, it is hypothesized that where T comprises a polynucleotide encoding an epitope capable of binding to an antibody or an antigen binding fragment thereof, the polypeptide of (i) comprising the 5′-(Am1-Bm2)n1-CasN-(Am3-Bm4)n2-CasC-Tp-3′ portion of Formula I, the 5′-(Am1-Bm2)n1-CasN-(Am3-Bm4)n2-CasC-Tp-3′ portion of Formula III, the 5′-(Am1-Bm2)-CasN-CasC-Tp-3′ portion of Formula IIIb, the 5′-(A-B)-CasN-CasC-Tp-3′ portion of Formula IIIb-1, the 5′-CasN-CasC-Tp-3′ portion of Formula IIIc, the 5′-CasN-CasC-Tp-3′ portion of Formula IIIc-1, or the 5′-CasN-CasC-Tp-3′ portion of Formula IIIc-2, the sgRNA of (ii), and multiple copies of the polypeptide of (i) comprising the 5′-E-3′ portion of Formula I, the 5′-E-3′ portion of Formula III, the 5′-E-3′ portion of Formula IIIb, the 5′-Kr-Dq-3′ portion of Formula IIIb-1, the 5′-E-3′ portion of Formula IIIc, the 5′-(Am5-Bm6)n3-Kr-Dq-3′ portion of Formula IIIc-1, or the 5′-Kr-Dq-(Am5-Bm6)n3-3′ portion of Formula IIIc-2 are recruited to a genomic locus via binding of the antibody or antigen binding fragment thereof to the epitope, thereby recruiting the polypeptides expressed by the construct to the genomic loci and modifying the expression of a gene product in a population of cells.

In some embodiments, the method further comprises (iii) introducing to the cells a second construct comprising the 5′-(Am5-Bm6)n3-Kr-Dq-3′ or 5′-Kr-Dq-(Am5-Bm6)n3-3′ of E or a polypeptide expressed by the second construct. Without wishing to be bound by theory, it is believed that the polypeptide of (i) comprising the 5′-CasN-CasC-Tp-3′ portion of Formula IIIa, the sgRNA, and multiple copies of the polypeptide of (iii) are recruited to a genomic locus via binding of the antibody or antigen binding fragment thereof to the epitope, thereby recruiting the polypeptides expressed by the construct of (i) and the construct of (iii) to the genomic loci, and modifying the expression of a gene product in a population of cells.

Without wishing to be bound by theory, it is believed that where T comprises a polypeptide sequence capable of binding to a nucleic acid structural element, the polypeptide of (i) comprising the 5′-(Am1-Bm2)n1-CasN-(Am3-Bm4)n2-CasC-Tp-3′ portion of Formula I, the 5′-(Am1-Bm2)n1-CasN-(Am3-Bm4)n2-CasC-Tp-3′ portion of Formula III, the 5′-(Am1-Bm2)-CasN-CasC-Tp-3′ portion of Formula IIIb, the 5′-(A-B)-CasN-CasC-Tp-3′ portion of Formula IIIb-1, the 5′-CasN-CasC-Tp-3′ portion of Formula IIIc, the 5′-CasN-CasC-Tp-3′ portion of Formula IIIc-1, or the 5′-CasN-CasC-Tp-3′ portion of Formula IIIc-2, the sgRNA of (ii), and multiple copies of the polypeptide of (i) comprising the 5′-E-3′ portion of Formula I, the 5′-E-3′ portion of Formula III, the 5′-E-3′ portion of Formula IIIb, the 5′-Kr-Dq-3′-portion of Formula IIIb-1, the 5′-E-3′-portion of Formula IIIc, the 5′-(Am5-Bm6)n3-Kr-Dq-3′ portion of Formula IIIc-1, or the 5′-Kr-Dq-(Am5-Bm6)n3-3′ portion of Formula IIIc-2 are recruited to a genomic loci via binding of the polypeptide sequence capable of binding to a nucleic acid structural element to the nucleic acid structural element, thereby recruiting the polypeptides expressed by the construct to the genomic loci and modifying the expression of a gene product in a population of cells.

In some embodiments, the method further comprises (iii) introducing to the cells a second construct comprising the 5′-(Am5-Bm6)n3-Kr-Dq-3′ or 5′-Kr-Dq-(Am5-Bm6)n3-3′ of E or a polypeptide expressed by the second construct. Without wishing to be bound by theory, it is believed that the polypeptide of (i) comprising the 5′-CasN-CasC-Tp-3′ portion of Formula IIIa, the sgRNA of (ii), and multiple copies of the polypeptide of (iii) are recruited to a genomic loci via binding of the polypeptide sequence capable of binding to a nucleic acid structural element to the nucleic acid structural element, thereby recruiting the polypeptides expressed by the construct to the genomic loci and modifying the expression of a gene product in a population of cells.

The methods provided herein may be used to modify gene expression in a cell in culture, in an isolated cell, or a cell in vivo, e.g., a cell in a subject. In vivo methods of modifying gene expression may be used to treat diseases as further described below.

In some embodiments, a method provided herein reduces the expression of the target gene. The reduction of the expression of a target gene may be measured, for example, in comparison the gene expression of the target gene product in the cell prior to exposure to the constructs provided herein, or in comparison to an unmodified cell. In some embodiments, a method provided herein reduces the expression of the target gene by 10%-20%, 20%-30%, 30%-40%, 40%-50%, 50%-60%, 60%-70%, 70%-80%, 80%-90%, 90%-100%, at least 10%, at least 20%, at least 30%, at least 40%, at least 50%, at least 60%, at least 70%, at least 80%, at least 90%, or 100% in the plurality of modified cells in comparison to a wildtype population of cells. In some embodiments, a ratio of on-site modification of the gene product to off-site modification of the gene product is about 10:1, 9:1, 8:1, 7:1, 6:1, 5:1, 4:1, 3:1 or 2:1. In some embodiments, the ratio of on-site modifications to off-site modifications is no more than 10:1.

The modification of at least one nucleotide introduced by KRAB and/or the modulator of gene expression may be, for example, a DNA methylation or a histone modification. The modification may be located in any region of the target gene where the modification achieves the desired effect (e.g., decrease of gene expression), including, for example, the coding sequence of a gene or a regulatory element. In some embodiments, the modification occurs in a core promoter, a proximal promoter, a distal enhancer, a silencer, an insulator element, a boundary element or a locus control region.

The modification may occur in a nucleotide that is located upstream or downstream of the transcription start site of the target gene. In some embodiments, the modification occurs at a nucleotide located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the transcription start site of the gene. In some embodiments, the modification occurs at a nucleotide located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the transcription start site of the gene.

### Exemplary Therapies

In another aspect, provided herein is a method for treating or alleviating a symptom of a gene product related disorder in a subject, comprising the step of introducing to a cell of the subject: (i) a construct provided herein or a polypeptide(s) expressed by the construct; and (ii) at least one sgRNA, wherein the KRAB and/or modulator of gene expression provides a modification of at least one nucleotide near the gene and/or within a regulatory element of the gene, thereby modifying the expression of the gene product and treating or alleviating a symptom of the gene product related disorder in the subject.

The present disclosure provides a use of the CRISPR-Cas system for treatment in of a variety of diseases and disorders. In some embodiments, the disclosure described herein relates to a method for therapy in which cells are edited ex vivo by CRISPR or the base editor to modulate at least one gene, with subsequent administration of the edited cells to a patient in need thereof. In some embodiments, the editing involves knocking in, knocking out or knocking down expression of at least one target gene in a cell. In particular embodiments, the editing inserts an exogenous, gene, minigene or sequence, which may comprise one or more exons and in trans or natural or synthetic in trans into the locus of a target gene, a hot-spot locus, a safe harbor locus of the gene genomic locations where new genes or genetic elements can be introduced without disrupting the expression or regulation of adjacent genes, or correction by insertions or deletions one or more mutations in DNA sequences that encode regulatory elements of a target gene. In some embodiment, the editing comprise introducing one or more point mutations in a nucleic acid (e.g., a genomic DNA) in a target cell.

In some embodiments, the treatment is for disease/disorder of an organ, including liver disease, eye disease, muscle disease, heart disease, blood disease, brain disease, kidney disease, or may comprise treatment for an autoimmune disease, central nervous system disease, cancer and other proliferative diseases, neurodegenerative disorders, inflammatory disease, metabolic disorder, musculoskeletal disorder and the like. In some embodiments, the disease is liver a liver disease. In some embodiments, the disease is an eye disease. In some embodiments, the disease is a CNS disease. In some embodiments, the disease is cancer. In some embodiments, the disease is selected from the group consisting of Familial hypercholesterolemia (FH), non-alcoholic steatohepatitis (NASH), Parkinson disease, hepatic fibrosis (HF), age-related macular disease (AMD), Angelman Syndrome (AS), Type II diabetes, 0-thalassemia, and hepatocellular carcinoma.

In some embodiments, regulation is affected by modification in the target gene VEGFA, PCSK9, ANGPTL3, PTBP1, TTR, Ube3a-ATS, Ptp1b, APOC3, hsd17b13, bcl11a, or TGF-beta. VEGFA. VEGFA plays an important role in angiogenesis and is overexpressed in several cancers. PCSK9 plays a key role in cholersterol management. ANGPTL3 plays a role in the metabolism of triglycerides. PTBP1 encodes a nuclear riboprotein and is involved in the regulation of transcription. TTR encodes the transporter protein transthyretin. Ube3a-ATS encodes a ubiquitin ligase and mutations in Ube3a-ATS are associated with Angelman syndrome. Ptp1b encodes Protein tyrosine phosphatase 1, which regulates insulin signaling. APOC3 encodes Apolipoprotein C3, which plays a key role in lipid transport and metabolism. hsd17b13 encodes the liver-specific hydroxysteroid 17p-dehydrogenase 13. BCL11A is a transcriptional repressor. TGF beta regulates several cellular pathways, importantly those associated with proliferation and differentiation.

The terms “subject” and “patient” are used interchangeably herein. In some embodiments, the subject is human.

### EXAMPLES

### Design and Construction of Plasmids Containing the Constructs of the Disclosure

Epigenetic modification editors with HA epitopes, P2A and BFP (e.g., including Dnmt3a CD, Dnmt31 CD, dSpCas9, KRAB and/or other histone modifiers) were optimized by Genscript into nucleic acid sequences suitable for mammalian expression and synthesis, and then cloned into the pLV-CAG vector with CAG promoter and WPRE, which expresses the complete epigenetic modification editor and self-sheared BFP.

In optimizing the different functional elements and the order of different functional elements, the different functional elements were optimized by Genscript into nucleic acid sequences suitable for mammalian expression and synthesis. The vector other than the elements to be replaced is first amplified by PCR, then the elements to be replaced are amplified from the sequences synthesized by the company while introducing the homologous arm sequence, and finally the different elements are recombined into the vector by NEBuilder reagent to construct the final expression plasmid.

### Example 1: Constructs for Epigenetic Modification of CD151 Gene and CD81 Gene

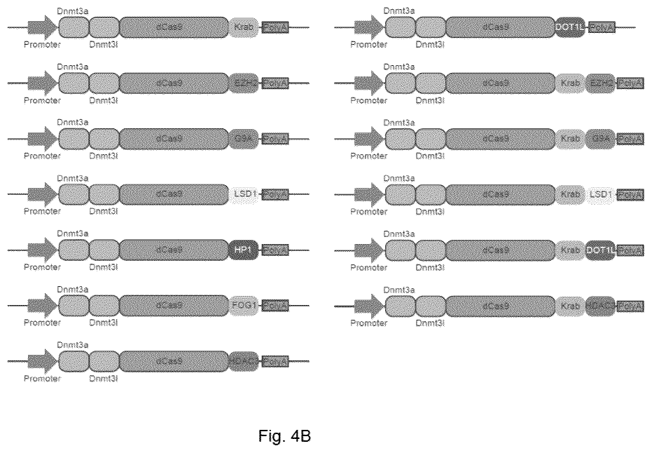

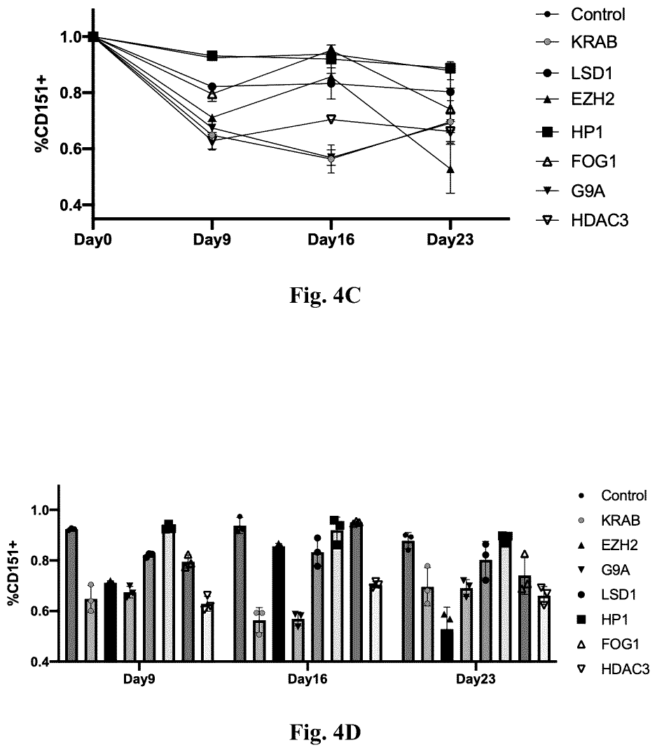

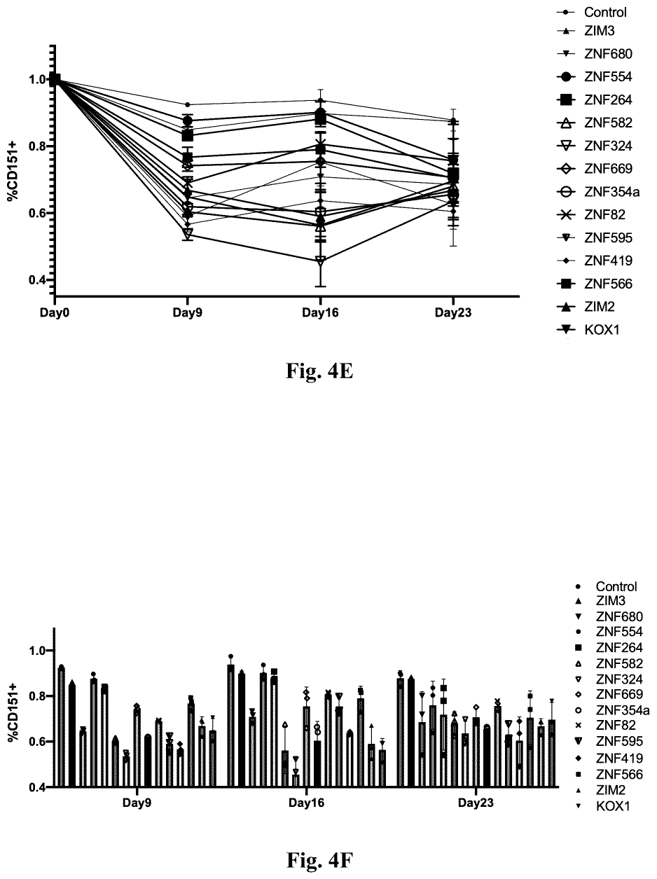

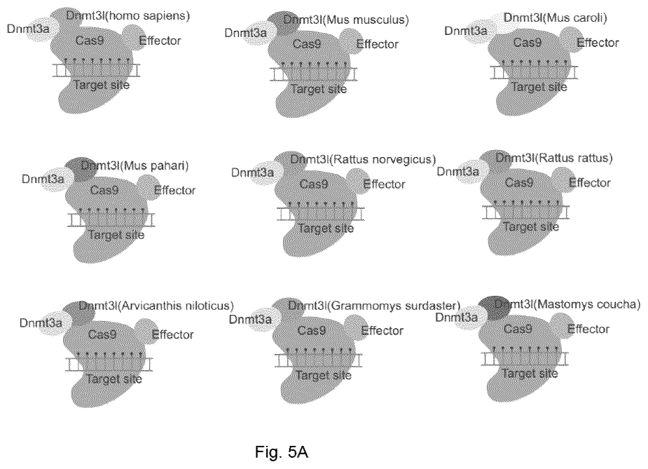

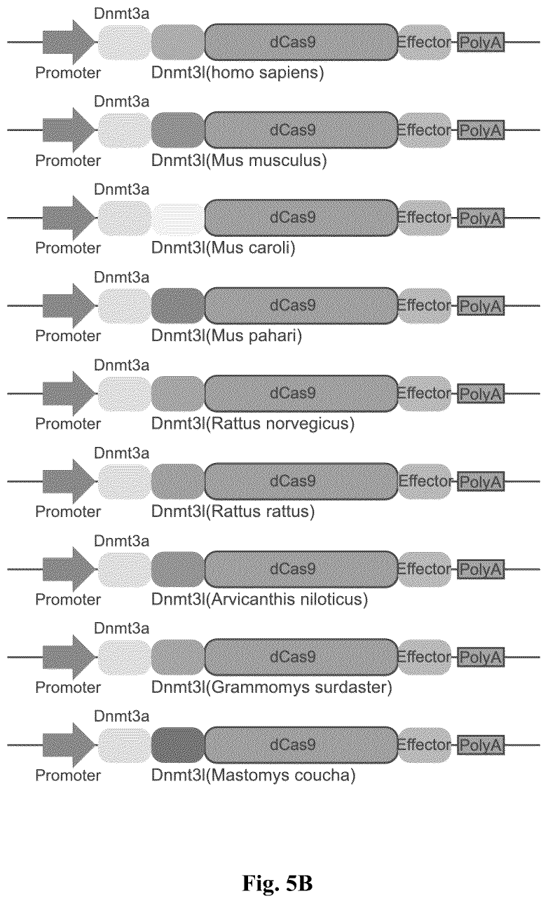

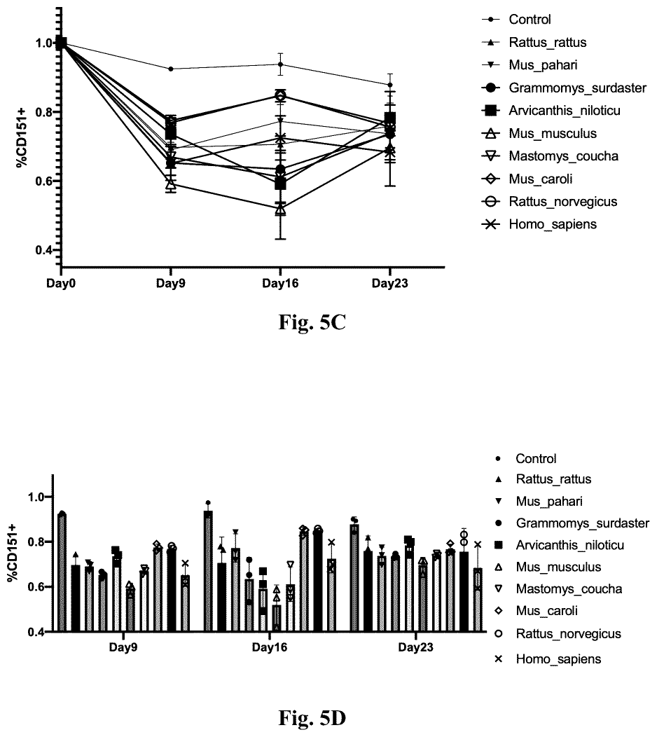

Different sets of versions of constructs targeting the CD151 gene and/or CD81 were generated according to the methods described above, and the structures of these constructs are depicted in FIG. 1B, FIG. 2B, FIG. 4B, and FIG. 5B. The constructs shown in FIG. 2E were generated as described: (1) Dnmt-dCas9+scFv-Krab, generated by fusions of Dnmt3A-Dnmt3L-dCas9-10×GCN4 (SEQ ID NOs: 151 and 179), T2A (SEQ ID NOs: 99 and 103) and scFv-Krab (SEQ ID NOs: 152 and 180); (2) dCas9+scFv-Dnmt-Krab, generated by fusions of dCas9-10×GCN4 (SEQ ID NOs: 153 and 181), T2A (SEQ ID NOs: 99 and 103) and scFv-Dnmt3A-Dnmt3L-Krab (SEQ ID NOs: 154 and 182); (3) dCas9+scFv-Dnmt+scFv-Krab, generated by fusions of dCas9-10×GCN4 (SEQ ID NOs: 153 and 181), T2A (SEQ ID NOs: 99 and 103), scFv-Dnmt3A-Dnmt3L (SEQ ID NOs: 200 and 201) and scFv-Krab (SEQ ID NOs: 202 and 203).

The repression of the constructs on CD151 and/or CD81 using different targeting sgRNAs (Table 4) was measured by FACS. The results are shown in FIG. 1C, FIG. 2C-2E, FIG. 4C-4F and FIG. 5C-5D. Editors with different effectors or conformations vary widely in repressing gene expressions, strongly suggesting that various modifications must be performed when optimizing epigenetic editors.

### Example 2: EPICAS Constructs for VEGFA Knockdown

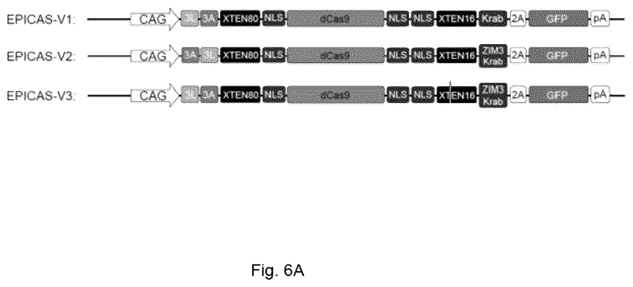

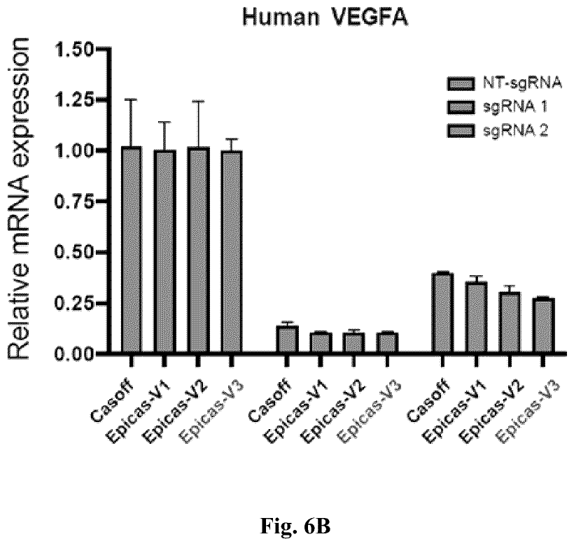

Three versions of EPICAS specific to VEGFA (EPICAS-V1, -V2, and -V3) were generated. The structure of these constructs is depicted in FIG. 6A. EPICAS-V1 comprises a KRAB domain, while V2 and V3 comprise a ZIM3-KRAB domain, and the orientation of DNMT3L and DNMT3A was reversed in V2 (3A-3L instead of 3L-3A as in V1 and V3).

The editing efficiency of the constructs on VEGFA using two different sgRNAs targeting human VEGFA (Table 4) was measured by qPCR. Results are shown in FIG. 6B. Decrease of mRNA expression with sgRNA1 was comparable across the constructs.

### Example 3: Constructs for In Vivo Epigenetic Modification of PCSK9 of Mice

The following four versions of constructs were generated for testing the editing efficiency in vivo experiment: (1) DNMT3A-DNMT3L-dCas9-KRAB (SEQ ID NO: 138 and 166); (2) DNMT3A-DNMT3L-dCas9-EZH2 (SEQ ID NO: 139 and 167); (3) DNMT3A-DNMT3L-dCas9-G9A (SEQ ID NO: 140 and 168); (4) DNMT3A-DNMT3L-dCas9-HDAC3 (SEQ ID NO: 144 and 172); (5) dCas9N-7-Dnmt3A-Dnmt3L-dCas9C-7-Krab (this construct is generated by fusions of dCas9N-7 (SEQ ID NO:14), Dnmt3A-Dnmt3L (SEQ ID NO:71), dCas9C-7 (SEQ ID NO:15) and Krab (SEQ ID NO:51)).

The DNA sequences corresponding to each version were transcribed in vitro to obtain mRNA and then mixed with two gRNAs targeting to PCSK9 (SEQ ID NOs: 198-199) in a 2:1:1 ratio to prepare LNP (molar ratio of LNP elements: MC3:cholesterol:DSPC:DMG-PEG2000=50:38.5:10:1.5), which was injected into wild-type C57 mice by tail vein injection. The injected dose was 4.5 mg (RNA mass) per kilogram of body weight per mouse. Blood sample was collected from mice on days 7, 14, and 21 of injection, and the expression of PCSK9 protein in blood was measured by Elisa, with comparison of mice without injection with LNP and mice injected with mRNA+control gRNA (sequence GAAGAGCCTGAGGCTCTTCT).

Results shows that the protein expression of PCSK9 in the serum of mice of experimental group was significantly lower than that of the control group at all three time points, with an approximate reduction of more than 50%.

What is claimed is:

1. A construct of Formula III: 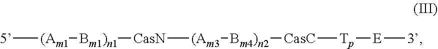
wherein:
one of A and B is a polynucleotide encoding DNMT3A; and the other of A and B is a polynucleotide encoding DNMT3L;
CasN is a polynucleotide encoding a N-terminal portion of dCas9;
CasC is a polynucleotide encoding a C-terminal portion of dCas9;
E is 5′-(Aₘ₅-Bₘ₆)ₙ₃-Kᵣ-D_{q}-3′;
K is a polynucleotide encoding KRAB;
D is a polynucleotide encoding a modulator of gene expression;
T comprises (a) a polynucleotide encoding an epitope capable of binding to an antibody or antigen binding fragment thereof, and further comprises (b) a polynucleotide encoding a self cleaving peptide at the 3′ end of the polynucleotide in (a), and at the 3′ end of the nucleotide encoding the self cleaving peptide in (b) further comprises (c) polynucleotide encoding an antibody or antigen binding fragment thereof that is capable of binding to the epitope;
m1, m2, m3, m4, m5, and m6 are each independently 1;
n2 is 0; n1 is 1 and n3 is 0; or n1 is 0 and n3 is 1;
p is 1;
q is 0;
r is 1.

2. The construct of claim 1, of Formula IIIb-1:

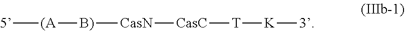

3. The construct of claim 1, of Formula IIIc-1:

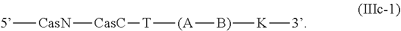

4. The construct of claim 1, wherein the self cleaving peptide is T2A.

5. The construct of claim 1, wherein the DNMT3A comprises the amino acid sequence of SEQ ID NO: 69.

6. The construct of claim 5, wherein the polynucleotide encoding the DNMT3A comprises the nucleic acid sequence of SEQ ID NO: 83.

7. The construct of claim 1, wherein the DNMT3L comprises the amino acid sequence of any one of SEQ ID NOs: 74-82.

8. The construct of claim 7, wherein the polynucleotide encoding the DNMT3L comprises the nucleic acid sequence of any one of SEQ ID NOs: 84-92.

9. The construct of claim 1, wherein the KRAB comprises the amino acid sequence of SEQ ID NO: 51, 53, or 230-241.

10. The construct of claim 9, wherein the polynucleotide encoding the KRAB comprises the nucleic acid sequence of SEQ ID NO: 52, 54, 206, 208, 210, 212, 214, 216, 218, 220, 222, 224, 226 or 228.

11. The construct of claim 1, wherein the dCas9 comprises a Staphylococcus pyogenes dCas9.

12. The construct of claim 11, wherein the dCas9 comprises the amino acid sequence of SEQ ID NO: 1.

13. The construct of claim 11, wherein the polynucleotide encoding the dCas9 comprises the nucleic acid sequence of SEQ ID NO: 26.

14. The construct of claim 1, wherein the epitope capable of binding to an antibody or an antigen binding fragment thereof is selected from a group consisting of GCN4, 10×GCN4.

15. The construct of claim 14, wherein the GCN4 comprises the amino acid sequence of SEQ ID NO: 97.

16. The construct of claim 15, wherein the polynucleotide encoding the GCN4 comprises the nucleic acid sequence of SEQ ID NO: 101.

17. The construct of claim 16, wherein the antibody or antigen binding fragment is a single domain antibody, a scFv, a Fab, a VH, a VHH or an antibody mimetic.

18. The construct of claim 17, wherein the antigen binding fragment is a scFv.

19. The construct of claim 1, comprising the nucleic acid sequence of any one of SEQ ID NOs:
162-189.

20. A polypeptide expressed by the construct of claim 1.


SEQUENCE LISTING (241 sequences)

SEQ ID NO: 1 - protein, 1368 aa, synthetic construct
  REGION 1..1368: note = dCas9
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 531 aa, synthetic construct
  REGION 1..531: note = dCas9N-1 531aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVT

SEQ ID NO: 3 - protein, 837 aa, synthetic construct
  REGION 1..837: note = dCas9C-1 531aa
EGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 768 aa, synthetic construct
  REGION 1..768: note = dCas9N-2 768aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQ

SEQ ID NO: 5 - protein, 600 aa, synthetic construct
  REGION 1..600: note = dCas9C-2 768aa
TTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 776 aa, synthetic construct
  REGION 1..776: note = dCas9N-3 776aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKN

SEQ ID NO: 7 - protein, 592 aa, synthetic construct
  REGION 1..592: note = dCas9C-3 776aa
SRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 8 - protein, 1009 aa, synthetic construct
  REGION 1..1009: note = dCas9N-4 1009aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFV

SEQ ID NO: 9 - protein, 359 aa, synthetic construct
  REGION 1..359: note = dCas9C-4 1009aa
YGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 10 - protein, 1047 aa, synthetic construct
  REGION 1..1047: note = dCas9N-5 1048-1063aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFK

SEQ ID NO: 11 - protein, 305 aa, synthetic construct
  REGION 1..305: note = dCas9C-5 1048-1063aa
ETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 12 - protein, 1073 aa, synthetic construct
  REGION 1..1073: note = dCas9N-6 1072aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIM

SEQ ID NO: 13 - protein, 296 aa, synthetic construct
  REGION 1..296: note = dCas9C-6 1072aa
VWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 14 - protein, 1246 aa, synthetic construct
  REGION 1..1246: note = dCas9N-7 1246aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLK

SEQ ID NO: 15 - protein, 122 aa, synthetic construct
  REGION 1..122: note = dCas9C-7 1246aa
GSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 16 - protein, 1248 aa, synthetic construct
  REGION 1..1248: note = dCas9N-8 1248aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGS

SEQ ID NO: 17 - protein, 120 aa, synthetic construct
  REGION 1..120: note = dCas9C-8 1248aa
PEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 18 - protein, 1253 aa, synthetic construct
  REGION 1..1253: note = dCas9N-9 1253aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNE

SEQ ID NO: 19 - protein, 115 aa, synthetic construct
  REGION 1..115: note = dCas9C-9 1253aa
QKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 20 - protein, 1260 aa, synthetic construct
  REGION 1..1260: note = dCas9N-10 1260aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVE

SEQ ID NO: 21 - protein, 108 aa, synthetic construct
  REGION 1..108: note = dCas9C-10 1260aa
QHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 22 - protein, 1263 aa, synthetic construct
  REGION 1..1263: note = dCas9N-11 1263aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHK

SEQ ID NO: 23 - protein, 105 aa, synthetic construct
  REGION 1..105: note = dCas9C-11 1263aa
HYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 24 - protein, 1276 aa, synthetic construct
  REGION 1..1276: note = dCas9N-12 1276aa
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEF

SEQ ID NO: 25 - protein, 92 aa, synthetic construct
  REGION 1..92: note = dCas9C-12 1276aa
SKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 26 - DNA, 4104 bp, synthetic construct
  misc_feature 1..4104: note = dCas9
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 27 - DNA, 1593 bp, synthetic construct
  misc_feature 1..1593: note = dCas9N-1 531aa
  source 1..1593: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgacc

SEQ ID NO: 28 - DNA, 2511 bp, synthetic construct
  misc_feature 1..2511: note = dCas9C-1 531aa
  source 1..2511: mol_type = other DNA
gagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 29 - DNA, 2305 bp, synthetic construct
  misc_feature 1..2305: note = dCas9N-2 768aa
  source 1..2305: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccaga

SEQ ID NO: 30 - DNA, 1800 bp, synthetic construct
  misc_feature 1..1800: note = dCas9C-2 768aa
  source 1..1800: mol_type = other DNA
accacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 31 - DNA, 2329 bp, synthetic construct
  misc_feature 1..2329: note = dCas9N-3 776aa
  source 1..2329: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaaca

SEQ ID NO: 32 - DNA, 1776 bp, synthetic construct
  misc_feature 1..1776: note = dCas9C-3 776aa
  source 1..1776: mol_type = other DNA
agccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 33 - DNA, 3027 bp, synthetic construct
  misc_feature 1..3027: note = dCas9N-4 1009aa
  source 1..3027: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtg

SEQ ID NO: 34 - DNA, 1077 bp, synthetic construct
  misc_feature 1..1077: note = dCas9C-4 1009aa
  source 1..1077: mol_type = other DNA
tacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 35 - DNA, 3142 bp, synthetic construct
  misc_feature 1..3142: note = dCas9N-5 1048-1063aa
  source 1..3142: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaaga

SEQ ID NO: 36 - DNA, 915 bp, synthetic construct
  misc_feature 1..915: note = dCas9C-5 1048-1063aa
  source 1..915: mol_type = other DNA
gagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 37 - DNA, 3219 bp, synthetic construct
  misc_feature 1..3219: note = dCas9N-6 1072aa
  source 1..3219: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcatg

SEQ ID NO: 38 - DNA, 888 bp, synthetic construct
  misc_feature 1..888: note = dCas9C-6 1072aa
  source 1..888: mol_type = other DNA
gtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 39 - DNA, 3738 bp, synthetic construct
  misc_feature 1..3738: note = dCas9N-7 1246aa
  source 1..3738: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaag

SEQ ID NO: 40 - DNA, 366 bp, synthetic construct
  misc_feature 1..366: note = dCas9C-7 1246aa
  source 1..366: mol_type = other DNA
ggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 41 - DNA, 3744 bp, synthetic construct
  misc_feature 1..3744: note = dCas9N-8 1248aa
  source 1..3744: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcc

SEQ ID NO: 42 - DNA, 360 bp, synthetic construct
  misc_feature 1..360: note = dCas9C-8 1248aa
  source 1..360: mol_type = other DNA
cccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 43 - DNA, 3759 bp, synthetic construct
  misc_feature 1..3759: note = dCas9N-9 1253aa
  source 1..3759: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgag

SEQ ID NO: 44 - DNA, 345 bp, synthetic construct
  misc_feature 1..345: note = dCas9C-9 1253aa
  source 1..345: mol_type = other DNA
cagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 45 - DNA, 3780 bp, synthetic construct
  misc_feature 1..3780: note = dCas9N-10 1260aa
  source 1..3780: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaa

SEQ ID NO: 46 - DNA, 324 bp, synthetic construct
  misc_feature 1..324: note = dCas9C-10 1260aa
  source 1..324: mol_type = other DNA
cagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 47 - DNA, 3789 bp, synthetic construct
  misc_feature 1..3789: note = dCas9N-11 1263aa
  source 1..3789: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaa

SEQ ID NO: 48 - DNA, 315 bp, synthetic construct
  misc_feature 1..315: note = dCas9C-11 1263aa
  source 1..315: mol_type = other DNA
cactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 49 - DNA, 3828 bp, synthetic construct
  misc_feature 1..3828: note = dCas9N-12 1276aa
  source 1..3828: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttc

SEQ ID NO: 50 - DNA, 276 bp, synthetic construct
  misc_feature 1..276: note = dCas9C-12 1276aa
  source 1..276: mol_type = other DNA
tccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 51 - protein, 62 aa, synthetic construct
  REGION 1..62: note = KRAB
RTLVTFKDVFVDFTREEWKLLDTAQQIVYRNVMLENYKNLVSLGYQLTKPDVILRLEKGEEP

SEQ ID NO: 52 - DNA, 186 bp, synthetic construct
  misc_feature 1..186: note = KRAB
  source 1..186: mol_type = other DNA
cggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 53 - protein, 100 aa, synthetic construct
  REGION 1..100: note = ZIM3 KRAB
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 54 - DNA, 300 bp, synthetic construct
  misc_feature 1..300: note = ZIM3 KRAB
  source 1..300: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctc

SEQ ID NO: 55 - protein, 751 aa, synthetic construct
  REGION 1..751: note = EZH2
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 56 - DNA, 2256 bp, synthetic construct
  misc_feature 1..2256: note = EZH2
  source 1..2256: mol_type = other DNA
atgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttga

SEQ ID NO: 57 - protein, 1233 aa, synthetic construct
  REGION 1..1233: note = G9A EHMT2
MRGLPRGRGLMRARGRGRAAPPGSRGRGRGGPHRGRGRPRSLLSLPRAQASWTPQLSTGLTSPPVPCLPSQGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 58 - DNA, 3702 bp, synthetic construct
  misc_feature 1..3702: note = G9A EHMT2
  source 1..3702: mol_type = other DNA
atgcggggtctaccgagagggagggggttgatgcgggcccgggggaggggtcgtgcggcccctccgggcagccgaggccgcggaaggggggggccccacagaggaagaggtaggccccggagcctactctctcttcccagggcccaggcatcctggaccccccaactctctactgggctgaccagccctcctgtcccttgtctcccctcccagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatga

SEQ ID NO: 59 - protein, 876 aa, synthetic construct
  REGION 1..876: note = LSD1 KDM1A
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGQAGGLQDDSSGGYGDGQASGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQADTVKVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 60 - DNA, 2631 bp, synthetic construct
  misc_feature 1..2631: note = LSD1 KDM1A
  source 1..2631: mol_type = other DNA
atgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcggggcaagcaggaggacttcaagacgacagttctggagggtatggagacggccaagcatcaggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgacactgtcaaggttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtga

SEQ ID NO: 61 - protein, 191 aa, synthetic construct
  REGION 1..191: note = HP1 CBX5
MGKKTKRTADSSSSEDEEEYVVEKVLDRRVVKGQVEYLLKWKGFSEEHNTWEPEKNLDCPELISEFMKKYKKMKEGENNKPREKSESNKRKSNFSNSADDIKSKKKREQSNDIARGFERGLEPEKIIGATDSCGDLMFLMKWKDTDEADLVLAKEANVKCPQIVIAFYEERLTWHAYPEDAENKEKETAKS

SEQ ID NO: 62 - DNA, 576 bp, synthetic construct
  misc_feature 1..576: note = HP1 CBX5
  source 1..576: mol_type = other DNA
atgggaaagaaaaccaagcggacagctgacagttcttcttcagaggatgaggaggagtatgttgtggagaaggtgctagacaggcgcgtggttaagggacaagtggaatatctactgaagtggaaaggcttttctgaggagcacaatacttgggaacctgagaaaaacttggattgccctgagctaatttctgaatttatgaaaaagtataagaagatgaaggagggtgaaaataataaacccagggagaagtcagaaagtaacaagaggaaatccaatttctcaaacagtgccgatgacatcaaatctaaaaaaaagagagagcagagcaatgatatcgctcggggctttgagagaggactggaaccagaaaagatcattggggcaacagattcctgtggtgatttaatgttcctaatgaaatggaaagacacagatgaagctgacctggttcttgcaaaagaagctaatgtgaaatgtccacaaattgtgatagcattttatgaagagagactgacatggcatgcatatcctgaggatgcggaaaacaaagagaaagaaacagcaaagagctaa

SEQ ID NO: 63 - protein, 1052 aa, synthetic construct
  REGION 1..1052: note = FOG1 ZFPM1
MDAGPTVQGGPTRSSRAARAGGGLSPGAGGSRWERDTGSAALLWQEWNPWAWWRPRVTAGSLGDMEAREEVQLVGASHMEQKATAPEAPSPPSADVNSPPPLPPPTSPGGPKELEGQEPEPRPTEEEPGSPWSGPDELEPVVQDGQRRIRARLSLATGLSWGPFHGSVQTRASSPRQAEPSPALTLLLVDEACWLRTLPQALTEAEANTEIHRKDDALWCRVTKPVPAGGLLSVLLTAEPHSTPGHPVKKEPAEPTCPAPAHDLQLLPQQAGMASILATAVINKDVFPCKDCGIWYRSERNLQAHLLYYCASRQGTGSPAAAATDEKPKETYPNERVCPFPQCRKSCPSASSLEIHMRSHSGERPFVCLICLSAFTTKANCERHLKVHTDTLSGVCHSCGFISTTRDILYSHLVTNHMVCQPGSKGEIYSPGAGHPATKLPPDSLGSFQQQHTALQGPLASADLGLAPTPSPGLDRKALAEATNGEARAEPLAQNGGSSEPPAAPRSIKVEAVEEPEAAPILGPGEPGPQAPSRTPSPRSPAPARVKAELSSPTPGSSPVPGELGLAGALFLPQYVFGPDAAPPASEILAKMSELVHSRLQQGAGAGAGGAQTGLFPGAPKGATCFECEITFSNVNNYYVHKRLYCSGRRAPEDAPAARRPKAPPGPARAPPGQPAEPDAPRSSPGPGAREEGAGGAATPEDGAGGRGSEGSQSPGSSVDDAEDDPSRTLCEACNIRFSRHETYTVHKRYYCASRHDPPPRRPAAPPGPPGPAAPPAPSPAAPVRTRRRRKLYELHAAGAPPPPPPGHAPAPESPRPGSGSGSGPGLAPARSPGPAADGPIDLSKKPRRPLPGAPAPALADYHECTACRVSFHSLEAYLAHKKYSCPAAPPPGALGLPAAACPYCPPNGPVRGDLLEHFRLAHGLLLGAPLAGPGVEARTPADRGPSPAPAPAASPQPGSRGPRDGLGPEPQEPPPGPPPSPAAAPEAVPPPPAPPSYSDKGVQTPSKGTPAPLPNGNHRYCRLCNIKFSSLSTFIAHKKYYCSSHAAEHVK

SEQ ID NO: 64 - DNA, 3159 bp, synthetic construct
  misc_feature 1..3159: note = FOG1 ZFPM1
  source 1..3159: mol_type = other DNA
atggacgctgggcctacagtacagggagggcccactcgcagctccagggctgccagagcaggtgggggcctgtcccctggtgctggggggagcaggtgggaaagagacacggggtccgcggcccttctctggcaagagtggaatccgtgggcgtggtggaggccaagggtcacggcaggttccctcggagacatggaggccagagaggaggtgcagttggtgggtgccagccacatggagcaaaaggccacggcacctgaagccccgagccctcccagcgcagatgttaactcacccccaccgctgccgccccccacatccccaggaggccccaaggagctggaaggacaggaaccagaacccaggcccacggaggaagagccgggcagtccctggagcgggccagacgagctggagccggtggtgcaggatgggcagaggcgcatacgggcccgactcagcctcgccacgggcctgtcctggggcccgttccatgggagtgtccagaccagagcctcatcccccaggcaggcggagccgagcccagccctgaccctgctgctggtggacgaggcctgctggctgaggacgctgccccaggccctgactgaggccgaggccaacacagagatccacaggaaggatgacgcactctggtgcagggtcaccaagccggtgcctgcggggggactcctgagcgtgctcctcacggccgagccccacagcacccccggccaccctgtgaagaaggagccagcagagcccacgtgcccggcccctgcacacgacctccagctcctgccccagcaggccgggatggcctccatccttgccaccgcagtgatcaacaaagacgtcttcccctgcaaggactgtggcatctggtaccgcagcgagcgcaacctgcaggcgcacctgctctactactgcgccagccgccagggcaccggctccccggccgcagccgccacagacgagaagcccaaagagacctaccccaacgagcgcgtctgccccttcccccagtgccgcaagagctgccccagcgccagctccctggagatccacatgcgcagccacagcggagagcggcccttcgtgtgcctgatctgcctgtcggccttcaccaccaaggccaactgcgagcggcacctcaaggtgcacacggacacgctgagcggtgtctgccacagctgtggcttcatctccaccacaagggacatcctctacagccacttggtcaccaaccacatggtctgccagcctggctccaagggtgagatctactcgccaggggccggacacccagcaaccaagctgcccccagacagtctgggcagcttccagcagcagcacacggccctgcaaggccccctggcctccgcggacctgggcctggcgcccaccccatcgccaggactggacagaaaggccctggccgaggccaccaacggagaggccagagcggagcctctggcccagaatggaggcagcagcgagcccccggcggcccccaggagcatcaaggtggaggcggtggaggagccggaggcggcccccatcctgggccccggagagcctgggccccaggccccgtcgcggacgccgtcgccgcgcagccccgccccggccagggtcaaggccgagctgtccagccccacgccgggctccagcccggtgcccggcgagctgggcctggccggggccctgttccttccgcagtacgtgttcgggcccgacgcggcgccccccgcctcggagatcctggccaagatgtccgagctggtgcacagccggctgcagcagggcgcgggcgcgggcgccggcggcgcgcagaccgggctcttccccggggcccccaagggcgctacgtgcttcgagtgcgagatcaccttcagcaacgtcaacaactactacgtgcacaagcgcctctactgttcaggccgccgtgcgcccgaggacgcgcctgccgcgcgcaggcccaaggcgccccccggcccggcccgcgcgccccccggccagcccgccgaacccgacgcgccgcgctcgtccccgggccccggagcgcgcgaggagggggctgggggcgcggccacgcccgaggacggcgcgggcggccggggcagcgagggcagccagagcccgggtagctccgtggacgacgcggaggacgaccccagccgcacgctgtgcgaggcctgcaacatccgcttcagccgccacgagacctacaccgtgcacaagcggtactactgcgcctcgcgccacgacccgccgccgcgccgaccggccgcgcccccgggaccccctgggccggccgcgcccccggccccctctcccgccgcgcctgtgcgcacgcgcagacgccgcaagctctacgagctgcacgcggccggcgccccgccccccccgccgcccggccacgcccccgcgcccgagtcgccgcggcccggaagcggaagcggaagcggccccggcctcgcccctgcgcgctcgcccggccccgcggccgacggccccatcgacctgagcaagaagccgcggcgcccgctccccggagccccggcaccggcgctggccgactaccacgagtgcacggcctgccgcgtgagcttccacagcctcgaggcctacctggcgcacaagaagtactcgtgccccgctgcgccaccgcccggcgcgctcggcctgcccgccgccgcctgcccctactgccccccgaacggcccggtgcgcggggacctgctggagcatttccgcctggcgcacggcctgctgctcggcgcgcccctggccggcccgggggtcgaggcccggacgccggccgaccgcggcccctcgcccgctcccgcccccgccgcctccccgcagcccgggtcccgtggcccccgggacggcctcggcccggagccccaggagccgccgcccggcccgcccccgtccccggccgccgcgcccgaggccgtgccgcccccgccggcgcccccctcctactcggacaagggcgtccagactcccagcaagggcacgccggcgccgctgcccaacggcaaccaccggtactgccgtctttgcaacatcaagttcagcagcctgtccaccttcatcgcccacaagaagtattactgctcctcgcacgccgccgagcacgtgaagtga

SEQ ID NO: 65 - protein, 434 aa, synthetic construct
  REGION 1..434: note = HDAC3
MAKTVAYFYDPDVGNFHYGAGHPMKPHRLALTHSLVLHYGLYKKMIVFKPYQASQHDMCRFHSEDYIDFLQRVSPTNMQGFTKSLNAFNVGDDCPVFPGLFEFCSRYTGASLQGATQLNNKICDIAINWAGGLHHAKKFEASGFCYVNDIVIGILELLKYHPRVLYIDIDIHHGDGVQEAFYLTDRVMTVSFHKYGNYFFPGTGDMYEVGAESGRYYCLNVPLRDGIDDQSYKHLFQPVINQVVDFYQPTCIVLQCGADSLGCDRLGCFNLSIRGHGECVEYVKSFNIPLLVLGGGGYTVRNVARCWTYETSLLVEEAISEELPYSEYFEYFAPDFTLHPDVSTRIENQNSRQYLDQIRQTIFENLKMLNHAPSVQIHDVPADLLTYDRTDEADAEERGPEENYSRSGSRDFSLLSIGLLGRFKREKVNGKVLV

SEQ ID NO: 66 - DNA, 1305 bp, synthetic construct
  misc_feature 1..1305: note = HDAC3
  source 1..1305: mol_type = other DNA
atggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccaacatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggtctgggagcagggacttcagcctactttcaataggcttgctggggaggtttaaaagggaaaaagtaaatgggaaggtattagtttaa

SEQ ID NO: 67 - protein, 1738 aa, synthetic construct
  REGION 1..1738: note = DOT1L
MGEKLELRLKSPVGAEPAVYPWPLPVYDKHHDAAHEIIETIRWVCEEIPDLKLAMENYVLIDYDTKSFESMQRLCDKYNRAIDSIHQLWKGTTQPMKLNTRPSTGLLRHILQQVYNHSVTDPEKLNNYEPFSPEVYGETSFDLVAQMIDEIKMTDDDLFVDLGSGVGQVVLQVAAATNCKHHYGVEKADIPAKYAETMDREFRKWMKWYGKKHAEYTLERGDFLSEEWRERIANTSVIFVNNFAFGPEVDHQLKERFANMKEGGRIVSSKPFAPLNFRINSRNLSDIGTIMRVVELSPLKGSVSWTGKPVSYYLHTIDRTILENYFSSLKNPKLREEQEAARRRQQRESKSNAATPTKGPEGKVAGPADAPMDSGAEEEKAGAATVKKPSPSKARKKKLNKKGRKMAGRKRGRPKKMNTANPERKPKKNQTALDALHAQTVSQTAASSPQDAYRSPHSPFYQLPPSVQRHSPNPLLVAPTPPALQKLLESFKIQYLQFLAYTKTPQYKASLQELLGQEKEKNAQLLGAAQQLLSHCQAQKEEIRRLFQQKLDELGVKALTYNDLIQAQKEISAHNQQLREQSEQLEQDNRALRGQSLQLLKARCEELQLDWATLSLEKLLKEKQALKSQISEKQRHCLELQISIVELEKSQRQQELLQLKSCVPPDDALSLHLRGKGALGRELEPDASRLHLELDCTKFSLPHLSSMSPELSMNGQAAGYELCGVLSRPSSKQNTPQYLASPLDQEVVPCTPSHVGRPRLEKLSGLAAPDYTRLSPAKIVLRRHLSQDHTVPGRPAASELHSRAEHTKENGLPYQSPSVPGSMKLSPQDPRPLSPGALQLAGEKSSEKGLRERAYGSSGELITSLPISIPLSTVQPNKLPVSIPLASVVLPSRAERARSTPSPVLQPRDPSSTLEKQIGANAHGAGSRSLALAPAGFSYAGSVAISGALAGSPASLTPGAEPATLDESSSSGSLFATVGSRSSTPQHPLLLAQPRNSLPASPAHQLSSSPRLGGAAQGPLPEASKGDLPSDSGFSDPESEAKRRIVFTITTGAGSAKQSPSSKHSPLTASARGDCVPSHGQDSRRRGRRKRASAGTPSLSAGVSPKRRALPSVAGLFTQPSGSPLNLNSMVSNINQPLEITAISSPETSLKSSPVPYQDHDQPPVLKKERPLSQTNGAHYSPLTSDEEPGSEDEPSSARIERKIATISLESKSPPKTLENGGGLAGRKPAPAGEPVNSSKWKSTFSPISDIGLAKSADSPLQASSALSQNSLFTFRPALEEPSADAKLAAHPRKGFPGSLSGADGLSPGTNPANGCTFGGGLAADLSLHSFSDGASLPHKGPEAAGLSSPLSFPSQRGKEGSDANPFLSKRQLDGLAGLKGEGSRGKEAGEGGLPLCGPTDKTPLLSGKAAKARDREVDLKNGHNLFISAAAVPPGSLLSGPGLAPAASSAGGAASSAQTHRSFLGPFPPGPQFALGPMSLQANLGSVAGSSVLQSLFSSVPAAAGLVHVSSAATRLTNSHAMGSFSGVAGGTVGGVFNHAVPSASAHPFGARVGRGAACGSATLGPSPLQAAASASASSFQAPASVETRPPPPPPPPPPPLPPPAHLGRSPAGPPVLHAPPPPNAALPPPPTLLASNPEPALLQSLASLPPNQAFLPPTSAASLPPANASLSIKLTSLPHKGARPSFTVHHQPLPRLALAQAAPGIPQASATGPSAVWVSLGMPPPYAAHLSGVKPR

SEQ ID NO: 68 - DNA, 5217 bp, synthetic construct
  misc_feature 1..5217: note = DOT1L
  source 1..5217: mol_type = other DNA
atgggggagaagctggagctgagactgaagtcgcccgtgggggctgagcccgccgtctacccgtggccgctgccggtctacgataaacatcacgatgctgctcatgaaatcatcgagaccatccgatgggtctgtgaagaaatcccggatctcaagctcgctatggagaattacgttttaattgactatgacaccaaaagcttcgagagcatgcagaggctctgcgacaagtacaaccgtgccatcgacagcatccaccagctgtggaagggcaccacgcagcccatgaagctgaacacgcggccgtccactggactcctgcgccatatcctgcagcaggtctacaaccactcggtgaccgaccccgagaagctcaacaactacgagcccttctcccccgaggtgtacggggagacctccttcgacctggtggcccagatgattgatgagatcaagatgaccgacgacgacctgtttgtggacttggggagcggtgtgggccaggtcgtgctccaggttgctgctgccaccaactgcaaacatcactatggcgtcgagaaagcagacatcccggccaagtatgcggagaccatggaccgcgagttcaggaagtggatgaaatggtatggaaaaaagcatgcagaatacacattggagagaggcgatttcctctcagaagagtggagggagcgaatcgccaacacgagtgttatatttgtgaataattttgcctttggtcctgaggtggatcaccagctgaaggagcggtttgcaaacatgaaggaaggtggcagaatcgtgtcctcgaaaccctttgcacctctgaacttcagaataaacagtagaaacttgagtgacatcggcaccatcatgcgcgtggtggagctctcgcccctgaagggctcggtgtcgtggacggggaagccagtctcctactacctgcacactatcgaccgcaccatacttgaaaactatttttctagtctgaaaaacccaaaactcagggaggaacaggaggcagcccggcgccgccagcagcgcgagagcaagagcaacgcggccacgcccactaagggcccagagggcaaggtggccggccccgccgacgcccccatggactctggtgctgaggaagagaaggcgggagcagccaccgtgaagaagccgtctccctccaaagcccgcaagaagaagctaaacaagaaggggaggaagatggctggccgcaagcgcgggcgccccaagaagatgaacactgcgaaccccgagcggaagcccaagaagaaccaaactgcactggatgccctgcacgctcagaccgtgtctcagacggcggcctcctcaccccaggatgcctacagatcccctcacagcccgttctaccagctacctccgagcgtgcagcggcactcccccaacccgctgctggtggcgcccaccccgcccgcgctgcagaagcttctagagtccttcaagatccagtacctgcagttcctggcatacacaaagaccccccagtacaaggccagcctgcaggagctgctgggccaggagaaggagaagaacgcccagctcctgggtgcggctcagcagctcctcagccactgccaggcccagaaggaggagatcaggaggctgtttcagcaaaaattggatgagctgggtgtgaaggcgctgacctacaacgacctgattcaagcgcagaaggagatctccgcccataaccagcagctgcgggagcagtcggagcagctggagcaggacaaccgcgcgctccgcggccagagcttgcagctgctcaaggctcgctgcgaggagctgcagctggactgggccacgctgtcgctggagaagctgttgaaggagaagcaggccctgaagagccagatctcggagaagcagaggcactgcctggagctgcagatcagcattgtggagctagagaagagccagcggcagcaggagctcctgcagctcaagtcctgtgtgccgcctgacgacgccctgtccctgcacctgcgtgggaagggcgccctgggccgcgagctggagcctgacgccagccggctgcacctggagctggactgcaccaagttctcgctgcctcacttgagcagcatgagcccggagctctccatgaacggccaggctgctggctatgagctctgcggtgtgctgagccggccttcgtcgaagcagaacacgccccagtacctggcctcacccctggaccaggaggtggtgccctgtacccctagccacgtcggccggccgcgcctggagaagctgtctggcctagccgcacccgactacactaggctgtccccggccaagattgtgctgaggcggcacctgagccaggaccacacggtgcccggcaggccggctgccagtgagctgcattcgagagctgagcacaccaaggagaacggccttccctaccagagccccagcgtgcctggcagcatgaagctgagccctcaggacccgcggcccctgtcccctggggccttgcagcttgctggagagaagagcagtgagaagggcctgagagagcgcgcctacggcagcagcggggagctcatcaccagcctgcccatcagcatcccgctcagcaccgtgcagcccaacaagctcccggtcagcattcccctggccagcgtggtgctgcccagccgcgccgagagggcgaggagcacccccagtcccgtgctgcagccccgtgacccctcgtccacacttgaaaagcagattggtgctaatgcccacggtgctgggagcagaagccttgccctggcccccgcaggcttctcctacgctggctcggtggccatcagcggggccttggcgggcagcccggcctctctcacacctggagccgagccggccaccttggatgagtcctccagctctgggagcctttttgccaccgtggggtcccgcagctccacgccacagcaccccctgctgctggcacagccccggaactcgcttcctgcctctcccgcccaccagctctcctccagtccccggcttggtggggccgcccagggcccgttgcccgaggccagcaagggagacctgccctccgattccggcttctcagatcctgagagtgaagccaagaggaggattgtgttcaccatcaccactggtgcgggcagtgccaagcagtcgccctccagcaagcacagccccctgaccgccagcgcccgtggggactgtgtgccgagccacgggcaggacagtcgcaggcgcggccggcggaagcgagcatctgcggggacgcccagcttgagcgcaggcgtgtcccccaagcgccgagccctgccgtccgtcgctggccttttcacacagccttcggggtctcccctcaacctcaactccatggtcagtaacatcaaccagcccctggagattacagccatctcgtccccggagacctccctgaagagctcccctgtgccctaccaggaccacgaccagccccccgtgctcaagaaggagcggcctctgagccagaccaatggggcacactactccccactcacctcagacgaggagccaggctctgaggacgagcccagcagtgctcgaattgagagaaaaattgcaacaatctccttagaaagcaaatctcccccgaaaaccttggaaaatggtggtggcttggcgggaaggaagcccgcgcccgccggcgagccagtcaatagcagcaagtggaagtccaccttctcgcccatctccgacatcggcctggccaagtcggcggacagcccgctgcaggccagctccgccctcagccagaactccctgttcacgttccggcccgccctggaggagccctctgccgatgccaagctggccgctcaccccaggaaaggctttcccggctccctgtcgggggctgacggactcagcccgggcaccaaccctgccaacggctgcaccttcggcgggggcctggccgcggacctgagtttacacagcttcagtgatggtgcttctcttccccacaagggccccgaggcggccggcctgagctccccgctgagcttcccctcgcagcgcggcaaggagggctcggacgccaaccctttcctgagcaagaggcagctggacggcctggctgggctgaagggcgagggcagccgcggcaaggaggcaggggagggcggcctaccgctgtgcgggcccacggacaagaccccactgctgagcggcaaggccgccaaggcccgggaccgcgaggtcgacctcaagaatggccacaacctcttcatctctgcggcggccgtgcctcccggaagcctcctcagcggccccggcctggccccggcggcgtcctccgcaggcggcgcggcgtcctccgcccagacgcaccggtccttcctgggccccttcccgccgggaccgcagttcgcgctcggccccatgtccctgcaggccaacctcggctccgtggccggctcctccgtgctgcagtcgctgttcagctctgtgccggccgccgcaggcctggtgcacgtgtcgtccgctgccaccagactgaccaactcgcacgccatgggcagcttttccggggtggcaggcggcacagttggaggtgtctttaaccacgcggtgccctccgcctctgctcatccgtttggagcccgtgtcggccgcggggctgcatgtggcagcgccacactgggcccgagcccgctgcaggcggcggccagcgcctcggcctcttcctttcaggccccggcctcggttgagacccggccgccccctccgcctccgcctccgcctcccccgctgcccccgcctgcgcacctgggccggtcccccgcggggccgcccgtcctccacgccccccctccacctaacgccgccttgcctcctcccccaacgctgctggcctctaaccctgagcccgcgcttctgcagagcctcgcgtccctcccgcctaaccaagctttcttgccccccacctctgctgcctctctgccgcctgctaacgcctctttgtctatcaagctcacctccctcccgcacaagggcgcccgcccctccttcacggtgcaccaccagcccctgccccggctggccctggcccaggccgcgcccgggatcccacaggccagcgccacggggccgtccgcggtgtgggtgtccctcggcatgccgcctccctatgccgcgcacctttcgggggttaagccgcgataa

SEQ ID NO: 69 - protein, 302 aa, synthetic construct
  REGION 1..302: note = DNMT3A
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 70 - protein, 215 aa, synthetic construct
  REGION 1..215: note = DNMT3L
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 71 - protein, 544 aa, synthetic construct
  REGION 1..544: note = DNMT3A-DNMT3L fusion peptide
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 72 - protein, 16 aa, synthetic construct
  REGION 1..16: note = NLS
APKKKRKVGIHGVPAA

SEQ ID NO: 73 - protein, 16 aa, synthetic construct
  REGION 1..16: note = NLS
KRPAATKKAGQAKKKK

SEQ ID NO: 74 - protein, 387 aa, synthetic construct
  REGION 1..387: note = Homo sapiens DNMT3L
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSSRHWALVSEEELSLLAQNKQSSKLAAKWPTKLVKNCFLPLREYFKYFSTELTSSL

SEQ ID NO: 75 - protein, 215 aa, synthetic construct; identical to SEQ ID NO: 70
  REGION 1..215: note = Mus musculus DNMT3L

SEQ ID NO: 76 - protein, 415 aa, synthetic construct
  REGION 1..415: note = Mus caroli DNMT3L
MGSRETPSSFSKTLETLDLETSDSSSPDADSPLEEQWLKSSPALKEDNVDMVLEDCKEPLSPSSPPTGREMIRYEVKVNRRSIEDICLCCGTLQVYTQHPLFEGGICAPCKDKFLESLFLYDDDGHQSYCTICCSGGTLFICESPDCTRCYCFECVDILVGPGTSERINAMACWVCFLCLPFSRSGLLQRRKRWRHQLKAFHDQEGAGPMEIYKTVSTWKRQPVRVLSLFGNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLMTEDDQETTARFLQTEAVTLQDVRGRDYQNVMRVWSNIPGLKSKHVPLTPKEEEYLQAQVRTRSKLDAQKVDLLVKNCLLPLREYFKYFS

SEQ ID NO: 77 - protein, 422 aa, synthetic construct
  REGION 1..422: note = Mus Pahari DNMT3L
MGSRETPSSCSKTFETVNLETSDSSNPDADSPLEEQWLKSSPDLKEEDSVDVVLEDCEEPLSPSSPPEGREIIRYEVKVNQRNIEDICLGCGTLQVYTQHPLFEGGICALCKDKFLESLFLYDDDGHQSYCTICCSGGTLFICESPDCTRCYCFECVDILVAPGTSERINAMACWVCFLCLPFSRSGLLQRRKRWRHQLKAFHDQEVAGPIEIYKTVSAWKRQPVRVLSLFGNIDKVLKSLGFLESGSGSEGGMLKYVEDVTNVVRRDVEKWGPFDLVYGSTQPLGSSCDRCPGWYMFQFHRILQYALPRQESQRPFFWIFMDNLLLTEDDQETTVRFLQTETVTLQYVRGRVLQNAVRVWSNIPGLKSKHAVLTPQEEETLQAQIRTRNKPDTQKVDPLVKSCLLPLREYFKYFSQNSLPL

SEQ ID NO: 78 - protein, 422 aa, synthetic construct
  REGION 1..422: note = Rattus norvegicus DNMT3L
MGSRETPSSCSKTHETLNLETPESSSTDPDSPLEEQWPKSSPDLKEEDSVDMVLEDSKEPLTPSSPPTGREVIRYEVNVNQRNIEDICLCCGSLQVYAQHPLFEGGICAPCKDKFLETLFLYDEDGHQSYCTICCSGHTLFICESPDCTRCYCFECVDILVGPGTSERINAMACWVCFLCLPFSRSGLLQRRKKWRHQLKAFHDREGASPVEIYKTVSAWKRQPVRVLSLFGNIDKELKSLGFLESSSGSEGGTLKYVEDVTNVVRREVEKWGPFDLVYGSTQPLGYSCDRCPGWYMFQFHRILQYARPRQDSQQPFFWIFVDNLLLTEDDQETTVRFLQTEAVTLQDVRGRVLQNAMRVWSNIPGLKSKHADLTPKEEQSLQTQVRTRSKLAAQKVDSLVKYCLLPLREYFKYFSQNSLPL

SEQ ID NO: 79 - protein, 422 aa, synthetic construct
  REGION 1..422: note = Rattus Rattus DNMT3L
MGSRETPSSCSKTHETLNLETPESSSTDPDSPLEEQWPKSSPDLKEEDSVDMVLEDSKEPLTPSSPPTGREIIRYEVNVNQRNIEDICLCCGSLQVYAQHPLFEGGICAPCKDKFLETLFLYDEDGHQSYCTICCSGHTLFICESPDCTRCYCFECVDILVGPGTSERINAMACWVCFLCLPFSRSGLLQRRKKWRHQLKAFHDREGASPVEIYKTVSAWKRQPVRVLSLFGNIDKELKSLGFLESGSGSEGGTLKYVEDVTNVVRREVEKWGPFDLVYGSTQPLGYSCDRCPGWYMFQFHRILQYARPRQDSQQPFFWIFVDNLLLTEDDQETTVRFLQTEAVTLQDVRGRVLQNAVRVWSNIPGLKSKHADLTPKEEQSLRTQVRTRSKLAAQKVDSLVKYCLLPLREYFKYFSQNSLPL

SEQ ID NO: 80 - protein, 425 aa, synthetic construct
  REGION 1..425: note = Arvicanthis niloticus DNMT3L
MGSRETPSSCSKVLETLNLETSDSSSPDPESPLEEQWLESPPDREEEDDDDSVIVVLEDSKEPLSPSSRPTGREVIRYEVNVNQRNIEDICLCCGTLQVYTKHPLFEGGLCVSCKDKFLESLFLYDEDGHQSYCTICCSGGTLFICESPDCTRCYCFECVDILVGPGTSERINAMACWVCFLCLPFSQSGLLQRRKKWRHQLKAFHDREGASPMEIYKTVSAWKRQPVRVLSLFGNIEKELKSLGFLEIGSDSEGGTLKYVEDVTNVVRRDVEKWGPFDLVYGSTNPLGYSCDRCPGWYMFQFHRILQYARPRQESQKPFFWIFMDNLLLTEDDQVTTVRFLQTEAVTLQDVRGRVLQNAVRVWSNIPGLKSKHAVLTPKEEQSLQAQVRTRSKLAAQKDNPLVKICLLPLREYFKCFSQNSLPL

SEQ ID NO: 81 - protein, 421 aa, synthetic construct
  REGION 1..421: note = Grammomys surdaster DNMT3L
MGSRETPSSCSKALETLNLETSDSSSPDPESPLEEQWLESSPDPEEENSMNVGPEDSKEPLSPSSRPTGREVIRYEVNVNQRNIEDICLCCGTLEVYAQHPLFEGGICVSCKDKFLESLFLYDEDGHQSYCTICCSGGTLFICESPDCTRCYCFECVDILVGPGTSERINAMACWVCFLCLPFSQSGLLQRRKKWRQQLKAFHDREGASPMEIYKTVSAWKRQPVRVLSLFGNIDKELKSLGFLEIGSDSEGGTLKYVEDVTNVVRRDVEKWGPFDLVYGSTNPLGNSCDRCPGWYMFQFHRILQYARPRQDSQKPFFWIFMDNLLLTEDDQVTTVRFLQTEAVTLQDVRGRVLQNAVRVWSNIPGLKSKHSVLTPKEEQSLQAQVRTRSKLPTQVNPLVKTCLLPLREYFKCFSQNSLPL

SEQ ID NO: 82 - protein, 422 aa, synthetic construct
  REGION 1..422: note = Mastomys coucha DNMT3L
MGSRETPSSCSKALETLNLETSDSSSPDPDSPLEEQWLKSSPDLKEEVSIEVILEDSNEPLSPPSPPTGREIIWHEVKVNQRNIEDICLGCGTLQVYAQHPLFEGGICAPCKDKFLETLFLYDEDGHQSYCTICSSGGTLFICESPDCTRCYCFECVDILVGPGTSERISAMTCWVCFLCLPFSRSGLLQRRKRWRHQLKAFHDREGASPIEIYKTVSPWKRQPVRVLSLFGKIDKELKSLGFLESDAGSEGGTLKYVEDVTNVVRRDVEKWGPFDLVYGSTQPFSCSCDHCPGWYMFQFHRILQYARPRQESQRPFFWLFMDHLLLTEDDQVTTTRFLQTEAVTLQDIRGRVLQNAVRVWSNIPGLKSKHTVLTPKEEESLQCQGRTRSKMAAQKLDSLVKHCLIPLREYFKYFSQSSLPL

SEQ ID NO: 83 - DNA, 906 bp, synthetic construct
  source 1..906: mol_type = other DNA
  misc_feature 1..906: note = DNMT3A
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtg

SEQ ID NO: 84 - DNA, 1164 bp, synthetic construct
  source 1..1164: mol_type = other DNA
atggcggccatcccagccctggacccagaggccgagcccagcatggacgtgattttggtgggatccagtgagctctcaagctccgtttcacccgggacaggcagagatcttattgcatatgaagtcaaggctaaccagcgaaatatagaagacatctgcatctgctgcggaagtctccaggttcacacacagcaccctctgtttgagggagggatctgcgccccatgtaaggacaagttcctggatgccctcttcctgtacgacgatgacgggtaccaatcctactgctccatctgctgctccggagagacgctgctcatctgcggaaaccctgattgcacccgatgctactgcttcgagtgtgtggatagcctggtcggccccgggacctcggggaaggtgcacgccatgagcaactgggtgtgctacctgtgcctgccgtcctcccgaagcgggctgctgcagcgtcggaggaagtggcgcagccagctcaaggccttctacgaccgagagtcggagaatccccttgagatgttcgaaaccgtgcctgtgtggaggagacagccagtccgggtgctgtccctttttgaagacatcaagaaagagctgacgagtttgggctttttggaaagtggttctgacccgggacaactgaagcatgtggttgatgtcacagacacagtgaggaaggatgtggaggagtggggacccttcgatcttgtgtacggcgccacacctcccctgggccacacctgtgaccgtcctcccagctggtacctgttccagttccaccggctcctgcagtacgcacggcccaagccaggcagccccaggcccttcttctggatgttcgtggacaatctggtgctgaacaaggaagacctggacgtcgcatctcgcttcctggagatggagccagtcaccatcccagatgtccacggcggatccttgcagaatgctgtccgcgtgtggagcaacatcccagccataaggagcagcaggcactgggctctggtttcggaagaagaattgtccctgctggcccagaacaagcagagctcgaagctcgcggccaagtggcccaccaagctggtgaagaactgctttctccccctaagagaatatttcaagtatttttcaacagaactcacttcctctttataa

SEQ ID NO: 85 - DNA, 645 bp, synthetic construct
  source 1..645: mol_type = other DNA
atgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctctt

SEQ ID NO: 86 - DNA, 1248 bp, synthetic construct
  source 1..1248: mol_type = other DNA
atgggttcccgggagacaccttcttctttctctaagacccttgaaaccttggacctggagacttccgacagctctagccctgacgctgacagtcctctggaagagcaatggctgaaatcctccccagccctgaaggaggacaatgtggatatggtactggaagactgcaaagagcctctgtccccctcctcacccccgacaggcagagagatgatcaggtacgaagtcaaagtgaaccgacggagcattgaagacatctgcctctgctgcggaactctccaggtgtacactcagcaccccttgtttgagggggggatatgtgccccatgtaaggataagttcctggagtccctcttcctgtacgatgatgatggacaccagagttactgcaccatctgctgttccgggggtaccctgttcatctgtgagagccccgactgtaccagatgctactgtttcgagtgtgtggatatcctggtgggcccagggacctcagagcggatcaatgccatggcctgctgggtttgcttcctgtgcctgcccttctcacggagtggactcctgcagaggcgcaagaggtggcggcaccagctgaaggccttccatgatcaagagggagcaggccctatggagatatacaagacagtgtccacatggaagagacagccagtgcgggtactgagcctttttgggaatattgataaagtactaaagagtttgggctttttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcctggctggtacatgttccaattccaccggatcctgcagtatgcactgcctcgccaggagagtcagcggcccttcttctggatatttatggacaatctactgatgactgaggatgaccaagagacaactgcccgcttccttcagacagaggctgtgaccctccaggatgtccgtggtagagactaccagaatgttatgcgggtgtggagcaacattccagggctgaagagcaagcatgtgcccttgaccccaaaggaagaagagtatctgcaagcccaagtcaggaccagaagcaagctggacgcccagaaagttgacctcctggtgaagaactgccttctccccctgagagagtacttcaagtatttttcttaa

SEQ ID NO: 87 - DNA, 1269 bp, synthetic construct
  source 1..1269: mol_type = other DNA
atgggttcccgggagacaccttcttcttgctctaagacctttgaaaccgtgaacctggagacttccgacagctctaatcccgacgctgacagtcctctggaagagcaatggctgaaatcctccccagacctgaaggaggaggacagtgtggatgtggtactggaagactgcgaggagcctctgtccccctcctcaccccccgaaggcagagagatcatcaggtacgaagtcaaagtgaaccaacggaacattgaagacatctgcctcggctgcggaactctccaggtgtacacccagcaccccttgtttgagggggggatatgtgccctgtgtaaggataagttcctggagtccctcttcctgtatgatgatgatggacaccagagttactgcaccatctgctgttccgggggtaccctgttcatctgtgagagccctgactgtaccagatgctactgtttcgagtgtgtggacattctggtggcccccggaacctcagagcggatcaatgccatggcctgctgggtttgcttcctgtgcctgcccttctcacggagcggactgctgcagaggcgcaagaggtggcggcaccagctgaaggccttccatgatcaagaggtagcaggccctatagagatatacaagacagtgtccgcgtggaagagacagccagtgcgggtactgagcctttttgggaatattgataaagtactaaagagtttgggctttttggaaagcggttctggttctgagggaggaatgctgaagtacgtggaagatgtcacaaatgtcgtgaggagagacgtggagaaatggggcccctttgacctggtgtatggctcgacgcagcccctaggcagctcttgtgatcgctgtcctggctggtacatgttccaattccaccggatcctgcagtatgcgctgcctcgccaggagagtcagcggcccttcttctggatatttatggacaatctgttgctgactgaggatgaccaggagacaactgtccgcttccttcagacagagactgtgaccctccagtatgtccgtggcagagtcctccagaatgctgtgcgggtgtggagcaacattcctgggctgaagagcaagcacgcggtcctgaccccacaggaagaagagactctgcaagcccaaatcagaaccagaaacaagccggacacccagaaagttgaccccctggtgaagagctgccttctgcccctgagagagtacttcaagtatttttctcagaactcacttcctctttag

SEQ ID NO: 88 - DNA, 1269 bp, synthetic construct
  source 1..1269: mol_type = other DNA
atgggttcccgggagacaccttcttcctgctctaagacccatgaaaccttgaacctggagactccggagagctctagcactgaccctgacagtcccctggaagagcaatggccgaaatcttccccagatctgaaagaggaagacagcgtggatatggtactggaagactccaaggagcctctaaccccttcctcaccgccgacaggcagagaggtcatcaggtacgaagtcaacgtgaaccagcggaacatcgaagacatctgcctctgttgcggatctctccaggtgtacgctcagcaccccttgtttgaggggggaatttgtgccccgtgtaaggacaagttcctggagaccctcttcctatacgacgaggatggacaccagagctactgtaccatctgctgctccgggcataccctgttcatctgcgagagccccgactgtaccagatgctactgtttcgagtgtgtggatatcctggtgggccccgggacctcggagcgcatcaatgccatggcctgctgggtttgcttcctgtgcctgcctttctcccggagcggactgctgcagaggcgcaagaagtggcgtcaccagctgaaggccttccatgaccgagagggggcaagccctgtggagatatacaagactgtgtctgcatggaaaagacagccagtgagggtgctgagtctttttgggaatattgataaagaactaaagagtttgggctttttggaaagcagttctggttctgagggaggaacgctgaagtacgtggaagatgtcaccaatgtcgtgaggagagaagtggagaaatggggtccctttgacttggtgtatggctcaacccagcccctaggctattcttgtgaccgctgtcctggctggtacatgttccagttccaccggatcctgcagtatgcccggcctcgccaagacagccagcagcccttcttctggatatttgtggacaatctgctgctgactgaagatgaccaagagacaactgtccgcttccttcagacagaggctgtgaccctccaggatgtccgcggcagagtcctccagaatgctatgagggtgtggagcaacattccagggctgaagagcaagcacgcggacctgacccctaaggaagagcagtctctgcaaacccaagtcagaaccagaagcaagctggccgcccagaaagttgactccctagtgaagtactgccttctccccctgagagagtacttcaagtatttttctcagaactcacttcctctttag

SEQ ID NO: 89 - DNA, 1269 bp, synthetic construct
  source 1..1269: mol_type = other DNA
atgggttcccgggagacaccttcttcctgctctaagacccatgaaaccttgaacctggagactccggagagctctagcactgaccctgacagtcccctggaagagcaatggccgaaatcttccccagatctgaaagaggaagacagcgtggatatggtactggaagactccaaggagcctctaaccccttcctcaccgccgacaggcagagagatcatcaggtacgaagtcaacgtgaaccagcggaacatcgaagacatctgcctctgttgcggatctctccaggtgtacgctcagcaccccttgtttgaggggggaatttgtgctccgtgtaaggacaagttcctggagaccctcttcctatacgatgaggatggacaccagagctactgtaccatctgctgctccgggcataccctgttcatctgtgagagccccgactgtaccagatgctactgtttcgagtgtgtggatatcctggtgggccccgggacctcggagcgcatcaatgccatggcctgctgggtttgcttcctgtgcctgcctttctcccggagcggactgctgcagaggcgcaagaagtggcgtcaccagctgaaggccttccatgaccgagagggggcaagccctgtggagatatacaagactgtgtctgcatggaagagacagccagtgagggtgctgagtctttttgggaatattgataaagaactaaagagtttgggctttttggaaagcggttctggttctgagggaggaacgctgaagtacgtggaagatgtcaccaatgtcgtgaggagagaagtggagaaatggggcccctttgacttggtgtatggctcaacccagcccctaggctactcttgtgaccgctgtcctggctggtacatgttccagttccaccggatcctgcagtatgcccggcctcgccaagacagccagcagcccttcttctggatatttgtggacaatctgctgctgactgaagatgaccaagagacaactgtccgcttccttcagacagaggctgtgaccctccaggatgtccgcggcagagtcctccagaatgctgtgagggtgtggagcaacattccagggctgaagagcaagcacgcggacctgacccctaaggaagagcagtctctgcgaacccaagtcagaaccagaagcaagctggccgcccagaaagttgactccctagtgaagtactgccttctccccctgagagagtacttcaagtatttttctcagaactcacttcctctttag

SEQ ID NO: 90 - DNA, 1278 bp, synthetic construct
  source 1..1278: mol_type = other DNA
atgggttccagggagacaccttcttcttgctctaaggtcctggaaaccttgaacctggagacttcggacagctctagccctgaccctgagagtccactggaagagcaatggctggaatcccccccagaccgggaggaggaggacgacgacgacagcgtgattgtggtactggaagactctaaggagcctctgtctccctcctcacgcccgacaggcagagaggtcatcaggtacgaagtaaacgtgaaccaacggaacatcgaagacatttgcctctgctgtgggactctccaggtgtacactaagcaccccttgtttgaggggggactatgtgtctcatgtaaggacaagttcctggagagcctcttcctgtatgacgaggatgggcaccagagctactgcaccatctgctgctcagggggtaccctgttcatctgtgagagccccgactgcaccagatgctactgttttgagtgtgtggacatcctggtgggccccgggacctcagagcgcatcaatgccatggcctgctgggtttgcttcttgtgcctgcctttctctcagagcggactgctgcagaggcgcaagaagtggcgtcaccagctgaaggccttccatgatcgagagggggcaagccctatggagatatacaagacagtgtccgcttggaagagacagccagtgagggtgctgagcctttttgggaatattgagaaagaactaaagagtttgggctttttggaaatcggttctgattctgagggaggaacactgaagtacgtggaagatgtcacgaatgtcgtgaggagagatgtggagaaatggggcccctttgacctggtgtatggctcgacaaatcccctaggctactcttgtgaccgctgtcctggctggtacatgttccagttccaccggatcctgcagtatgcgcggcctcgccaagagagtcagaagcccttcttctggatatttatggacaatctgctgctgactgaggatgaccaagtgacaactgtccgcttccttcagacagaggctgtgaccctccaggatgtccgtggcagagtcctccagaatgctgtgagggtatggagcaacattccagggctgaagagcaagcacgcagtcctcacaccaaaggaagaacagtctctgcaagcccaagtcagaaccagaagcaagctggccgcccagaaagacaaccccctggtgaagatctgccttctccccctgagagagtacttcaagtgtttttctcagaactcacttcctctttag

SEQ ID NO: 91 - DNA, 1266 bp, synthetic construct
  source 1..1266: mol_type = other DNA
atgggctcccgggagacaccttcttcttgctcgaaggccctcgaaaccttgaacctggagacttcggacagctctagccctgaccctgagagtcctctcgaagagcaatggctggaatcctccccagacccggaggaggagaacagcatgaatgtgggaccggaagattccaaggaacctctgtccccttcctcacgtccgacaggcagagaggtcatcaggtacgaagtcaatgtgaaccaacggaacatcgaagatatttgcctctgctgtgggactctcgaggtgtatgctcagcaccccttgtttgagggggggatatgtgtctcatgtaaggacaagttcctggagagcctcttcctgtatgatgaggatggccaccagagctactgcaccatctgctgctcggggggtaccctgttcatctgtgagagccctgactgtaccagatgctactgctttgagtgtgtggacatcctggtgggccccgggacctcagagcggatcaatgccatggcctgctgggtttgcttcctgtgcctgcctttctctcagagcggactgctgcagaggcgcaagaagtggcgtcagcagctgaaggccttccatgatcgagagggggcaagccctatggagatatacaagacagtgtctgcatggaagagacagccagtgagggtgctgagcctttttgggaatattgataaagaactaaagagtttgggctttttggaaatcggttctgattctgagggaggaacactgaagtacgtggaagatgtcacgaatgtcgtgaggagagacgtggagaaatggggcccctttgacctggtgtatggctcgacgaatcccctaggcaactcttgtgaccgctgtcctggctggtacatgttccaattccaccggatcctgcagtatgcgcggcctcgccaagacagtcagaagcccttcttctggatatttatggacaatctgctgctgactgaggatgatcaagtgacaactgtccgcttccttcagacagaggctgtgaccctccaggatgtccgtggcagagtcctccagaatgctgtgagggtatggagcaacattccaggactgaagagcaagcactcagtcctgacgccaaaggaagaacagtctctgcaagcccaagtcagaaccagaagcaagctgcccacccaggttaaccccctggtgaagacctgccttctccccctgagagagtacttcaagtgtttttctcagaattcacttcctctttag

SEQ ID NO: 92 - DNA, 1841 bp, synthetic construct
  source 1..1841: mol_type = other DNA
aacagaaaggcaatcaaaaccacctgcctgcctcaccggccaggaactcagccttcgggacagtaagacgctgagaggctgtgggcctcctccaatgctcagatactgagaaggaagtgttgggtgatgccaaaatgggcttctgctccctgagagacacctcctcttcttcatccccaaaggaggttgctgctcactggcctctcccactgtctccggcagggttctgacgaccctgctgacacacccgccatcccttggacaccgatccttctagccgactacatcaatgggttcccgggagacaccttcttcttgctctaaggcccttgaaaccttgaacctggagacttcggacagctctagccctgaccctgacagtcctctggaagagcaatggctgaaatcctccccagacctgaaggaggaggtcagcatcgaagtgatattggaagactccaatgagcctctgtccccaccctcaccccctacaggcagagagatcatctggcacgaagtcaaagtgaaccaacgcaacatcgaagacatctgcctcggctgtgggactctccaggtgtatgctcagcaccccttgtttgaggggggaatatgtgccccgtgtaaggataagttcctggagaccctcttcctgtatgatgaggatggacaccagagctactgcaccatctgctcttccgggggtaccctgttcatctgtgagagccccgactgtaccagatgctactgttttgagtgtgtggatatcctggtgggccccgggacctcggagcggatcagtgccatgacctgctgggtatgtttcctgtgcctgcctttctcacggagcggactgctgcagaggcgcaagaggtggcgacaccagctgaaggccttccacgatcgagagggggcaagccctatagagatctataagacagtgtccccgtggaagagacagccagtccgggtgctgagcctttttgggaagattgataaagagctaaagagtttgggctttttggaaagcgatgctggttctgagggaggaacactgaagtacgtggaagatgtcaccaatgttgtgaggagagacgtagagaaatggggcccctttgacctggtgtatggctcaacgcagcccttcagctgctcttgtgaccactgtcctggctggtacatgttccagttccaccggatcctgcagtatgcgcggcctcgccaagagagtcagcgacctttcttctggctatttatggaccatctgctgctgactgaggacgaccaagtgacaactacccgtttccttcagacagaggctgtgaccctccaggatatccgtggcagagtcctccagaatgctgtgagggtgtggagcaacattccagggctgaagagcaagcacacggtcctgaccccaaaggaagaagagtctctgcaatgccaaggaagaaccagaagcaagatggctgcccagaaacttgactccctggtgaagcactgccttatccccctgagagagtacttcaagtatttttctcagagctcacttcctctttagaaatgagtcaccccacgatgaaagtctttcctagaaccatggcagtccttcttcctatggcattttccctctgcagagctttctctggttggcttgccagcctttggggttctccctcctgttttgagtccccaggatgcagatgcccctgtgcagctcactgtggggagtgggaggtacccatggctgtggacatcctgatgggataacctgccatgcttctctgaaactgtgtgtacctgttgtgaagtttttcaaatatatcataggattgttactggta

SEQ ID NO: 93 - protein, 27 aa, synthetic construct
  REGION 1..27: note = Linker
SSGNSNANSRGPSFSSGLVPLSLRGSH

SEQ ID NO: 94 - protein, 80 aa, synthetic construct
  REGION 1..80: note = XTEN80 linker
GGPSSGAPPPSGGSPAGSPTSTEEGTSESATPESGPGTSTEPSEGSAPGSPAGSPTSTEEGTSTEPSEGSAPGTSTEPSE

SEQ ID NO: 95 - protein, 16 aa, synthetic construct
  REGION 1..16: note = XTEN16 linker
SGSETPGTSESATPES

SEQ ID NO: 96 - protein, 2111 aa, synthetic construct
  REGION 1..2111: note = DNMT3A-DNMT3L-dCas9-KRAB
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFKDVFVDFTREEWKLLDTAQQIVYRNVMLENYKNLVSLGYQLTKPDVILRLEKGEEP

SEQ ID NO: 97 - protein, 351 aa, synthetic construct
  REGION 1..351: note = GCN4 ATF4 Linker
MTEMSFLSSEVLVGDLMSPFDQSGLGAEESLGLLDDYLEVAKHFKPHGFSSDKAKAGSSEWLAVDGLVSPSNNSKEDAFSGTDWMLEKMDLKEFDLDALLGIDDLETMPDDLLTTLDDTCDLFAPLVQETNKQPPQTVNPIGHLPESLTKPDQVAPFTFLQPLPLSPGVLSSTPDHSFSLELGSEVDITEGDRKPDYTAYVAMIPQCIKEEDTPSDNDSGICMSPESYLGSPQHSPSTRGSPNRSLPSPGVLCGSARPKPYDPPGEKMVAAKVKGEKLDKKLKKMEQNKTAATRYRQKKRAEQEALTGECKELEKKNEALKERADSLAKEIQYLKDLIEEVRKARGKKRVP

SEQ ID NO: 98 - protein, 438 aa, synthetic construct
  REGION 1..438: note = 10X GCN4 Linker
EELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSG

SEQ ID NO: 99 - protein, 18 aa, synthetic construct
  REGION 1..18: note = T2A Linker
EGRGSLLTCGDVEENPGP

SEQ ID NO: 100 - protein, 130 aa, synthetic construct
  REGION 1..130: note = MCP Linker
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 101 - DNA, 1056 bp, synthetic construct
  misc_feature 1..1056: note = GCN4 ATF4 Linker
  source 1..1056: mol_type = other DNA
atgaccgaaatgagcttcctgagcagcgaggtgttggtgggggacttgatgtcccccttcgaccagtcgggtttgggggctgaagaaagcctaggtctcttagatgattacctggaggtggccaagcacttcaaacctcatgggttctccagcgacaaggctaaggcgggctcctccgaatggctggctgtggatgggttggtcagtccctccaacaacagcaaggaggatgccttctccgggacagattggatgttggagaaaatggatttgaaggagttcgacttggatgccctgttgggtatagatgacctggaaaccatgccagatgaccttctgaccacgttggatgacacttgtgatctctttgcccccctagtccaggagactaataagcagcccccccagacggtgaacccaattggccatctcccagaaagtttaacaaaacccgaccaggttgcccccttcaccttcttacaacctcttcccctttccccaggggtcctgtcctccactccagatcattcctttagtttagagctgggcagtgaagtggatatcactgaaggagataggaagccagactacactgcttacgttgccatgatccctcagtgcataaaggaggaagacaccccttcagataatgatagtggcatctgtatgagcccagagtcctatctggggtctcctcagcacagcccctctaccaggggctctccaaataggagcctcccatctccaggtgttctctgtgggtctgcccgtcccaaaccttacgatcctcctggagagaagatggtagcagcaaaagtaaagggtgagaaactggataagaagctgaaaaaaatggagcaaaacaagacagcagccactaggtaccgccagaagaagagggcggagcaggaggctcttactggtgagtgcaaagagctggaaaagaagaacgaggctctaaaagagagggcggattccctggccaaggagatccagtacctgaaagatttgatagaagaggtccgcaaggcaagggggaagaaaagggtcccctag

SEQ ID NO: 102 - DNA, 1314 bp, synthetic construct
  misc_feature 1..1314: note = 10X GCN4 Linker
  source 1..1314: mol_type = other DNA
gaggaactgctgagcaagaactaccacctggaaaacgaggtggccagactgaagaaaggctctggctctggcggaagcggttctggcggatcaggatctggaagtggcggctctggaagcggaggttctggatctggcgaagaactgctgtctaagaattatcacctcgagaacgaagtggctcggctcaagaaaggcagtggcagcggaggaagtggatccggcggtagtggtagtggaagcggcggatccggctcaggcggatctggttcaggggaagaactcctctccaaaaactaccatctcgagaatgaggtcgcccgcctgaaaaaaggatcaggttccggtggttctggcagcggtggttcaggctcaggcagcggcggtagcggtagcggtggaagcggaagcggagaagaacttctcagcaaaaattaccacttggagaatgaagttgcaagactcaaaaaaggttccggcagtggcggcagcggcagcggcggatctggtagtggatctggtggcagtggttcaggcggaagtggtagcggagaggaattgctctcaaagaactatcatttggagaacgaggttgcacgcttgaagaaaggcagcggatcaggcggatctggcagcggtggatctggttctggatccggcggctccggtagtggtggaagtggctctggggaagaattgcttagcaagaattatcatcttgaaaatgaggttgccaggcttaaaaaaggcagtggctccggcggatccggaagcggaggcagcggatctggatctggtggttcaggatctggcggttctggtagcggggaagaactgttgagtaaaaactatcaccttgagaacgaggtcgcaaggttgaaaaaaggatccggctctggcggctccggaagtggcggatctggctccggtagcggaggatcaggatccggcggaagcggatcaggcgaggaactgctttccaaaaattaccaccttgaaaacgaagtcgcccgcctcaagaaaggttctggcagcggaggctctggcagtggtggtagcggaagtggaagtggtggcagtggtagcggtggatctggaagcggcgaggaactcctgtcaaagaattaccatctcgaaaacgaggtcgcaaggctcaagaaaggctcaggatcaggcggctctggatccggcggttctggttccggctcaggtggaagtggatctggcggctcaggttccggcgaagaattgctttccaagaactaccatttggaaaatgaagtcgctcgtttgaagaaaggttcaggctccggc

SEQ ID NO: 103 - DNA, 54 bp, synthetic construct
  misc_feature 1..54: note = T2A Linker
  source 1..54: mol_type = other DNA
gagggcaggggaagtcttctaacatgcggggacgtggaggaaaatcccggcccc

SEQ ID NO: 104 - DNA, 15 bp, synthetic construct
  misc_feature 1..15: note = MS2 Linker
  source 1..15: mol_type = other DNA
ccatgtctgcagggc

SEQ ID NO: 105 - DNA, 390 bp, synthetic construct
  misc_feature 1..390: note = MCP Linker
  source 1..390: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 106 - protein, 1053 aa, synthetic construct
  REGION 1..1053: note = Staphylococcus aureus dCas9
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 107 - protein, 1053 aa, synthetic construct
  REGION 1..1053: note = Staphylococcus aureus dCas9
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVHAIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 108 - protein, 1053 aa, synthetic construct; identical to SEQ ID NO: 107
  REGION 1..1053: note = Streptococcus pyogenes dCas9

SEQ ID NO: 109 - protein, 984 aa, synthetic construct
  REGION 1..984: note = Campylobacter jejuni dCas9
MARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDAIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 110 - protein, 1084 aa, synthetic construct
  REGION 1..1084: note = Corynebacterium diphtheria dCas9
MKYHVGIAVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDAIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 111 - protein, 1107 aa, synthetic construct
  REGION 1..1107: note = Eubacterium ventriosum dCas9
MGYTVGLAIGVASVGVAVLDENDNIVEAVSNIFDEADTSNNKVRRTLREGRRTKRRQKTRIEDFKQLWETSGYIIPHKLHLNIIELRNKGLTELLSLDELYCVLLSMLKHRGISYLEDADDGEKGNAYKKGLAFNEKQLKEKMPCEIQLERMKKYGKYHGEFIIEINDEKEYQSNVFTTKAYKKELEKIFETQRCNGNKINTKFIKKYMEIYERKREYYIGPGNEKSRTDYGIYTTRTDEEGNFIDEKNIFGKLIGKCSVYPEEYRASSASYTAQEFNLLNDLNNLKINNEKLTEFQKKEIVEIIKDASSVNMRKIIKKVIDEDIEQYSGARIDKKGKEIYHTFEIYRKLKKELKTINVDIDSFTREELDKTMDILTLNTERESIVKAFDEQKFVYEENLIKKLIEFRKNNQRLFSGWHSFSYKAMLQLIPVMYKEPKEQMQLLTEMNVFKSKKEKYVNYKYIPENEVVKEIYNPVVVKSIRTTVKILNALIKKYGYPESVVIEMPRDKNSDDEKEKIDMNQKKNQEEYEKILNKIYDEKGIEITNKDYKKQKKLVLKLKLWNEQEGLCLYSGKKIAIEDLLNHPEFFEIDAIIPKSISLDDSRSNKVLVYKTENSIKENDTPYHYLTRINGKWGFDEYKANVLELRRRGKIDDKKVNNLLCMEDITKIDVVKGFINRNLNDTRYASRVVLNEMQSFFESRKYCNTKVKVIRGSLTYQMRQDLHLKKNREESYSHHAVDAMLIAFSQKGYEAYRKIQKDCYDFETGEILDKEKWNKYIDDDEFDDILYKERMNEIRKKIIEAEEKVKYNYKIDKKCNRGLCNQTIYGTREKDGKIHKISSYNIYDDKECNSLKKMINSGKGSDLLMYNNDPKTYRDMLKILETYSSEKNPFVAYNKETGDYFRKYSKNHNGPKVEKVKYYSGQINSCIDISHKYGHAKNSKKVVLVSLNPYRTDVYYDNDTGKYYLVGVKYNHIKCVGNKYVIDSETYNELLRKEGVLNSDENLEDLNSKNITYKFSLYKNDIIQYEKGGEYYTERFLSRIKEQKNLIETKPINKPNFQRKNKKGEWENTRNQIALAKTKYVGKLVTDVLGNCYIVNMEKFSLVVDK

SEQ ID NO: 112 - protein, 1130 aa, synthetic construct
  REGION 1..1130: note = Streptococcus pasteurianus dCas9
MTNGKILGLAIGIASVGVGIIEAKTGKVVHANSRLFSAANAENNAERRGFRGSRRLNRRKKHRVKRVRDLFEKYGIVTDFRNLNLNPYELRVKGLTEQLKNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVSTETGKLSTEQKESLVEFAKNTATLGPAKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGAKQLETKIRLWYQQGERCLYSGKPISIQELVHNSNNFEIDHILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKVSVVRGQFTSQLRRKWKIDKSRETYHHHAVDALIIAASSQLKLWEKQDNPMFVDYGKNQVVDKQTGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDSLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGNYHISQEKYDAIKEKEGIGKKSEFKFTLYRNDLILIKDIASGEQEIYRFLSRTMPNVNHYVELKPYDKEKFDNVQELVEALGEADKVGRCIKGLNKPNISIYKVRTDVLGNKYFVKKKGDKPKLDFKNNKK

SEQ ID NO: 113 - protein, 1125 aa, synthetic construct
  REGION 1..1125: note = Lactobacillus farciminis dCas9
MTDRISLGLAIGVASVGFSVLDIDKGKVIELGARLFNATVAAGNQDRRDMRGSRRLLNRNKQRRKDVSKLFQEYGLLDNFDRDNFHTAFNNNWNPYELRVKGLTKQLNKEELADSLYQIIKRRGISYALKDADANEDGTDYSSSLKINSQELIEKTPAQIQLQRLNDYGKVRGKVIVGDDIDDQKVLLNVFPTNAYEKEARQIIATQQQFYPEILTGEFVKEYCQILTRKRDYFVGPGNEKSRTDYGIYKTDGRTLDNLFEELIGHDKIYPDELRASAASYTAQLFNVLNDLNNLRILSYEDGKLTQIDKEKIIAEIKNNTTVVNMLNVIKKVTGCQKDDIKGFRLNEKDKPEISSMPIYRKAHKDFLKVGIDITDWPIDFIDRLSFILTLNTENGEIRKQINKRLVPDFDFLNDDLVQLIIDNKDSFDIKTNNKWHRFSLKTMNKLIPTMIERPVEQMTLLTEMGLIKKDTKRFEGNKYLPYKEIANDIFNPVASKSVREALKIVNAVLKKYGHIEYIVIEMPRDKNLDEERKQIADFQKKNKKIKDAAFNAFVKAVGSKKNVKIALSKNRKLQMGIRLWHQQQGIDPYNGEEINANDLISNPDKFEIDAIIPQSISFDDGINNKTLCFASMNQVKGQKTPYEFLNEGHGQGYAKFKAIVSKNKNFSKAKRDNYLFEENVSNIETRKRFLSRNLVDTRYSSRVVLNSLQEFFHEKSDDTKVTVIRGKFTSNMRKHWHIDKTRDTFHHHAIDASIIAATPFLRMWKKGSTIFPTKINETSIDIETGEILDDKNFDKVMYEEPYSGFISEIMNADDRIKFSHQVDKKMNRKVSDATIYSTRIGKLSKDKKDAEYVVAKVKDIYSVDGYENFKKVYKKDKTKFLLYKYDPRTFSELEKIVIDYPDKIEKVQTNGKIKAVDISPFELYRRDNGMVRKYSKKTGPAIKQLKYLDKKLGSHIDITPKNANNKHVVLQSLKPWRTDVYLNHETGEYEIMGIKYSDLKFNKNEGYGIKKDKYLKIKNIEGVSENSEFMFSLYRKDRIKVQDMETNESVELLFWSRNFSNKKYAELKPISQIDNDKVLPIYGRGRLIKRLIPKNCKIWKVNTSILGDTYYLEKESNYPQKILD

SEQ ID NO: 114 - protein, 1179 aa, synthetic construct
  REGION 1..1179: note = Sphaerochaeta globus dCas9
MSKKVSRRYEEQAQEICQRLGSRPYSIGLALGVGSIGVAVAAYDPIKKQPSDLVFVSSRIFIPSTGAAERRQKRGQRNSLRHRANRLKFLWKLLAERNLMLSYSEQDVPDPARLRFEDAVVRANPYELRLKGLNEQLTLSELGYALYHIANHRGSSSVRTFLDEEKSSDDKKLEEQQAMTEQLAKEKGISTFIEVLTAFNTNGLIGYRNSESVKSKGVPVPTRDIISNEIDVLLQTQKQFYQEILSDEYCDRIVSAILFENEKIVPEAGCCPYFPDEKKLPRCHFLNEERRLWEAINNARIKMPMQEGAAKRYQSASFSDEQRHILFHIARSGTDITPKLVQKEFPALKTSIIVLQGKEKAIQKIAGFRFRRLEEKSFWKRLSEEQKDDFFSAWTNTPDDKRLSKYLMKHLLLTENEVVDALKTVSLIGDYGPIGKTATQLLMKHLEDGLTYTEALERGMETGEFQELSVWEQQSLLPYYGQILTGSTQALMGKYWHSAFKEKRDSEGFFKPNTNSDEEKYGRIANPVVHQTLNELRKLMNELITILGAKPQEITVELARELKVGAEKREDIIKQQTKQEKEAVLAYSKYCEPNNLDKRYIERFRLLEDQAFVCPYCLEHISVADIAAGRADVDAIFPRDDTADNSYGNKVVAHRQCNDIKGKRTPYAAFSNTSAWGPIMHYLDETPGMWRKRRKFETNEEEYAKYLQSKGFVSRFESDNSYIAKAAKEYLRCLFNPNNVTAVGSLKGMETSILRKAWNLQGIDDLLGSRHWSKDADTSPTMRKNRDDNRHHGLDAIVALYCSRSLVQMINTMSEQGKRAVEIEAMIPIPGYASEPNLSFEAQRELFRKKILEFMDLHAFVSMKTDNDANGALLKDTVYSILGADTQGEDLVFVVKKKIKDIGVKIGDYEEVASAIRGRITDKQPKWYPMEMKDKIEQLQSKNEAALQKYKESLVQAAAVLEESNRKLIESGKKPIQLSEKTISKKALELVGGYYYLISNNKRTKTFVVKEPSNEVKGFAFDTGSNLCLDFYHDAQGKLCGEIIRKIQAMNPSYKPAYMKQGYSLYVRLYQGDVCELRASDLTEAESNLAKTTHVRLPNAKPGRTFVIIITFTEMGSGYQIYFSNLAKSKKGQDTSFTLTTIKNYDVRKVQLSSAGLVRYVSPLLVDKIEKDEVALCGE

SEQ ID NO: 115 - protein, 1168 aa, synthetic construct
  REGION 1..1168: note = Azospirillum (strain B510) dCas9
MARPAFRAPRREHVNGWTPDPHRISKPFFILVSWHLLSRVVIDSSSGCFPGTSRDHTDKFAEWECAVQPYRLSFALGTNSIGWGLLNLDRQGKPREIRALGSRIFSDGRDPQDKASLAVARRLARQMRRRRDRYLTRRTRLMGALVRFGLMPADPAARKRLEVAVDPYLARERATRERLEPFEIGRALFHLNQRRGYKPVRTATKPDEEAGKVKEAVERLEAAIAAAGAPTLGAWFAWRKTRGETLRARLAGKGKEAAYPFYPARRMLEAEFDTLWAEQARHHPDLLTAEAREILRHRIFHQRPLKPPPVGRCTLYPDDGRAPRALPSAQRLRLFQELASLRVIHLDLSERPLTPAERDRIVAFVQGRPPKAGRKPGKVQKSVPFEKLRGLLELPPGTGFSLESDKRPELLGDETGARIAPAFGPGWTALPLEEQDALVELLLTEAEPERAIAALTARWALDEATAAKLAGATLPDFHGRYGRRAVAELLPVLERETRGDPDGRVRPIRLDEAVKLLRGGKDHSDFSREGALLDALPYYGAVLERHVAFGTGNPADPEEKRVGRVANPTVHIALNQLRHLVNAILARHGRPEEIVIELARDLKRSAEDRRREDKRQADNQKRNEERKRLILSLGERPTPRNLLKLRLWEEQGPVENRRCPYSGETISMRMLLSEQVDIDAILPFSVSLDDSAANKVVCLREANRIKRNRSPWEAFGHDSERWAGILARAEALPKNKRWRFAPDALEKLEGEGGLRARHLNDTRHLSRLAVEYLRCVCPKVRVSPGRLTALLRRRWGIDAILAEADGPPPEVPAETLDPSPAEKNRADHRHHALDAVVIGCIDRSMVQRVQLAAASAEREAAAREDNIRRVLEGFKEEPWDGFRAELERRARTIVVSHRPEHGIGGALHKETAYGPVDPPEEGFNLVVRKPIDGLSKDEINSVRDPRLRRALIDRLAIRRRDANDPATALAKAAEDLAAQPASRGIRRVRVLKKESNPIRVEHGGNPSGPRSGGPFHKLLLAGEVHHVDVALRADGRRWVGHWVTLFEAHGGRGADGAAAPPRLGDGERFLMRLHKGDCLKLEHKGRVRVMQVVKLEPSSNSVVVVEPHQVKTDRSKHVKISCDQLRARGARRVTVDPLGRVRVHAPGARVGIGGDAGRTAMEPAEDIS

SEQ ID NO: 116 - protein, 1003 aa, synthetic construct
  REGION 1..1003: note = Gluconacetobacter diazotrophicus dCas9
MIDESLTFGIALGIGSCGWAVLRRPSAFGRKGVIEGMGSWCFDVPETSKERTPTNQIRRSNRLLRRVIRRRRNRMAAIRRLLHAAGLLPSTDSDALKRPGHDPWELRARGLDKPLKPVEFAVVLGHIAKRRGFKSAAKRKATNISSDDKKMLTALEATRERLGRYRTVGEMFARDPDFASRRRNREGKYDRTTARDDLEHEVHALFAAQRRLGQGFASPELEEAFTASAFHQRPMQDSERLVGFCPFERTEKRAAKLTPSFERFRLLARLLNLRITTPDGERPLTVDEIALVTRDLGKTAKLSIKRVRTLIGLEDNQRFTTIRPEDEDRDIVARTGGAMTGTATLRKALGEALWTDMQERPEQLDAIVQVLSFFEANETITEKLREIGLTLAVLDVLLTALDAGVFAKFKGAAHISTKAARNLLPHLEQGRRYDEACTMAGYDHAASRLSHHGQIVAKTQFNALVTEIGESIANPIARKALIEGLKQIWAMRNHWGLPGSIHVELARDVGNSIEKRREIEKHIEKNTALRARERREVHDLLDLEDVNGDTLLRYRLWKEQGGKCLYTGKAIHIRQIAATDNSVQVDAILPWSRFGDDSFNNKTLCLASANQQKKRSTPYEWLSGQTGDAWNAFVQRIETNKELRGFKKRNYLLKNAKEAEEKFRSRNLNDTRYAARLFAEAVKLLYAFGERQEKGGNRRVFTRPGALTAALRQAWGVESLKKQDGKRINDDRHHALDALTVAAVDEAEIQRLTKSFHEWEQQGLGRPLRRVEPPWESFRADVEATYPEVFVARPERRRARGEGHAATIRQVKERECTPIVFERKAVSSLKEADLERIKDGERNEAIVEAIRSWIATGRPADAPPRSPRGDIITKIRLATTIKAAVPVRGGTAGRGEMVRADVFSKPNRRGKDEWYLVPVYPHQIMNRKAWPKPPMRSIVANKDEDEWTEVGPEHQFRFSLYPRSNIEIIRPSGEVIEGYFVGLHRNTGALIPTPVGPDSYVIA

SEQ ID NO: 117 - protein, 1082 aa, synthetic construct
  REGION 1..1082: note = Neisseria cinerea dCas9
MAAFKPNPMNYILGLAIGIASVGWAIVEIDEEENPIRLIDLGVRVFERAEVPKTGDSLAAARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNTHALQTGDFRTPAELALNKFEKESGHIRNQRGDYSHTFNRKDLQAELNLLFEKQKEFGNPHVSDGLKEGIETLLMTQRPALSGDAVQKMLGHCTFEPTEPKAAKNTYTAERFVWLTKLNNLRILEQGSERPLTDTERATLMDEPYRKSKLTYAQARKLLDLDDTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGNRYDEACTEIYGDHYGKKNTEEKIYLPPIPADEIRNPVVLRALSQARKVINGVVRRYGSPARIHIETAREVGKSFKDRKEIEKRQEENRKDREKSAAKFREYFPNFVGEPKSKDILKLRLYEQQHGKCLYSGKEINLGRLNEKGYVEIDAALPFSRTWDDSFNNKVLALGSENQNKGNQTPYEYFNGKDNSREWQEFKARVETSRFPRSKKQRILLQKFDEDGFKERNLNDTRYINRFLCQFVADHMLLTGKGKRRVFASNGQITNLLRGFWGLRKVRAENDRHHALDAVVVACSTIAMQQKITRFVRYKEMNAFDGKTIDKETGEVLHQKAHFPQPWEFFAQEVMIRVFGKPDGKPEFEEADTPEKLRTLLAEKLSSRPEAVHKYVTPLFISRAPNRKMSGQGHMETVKSAKRLDEGISVLRVPLTQLKLKDLEKMVNREREPKLYEALKARLEAHKDDPAKAFAEPFYKYDKAGNRTQQVKAVRVEQVQKTGVWVHNHNGIADNATIVRVDVFEKGGKYYLVPIYSWQVAKGILPDRAVVQGKDEEDWTVMDDSFEFKFVLYANDLIKLTAKKNEFLGYFVSLNRATGAIDIRTHDTDSTKGKNGIFQSVGVKTALSFQKYQIDELGKEIRPCRLKKRPPVR

SEQ ID NO: 118 - protein, 1140 aa, synthetic construct
  REGION 1..1140: note = Roseburia intestinalis dCas9
MRENGSDERRRNMDEKMDYRIGLAIGIASVGWAVLQNNSDDEPVRIVDLGVRIFDTAEIPKTGESLAGPRRAARTTRRRLRRRKHRLDRIKWLFENQGLINIDDFLKRYNMAGLPDVYQLRYEALDRKLTDEELAQVLLHIAKHRGFRSTRKAETAAKENGAVLKATDENQKRMQEKGYRTVGEMIYLDEAFRTGCSWSEKGYILTPRNKAENYQHTMLRAMLVEEVKEIFSSQRRLGNEKATEELEEKYLEIMTSQRSFDLGPGMQPDGKPSPYAMEGFSDRVGKCTFLGDQGELRGAKGTYTAEYFVALQKINHTKLVNQDGETRNFTEEERRALTLLLFTQKEVKYAAVRKKLGLPEDILFYNLNYKKAATKEEQQKENQNTEKAKFIGMPYYHDYKKCLEERVKYLTENEVRDLFDEIGMILTCYKNDDSRTERLAKLGLVPIEMEGLLAYTPTKFQHLSMKAMRNIIPFLEKGMTYDKACEEAGYDFKADSKGTKQKLLTGENVNQTINEITNPVVKRSVSQTVKVINAIIRTYGSPQAINIELAREMSKTFEERRKIKGDMEKRQKNNEDVKKQIQELGKLSPTGQDILKYRLWQEQQGICMYSGKTIPLEELFKPGYDIDAILPYSITFDDSFRNKVLVTSQENRQKGNRTPYEYMGNDEQRWNEFETRVKTTIRDYKKQQKLLKKHFSEEERSEFKERNLTDTKYITTVIYNMIRQNLEMAPLNRPEKKKQVRAVNGAITAYLRKRWGLPQKNRETDTHHAMDAVVIACCTDGMIQKISRYTKVRERCYSKGTEFVDAETGEIFRPEDYSRAEWDEIFGVHIPKPWETFRAELDVRMGDDPKGFLDTHSDVALELDYPEYIYENLRPIFVSRMPNHKVTGAAHADTIRSPRHFKDEGIVLTKTALTDLKLDKDGEIDGYYNPQSDLLLYEALKKQLLLYGNDAKKAFAQDFHKPKADGTEGPVVRKVKIQKKQTMGVFVDSGNGIAENGGMVRIDVFRVNGKYYFVPVYTADVVKKVLPNRASTAHKPYGEWKVMEDKDFLFSLYSRDLIHIKSKKDIPIKMVNGGMEGIKETYAYYIGADISAANIQGIAHDSRYKFRGLGIQSLDVLEKCQIDVLGHVSVVRSEKRMGFS

SEQ ID NO: 119 - protein, 1037 aa, synthetic construct
  REGION 1..1037: note = Parvibaculum lavamentivorans dCas9
MERIFGFAIGTTSIGFSVIDYSSTQSAGNIQRLGVRIFPEARDPDGTPLNQQRRQKRMMRRQLRRRRIRRKALNETLHEAGFLPAYGSADWPVVMADEPYELRRRGLEEGLSAYEFGRAIYHLAQHRHFKGRELEESDTPDPDVDDEKEAANERAATLKALKNEQTTLGAWLARRPPSDRKRGIHAHRNVVAEEFERLWEVQSKFHPALKSEEMRARISDTIFAQRPVFWRKNTLGECRFMPGEPLCPKGSWLSQQRRMLEKLNNLAIAGGNARPLDAEERDAILSKLQQQASMSWPGVRSALKALYKQRGEPGAEKSLKFNLELGGESKLLGNALEAKLADMFGPDWPAHPRKQEIRHAVHERLWAADYGETPDKKRVIILSEKDRKAHREAAANSFVADFGITGEQAAQLQALKLPTGWEPYSIPALNLFLAELEKGERFGALVNGPDWEGWRRTNFPHRNQPTGEILDKLPSPASKEERERISQLRNPTVVRTQNELRKVVNNLIGLYGKPDRIRIEVGRDVGKSKREREEIQSGIRRNEKQRKKATEDLIKNGIANPSRDDVEKWILWKEGQERCPYTGDQIGFNALFREGRYEVEAIWPRSRSFDNSPRNKTLCRKDVNIEKGNRMPFEAFGHDEDRWSAIQIRLQGMVSAKGGTGMSPGKVKRFLAKTMPEDFAARQLNDTRYAAKQILAQLKRLWPDMGPEAPVKVEAVTGQVTAQLRKLWTLNNILADDGEKTRADHRHHAIDALTVACTHPGMTNKLSRYWQLRDDPRAEKPALTPPWDTIRADAEKAVSEIVVSHRVRKKVSGPLHKETTYGDTGTDIKTKSGTYRQFVTRKKIESLSKGELDEIRDPRIKEIVAAHVAGRGGDPKKAFPPYPCVSPGGPEIRKVRLTSKQQLNLMAQTGNGYADLGSNHHIAIYRLPDGKADFEIVSLFDASRRLAQRNPIVQRTRADGASFVMSLAAGEAIMIPEGSKKGIWIVQGVWASGQVVLERDTDADHSTTTRPMPNPILKDDAKKVSIDPIGRVRPSND

SEQ ID NO: 120 - protein, 1132 aa, synthetic construct
  REGION 1..1132: note = Nitratifractor salsuginis (strain DSM 16511) dCas9
MKKILGVALGITSFGYAILQETGKDLYRCLDNSVVMRNNPYDEKSGESSQSIRSTQKSMRRLIEKRKKRIRCVAQTMERYGILDYSETMKINDPKNNPIKNRWQLRAVDAWKRPLSPQELFAIFAHMAKHRGYKSIATEDLIYELELELGLNDPEKESEKKADERRQVYNALRHLEELRKKYGGETIAQTIHRAVEAGDLRSYRNHDDYEKMIRREDIEEEIEKVLLRQAELGALGLPEEQVSELIDELKACITDQEMPTIDESLFGKCTFYKDELAAPAYSYLYDLYRLYKKLADLNIDGYEVTQEDREKVIEWVEKKIAQGKNLKKITHKDLRKILGLAPEQKIFGVEDERIVKGKKEPRTFVPFFFLADIAKFKELFASIQKHPDALQIFRELAEILQRSKTPQEALDRLRALMAGKGIDTDDRELLELFKNKRSGTRELSHRYILEALPLFLEGYDEKEVQRILGFDDREDYSRYPKSLRHLHLREGNLFEKEENPINNHAVKSLASWALGLIADLSWRYGPFDEIILETTRDALPEKIRKEIDKAMREREKALDKIIGKYKKEFPSIDKRLARKIQLWERQKGLDLYSGKVINLSQLLDGSADIEAIVPQSLGGLSTDYNTIVTLKSVNAAKGNRLPGDWLAGNPDYRERIGMLSEKGLIDWKKRKNLLAQSLDEIYTENTHSKGIRATSYLEALVAQVLKRYYPFPDPELRKNGIGVRMIPGKVTSKTRSLLGIKSKSRETNFHHAEDALILSTLTRGWQNRLHRMLRDNYGKSEAELKELWKKYMPHIEGLTLADYIDEAFRRFMSKGEESLFYRDMFDTIRSISYWVDKKPLSASSHKETVYSSRHEVPTLRKNILEAFDSLNVIKDRHKLTTEEFMKRYDKEIRQKLWLHRIGNTNDESYRAVEERATQIAQILTRYQLMDAQNDKEIDEKFQQALKELITSPIEVTGKLLRKMRFVYDKLNAMQIDRGLVETDKNMLGIHISKGPNEKLIFRRMDVNNAHELQKERSGILCYLNEMLFIFNKKGLIHYGCLRSYLEKGQGSKYIALFNPRFPANPKAQPSKFTSDSKIKQVGIGSATGIIKAHLDLDGHVRSYEVFGTLPEGSIEWFKEESGYGRVEDDPHH

SEQ ID NO: 121 - protein, 997 aa, synthetic construct
  REGION 1..997: note = Campylobacter lari (strain CF89-12) dCas9
MKILGFAIGINSIGWAFVENDELKDCGVRIFTEAENPSNKESLALPRRNARSSRRRLKRRKARLVAIKRILAKELKLNFKDYVAADGELPKAYEGKLTSIYELRYKALIQQLETKDLARVILHIAKHRGYMNKNEKKSNDTKKGKILSALKNNALKLENYQSVGEYFYKEFFQKYKENTKNFIKIRNTKDNYNNCVLSSDLEKELKLILEKQKEFGYNYSEDFINEILKVAFFQRPLKDFSHLVGACTFFEEEKRACKNSYSAWEFVALTKIINEIKSLEKISGEIVPTQTINEILNLILDKGSITYKKFRSCINLHESISFKSLKYDKENAENAKLIDFRKLVEFKKALGVHSLSRQELDQISTHITLIKDNVKLKTVLEKYNLSNEQINNLLEIEFNDYINLSFKALGMILPLMREGKRYYEACEITNLKPKTVDEKEDFLPAFCDSIFAHELSNPVVNRAISEYRKVLNALLKKYGKVHKIHLELARDVGLSKKAREKIEDQQKNNKAINDWALKECENIGIKASAKNILKLKLWKEQKEICIYSGNKISIEHLKDEKALEVDAIYPYSRSFDDSFINKVLVFTKENQEKLNKTPFEAFGKNIEKWSKIQTLAQNLPYKKKNKILDENFKDKQQQDFISRNLNDTRYITTLIAKYTKEYLNFLPLSENENTNLKSGEKGSKIHIQTISGMLTSVLRHTWGFDKKDRNNHLHHALDAIIVAYSTSSIIKAFSDFKKNQELLKARFYAKELTSDNYKHQVKFFEPFKGFREKILSKIDEIFVSTPPRKRARGALHEETFYSEDKMIKKYNSKEGLQIALSCGRVRKIGTKYVENDTMVRVDIFKKQNKFYAIPIYVMDFALGILPNKIVITGKDKNNNPKQWQTIDESYEFCFSLYKNDLILLQKKNMQEPEFAYYNSFDIDRSRIKIKKHDNKFENLTSNQKLLFTNKDKGKNRTGIQNLKIFEKYIVTPLGDKIKADFQPRENISLKTSKKHGL

SEQ ID NO: 122 - protein, 1388 aa, synthetic construct
  REGION 1..1388: note = Streptococcus thermophilus (strain LMD-9) dCas9
MTKPYSIGLAIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDAIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 123 - protein, 134 aa, synthetic construct
  REGION 1..134: note = Link-8
RVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKV

SEQ ID NO: 124 - protein, 50 aa, synthetic construct
  REGION 1..50: note = Link-9
AYPYDVPDYASLGSGSPKKKRKVEDPKKKRKVDGSGSETPGTSESATPES

SEQ ID NO: 125 - protein, 18 aa, synthetic construct
  REGION 1..18: note = BPNLS
KRTADGSEFESPKKKRKV

SEQ ID NO: 126 - protein, 7 aa, synthetic construct
  REGION 1..7: note = SV40 NLS
PKKKRKV

SEQ ID NO: 127 - protein, 10 aa, synthetic construct
  REGION 1..10: note = Link-1
VRHKLKRVGS

SEQ ID NO: 128 - protein, 10 aa, synthetic construct
  REGION 1..10: note = Link-2
MKIIEQLPSA

SEQ ID NO: 129 - protein, 15 aa, synthetic construct
  REGION 1..15: note = Link-3
VPFLLEPDNINGKTC

SEQ ID NO: 130 - protein, 12 aa, synthetic construct
  REGION 1..12: note = Link-4
GHGTGSTGSGSS

SEQ ID NO: 131 - protein, 7 aa, synthetic construct
  REGION 1..7: note = Link-5
MSRPDPA

SEQ ID NO: 132 - protein, 16 aa, synthetic construct
  REGION 1..16: note = Link-6
SGSETPGTSESATPES

SEQ ID NO: 133 - protein, 21 aa, synthetic construct
  REGION 1..21: note = Link-7
SGSETPGTSESATPEGGSGGS

SEQ ID NO: 134 - protein, 2111 aa, synthetic construct
  REGION 1..2111: note = DNMT3A-DNMT3L-dCas9-Krab
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFKDVFVDFTREEWKLLDTAQQIVYRNVMLENYKNLVSLGYQLTKPDVILRLEKGEEP

SEQ ID NO: 135 - protein, 2190 aa, synthetic construct
  REGION 1..2190: note = dCas9N-1-DNMT3A-DNMT3L-dCas9C-1-KRAB
PKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTGGPSSGAPPPSGGSPAGSPTSTEEGTSESATPESGPGTSTEPSEGSAPGSPAGSPTSTEEGTSTEPSEGSAPGTSTEPSEMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDYPYDVPDYASLGSGSPKKKRKVEDPKKKRKVDGSGSETPGTSESATPESRTLVTFKDVFVDFTREEWKLLDTAQQIVYRNVMLENYKNLVSLGYQLTKPDVILRLEKGEEP

SEQ ID NO: 136 - protein, 2190 aa, synthetic construct
  REGION 1..2190: note = dCas9N-2-DNMT3A-DNMT3L-dCas9C-2-KRAB
PKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQGGPSSGAPPPSGGSPAGSPTSTEEGTSESATPESGPGTSTEPSEGSAPGSPAGSPTSTEEGTSTEPSEGSAPGTSTEPSEMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSETTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDYPYDVPDYASLGSGSPKKKRKVEDPKKKRKVDGSGSETPGTSESATPESRTLVTFKDVFVDFTREEWKLLDTAQQIVYRNVMLENYKNLVSLGYQLTKPDVILRLEKGEEP

SEQ ID NO: 137 - protein, 2190 aa, synthetic construct
  REGION 1..2190: note = dCas9N-3-DNMT3A-DNMT3L-dCas9C-3-KRAB
PKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNGGPSSGAPPPSGGSPAGSPTSTEEGTSESATPESGPGTSTEPSEGSAPGSPAGSPTSTEEGTSTEPSEGSAPGTSTEPSEMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSESRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDYPYDVPDYASLGSGSPKKKRKVEDPKKKRKVDGSGSETPGTSESATPESRTLVTFKDVFVDFTREEWKLLDTAQQIVYRNVMLENYKNLVSLGYQLTKPDVILRLEKGEEP

SEQ ID NO: 138 - protein, 2111 aa, synthetic construct; identical to SEQ ID NO: 134
  REGION 1..2111: note = DNMT3A-DNMT3L-dCas9-Krab

SEQ ID NO: 139 - protein, 2800 aa, synthetic construct
  REGION 1..2800: note = DNMT3A-DNMT3L-dCas9-EZH2
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESMGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 140 - protein, 3282 aa, synthetic construct
  REGION 1..3282: note = DNMT3A-DNMT3L-dCas9-G9A
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESMRGLPRGRGLMRARGRGRAAPPGSRGRGRGGPHRGRGRPRSLLSLPRAQASWTPQLSTGLTSPPVPCLPSQGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 141 - protein, 2925 aa, synthetic construct
  REGION 1..2925: note = DNMT3A-DNMT3L-dCas9-LSD1
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESMLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGQAGGLQDDSSGGYGDGQASGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQADTVKVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 142 - protein, 2240 aa, synthetic construct
  REGION 1..2240: note = DNMT3A-DNMT3L-dCas9-HP1
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESMGKKTKRTADSSSSEDEEEYVVEKVLDRRVVKGQVEYLLKWKGFSEEHNTWEPEKNLDCPELISEFMKKYKKMKEGENNKPREKSESNKRKSNFSNSADDIKSKKKREQSNDIARGFERGLEPEKIIGATDSCGDLMFLMKWKDTDEADLVLAKEANVKCPQIVIAFYEERLTWHAYPEDAENKEKETAKS

SEQ ID NO: 143 - protein, 3101 aa, synthetic construct
  REGION 1..3101: note = DNMT3A-DNMT3L-dCas9-FOG1
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESMDAGPTVQGGPTRSSRAARAGGGLSPGAGGSRWERDTGSAALLWQEWNPWAWWRPRVTAGSLGDMEAREEVQLVGASHMEQKATAPEAPSPPSADVNSPPPLPPPTSPGGPKELEGQEPEPRPTEEEPGSPWSGPDELEPVVQDGQRRIRARLSLATGLSWGPFHGSVQTRASSPRQAEPSPALTLLLVDEACWLRTLPQALTEAEANTEIHRKDDALWCRVTKPVPAGGLLSVLLTAEPHSTPGHPVKKEPAEPTCPAPAHDLQLLPQQAGMASILATAVINKDVFPCKDCGIWYRSERNLQAHLLYYCASRQGTGSPAAAATDEKPKETYPNERVCPFPQCRKSCPSASSLEIHMRSHSGERPFVCLICLSAFTTKANCERHLKVHTDTLSGVCHSCGFISTTRDILYSHLVTNHMVCQPGSKGEIYSPGAGHPATKLPPDSLGSFQQQHTALQGPLASADLGLAPTPSPGLDRKALAEATNGEARAEPLAQNGGSSEPPAAPRSIKVEAVEEPEAAPILGPGEPGPQAPSRTPSPRSPAPARVKAELSSPTPGSSPVPGELGLAGALFLPQYVFGPDAAPPASEILAKMSELVHSRLQQGAGAGAGGAQTGLFPGAPKGATCFECEITFSNVNNYYVHKRLYCSGRRAPEDAPAARRPKAPPGPARAPPGQPAEPDAPRSSPGPGAREEGAGGAATPEDGAGGRGSEGSQSPGSSVDDAEDDPSRTLCEACNIRFSRHETYTVHKRYYCASRHDPPPRRPAAPPGPPGPAAPPAPSPAAPVRTRRRRKLYELHAAGAPPPPPPGHAPAPESPRPGSGSGSGPGLAPARSPGPAADGPIDLSKKPRRPLPGAPAPALADYHECTACRVSFHSLEAYLAHKKYSCPAAPPPGALGLPAAACPYCPPNGPVRGDLLEHFRLAHGLLLGAPLAGPGVEARTPADRGPSPAPAPAASPQPGSRGPRDGLGPEPQEPPPGPPPSPAAAPEAVPPPPAPPSYSDKGVQTPSKGTPAPLPNGNHRYCRLCNIKFSSLSTFIAHKKYYCSSHAAEHVK

SEQ ID NO: 144 - protein, 2483 aa, synthetic construct
  REGION 1..2483: note = DNMT3A-DNMT3L-dCas9-DHAC3
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESMAKTVAYFYDPDVGNFHYGAGHPMKPHRLALTHSLVLHYGLYKKMIVFKPYQASQHDMCRFHSEDYIDFLQRVSPTNMQGFTKSLNAFNVGDDCPVFPGLFEFCSRYTGASLQGATQLNNKICDIAINWAGGLHHAKKFEASGFCYVNDIVIGILELLKYHPRVLYIDIDIHHGDGVQEAFYLTDRVMTVSFHKYGNYFFPGTGDMYEVGAESGRYYCLNVPLRDGIDDQSYKHLFQPVINQVVDFYQPTCIVLQCGADSLGCDRLGCFNLSIRGHGECVEYVKSFNIPLLVLGGGGYTVRNVARCWTYETSLLVEEAISEELPYSEYFEYFAPDFTLHPDVSTRIENQNSRQYLDQIRQTIFENLKMLNHAPSVQIHDVPADLLTYDRTDEADAEERGPEENYSRSGSRDFSLLSIGLLGRFKREKVNGKVLV

SEQ ID NO: 145 - protein, 3787 aa, synthetic construct
  REGION 1..3787: note = DNMT3A-DNMT3L-dCas9-DOT1L
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESMGEKLELRLKSPVGAEPAVYPWPLPVYDKHHDAAHEIIETIRWVCEEIPDLKLAMENYVLIDYDTKSFESMQRLCDKYNRAIDSIHQLWKGTTQPMKLNTRPSTGLLRHILQQVYNHSVTDPEKLNNYEPFSPEVYGETSFDLVAQMIDEIKMTDDDLFVDLGSGVGQVVLQVAAATNCKHHYGVEKADIPAKYAETMDREFRKWMKWYGKKHAEYTLERGDFLSEEWRERIANTSVIFVNNFAFGPEVDHQLKERFANMKEGGRIVSSKPFAPLNFRINSRNLSDIGTIMRVVELSPLKGSVSWTGKPVSYYLHTIDRTILENYFSSLKNPKLREEQEAARRRQQRESKSNAATPTKGPEGKVAGPADAPMDSGAEEEKAGAATVKKPSPSKARKKKLNKKGRKMAGRKRGRPKKMNTANPERKPKKNQTALDALHAQTVSQTAASSPQDAYRSPHSPFYQLPPSVQRHSPNPLLVAPTPPALQKLLESFKIQYLQFLAYTKTPQYKASLQELLGQEKEKNAQLLGAAQQLLSHCQAQKEEIRRLFQQKLDELGVKALTYNDLIQAQKEISAHNQQLREQSEQLEQDNRALRGQSLQLLKARCEELQLDWATLSLEKLLKEKQALKSQISEKQRHCLELQISIVELEKSQRQQELLQLKSCVPPDDALSLHLRGKGALGRELEPDASRLHLELDCTKFSLPHLSSMSPELSMNGQAAGYELCGVLSRPSSKQNTPQYLASPLDQEVVPCTPSHVGRPRLEKLSGLAAPDYTRLSPAKIVLRRHLSQDHTVPGRPAASELHSRAEHTKENGLPYQSPSVPGSMKLSPQDPRPLSPGALQLAGEKSSEKGLRERAYGSSGELITSLPISIPLSTVQPNKLPVSIPLASVVLPSRAERARSTPSPVLQPRDPSSTLEKQIGANAHGAGSRSLALAPAGFSYAGSVAISGALAGSPASLTPGAEPATLDESSSSGSLFATVGSRSSTPQHPLLLAQPRNSLPASPAHQLSSSPRLGGAAQGPLPEASKGDLPSDSGFSDPESEAKRRIVFTITTGAGSAKQSPSSKHSPLTASARGDCVPSHGQDSRRRGRRKRASAGTPSLSAGVSPKRRALPSVAGLFTQPSGSPLNLNSMVSNINQPLEITAISSPETSLKSSPVPYQDHDQPPVLKKERPLSQTNGAHYSPLTSDEEPGSEDEPSSARIERKIATISLESKSPPKTLENGGGLAGRKPAPAGEPVNSSKWKSTFSPISDIGLAKSADSPLQASSALSQNSLFTFRPALEEPSADAKLAAHPRKGFPGSLSGADGLSPGTNPANGCTFGGGLAADLSLHSFSDGASLPHKGPEAAGLSSPLSFPSQRGKEGSDANPFLSKRQLDGLAGLKGEGSRGKEAGEGGLPLCGPTDKTPLLSGKAAKARDREVDLKNGHNLFISAAAVPPGSLLSGPGLAPAASSAGGAASSAQTHRSFLGPFPPGPQFALGPMSLQANLGSVAGSSVLQSLFSSVPAAAGLVHVSSAATRLTNSHAMGSFSGVAGGTVGGVFNHAVPSASAHPFGARVGRGAACGSATLGPSPLQAAASASASSFQAPASVETRPPPPPPPPPPPLPPPAHLGRSPAGPPVLHAPPPPNAALPPPPTLLASNPEPALLQSLASLPPNQAFLPPTSAASLPPANASLSIKLTSLPHKGARPSFTVHHQPLPRLALAQAAPGIPQASATGPSAVWVSLGMPPPYAAHLSGVKPR

SEQ ID NO: 146 - protein, 2912 aa, synthetic construct
  REGION 1..2912: note = DNMT3A-DNMT3L-dCas9-Krab-EZH2
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFKDVFVDFTREEWKLLDTAQQIVYRNVMLENYKNLVSLGYQLTKPDVILRLEKGEEPAYPYDVPDYASLGSGSPKKKRKVEDPKKKRKVDGSGSETPGTSESATPESMGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 147 - protein, 3394 aa, synthetic construct
  REGION 1..3394: note = DNMT3A-DNMT3L-dCas9-Krab-G9A
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFKDVFVDFTREEWKLLDTAQQIVYRNVMLENYKNLVSLGYQLTKPDVILRLEKGEEPAYPYDVPDYASLGSGSPKKKRKVEDPKKKRKVDGSGSETPGTSESATPESMRGLPRGRGLMRARGRGRAAPPGSRGRGRGGPHRGRGRPRSLLSLPRAQASWTPQLSTGLTSPPVPCLPSQGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 148 - protein, 3037 aa, synthetic construct
  REGION 1..3037: note = DNMT3A-DNMT3L-dCas9-Krab-LSD1
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFKDVFVDFTREEWKLLDTAQQIVYRNVMLENYKNLVSLGYQLTKPDVILRLEKGEEPAYPYDVPDYASLGSGSPKKKRKVEDPKKKRKVDGSGSETPGTSESATPESMLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGQAGGLQDDSSGGYGDGQASGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQADTVKVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 149 - protein, 2595 aa, synthetic construct
  REGION 1..2595: note = DNMT3A-DNMT3L-dCas9-Krab-DOT1L
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFKDVFVDFTREEWKLLDTAQQIVYRNVMLENYKNLVSLGYQLTKPDVILRLEKGEEPAYPYDVPDYASLGSGSPKKKRKVEDPKKKRKVDGSGSETPGTSESATPESMAKTVAYFYDPDVGNFHYGAGHPMKPHRLALTHSLVLHYGLYKKMIVFKPYQASQHDMCRFHSEDYIDFLQRVSPTNMQGFTKSLNAFNVGDDCPVFPGLFEFCSRYTGASLQGATQLNNKICDIAINWAGGLHHAKKFEASGFCYVNDIVIGILELLKYHPRVLYIDIDIHHGDGVQEAFYLTDRVMTVSFHKYGNYFFPGTGDMYEVGAESGRYYCLNVPLRDGIDDQSYKHLFQPVINQVVDFYQPTCIVLQCGADSLGCDRLGCFNLSIRGHGECVEYVKSFNIPLLVLGGGGYTVRNVARCWTYETSLLVEEAISEELPYSEYFEYFAPDFTLHPDVSTRIENQNSRQYLDQIRQTIFENLKMLNHAPSVQIHDVPADLLTYDRTDEADAEERGPEENYSRSGSRDFSLLSIGLLGRFKREKVNGKVLV

SEQ ID NO: 150 - protein, 2595 aa, synthetic construct; identical to SEQ ID NO: 149
  REGION 1..2595: note = DNMT3A-DNMT3L-dCas9-Krab-DHAC3

SEQ ID NO: 151 - protein, 2496 aa, synthetic construct
  REGION 1..2496: note = DNMT3A-DNMT3L-dCas9-10X GCN4
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGQRPQGGGGPKKKRKV

SEQ ID NO: 152 - protein, 368 aa, synthetic construct
  REGION 1..368: note = SCFV-KRAB
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGRTLVTFKDVFVDFTREEWKLLDTAQQIVYRNVMLENYKNLVSLGYQLTKPDVILRLEKGEEPGSGKRTADGSEFESPKKKRK

SEQ ID NO: 153 - protein, 1865 aa, synthetic construct
  REGION 1..1865: note = dCas9-10X GCN4
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGQRPQGGGGPKKKRKV

SEQ ID NO: 154 - protein, 999 aa, synthetic construct
  REGION 1..999: note = SCFV-DNMT3A-DNMT3L-KRAB
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVRTLVTFKDVFVDFTREEWKLLDTAQQIVYRNVMLENYKNLVSLGYQLTKPDVILRLEKGEEPGSGKRTADGSEFESPKKKRK

SEQ ID NO: 155 - protein, 999 aa, synthetic construct
  REGION 1..999: note = SCFV-DNMT3L-DNMT3A-KRAB
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSSGNSNANSRGPSFSSGLVPLSLRGSHMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVRTLVTFKDVFVDFTREEWKLLDTAQQIVYRNVMLENYKNLVSLGYQLTKPDVILRLEKGEEPGSGKRTADGSEFESPKKKRK

SEQ ID NO: 156 - protein, 1999 aa, synthetic construct
  REGION 1..1999: note = DNMT3A-DNMT3L-dCas9
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 157 - protein, 208 aa, synthetic construct
  REGION 1..208: note = MCP-KRAB
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYLDPGGGGSGRTLVTFKDVFVDFTREEWKLLDTAQQIVYRNVMLENYKNLVSLGYQLTKPDVILRLEKGEEPGSGKRTADGSEFESPKKKRK

SEQ ID NO: 158 - protein, 839 aa, synthetic construct
  REGION 1..839: note = MCP-KRAB-DNMT3A-DNMT3L
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVRTLVTFKDVFVDFTREEWKLLDTAQQIVYRNVMLENYKNLVSLGYQLTKPDVILRLEKGEEPLDPGGGGSGMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGSGKRTADGSEFESPKKKRK

SEQ ID NO: 159 - protein, 839 aa, synthetic construct
  REGION 1..839: note = MCP-KRAB-DNMT3L-DNMT3A
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVRTLVTFKDVFVDFTREEWKLLDTAQQIVYRNVMLENYKNLVSLGYQLTKPDVILRLEKGEEPGSGLDPGGGGSGMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSSGNSNANSRGPSFSSGLVPLSLRGSHMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMEKRTADGSEFESPKKKRK

SEQ ID NO: 160 - protein, 2149 aa, synthetic construct
  REGION 1..2149: note = EPICAS-V2 DNMT3A-DNMT3L-dCas9-ZIM3 Krab
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSSGNSNANSRGPSFSSGLVPLSLRGSHMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 161 - protein, 2149 aa, synthetic construct
  REGION 1..2149: note = EPICAS-V3 DNMT3L-DNMT3A-dCas9-ZIM3 Krab
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 162 - DNA, 6333 bp, synthetic construct
  misc_feature 1..6333: note = DNMT3A-DNMT3L-dCas9-Krab
  source 1..6333: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 163 - DNA, 6573 bp, synthetic construct
  misc_feature 1..6573: note = dCas9N-1-DNMT3A-DNMT3L-dCas9C-1-KRAB
  source 1..6573: mol_type = other DNA
ccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagatgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgaggagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctaa

SEQ ID NO: 164 - protein, 6573 aa, synthetic construct
  REGION 1..6573: note = dCas9N-2-DNMT3A-DNMT3L-dCas9C-2-KRAB
CCAAAAAAGAAGAGAAAGGTAATGGACAAGAAGTACAGCATCGGCCTGGCCATCGGCACCAACTCTGTGGGCTGGGCCGTGATCACCGACGAGTACAAGGTGCCCAGCAAGAAATTCAAGGTGCTGGGCAACACCGACCGGCACAGCATCAAGAAGAACCTGATCGGCGCCCTGCTGTTCGACAGCGGAGAAACAGCCGAGGCCACCCGGCTGAAGAGAACCGCCAGAAGAAGATACACCAGACGGAAGAACCGGATCTGCTATCTGCAAGAGATCTTCAGCAACGAGATGGCCAAGGTGGACGACAGCTTCTTCCACAGACTGGAAGAGTCCTTCCTGGTGGAAGAGGATAAGAAGCACGAGCGGCACCCCATCTTCGGCAACATCGTGGACGAGGTGGCCTACCACGAGAAGTACCCCACCATCTACCACCTGAGAAAGAAACTGGTGGACAGCACCGACAAGGCCGACCTGCGGCTGATCTATCTGGCCCTGGCCCACATGATCAAGTTCCGGGGCCACTTCCTGATCGAGGGCGACCTGAACCCCGACAACAGCGACGTGGACAAGCTGTTCATCCAGCTGGTGCAGACCTACAACCAGCTGTTCGAGGAAAACCCCATCAACGCCAGCGGCGTGGACGCCAAGGCCATCCTGTCTGCCAGACTGAGCAAGAGCAGACGGCTGGAAAATCTGATCGCCCAGCTGCCCGGCGAGAAGAAGAATGGCCTGTTCGGCAACCTGATTGCCCTGAGCCTGGGCCTGACCCCCAACTTCAAGAGCAACTTCGACCTGGCCGAGGATGCCAAACTGCAGCTGAGCAAGGACACCTACGACGACGACCTGGACAACCTGCTGGCCCAGATCGGCGACCAGTACGCCGACCTGTTTCTGGCCGCCAAGAACCTGTCCGACGCCATCCTGCTGAGCGACATCCTGAGAGTGAACACCGAGATCACCAAGGCCCCCCTGAGCGCCTCTATGATCAAGAGATACGACGAGCACCACCAGGACCTGACCCTGCTGAAAGCTCTCGTGCGGCAGCAGCTGCCTGAGAAGTACAAAGAGATTTTCTTCGACCAGAGCAAGAACGGCTACGCCGGCTACATCGATGGCGGAGCCAGCCAGGAAGAGTTCTACAAGTTCATCAAGCCCATCCTGGAAAAGATGGACGGCACCGAGGAACTGCTCGTGAAGCTGAACAGAGAGGACCTGCTGCGGAAGCAGCGGACCTTCGACAACGGCAGCATCCCCCACCAGATCCACCTGGGAGAGCTGCACGCCATTCTGCGGCGGCAGGAAGATTTTTACCCATTCCTGAAGGACAACCGGGAAAAGATCGAGAAGATCCTGACCTTCCGCATCCCCTACTACGTGGGCCCTCTGGCCAGGGGAAACAGCAGATTCGCCTGGATGACCAGAAAGAGCGAGGAAACCATCACCCCCTGGAACTTCGAGGAAGTGGTGGACAAGGGCGCCAGCGCCCAGAGCTTCATCGAGCGGATGACCAACTTCGATAAGAACCTGCCCAACGAGAAGGTGCTGCCCAAGCACAGCCTGCTGTACGAGTACTTCACCGTGTACAACGAGCTGACCAAAGTGAAATACGTGACCGAGGGAATGAGAAAGCCCGCCTTCCTGAGCGGCGAGCAGAAAAAAGCCATCGTGGACCTGCTGTTCAAGACCAACCGGAAAGTGACCGTGAAGCAGCTGAAAGAGGACTACTTCAAGAAAATCGAGTGCTTCGACTCCGTGGAAATCTCCGGCGTGGAAGATCGGTTCAACGCCTCCCTGGGCACATACCACGATCTGCTGAAAATTATCAAGGACAAGGACTTCCTGGACAATGAGGAAAACGAGGACATTCTGGAAGATATCGTGCTGACCCTGACACTGTTTGAGGACAGAGAGATGATCGAGGAACGGCTGAAAACCTATGCCCACCTGTTCGACGACAAAGTGATGAAGCAGCTGAAGCGGCGGAGATACACCGGCTGGGGCAGGCTGAGCCGGAAGCTGATCAACGGCATCCGGGACAAGCAGTCCGGCAAGACAATCCTGGATTTCCTGAAGTCCGACGGCTTCGCCAACAGAAACTTCATGCAGCTGATCCACGACGACAGCCTGACCTTTAAAGAGGACATCCAGAAAGCCCAGGTGTCCGGCCAGGGCGATAGCCTGCACGAGCACATTGCCAATCTGGCCGGCAGCCCCGCCATTAAGAAGGGCATCCTGCAGACAGTGAAGGTGGTGGACGAGCTCGTGAAAGTGATGGGCCGGCACAAGCCCGAGAACATCGTGATCGAAATGGCCAGAGAGAACCAGGGAGGGCCGAGCTCTGGCGCACCCCCACCAAGTGGAGGGTCTCCTGCCGGGTCCCCAACATCTACTGAAGAAGGCACCAGCGAATCCGCAACGCCCGAGTCAGGCCCTGGTACCTCCACAGAACCATCTGAAGGTAGTGCGCCTGGTTCCCCAGCTGGAAGCCCTACTTCCACCGAAGAAGGCACGTCAACCGAACCAAGTGAAGGATCTGCCCCTGGGACCAGCACTGAACCATCTGAGATGAACCATGACCAGGAATTTGACCCCCCAAAGGTTTACCCACCTGTGCCAGCTGAGAAGAGGAAGCCCATCCGCGTGCTGTCTCTCTTTGATGGGATTGCTACAGGGCTCCTGGTGCTGAAGGACCTGGGCATCCAAGTGGACCGCTACATTGCCTCCGAGGTGTGTGAGGACTCCATCACGGTGGGCATGGTGCGGCACCAGGGAAAGATCATGTACGTCGGGGACGTCCGCAGCGTCACACAGAAGCATATCCAGGAGTGGGGCCCATTCGACCTGGTGATTGGAGGCAGTCCCTGCAATGACCTCTCCATTGTCAACCCTGCCCGCAAGGGACTTTATGAGGGTACTGGCCGCCTCTTCTTTGAGTTCTACCGCCTCCTGCATGATGCGCGGCCCAAGGAGGGAGATGATCGCCCCTTCTTCTGGCTCTTTGAGAATGTGGTGGCCATGGGCGTTAGTGACAAGAGGGACATCTCGCGATTTCTTGAGTCTAACCCCGTGATGATTGACGCCAAAGAAGTGTCTGCTGCACACAGGGCCCGTTACTTCTGGGGTAACCTTCCTGGCATGAACAGGCCTTTGGCATCCACTGTGAATGATAAGCTGGAGCTGCAAGAGTGTCTGGAGCACGGCAGAATAGCCAAGTTCAGCAAAGTGAGGACCATTACCACCAGGTCAAACTCTATAAAGCAGGGCAAAGACCAGCATTTCCCCGTCTTCATGAACGAGAAGGAGGACATCCTGTGGTGCACTGAAATGGAAAGGGTGTTTGGCTTCCCCGTCCACTACACAGACGTCTCCAACATGAGCCGCTTGGCGAGGCAGAGACTGCTGGGCCGATCGTGGAGCGTGCCGGTCATCCGCCACCTCTTCGCTCCGCTGAAGGAATATTTTGCTTGTGTGTCTAGCGGCAATAGTAACGCTAACAGCCGCGGGCCGAGCTTCAGCAGCGGCCTGGTGCCGTTAAGCTTGCGCGGCAGCCATATGGGCCCTATGGAGATATACAAGACAGTGTCTGCATGGAAGAGACAGCCAGTGCGGGTACTGAGCCTCTTCAGAAACATCGACAAGGTACTAAAGAGTTTGGGCTTCTTGGAAAGCGGTTCTGGTTCTGGGGGAGGAACGCTGAAGTACGTGGAAGATGTCACAAATGTCGTGAGGAGAGACGTGGAGAAATGGGGCCCCTTTGACCTGGTGTACGGCTCGACGCAGCCCCTAGGCAGCTCTTGTGATCGCTGTCCCGGCTGGTACATGTTCCAGTTCCACCGGATCCTGCAGTATGCGCTGCCTCGCCAGGAGAGTCAGCGGCCCTTCTTCTGGATATTCATGGACAATCTGCTGCTGACTGAGGATGACCAAGAGACAACTACCCGCTTCCTTCAGACAGAGGCTGTGACCCTCCAGGATGTCCGTGGCAGAGACTACCAGAATGCTATGCGGGTGTGGAGCAACATTCCAGGGCTGAAGAGCAAGCATGCGCCCCTGACCCCAAAGGAAGAAGAGTATCTGCAAGCCCAAGTCAGAAGCAGGAGCAAGCTGGACGCCCCGAAAGTTGACCTCCTGGTGAAGAACTGCCTTCTCCCGCTGAGAGAGTACTTCAAGTATTTTTCTCAAAACTCACTTCCTCTTGGAGGGCCGAGCTCTGGCGCACCCCCACCAAGTGGAGGGTCTCCTGCCGGGTCCCCAACATCTACTGAAGAAGGCACCAGCGAATCCGCAACGCCCGAGTCAGGCCCTGGTACCTCCACAGAACCATCTGAAGGTAGTGCGCCTGGTTCCCCAGCTGGAAGCCCTACTTCCACCGAAGAAGGCACGTCAACCGAACCAAGTGAAGGATCTGCCCCTGGGACCAGCACTGAACCATCTGAGACCACCCAGAAGGGACAGAAGAACAGCCGCGAGAGAATGAAGCGGATCGAAGAGGGCATCAAAGAGCTGGGCAGCCAGATCCTGAAAGAACACCCCGTGGAAAACACCCAGCTGCAGAACGAGAAGCTGTACCTGTACTACCTGCAGAATGGGCGGGATATGTACGTGGACCAGGAACTGGACATCAACCGGCTGTCCGACTACGATGTGGACGCTATCGTGCCTCAGAGCTTTCTGAAGGACGACTCCATCGATAACAAAGTGCTGACTCGGAGCGACAAGAACCGGGGCAAGAGCGACAACGTGCCCTCCGAAGAGGTCGTGAAGAAGATGAAGAACTACTGGCGCCAGCTGCTGAATGCCAAGCTGATTACCCAGAGGAAGTTCGACAATCTGACCAAGGCCGAGAGAGGCGGCCTGAGCGAACTGGATAAGGCCGGCTTCATCAAGAGACAGCTGGTGGAAACCCGGCAGATCACAAAGCACGTGGCACAGATCCTGGACTCCCGGATGAACACTAAGTACGACGAGAACGACAAACTGATCCGGGAAGTGAAAGTGATCACCCTGAAGTCCAAGCTGGTGTCCGATTTCCGGAAGGATTTCCAGTTTTACAAAGTGCGCGAGATCAACAACTACCACCACGCCCACGACGCCTACCTGAACGCCGTCGTGGGAACCGCCCTGATCAAAAAGTACCCTAAGCTGGAAAGCGAGTTCGTGTACGGCGACTACAAGGTGTACGACGTGCGGAAGATGATCGCCAAGAGCGAGCAGGAAATCGGCAAGGCTACCGCCAAGTACTTCTTCTACAGCAACATCATGAACTTTTTCAAGACCGAGATTACCCTGGCCAACGGCGAGATCCGGAAGCGGCCTCTGATCGAGACAAACGGCGAAACAGGCGAGATCGTGTGGGATAAGGGCCGGGACTTTGCCACCGTGCGGAAAGTGCTGTCTATGCCCCAAGTGAATATCGTGAAAAAGACCGAGGTGCAGACAGGCGGCTTCAGCAAAGAGTCTATCCTGCCCAAGAGGAACAGCGACAAGCTGATCGCCAGAAAGAAGGACTGGGACCCTAAGAAGTACGGCGGCTTCGACAGCCCCACCGTGGCCTATTCTGTGCTGGTGGTGGCCAAAGTGGAAAAGGGCAAGTCCAAGAAACTGAAGAGTGTGAAAGAGCTGCTGGGGATCACCATCATGGAAAGAAGCAGCTTCGAGAAGAATCCCATCGACTTTCTGGAAGCCAAGGGCTACAAAGAAGTGAAAAAGGACCTGATCATCAAGCTGCCTAAGTACTCCCTGTTCGAGCTGGAAAACGGCCGGAAGAGAATGCTGGCCTCTGCCGGCGAACTGCAGAAGGGAAACGAACTGGCCCTGCCCTCCAAATATGTGAACTTCCTGTACCTGGCCAGCCACTATGAGAAGCTGAAGGGCTCCCCCGAGGATAATGAGCAGAAACAGCTGTTTGTGGAACAGCACAAACACTACCTGGACGAGATCATCGAGCAGATCAGCGAGTTCTCCAAGAGAGTGATCCTGGCCGACGCTAATCTGGACAAGGTGCTGAGCGCCTACAACAAGCACAGAGACAAGCCTATCAGAGAGCAGGCCGAGAATATCATCCACCTGTTTACCCTGACCAATCTGGGAGCCCCTGCCGCCTTCAAGTACTTTGACACCACCATCGACCGGAAGAGGTACACCAGCACCAAAGAGGTGCTGGACGCCACCCTGATCCACCAGAGCATCACCGGCCTGTACGAGACACGGATCGACCTGTCTCAGCTGGGAGGCGACTATCCCTATGACGTGCCCGATTATGCCAGCCTGGGCAGCGGCTCCCCCAAGAAAAAACGCAAGGTGGAAGATCCTAAGAAAAAGCGGAAAGTGGACGGCAGCGGCAGCGAGACCCCCGGTACCAGCGAGAGCGCCACCCCCGAGAGCCGGACACTGGTGACCTTCAAGGATGTATTTGTGGACTTCACCAGGGAGGAGTGGAAGCTGCTGGACACTGCTCAGCAGATCGTGTACAGAAATGTGATGCTGGAGAACTATAAGAACCTGGTTTCCTTGGGTTATCAGCTTACTAAGCCAGATGTGATCCTCCGGTTGGAGAAGGGAGAAGAGCCCTAA

SEQ ID NO: 165 - DNA, 6573 bp, synthetic construct
  misc_feature 1..6573: note = dCas9N-3-DNMT3A-DNMT3L-dCas9C-3-KRAB
  source 1..6573: mol_type = other DNA
ccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagatgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctaa

SEQ ID NO: 166 - DNA, 6333 bp, synthetic construct; identical to SEQ ID NO: 162
  misc_feature 1..6333: note = DNMT3A-DNMT3L-dCas9-Krab
  source 1..6333: mol_type = other DNA

SEQ ID NO: 167 - DNA, 8403 bp, synthetic construct
  misc_feature 1..8403: note = DNMT3A-DNMT3L-dCas9-EZH2
  source 1..8403: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttga

SEQ ID NO: 168 - DNA, 9849 bp, synthetic construct
  misc_feature 1..9849: note = DNMT3A-DNMT3L-dCas9-G9A
  source 1..9849: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagcatgcggggtctaccgagagggagggggttgatgcgggcccgggggaggggtcgtgcggcccctccgggcagccgaggccgcggaaggggggggccccacagaggaagaggtaggccccggagcctactctctcttcccagggcccaggcatcctggaccccccaactctctactgggctgaccagccctcctgtcccttgtctcccctcccagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatga

SEQ ID NO: 169 - DNA, 8778 bp, synthetic construct
  misc_feature 1..8778: note = DNMT3A-DNMT3L-dCas9-LSD1
  source 1..8778: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagcatgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcggggcaagcaggaggacttcaagacgacagttctggagggtatggagacggccaagcatcaggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgacactgtcaaggttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtga

SEQ ID NO: 170 - DNA, 6723 bp, synthetic construct
  misc_feature 1..6723: note = DNMT3A-DNMT3L-dCas9-HP1
  source 1..6723: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagcatgggaaagaaaaccaagcggacagctgacagttcttcttcagaggatgaggaggagtatgttgtggagaaggtgctagacaggcgcgtggttaagggacaagtggaatatctactgaagtggaaaggcttttctgaggagcacaatacttgggaacctgagaaaaacttggattgccctgagctaatttctgaatttatgaaaaagtataagaagatgaaggagggtgaaaataataaacccagggagaagtcagaaagtaacaagaggaaatccaatttctcaaacagtgccgatgacatcaaatctaaaaaaaagagagagcagagcaatgatatcgctcggggctttgagagaggactggaaccagaaaagatcattggggcaacagattcctgtggtgatttaatgttcctaatgaaatggaaagacacagatgaagctgacctggttcttgcaaaagaagctaatgtgaaatgtccacaaattgtgatagcattttatgaagagagactgacatggcatgcatatcctgaggatgcggaaaacaaagagaaagaaacagcaaagagctaa

SEQ ID NO: 171 - DNA, 9306 bp, synthetic construct
  misc_feature 1..9306: note = DNMT3A-DNMT3L-dCas9-FOG1
  source 1..9306: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagcatggacgctgggcctacagtacagggagggcccactcgcagctccagggctgccagagcaggtgggggcctgtcccctggtgctggggggagcaggtgggaaagagacacggggtccgcggcccttctctggcaagagtggaatccgtgggcgtggtggaggccaagggtcacggcaggttccctcggagacatggaggccagagaggaggtgcagttggtgggtgccagccacatggagcaaaaggccacggcacctgaagccccgagccctcccagcgcagatgttaactcacccccaccgctgccgccccccacatccccaggaggccccaaggagctggaaggacaggaaccagaacccaggcccacggaggaagagccgggcagtccctggagcgggccagacgagctggagccggtggtgcaggatgggcagaggcgcatacgggcccgactcagcctcgccacgggcctgtcctggggcccgttccatgggagtgtccagaccagagcctcatcccccaggcaggcggagccgagcccagccctgaccctgctgctggtggacgaggcctgctggctgaggacgctgccccaggccctgactgaggccgaggccaacacagagatccacaggaaggatgacgcactctggtgcagggtcaccaagccggtgcctgcggggggactcctgagcgtgctcctcacggccgagccccacagcacccccggccaccctgtgaagaaggagccagcagagcccacgtgcccggcccctgcacacgacctccagctcctgccccagcaggccgggatggcctccatccttgccaccgcagtgatcaacaaagacgtcttcccctgcaaggactgtggcatctggtaccgcagcgagcgcaacctgcaggcgcacctgctctactactgcgccagccgccagggcaccggctccccggccgcagccgccacagacgagaagcccaaagagacctaccccaacgagcgcgtctgccccttcccccagtgccgcaagagctgccccagcgccagctccctggagatccacatgcgcagccacagcggagagcggcccttcgtgtgcctgatctgcctgtcggccttcaccaccaaggccaactgcgagcggcacctcaaggtgcacacggacacgctgagcggtgtctgccacagctgtggcttcatctccaccacaagggacatcctctacagccacttggtcaccaaccacatggtctgccagcctggctccaagggtgagatctactcgccaggggccggacacccagcaaccaagctgcccccagacagtctgggcagcttccagcagcagcacacggccctgcaaggccccctggcctccgcggacctgggcctggcgcccaccccatcgccaggactggacagaaaggccctggccgaggccaccaacggagaggccagagcggagcctctggcccagaatggaggcagcagcgagcccccggcggcccccaggagcatcaaggtggaggcggtggaggagccggaggcggcccccatcctgggccccggagagcctgggccccaggccccgtcgcggacgccgtcgccgcgcagccccgccccggccagggtcaaggccgagctgtccagccccacgccgggctccagcccggtgcccggcgagctgggcctggccggggccctgttccttccgcagtacgtgttcgggcccgacgcggcgccccccgcctcggagatcctggccaagatgtccgagctggtgcacagccggctgcagcagggcgcgggcgcgggcgccggcggcgcgcagaccgggctcttccccggggcccccaagggcgctacgtgcttcgagtgcgagatcaccttcagcaacgtcaacaactactacgtgcacaagcgcctctactgttcaggccgccgtgcgcccgaggacgcgcctgccgcgcgcaggcccaaggcgccccccggcccggcccgcgcgccccccggccagcccgccgaacccgacgcgccgcgctcgtccccgggccccggagcgcgcgaggagggggctgggggcgcggccacgcccgaggacggcgcgggcggccggggcagcgagggcagccagagcccgggtagctccgtggacgacgcggaggacgaccccagccgcacgctgtgcgaggcctgcaacatccgcttcagccgccacgagacctacaccgtgcacaagcggtactactgcgcctcgcgccacgacccgccgccgcgccgaccggccgcgcccccgggaccccctgggccggccgcgcccccggccccctctcccgccgcgcctgtgcgcacgcgcagacgccgcaagctctacgagctgcacgcggccggcgccccgccccccccgccgcccggccacgcccccgcgcccgagtcgccgcggcccggaagcggaagcggaagcggccccggcctcgcccctgcgcgctcgcccggccccgcggccgacggccccatcgacctgagcaagaagccgcggcgcccgctccccggagccccggcaccggcgctggccgactaccacgagtgcacggcctgccgcgtgagcttccacagcctcgaggcctacctggcgcacaagaagtactcgtgccccgctgcgccaccgcccggcgcgctcggcctgcccgccgccgcctgcccctactgccccccgaacggcccggtgcgcggggacctgctggagcatttccgcctggcgcacggcctgctgctcggcgcgcccctggccggcccgggggtcgaggcccggacgccggccgaccgcggcccctcgcccgctcccgcccccgccgcctccccgcagcccgggtcccgtggcccccgggacggcctcggcccggagccccaggagccgccgcccggcccgcccccgtccccggccgccgcgcccgaggccgtgccgcccccgccggcgcccccctcctactcggacaagggcgtccagactcccagcaagggcacgccggcgccgctgcccaacggcaaccaccggtactgccgtctttgcaacatcaagttcagcagcctgtccaccttcatcgcccacaagaagtattactgctcctcgcacgccgccgagcacgtgaagtga

SEQ ID NO: 172 - DNA, 7452 bp, synthetic construct
  misc_feature 1..7452: note = DNMT3A-DNMT3L-dCas9-DHAC3
  source 1..7452: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagcatggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccaacatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggtctgggagcagggacttcagcctactttcaataggcttgctggggaggtttaaaagggaaaaagtaaatgggaaggtattagtttaa

SEQ ID NO: 173 - DNA, 11364 bp, synthetic construct
  misc_feature 1..11364: note = DNMT3A-DNMT3L-dCas9-DOT1L
  source 1..11364: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagcatgggggagaagctggagctgagactgaagtcgcccgtgggggctgagcccgccgtctacccgtggccgctgccggtctacgataaacatcacgatgctgctcatgaaatcatcgagaccatccgatgggtctgtgaagaaatcccggatctcaagctcgctatggagaattacgttttaattgactatgacaccaaaagcttcgagagcatgcagaggctctgcgacaagtacaaccgtgccatcgacagcatccaccagctgtggaagggcaccacgcagcccatgaagctgaacacgcggccgtccactggactcctgcgccatatcctgcagcaggtctacaaccactcggtgaccgaccccgagaagctcaacaactacgagcccttctcccccgaggtgtacggggagacctccttcgacctggtggcccagatgattgatgagatcaagatgaccgacgacgacctgtttgtggacttggggagcggtgtgggccaggtcgtgctccaggttgctgctgccaccaactgcaaacatcactatggcgtcgagaaagcagacatcccggccaagtatgcggagaccatggaccgcgagttcaggaagtggatgaaatggtatggaaaaaagcatgcagaatacacattggagagaggcgatttcctctcagaagagtggagggagcgaatcgccaacacgagtgttatatttgtgaataattttgcctttggtcctgaggtggatcaccagctgaaggagcggtttgcaaacatgaaggaaggtggcagaatcgtgtcctcgaaaccctttgcacctctgaacttcagaataaacagtagaaacttgagtgacatcggcaccatcatgcgcgtggtggagctctcgcccctgaagggctcggtgtcgtggacggggaagccagtctcctactacctgcacactatcgaccgcaccatacttgaaaactatttttctagtctgaaaaacccaaaactcagggaggaacaggaggcagcccggcgccgccagcagcgcgagagcaagagcaacgcggccacgcccactaagggcccagagggcaaggtggccggccccgccgacgcccccatggactctggtgctgaggaagagaaggcgggagcagccaccgtgaagaagccgtctccctccaaagcccgcaagaagaagctaaacaagaaggggaggaagatggctggccgcaagcgcgggcgccccaagaagatgaacactgcgaaccccgagcggaagcccaagaagaaccaaactgcactggatgccctgcacgctcagaccgtgtctcagacggcggcctcctcaccccaggatgcctacagatcccctcacagcccgttctaccagctacctccgagcgtgcagcggcactcccccaacccgctgctggtggcgcccaccccgcccgcgctgcagaagcttctagagtccttcaagatccagtacctgcagttcctggcatacacaaagaccccccagtacaaggccagcctgcaggagctgctgggccaggagaaggagaagaacgcccagctcctgggtgcggctcagcagctcctcagccactgccaggcccagaaggaggagatcaggaggctgtttcagcaaaaattggatgagctgggtgtgaaggcgctgacctacaacgacctgattcaagcgcagaaggagatctccgcccataaccagcagctgcgggagcagtcggagcagctggagcaggacaaccgcgcgctccgcggccagagcttgcagctgctcaaggctcgctgcgaggagctgcagctggactgggccacgctgtcgctggagaagctgttgaaggagaagcaggccctgaagagccagatctcggagaagcagaggcactgcctggagctgcagatcagcattgtggagctagagaagagccagcggcagcaggagctcctgcagctcaagtcctgtgtgccgcctgacgacgccctgtccctgcacctgcgtgggaagggcgccctgggccgcgagctggagcctgacgccagccggctgcacctggagctggactgcaccaagttctcgctgcctcacttgagcagcatgagcccggagctctccatgaacggccaggctgctggctatgagctctgcggtgtgctgagccggccttcgtcgaagcagaacacgccccagtacctggcctcacccctggaccaggaggtggtgccctgtacccctagccacgtcggccggccgcgcctggagaagctgtctggcctagccgcacccgactacactaggctgtccccggccaagattgtgctgaggcggcacctgagccaggaccacacggtgcccggcaggccggctgccagtgagctgcattcgagagctgagcacaccaaggagaacggccttccctaccagagccccagcgtgcctggcagcatgaagctgagccctcaggacccgcggcccctgtcccctggggccttgcagcttgctggagagaagagcagtgagaagggcctgagagagcgcgcctacggcagcagcggggagctcatcaccagcctgcccatcagcatcccgctcagcaccgtgcagcccaacaagctcccggtcagcattcccctggccagcgtggtgctgcccagccgcgccgagagggcgaggagcacccccagtcccgtgctgcagccccgtgacccctcgtccacacttgaaaagcagattggtgctaatgcccacggtgctgggagcagaagccttgccctggcccccgcaggcttctcctacgctggctcggtggccatcagcggggccttggcgggcagcccggcctctctcacacctggagccgagccggccaccttggatgagtcctccagctctgggagcctttttgccaccgtggggtcccgcagctccacgccacagcaccccctgctgctggcacagccccggaactcgcttcctgcctctcccgcccaccagctctcctccagtccccggcttggtggggccgcccagggcccgttgcccgaggccagcaagggagacctgccctccgattccggcttctcagatcctgagagtgaagccaagaggaggattgtgttcaccatcaccactggtgcgggcagtgccaagcagtcgccctccagcaagcacagccccctgaccgccagcgcccgtggggactgtgtgccgagccacgggcaggacagtcgcaggcgcggccggcggaagcgagcatctgcggggacgcccagcttgagcgcaggcgtgtcccccaagcgccgagccctgccgtccgtcgctggccttttcacacagccttcggggtctcccctcaacctcaactccatggtcagtaacatcaaccagcccctggagattacagccatctcgtccccggagacctccctgaagagctcccctgtgccctaccaggaccacgaccagccccccgtgctcaagaaggagcggcctctgagccagaccaatggggcacactactccccactcacctcagacgaggagccaggctctgaggacgagcccagcagtgctcgaattgagagaaaaattgcaacaatctccttagaaagcaaatctcccccgaaaaccttggaaaatggtggtggcttggcgggaaggaagcccgcgcccgccggcgagccagtcaatagcagcaagtggaagtccaccttctcgcccatctccgacatcggcctggccaagtcggcggacagcccgctgcaggccagctccgccctcagccagaactccctgttcacgttccggcccgccctggaggagccctctgccgatgccaagctggccgctcaccccaggaaaggctttcccggctccctgtcgggggctgacggactcagcccgggcaccaaccctgccaacggctgcaccttcggcgggggcctggccgcggacctgagtttacacagcttcagtgatggtgcttctcttccccacaagggccccgaggcggccggcctgagctccccgctgagcttcccctcgcagcgcggcaaggagggctcggacgccaaccctttcctgagcaagaggcagctggacggcctggctgggctgaagggcgagggcagccgcggcaaggaggcaggggagggcggcctaccgctgtgcgggcccacggacaagaccccactgctgagcggcaaggccgccaaggcccgggaccgcgaggtcgacctcaagaatggccacaacctcttcatctctgcggcggccgtgcctcccggaagcctcctcagcggccccggcctggccccggcggcgtcctccgcaggcggcgcggcgtcctccgcccagacgcaccggtccttcctgggccccttcccgccgggaccgcagttcgcgctcggccccatgtccctgcaggccaacctcggctccgtggccggctcctccgtgctgcagtcgctgttcagctctgtgccggccgccgcaggcctggtgcacgtgtcgtccgctgccaccagactgaccaactcgcacgccatgggcagcttttccggggtggcaggcggcacagttggaggtgtctttaaccacgcggtgccctccgcctctgctcatccgtttggagcccgtgtcggccgcggggctgcatgtggcagcgccacactgggcccgagcccgctgcaggcggcggccagcgcctcggcctcttcctttcaggccccggcctcggttgagacccggccgccccctccgcctccgcctccgcctcccccgctgcccccgcctgcgcacctgggccggtcccccgcggggccgcccgtcctccacgccccccctccacctaacgccgccttgcctcctcccccaacgctgctggcctctaaccctgagcccgcgcttctgcagagcctcgcgtccctcccgcctaaccaagctttcttgccccccacctctgctgcctctctgccgcctgctaacgcctctttgtctatcaagctcacctccctcccgcacaagggcgcccgcccctccttcacggtgcaccaccagcccctgccccggctggccctggcccaggccgcgcccgggatcccacaggccagcgccacggggccgtccgcggtgtgggtgtccctcggcatgccgcctccctatgccgcgcacctttcgggggttaagccgcgataa

SEQ ID NO: 174 - DNA, 8637 bp, synthetic construct
  misc_feature 1..8637: note = DNMT3A-DNMT3L-dCas9-Krab-EZH2
  source 1..8637: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccagcggcagcgagacccccggtaccagcgagagcgccacccccgagagcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttga

SEQ ID NO: 175 - DNA, 10083 bp, synthetic construct
  misc_feature 1..10083: note = DNMT3A-DNMT3L-dCas9-Krab-G9A
  source 1..10083: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccagcggcagcgagacccccggtaccagcgagagcgccacccccgagagcatgcggggtctaccgagagggagggggttgatgcgggcccgggggaggggtcgtgcggcccctccgggcagccgaggccgcggaaggggggggccccacagaggaagaggtaggccccggagcctactctctcttcccagggcccaggcatcctggaccccccaactctctactgggctgaccagccctcctgtcccttgtctcccctcccagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatga

SEQ ID NO: 176 - DNA, 9012 bp, synthetic construct
  misc_feature 1..9012: note = DNMT3A-DNMT3L-dCas9-Krab-LSD1
  source 1..9012: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccagcggcagcgagacccccggtaccagcgagagcgccacccccgagagcatgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcggggcaagcaggaggacttcaagacgacagttctggagggtatggagacggccaagcatcaggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgacactgtcaaggttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtga

SEQ ID NO: 177 - DNA, 11598 bp, synthetic construct
  misc_feature 1..11598: note = DNMT3A-DNMT3L-dCas9-Krab-DOT1L
  source 1..11598: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccagcggcagcgagacccccggtaccagcgagagcgccacccccgagagcatgggggagaagctggagctgagactgaagtcgcccgtgggggctgagcccgccgtctacccgtggccgctgccggtctacgataaacatcacgatgctgctcatgaaatcatcgagaccatccgatgggtctgtgaagaaatcccggatctcaagctcgctatggagaattacgttttaattgactatgacaccaaaagcttcgagagcatgcagaggctctgcgacaagtacaaccgtgccatcgacagcatccaccagctgtggaagggcaccacgcagcccatgaagctgaacacgcggccgtccactggactcctgcgccatatcctgcagcaggtctacaaccactcggtgaccgaccccgagaagctcaacaactacgagcccttctcccccgaggtgtacggggagacctccttcgacctggtggcccagatgattgatgagatcaagatgaccgacgacgacctgtttgtggacttggggagcggtgtgggccaggtcgtgctccaggttgctgctgccaccaactgcaaacatcactatggcgtcgagaaagcagacatcccggccaagtatgcggagaccatggaccgcgagttcaggaagtggatgaaatggtatggaaaaaagcatgcagaatacacattggagagaggcgatttcctctcagaagagtggagggagcgaatcgccaacacgagtgttatatttgtgaataattttgcctttggtcctgaggtggatcaccagctgaaggagcggtttgcaaacatgaaggaaggtggcagaatcgtgtcctcgaaaccctttgcacctctgaacttcagaataaacagtagaaacttgagtgacatcggcaccatcatgcgcgtggtggagctctcgcccctgaagggctcggtgtcgtggacggggaagccagtctcctactacctgcacactatcgaccgcaccatacttgaaaactatttttctagtctgaaaaacccaaaactcagggaggaacaggaggcagcccggcgccgccagcagcgcgagagcaagagcaacgcggccacgcccactaagggcccagagggcaaggtggccggccccgccgacgcccccatggactctggtgctgaggaagagaaggcgggagcagccaccgtgaagaagccgtctccctccaaagcccgcaagaagaagctaaacaagaaggggaggaagatggctggccgcaagcgcgggcgccccaagaagatgaacactgcgaaccccgagcggaagcccaagaagaaccaaactgcactggatgccctgcacgctcagaccgtgtctcagacggcggcctcctcaccccaggatgcctacagatcccctcacagcccgttctaccagctacctccgagcgtgcagcggcactcccccaacccgctgctggtggcgcccaccccgcccgcgctgcagaagcttctagagtccttcaagatccagtacctgcagttcctggcatacacaaagaccccccagtacaaggccagcctgcaggagctgctgggccaggagaaggagaagaacgcccagctcctgggtgcggctcagcagctcctcagccactgccaggcccagaaggaggagatcaggaggctgtttcagcaaaaattggatgagctgggtgtgaaggcgctgacctacaacgacctgattcaagcgcagaaggagatctccgcccataaccagcagctgcgggagcagtcggagcagctggagcaggacaaccgcgcgctccgcggccagagcttgcagctgctcaaggctcgctgcgaggagctgcagctggactgggccacgctgtcgctggagaagctgttgaaggagaagcaggccctgaagagccagatctcggagaagcagaggcactgcctggagctgcagatcagcattgtggagctagagaagagccagcggcagcaggagctcctgcagctcaagtcctgtgtgccgcctgacgacgccctgtccctgcacctgcgtgggaagggcgccctgggccgcgagctggagcctgacgccagccggctgcacctggagctggactgcaccaagttctcgctgcctcacttgagcagcatgagcccggagctctccatgaacggccaggctgctggctatgagctctgcggtgtgctgagccggccttcgtcgaagcagaacacgccccagtacctggcctcacccctggaccaggaggtggtgccctgtacccctagccacgtcggccggccgcgcctggagaagctgtctggcctagccgcacccgactacactaggctgtccccggccaagattgtgctgaggcggcacctgagccaggaccacacggtgcccggcaggccggctgccagtgagctgcattcgagagctgagcacaccaaggagaacggccttccctaccagagccccagcgtgcctggcagcatgaagctgagccctcaggacccgcggcccctgtcccctggggccttgcagcttgctggagagaagagcagtgagaagggcctgagagagcgcgcctacggcagcagcggggagctcatcaccagcctgcccatcagcatcccgctcagcaccgtgcagcccaacaagctcccggtcagcattcccctggccagcgtggtgctgcccagccgcgccgagagggcgaggagcacccccagtcccgtgctgcagccccgtgacccctcgtccacacttgaaaagcagattggtgctaatgcccacggtgctgggagcagaagccttgccctggcccccgcaggcttctcctacgctggctcggtggccatcagcggggccttggcgggcagcccggcctctctcacacctggagccgagccggccaccttggatgagtcctccagctctgggagcctttttgccaccgtggggtcccgcagctccacgccacagcaccccctgctgctggcacagccccggaactcgcttcctgcctctcccgcccaccagctctcctccagtccccggcttggtggggccgcccagggcccgttgcccgaggccagcaagggagacctgccctccgattccggcttctcagatcctgagagtgaagccaagaggaggattgtgttcaccatcaccactggtgcgggcagtgccaagcagtcgccctccagcaagcacagccccctgaccgccagcgcccgtggggactgtgtgccgagccacgggcaggacagtcgcaggcgcggccggcggaagcgagcatctgcggggacgcccagcttgagcgcaggcgtgtcccccaagcgccgagccctgccgtccgtcgctggccttttcacacagccttcggggtctcccctcaacctcaactccatggtcagtaacatcaaccagcccctggagattacagccatctcgtccccggagacctccctgaagagctcccctgtgccctaccaggaccacgaccagccccccgtgctcaagaaggagcggcctctgagccagaccaatggggcacactactccccactcacctcagacgaggagccaggctctgaggacgagcccagcagtgctcgaattgagagaaaaattgcaacaatctccttagaaagcaaatctcccccgaaaaccttggaaaatggtggtggcttggcgggaaggaagcccgcgcccgccggcgagccagtcaatagcagcaagtggaagtccaccttctcgcccatctccgacatcggcctggccaagtcggcggacagcccgctgcaggccagctccgccctcagccagaactccctgttcacgttccggcccgccctggaggagccctctgccgatgccaagctggccgctcaccccaggaaaggctttcccggctccctgtcgggggctgacggactcagcccgggcaccaaccctgccaacggctgcaccttcggcgggggcctggccgcggacctgagtttacacagcttcagtgatggtgcttctcttccccacaagggccccgaggcggccggcctgagctccccgctgagcttcccctcgcagcgcggcaaggagggctcggacgccaaccctttcctgagcaagaggcagctggacggcctggctgggctgaagggcgagggcagccgcggcaaggaggcaggggagggcggcctaccgctgtgcgggcccacggacaagaccccactgctgagcggcaaggccgccaaggcccgggaccgcgaggtcgacctcaagaatggccacaacctcttcatctctgcggcggccgtgcctcccggaagcctcctcagcggccccggcctggccccggcggcgtcctccgcaggcggcgcggcgtcctccgcccagacgcaccggtccttcctgggccccttcccgccgggaccgcagttcgcgctcggccccatgtccctgcaggccaacctcggctccgtggccggctcctccgtgctgcagtcgctgttcagctctgtgccggccgccgcaggcctggtgcacgtgtcgtccgctgccaccagactgaccaactcgcacgccatgggcagcttttccggggtggcaggcggcacagttggaggtgtctttaaccacgcggtgccctccgcctctgctcatccgtttggagcccgtgtcggccgcggggctgcatgtggcagcgccacactgggcccgagcccgctgcaggcggcggccagcgcctcggcctcttcctttcaggccccggcctcggttgagacccggccgccccctccgcctccgcctccgcctcccccgctgcccccgcctgcgcacctgggccggtcccccgcggggccgcccgtcctccacgccccccctccacctaacgccgccttgcctcctcccccaacgctgctggcctctaaccctgagcccgcgcttctgcagagcctcgcgtccctcccgcctaaccaagctttcttgccccccacctctgctgcctctctgccgcctgctaacgcctctttgtctatcaagctcacctccctcccgcacaagggcgcccgcccctccttcacggtgcaccaccagcccctgccccggctggccctggcccaggccgcgcccgggatcccacaggccagcgccacggggccgtccgcggtgtgggtgtccctcggcatgccgcctccctatgccgcgcacctttcgggggttaagccgcgataa

SEQ ID NO: 178 - DNA, 7686 bp, synthetic construct
  misc_feature 1..7686: note = DNMT3A-DNMT3L-dCas9-Krab-DHAC3
  source 1..7686: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccagcggcagcgagacccccggtaccagcgagagcgccacccccgagagcatggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccaacatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggtctgggagcagggacttcagcctactttcaataggcttgctggggaggtttaaaagggaaaaagtaaatgggaaggtattagtttaa

SEQ ID NO: 179 - DNA, 7488 bp, synthetic construct
  misc_feature 1..7488: note = DNMT3A-DNMT3L-dCas9-10X GCN4
  source 1..7488: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcattggtagtgggagcaacggcagcagcggagaggaactgctgagcaagaactaccacctggaaaacgaggtggccagactgaagaaaggctctggctctggcggaagcggttctggcggatcaggatctggaagtggcggctctggaagcggaggttctggatctggcgaagaactgctgtctaagaattatcacctcgagaacgaagtggctcggctcaagaaaggcagtggcagcggaggaagtggatccggcggtagtggtagtggaagcggcggatccggctcaggcggatctggttcaggggaagaactcctctccaaaaactaccatctcgagaatgaggtcgcccgcctgaaaaaaggatcaggttccggtggttctggcagcggtggttcaggctcaggcagcggcggtagcggtagcggtggaagcggaagcggagaagaacttctcagcaaaaattaccacttggagaatgaagttgcaagactcaaaaaaggttccggcagtggcggcagcggcagcggcggatctggtagtggatctggtggcagtggttcaggcggaagtggtagcggagaggaattgctctcaaagaactatcatttggagaacgaggttgcacgcttgaagaaaggcagcggatcaggcggatctggcagcggtggatctggttctggatccggcggctccggtagtggtggaagtggctctggggaagaattgcttagcaagaattatcatcttgaaaatgaggttgccaggcttaaaaaaggcagtggctccggcggatccggaagcggaggcagcggatctggatctggtggttcaggatctggcggttctggtagcggggaagaactgttgagtaaaaactatcaccttgagaacgaggtcgcaaggttgaaaaaaggatccggctctggcggctccggaagtggcggatctggctccggtagcggaggatcaggatccggcggaagcggatcaggcgaggaactgctttccaaaaattaccaccttgaaaacgaagtcgcccgcctcaagaaaggttctggcagcggaggctctggcagtggtggtagcggaagtggaagtggtggcagtggtagcggtggatctggaagcggcgaggaactcctgtcaaagaattaccatctcgaaaacgaggtcgcaaggctcaagaaaggctcaggatcaggcggctctggatccggcggttctggttccggctcaggtggaagtggatctggcggctcaggttccggcgaagaattgctttccaagaactaccatttggaaaatgaagtcgctcgtttgaagaaaggttcaggctccggccagcggccgcaaggtggaggtggacccaagaagaagcgcaaggtg

SEQ ID NO: 180 - DNA, 1106 bp, synthetic construct
  misc_feature 1..1106: note = SCFV-KRAB
  source 1..1106: mol_type = other DNA
atgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaagcggtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccggaagcggaaagcgcaccgccgatggttccgagttcgaaagccccaaaaaaaagcgcaaggt

SEQ ID NO: 181 - DNA, 5616 bp, synthetic construct
  misc_feature 1..5616: note = dCas9-10X GCN4
  source 1..5616: mol_type = other DNA
ccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcattggtagtgggagcaacggcagcagcggagaggaactgctgagcaagaactaccacctggaaaacgaggtggccagactgaagaaaggctctggctctggcggaagcggttctggcggatcaggatctggaagtggcggctctggaagcggaggttctggatctggcgaagaactgctgtctaagaattatcacctcgagaacgaagtggctcggctcaagaaaggcagtggcagcggaggaagtggatccggcggtagtggtagtggaagcggcggatccggctcaggcggatctggttcaggggaagaactcctctccaaaaactaccatctcgagaatgaggtcgcccgcctgaaaaaaggatcaggttccggtggttctggcagcggtggttcaggctcaggcagcggcggtagcggtagcggtggaagcggaagcggagaagaacttctcagcaaaaattaccacttggagaatgaagttgcaagactcaaaaaaggttccggcagtggcggcagcggcagcggcggatctggtagtggatctggtggcagtggttcaggcggaagtggtagcggagaggaattgctctcaaagaactatcatttggagaacgaggttgcacgcttgaagaaaggcagcggatcaggcggatctggcagcggtggatctggttctggatccggcggctccggtagtggtggaagtggctctggggaagaattgcttagcaagaattatcatcttgaaaatgaggttgccaggcttaaaaaaggcagtggctccggcggatccggaagcggaggcagcggatctggatctggtggttcaggatctggcggttctggtagcggggaagaactgttgagtaaaaactatcaccttgagaacgaggtcgcaaggttgaaaaaaggatccggctctggcggctccggaagtggcggatctggctccggtagcggaggatcaggatccggcggaagcggatcaggcgaggaactgctttccaaaaattaccaccttgaaaacgaagtcgcccgcctcaagaaaggttctggcagcggaggctctggcagtggtggtagcggaagtggaagtggtggcagtggtagcggtggatctggaagcggcgaggaactcctgtcaaagaattaccatctcgaaaacgaggtcgcaaggctcaagaaaggctcaggatcaggcggctctggatccggcggttctggttccggctcaggtggaagtggatctggcggctcaggttccggcgaagaattgctttccaagaactaccatttggaaaatgaagtcgctcgtttgaagaaaggttcaggctccggccagcggccgcaaggtggaggtggacccaagaagaagcgcaaggtg

SEQ ID NO: 182 - DNA, 3000 bp, synthetic construct
  misc_feature 1..3000: note = SCFV-DNMT3A-DNMT3L-KRAB
  source 1..3000: mol_type = other DNA
atgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaagcggtatgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtacggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccggaagcggaaagcgcaccgccgatggttccgagttcgaaagccccaaaaaaaagcgcaaggtc

SEQ ID NO: 183 - DNA, 3000 bp, synthetic construct
  misc_feature 1..3000: note = SCFV-DNMT3L-DNMT3A-KRAB
  source 1..3000: mol_type = other DNA
atgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaagcggtatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctctttctagcggcaatagtaacgctaacagccgcgggccgagcttcagcagcggcctggtgccgttaagcttgcgcggcagccatatgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtacggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccggaagcggaaagcgcaccgccgatggttccgagttcgaaagccccaaaaaaaagcgcaaggtc

SEQ ID NO: 184 - DNA, 5997 bp, synthetic construct
  misc_feature 1..5997: note = DNMT3A-DNMT3L-dCas9
  source 1..5997: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 185 - DNA, 627 bp, synthetic construct
  misc_feature 1..627: note = MCP-KRAB
  source 1..627: mol_type = other DNA
atggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacttggatccaggtggaggtggaagcggtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccggaagcggaaagcgcaccgccgatggttccgagttcgaaagccccaaaaaaaagcgcaaggtc

SEQ ID NO: 186 - DNA, 2520 bp, synthetic construct
  misc_feature 1..2520: note = MCP-KRAB-DNMT3A-DNMT3L
  source 1..2520: mol_type = other DNA
atggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtacggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccggaagcggattggatccaggtggaggtggaagcggtatgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttaagcgcaccgccgatggttccgagttcgaaagccccaaaaaaaagcgcaaggtc

SEQ ID NO: 187 - DNA, 2520 bp, synthetic construct
  misc_feature 1..2520: note = MCP-KRAB-DNMT3L-DNMT3A
  source 1..2520: mol_type = other DNA
atggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtacggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccggaagcggattggatccaggtggaggtggaagcggtatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctctttctagcggcaatagtaacgctaacagccgcgggccgagcttcagcagcggcctggtgccgttaagcttgcgcggcagccatatgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgaagcgcaccgccgatggttccgagttcgaaagccccaaaaaaaagcgcaaggtc

SEQ ID NO: 188 - DNA, 6447 bp, synthetic construct
  misc_feature 1..6447: note = EPICAS-V2 DNMT3A-DNMT3L-dCas9-ZIM3 Krab
  source 1..6447: mol_type = other DNA
atgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctctttctagcggcaatagtaacgctaacagccgcgggccgagcttcagcagcggcctggtgccgttaagcttgcgcggcagccatatgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagcatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctc

SEQ ID NO: 189 - DNA, 6447 bp, synthetic construct
  misc_feature 1..6447: note = EPICAS-V3 DNMT3L-DNMT3A-dCas9-ZIM3 Krab
  source 1..6447: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcagcggcagcgagacccccggtaccagcgagagcgccacccccgagagcatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctc

SEQ ID NO: 190 - RNA, 20 nt, synthetic construct
  source 1..20: mol_type = other RNA
  misc_feature 1..20: note = sgRNA-1 VEGFA
gctcggaggtcgtggcgctg

SEQ ID NO: 191 - RNA, 20 nt, synthetic construct
  source 1..20: mol_type = other RNA
  misc_feature 1..20: note = sgRNA-2 VEGFA
ggctagcaccagcgctctgt

SEQ ID NO: 192 - RNA, 19 nt, synthetic construct
  source 1..19: mol_type = other RNA
  misc_feature 1..19: note = sgRNA-3 CD151
ccggactcggacgcgtggt

SEQ ID NO: 193 - RNA, 19 nt, synthetic construct
  source 1..19: mol_type = other RNA
  misc_feature 1..19: note = sgRNA-4 CD151
tgtccagggacaatgagca

SEQ ID NO: 194 - RNA, 19 nt, synthetic construct
  source 1..19: mol_type = other RNA
  misc_feature 1..19: note = sgRNA-5 CD151
gacaatgagcagggtgtcc

SEQ ID NO: 195 - RNA, 19 nt, synthetic construct
  source 1..19: mol_type = other RNA
  misc_feature 1..19: note = sgRNA-6 CD81
gagagcgagcgcgcaacgg

SEQ ID NO: 196 - RNA, 19 nt, synthetic construct
  source 1..19: mol_type = other RNA
  misc_feature 1..19: note = sgRNA-7 CD81
ggcctggcaggatgcgcgg

SEQ ID NO: 197 - RNA, 20 nt, synthetic construct
  source 1..20: mol_type = other RNA
  misc_feature 1..20: note = sgRNA-8 CD81
gccgaaacgcgccaagttgg

SEQ ID NO: 198 - RNA, 20 nt, synthetic construct
  source 1..20: mol_type = other RNA
  misc_feature 1..20: note = sgRNA-9 PCSK9
aacctgatcctttagtaccg

SEQ ID NO: 199 - RNA, 20 nt, synthetic construct
  source 1..20: mol_type = other RNA
  misc_feature 1..20: note = sgRNA-10 PCSK9
ggatcttccgatggggctcg

SEQ ID NO: 200 - protein, 833 aa, synthetic construct
  REGION 1..833: note = SCFV -DNMT3A-DNMT3L
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLQGGGGPKKKRKV

SEQ ID NO: 201 - DNA, 2499 bp, synthetic construct
  source 1..2499: mol_type = other DNA
  misc_feature 1..2499: note = SCFV -DNMT3A-DNMT3L
atgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcatgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttcaaggtggaggtggacccaagaagaagcgcaaggtg

SEQ ID NO: 202 - protein, 369 aa, synthetic construct
  REGION 1..369: note = SCFV -KRAB
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGRTLVTFKDVFVDFTREEWKLLDTAQQIVYRNVMLENYKNLVSLGYQLTKPDVILRLEKGEEPGSGKRTADGSEFESPKKKRKV

SEQ ID NO: 203 - DNA, 1107 bp, synthetic construct
  source 1..1107: mol_type = other DNA
  misc_feature 1..1107: note = SCFV -KRAB
atgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaagcggtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccggaagcggaaagcgcaccgccgatggttccgagttcgaaagccccaaaaaaaagcgcaaggtc

SEQ ID NO: 204 - protein, 2114 aa, synthetic construct
  REGION 1..2114: note = DNMT3A-DNMT3L-dCas9-KRAB(ZIM3)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFEDVTVNFTQGEWQRLNPEQRNLYRDVMLENYSNLVSVGQGETTKPDVILRLEQGKEPWL

SEQ ID NO: 205 - protein, 2113 aa, synthetic construct
  REGION 1..2113: note = DNMT3A-DNMT3L-dCas9-KRAB(ZNF680)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLLTFRDVAIEFSLEEWQCLDTAQRNLYRKVMFENYRNLVFLGIAVSKPHLITCLEQGKEPWN

SEQ ID NO: 206 - DNA, 192 bp, synthetic construct
  source 1..192: mol_type = other DNA
  misc_feature 1..192: note = KRAB(ZNF680)
cggacactgctgaccttcagagatgtggccatcgagttcagcctggaagagtggcagtgcctggacacagcccagcggaacctgtacagaaaagtgatgttcgagaactaccggaacctcgtgttcctgggaatcgccgtgtctaagccccacctgatcacctgtctcgagcaagggaaagaaccctggaac

SEQ ID NO: 207 - protein, 2114 aa, synthetic construct
  REGION 1..2114: note = DNMT3A-DNMT3L-dCas9-KRAB(ZNF554)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFEDVSMDFSQEEWELLEPAQKNLYREVMLENYRNVVSLEALKNQCTDVGIKEGPLSPAQT

SEQ ID NO: 208 - DNA, 195 bp, synthetic construct
  source 1..195: mol_type = other DNA
  misc_feature 1..195: note = KRAB(ZNF554)
cggacactggtgacatttgaggatgtctccatggacttcagccaagaggaatgggagctgctggaacccgctcagaagaacctgtatagggaagtcatgctcgaaaattaccggaatgtggtgtccctggaagccctgaagaaccagtgtaccgacgtgggcatcaaagagggcccactgtctccagctcagacc

SEQ ID NO: 209 - protein, 2113 aa, synthetic construct
  REGION 1..2113: note = DNMT3A-DNMT3L-dCas9-KRAB(ZNF264)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFDDVAVTFTKEEWGQLDLAQRTLYQEVMLENCGLLVSLGCPVPKAELICHLEHGQEPWT

SEQ ID NO: 210 - DNA, 192 bp, synthetic construct
  source 1..192: mol_type = other DNA
  misc_feature 1..192: note = KRAB(ZNF264)
cggacactggtgactttcgacgatgtggccgtgacctttaccaaagaagagtggggccagctggatctggcccagagaacactgtaccaagaagttatgctggagaactgcggcctgctggtgtctctgggatgtcctgtgcctaaggccgagctgatctgccacctggaacacggacaagagccttggacc

SEQ ID NO: 211 - protein, 2113 aa, synthetic construct
  REGION 1..2113: note = DNMT3A-DNMT3L-dCas9-KRAB(ZNF582)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLELFRDVAIVFSQEEWQWLAPAQRDLYRDVMLETYSNLVSLGLAVSKPDVISFLEQGKEPWM

SEQ ID NO: 212 - DNA, 192 bp, synthetic construct
  source 1..192: mol_type = other DNA
  misc_feature 1..192: note = KRAB(ZNF582)
cggacactggagctgttccgcgacgtggccattgtgttctctcaagaggaatggcagtggctggcccctgctcagagagatctctaccgcgacgttatgctggagacatactccaatctcgtgtccctcggcctggccgtgtccaaacctgatgtgatcagcttcctcgagcagggaaaagaaccttggatg

SEQ ID NO: 213 - protein, 2113 aa, synthetic construct
  REGION 1..2113: note = DNMT3A-DNMT3L-dCas9-KRAB(ZNF324)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLMAFEDVAVYFSQEEWGLLDTAQRALYRRVMLDNFALVASLGLSTSRPRVVIQLERGEEPWV

SEQ ID NO: 214 - DNA, 192 bp, synthetic construct
  source 1..192: mol_type = other DNA
  misc_feature 1..192: note = KRAB(ZNF324)
cggacactgatggcctttgaggacgtcgccgtgtatttttcccaagaagaatggggactgctcgacactgcccagagagccctgtatcgcagagtcatgctggacaacttcgccctggtggcctccctgggcctgtctacaagcagacccagagtggtcattcagctggaaagaggcgaggaaccctgggtc

SEQ ID NO: 215 - protein, 2114 aa, synthetic construct
  REGION 1..2114: note = DNMT3A-DNMT3L-dCas9-KRAB(ZNF669)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVAFEDVAVNFTQEEWALLDSSQKNLYREVMQETCRNLASVGSQWKDQNIEDHFEKPGKDIRN

SEQ ID NO: 216 - DNA, 195 bp, synthetic construct
  source 1..195: mol_type = other DNA
  misc_feature 1..195: note = KRAB(ZNF669)
cggacactggtcgcctttgaagatgtggctgtgaatttcacccaagaagagtgggctctgctggacagcagccagaagaatctctaccgcgaagtgatgcaagagacatgccggaacctggcctctgtgggctctcagtggaaggaccagaacatcgaggaccacttcgagaagcccggcaaggacatcagaaac

SEQ ID NO: 217 - protein, 2113 aa, synthetic construct
  REGION 1..2113: note = DNMT3A-DNMT3L-dCas9-KRAB(ZNF354A)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLLTFEDVAVLFTRDEWRKLAPSQRNLYRDVMLENYRNLVSLGLPFTKPKVISLLQQGEDPWE

SEQ ID NO: 218 - DNA, 192 bp, synthetic construct
  source 1..192: mol_type = other DNA
  misc_feature 1..192: note = KRAB(ZNF354A)
cggacactgctgacattcgaagatgtcgcagtgctgttcacccgggacgagtggaggaaactggctcccagccagcgcaatctgtatagagatgtgatgctggagaattacagaaatctcgtcagcctggggctgcccttcaccaagcctaaagtgatcagcctgctgcaacaaggcgaggatccttgggaa

SEQ ID NO: 219 - protein, 2113 aa, synthetic construct
  REGION 1..2113: note = DNMT3A-DNMT3L-dCas9-KRAB(ZNF82)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVMFSDVSIDFSPEEWEYLDLEQKDLYRDVMLENYSNLVSLGCFISKPDVISSLEQGKEPWK

SEQ ID NO: 220 - DNA, 192 bp, synthetic construct
  source 1..192: mol_type = other DNA
  misc_feature 1..192: note = KRAB(ZNF82)
cggacactggttatgttctccgacgtgtccatcgactttagccccgaagagtgggagtatctggacctggaacagaaggatctgtatcgcgacgtgatgctcgagaattactctaacctggtgagcctgggctgcttcatcagcaagcccgatgtgatctctagcctggagcaaggcaaagagccatggaaa

SEQ ID NO: 221 - protein, 2113 aa, synthetic construct
  REGION 1..2113: note = DNMT3A-DNMT3L-dCas9-KRAB(ZNF595)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFRDVAIEFSPEEWKCLDPAQQNLYRDVMLENYRNLVSLGFVISNPDLVTCLEQIKEPCN

SEQ ID NO: 222 - DNA, 192 bp, synthetic construct
  source 1..192: mol_type = other DNA
  misc_feature 1..192: note = KRAB(ZNF595)
cggacactggtcaccttccgggacgtcgcaatcgaattcagccccgaggaatggaaatgtctggaccccgcacagcaaaacctctacagagatgtcatgctcgaaaactataggaacctggtctccctgggcttcgtgatcagcaaccctgatctcgtgacctgcctcgaacagatcaaagagccctgcaac

SEQ ID NO: 223 - protein, 2113 aa, synthetic construct
  REGION 1..2113: note = DNMT3A-DNMT3L-dCas9-KRAB(ZNF419)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFEDVAVYFSQEEWRLLDDAQRLLYRNVMLENFTLLASLGLASSKTHEITQLESWEEPFM

SEQ ID NO: 224 - DNA, 192 bp, synthetic construct
  source 1..192: mol_type = other DNA
  misc_feature 1..192: note = KRAB(ZNF419)
cggacactggttaccttcgaagatgttgccgtgtacttcagccaagaagagtggcggctgctggatgacgcccagagactgctgtatcggaatgttatgctcgaaaacttcaccctgctggcttccctgggactcgccagctctaagacccacgagattacccagctggaatcctgggaagaacccttcatg

SEQ ID NO: 225 - protein, 2113 aa, synthetic construct
  REGION 1..2113: note = DNMT3A-DNMT3L-dCas9-KRAB(ZNF566)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVMFSDVSVDFSQEEWECLNDDQRDLYRDVMLENYSNLVSMGHSISKPNVISYLEQGKEPWL

SEQ ID NO: 226 - DNA, 192 bp, synthetic construct
  source 1..192: mol_type = other DNA
  misc_feature 1..192: note = KRAB(ZNF566)
cggacactggtcatgttcagcgacgtgtccgtggactttagccaagaggaatgggaatgcctgaacgacgaccagcgggacctctatagggatgtcatgctcgagaactacagcaatctcgtttccatgggccacagcatctccaagccaaacgtcatcagctatctcgaacaaggcaaagagccctggctg

SEQ ID NO: 227 - protein, 2113 aa, synthetic construct
  REGION 1..2113: note = DNMT3A-DNMT3L-dCas9-KRAB(ZIM2)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLRVFGFPVHYTDVSNMSRLARQRLLGRSWSVPVIRHLFAPLKEYFACVGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFEDVLVDFSPEELSSLSAAQRNLYREVMLENYRNLVSLGHQFSKPDIISRLEEEESYAM

SEQ ID NO: 228 - DNA, 192 bp, synthetic construct
  source 1..192: mol_type = other DNA
  misc_feature 1..192: note = KRAB(ZIM2)
cggacactggtcaccttcgaggatgtgctggtggatttcagccctgaggaactgagcagcctgtccgccgcacagagaaatctctatcgggaagtgatgctggagaactatcggaatctggtgtctctgggccaccagttcagcaagcctgacatcatcagcagactggaagaagaggaatcctacgccatg

SEQ ID NO: 229 - RNA, 20 nt, synthetic construct
  source 1..20: mol_type = other RNA
  misc_feature 1..20: note = Nt sgRNA
ctgaaggtgtctggcagagc

SEQ ID NO: 230 - protein, 64 aa, synthetic construct
  REGION 1..64: note = KRAB(ZNF680)
RTLLTFRDVAIEFSLEEWQCLDTAQRNLYRKVMFENYRNLVFLGIAVSKPHLITCLEQGKEPWN

SEQ ID NO: 231 - protein, 65 aa, synthetic construct
  REGION 1..65: note = KRAB(ZNF554)
RTLVTFEDVSMDFSQEEWELLEPAQKNLYREVMLENYRNVVSLEALKNQCTDVGIKEGPLSPAQT

SEQ ID NO: 232 - protein, 64 aa, synthetic construct
  REGION 1..64: note = KRAB(ZNF264)
RTLVTFDDVAVTFTKEEWGQLDLAQRTLYQEVMLENCGLLVSLGCPVPKAELICHLEHGQEPWT

SEQ ID NO: 233 - protein, 64 aa, synthetic construct
  REGION 1..64: note = KRAB(ZNF582)
RTLELFRDVAIVFSQEEWQWLAPAQRDLYRDVMLETYSNLVSLGLAVSKPDVISFLEQGKEPWM

SEQ ID NO: 234 - protein, 64 aa, synthetic construct
  REGION 1..64: note = KRAB(ZNF324)
RTLMAFEDVAVYFSQEEWGLLDTAQRALYRRVMLDNFALVASLGLSTSRPRVVIQLERGEEPWV

SEQ ID NO: 235 - protein, 65 aa, synthetic construct
  REGION 1..65: note = KRAB(ZNF669)
RTLVAFEDVAVNFTQEEWALLDSSQKNLYREVMQETCRNLASVGSQWKDQNIEDHFEKPGKDIRN

SEQ ID NO: 236 - protein, 64 aa, synthetic construct
  REGION 1..64: note = KRAB(ZNF354A)
RTLLTFEDVAVLFTRDEWRKLAPSQRNLYRDVMLENYRNLVSLGLPFTKPKVISLLQQGEDPWE

SEQ ID NO: 237 - protein, 64 aa, synthetic construct
  REGION 1..64: note = KRAB(ZNF82)
RTLVMFSDVSIDFSPEEWEYLDLEQKDLYRDVMLENYSNLVSLGCFISKPDVISSLEQGKEPWK

SEQ ID NO: 238 - protein, 64 aa, synthetic construct
  REGION 1..64: note = KRAB(ZNF595)
RTLVTFRDVAIEFSPEEWKCLDPAQQNLYRDVMLENYRNLVSLGFVISNPDLVTCLEQIKEPCN

SEQ ID NO: 239 - protein, 64 aa, synthetic construct
  REGION 1..64: note = KRAB(ZNF419)
RTLVTFEDVAVYFSQEEWRLLDDAQRLLYRNVMLENFTLLASLGLASSKTHEITQLESWEEPFM

SEQ ID NO: 240 - protein, 64 aa, synthetic construct
  REGION 1..64: note = KRAB(ZNF566)
RTLVMFSDVSVDFSQEEWECLNDDQRDLYRDVMLENYSNLVSMGHSISKPNVISYLEQGKEPWL

SEQ ID NO: 241 - protein, 64 aa, synthetic construct
  REGION 1..64: note = KRAB(ZIM2)
RTLVTFEDVLVDFSPEELSSLSAAQRNLYREVMLENYRNLVSLGHQFSKPDIISRLEEEESYAM